(19) United States
(12) Patent Application Publication
(10) Pub. No.: US 2026/0102487 A1
(43) Pub. Date: Apr. 16, 2026

(54) COMBINATION THERAPIES COMPRISING T-CELL REDIRECTING THERAPIES AND AGONISTIC ANTI-IL-2R ANTIBODIES OR FRAGMENTS THEREOF

(71) Applicant: AMGEN INC., Thousand Oaks, CA (US)

(72) Inventors: Katherine HARRIS, Fremont, CA (US); Kyle LORENTSEN, Dublin, CA (US); Harbani Kaur MALIK CHAUDHRY, Oakland, CA (US); Kaitlyn LOUGHLIN, San Mateo, CA (US); Deepali V. SAWANT, Newark, CA (US); Hayley MA, Newcastle, WA (US); Volha PRYSHCHEP, San Carlos, CA (US); Raul Gabriel LAZARO, Oxnard, CA (US)

(73) Assignee: AMGEN INC., Thousand Oaks, CA (US)

(21) Appl. No.: 19/115,975

(22) PCT Filed: Oct. 4, 2023
(86) PCT No.: PCT/US2023/075915

§ 371 Date: Mar. 27, 2025

Related U.S. Application Data
(60) Provisional application No. 63/413,339, filed on Oct. 5, 2022.

(51) Int. Cl. A61K 39/395; A61K 39/00; A61P 35/00; C07K 16/28; C07K 16/30

(52) U.S. Cl. CPC A61K 39/39541; A61K 39/39558; A61P 35/00; C07K 16/2809; C07K 16/2866; C07K 16/30; A61K 2039/507; C07K 2317/31; C07K 2317/51; C07K 2317/92; C07K 2317/94

(57) ABSTRACT

Methods of treating cancer or enhancing an anti-cancer effect associated with administration of a T-cell redirecting therapy in a subject in need thereof, comprising administering to the subject an anti-IL-2Rβγ heavy chain-only antibody or antigen binding fragment thereof in combination with a T-cell redirecting therapy are disclosed, along with uses of anti-IL-2Rβγ heavy chain-only antibodies and antigen binding fragments thereof and/or T-cell redirecting therapies in the manufacture of a medicament adapted for use in a method described herein; pharmaceutical compositions comprising anti-IL-2Rβγ heavy chain-only antibodies and antigen binding fragments thereof and/or T-cell redirecting therapies for use in a method described herein; and combination products.

### CROSS-REFERENCE TO RELATED APPLICATIONS

This application claims the benefit of priority to U.S. Provisional Patent Application No. 63/413,339, filed Oct. 5, 2022, which is hereby incorporated by reference in its entirety for all purposes.

### SEQUENCE LISTING

Incorporated by reference in its entirety is a computer-readable amino acid sequence listing submitted concurrently herewith and identified as follows: 100 kilobyte XML file named 10235-WO01-SEC_ST26; created on Oct. 3, 2023.

### FIELD

Disclosed herein are methods of treating cancer or enhancing an anti-cancer effect associated with administration of a T-cell redirecting therapy in a subject in need thereof, comprising administering to the subject an anti-IL-2Rβγ heavy chain-only antibody or antigen binding fragment thereof in combination with a T-cell redirecting therapy. Also disclosed herein are uses of anti-IL-2Rβγ heavy chain-only antibodies and antigen binding fragments thereof and/or T-cell redirecting therapies in the manufacture of a medicament adapted for use in a method described herein; pharmaceutical compositions comprising anti-IL-2Rβγ heavy chain-only antibodies and antigen binding fragments thereof and/or T-cell redirecting therapies for use in a method described herein; and combination products.

### BACKGROUND

Bispecific T-cell engaging molecules are a new class of immunotherapies being developed for the treatment of various cancers. These molecules are designed to direct a patient's T-cells to cancer cells to induce the T-cells to attack and kill the cancer cells. Bispecific T-cell engaging molecules typically have at least one binding domain that is specific for a cell-surface antigen expressed on cancer cells and at least another binding domain that is specific for CD3, a subunit of the T-cell receptor complex expressed on T-cells. The first generation of bispecific T-cell engaging molecules (see, e.g., WO 99/54440, WO 2005/040220, and WO 2008/119567) are typically administered by continuous intravenous infusion due to short half-lives of less than a day. A second generation of bispecific T cell engaging molecules (see, e.g., WO 2013/128027, WO 2014140358, WO 2014/144722, WO 2014/151910, WO 2017/134140, WO 2017/223111, and WO 2018/052503) has been designed, at least in part, to increase the serum half-life of the molecules to enable dosing paradigms that allow for administration at intermittent dosing intervals.

Chimeric antigen receptor (CAR) T-cell therapies are another novel class of immunotherapies in which autologous or allogenic T-cells are engineered to express a CAR specific for a cell-surface antigen expressed on cancer cells, which re-directs the patient's T-cells to attack and kill cancer cells that express the cell-surface antigen. CAR-T cells have shown significant promise in the treatment of hematologic malignancies such as leukemia, lymphoma, and myeloma and are also under investigation for the treatment of solid tumors.

Although CAR-T cell therapies and bispecific T-cell engaging molecules have greatly improved clinical outcomes for some patients diagnosed with cancer, the utility of these T-cell redirecting therapies is limited in patients with “cold tumors,” which are characterized by low T-cell infiltration. Thus, there remains a need in the art for strategies to effectively enhance the therapeutic benefit of T-cell redirecting therapies in patients with cold tumors.

Interleukin-2 (IL-2) therapy represents one potential strategy for transforming “cold tumors” into treatment-responsive “hot tumors” by increasing the population of effector T-cells that can be recruited to the tumor site. IL-2 is a key regulator of immune cells, inducing both T-cell and natural killer (NK) cell proliferation. The ability to harness the immune system against tumors has been firmly established, with IL-2 being one of the first recombinant proteins to be successfully used as a treatment for cancer nearly 40 years ago. Lotze et al., Journal of Immunology 135(4), 2865-75 (1985); Rosenberg, S. A. J Immunol 192, 5451-58 (2014). Specifically, high-dose IL-2 was developed and approved (Proleukin®) for the treatment of metastatic melanoma and metastatic renal cell carcinoma, with durable responses observed in 7-12% of patients. McDermott, D. F. et al., J Clin Oncol 23, 133-141 (2004); Payne, R. et al., J Immunother Cancer 2, 13 (2014); Atkins, M. B. et al., J Clin Oncol 17, 2105-2105 (1999); Rosenberg, S. A. et al., Ann Surg 228, 307-319 (1998). However, despite having potent immune-activating activity and the potential to induce durable tumor-regression in cancer patients, the success of IL-2 as an immunotherapeutic has been limited by adverse events. Specifically, Proleukin® has severe dose-limiting toxicities, including vascular leak syndrome, hypotension, and liver toxicities, that have limited its use in cancer immunotherapy. These side events are largely due to the preferential uptake of IL-2 by cells that express the high-affinity, trimeric receptor, IL-2Rαβγ, such as T-regulatory (T_{reg}) cells and endothelial cells. By contrast, the intermediate affinity receptor is composed of only IL-2Rβ and IL-2Rγ, and is expressed on resting T-cells, CD8+ memory effector T-cells, and NK cells. Choudhry, H. et al, Biomed Res Int 2018, 1⁻⁷ (2018). The IL-2Rα subunit is not required for downstream JAK-STAT signaling, but its association with IL-2Rβ and IL-2Rγ provides a 100-fold higher affinity to IL-2 compared to the heterodimeric receptor composed only of IL-2Rβ and IL-2Rγ.

### SUMMARY

The present disclosure provides anti-IL-2Rβγ heavy chain-only antibodies and antigen-binding fragments thereof that bind to and activate signaling through the dimeric IL-2Rβγ receptor complex that is expressed on resting T-cells and NK cells. By avoiding binding to IL-2Rα, these anti-IL-2Rβγ heavy chain-only antibodies and antigen-binding fragments thereof eliminate the preferential T_{reg} activation of native IL-2, while maintaining potent stimulatory effects on other T-cell subsets along with NK cells. Additionally, the presence of an Fc region in certain anti-IL-2Rβγ heavy chain-only antibodies and antigen-binding fragments described herein significantly extends in vivo half-life compared to recombinant IL-2, potentially enabling a more convenient therapeutic dosing schedule. In vivo studies in both mice and cynomolgus monkeys have confirmed the in vivo biological activity, extended pharmacokinetics, and enhanced safety profile of the anti-IL-2Rβγ heavy chain-only antibodies and antigen-binding fragments thereof described herein. Additionally, in vitro studies using a bispecific T-cell engaging molecule that specifically binds to both human CD3 and human EpCAM have demonstrated that combination treatment with an anti-IL-2Rβγ heavy chain-only antibody of the present disclosure increases maximum T-cell killing at low E:T ratios in multiple cell lines. Moreover, combination treatment with an anti-IL-2Rβγ heavy chain-only antibody described herein and a bispecific T-cell engaging molecule that specifically binds to both human CD3 and human EpCAM in a SHP77 Luc model in female NSG mice led to robust tumor regression. Taken together, these results indicate that the anti-IL-2Rβγ heavy chain-only antibodies and antigen-binding fragments thereof described herein may improve the therapeutic efficacy of T-cell redirecting therapies when used in combination therapies and combination products.

Disclosed herein is a method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   a VH complementarity determining region one (CDR1) sequence having at most two amino acid modifications relative to any one of SEQ ID NOs: 1-3; and
   a VH CDR2 sequence having at most two amino acid modifications relative to any one of SEQ ID NOs: 4-6; and
   a VH CDR3 sequence having at most two amino acid modifications relative to any one of SEQ ID NOs: 7-10; and
a second VH region that specifically binds to IL-2Rγ, comprising:
   a VH CDR1 sequence having at most two amino acid modifications relative to any one of SEQ ID NOs: 15-16; and
   a VH CDR2 sequence having at most two amino acid modifications relative to any one of SEQ ID NOs: 17-19; and
   a VH CDR3 sequence having at most two amino acid modifications relative to any one of SEQ ID NOs: 20-21.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is a method of enhancing an anti-cancer effect associated with administration of a T-cell redirecting therapy in a subject diagnosed with cancer, comprising administering to the subject an anti-IL-2Rβγ heavy chain-only antibody or antigen binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   a VH complementarity determining region one (CDR1) sequence having at most two amino acid modifications relative to any one of SEQ ID NOs: 1-3; and
   a VH CDR2 sequence having at most two amino acid modifications relative to any one of SEQ ID NOs: 4-6; and
   a VH CDR3 sequence having at most two amino acid modifications relative to any one of SEQ ID NOs: 7-10; and
a second VH region that specifically binds to IL-2Rγ, comprising:
   a VH CDR1 sequence having at most two amino acid modifications relative to any one of SEQ ID NOs: 15-16; and
   a VH CDR2 sequence having at most two amino acid modifications relative to any one of SEQ ID NOs: 17-19; and
   a VH CDR3 sequence having at most two amino acid modifications relative to any one of SEQ ID NOs: 20-21.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the VH CDR1 sequence of the first VH region has at most two amino acid modifications relative to SEQ ID NO: 1 or SEQ ID NO: 2; the VH CDR2 sequence of the first VH region has at most two amino acid modifications relative to SEQ ID NO: 4 or SEQ ID NO: 5; and the VH CDR3 sequence of the first VH region has at most two amino acid modifications relative to any one of SEQ ID NOs: 7-9.

In some embodiments, the VH CDR1 sequence of the first VH region has at most two amino acid modifications relative to SEQ ID NO: 3; the VH CDR2 sequence of the first VH region has at most two amino acid modifications relative to SEQ ID NO: 6; and the VH CDR3 sequence of the first VH region has at most two amino acid modifications relative to SEQ ID NO: 10.

In some embodiments, the VH CDR1 sequence of the first VH region has at most one amino acid modification relative to any one of SEQ ID NOs: 1-3. In some embodiments, the VH CDR2 sequence of the first VH region has at most one amino acid modification relative to any one of SEQ ID NOs: 4-6. In some embodiments, the VH CDR3 sequence of the first VH region has at most one amino acid modification relative to any one of SEQ ID NOs: 7-10.

In some embodiments, the VH CDR1 sequence of the first VH region has at most one amino acid modification relative to SEQ ID NO: 1 or SEQ ID NO: 2; the VH CDR2 sequence of the first VH region has at most one amino acid modification relative to SEQ ID NO: 4 or SEQ ID NO: 5; and the VH CDR3 sequence of the first VH region has at most one amino acid modification relative to any one of SEQ ID NOs: 7-9.

In some embodiments, the VH CDR1 sequence of the first VH region has at most one amino acid modification relative to SEQ ID NO: 3; the VH CDR2 sequence of the first VH region has at most one amino acid modification relative to SEQ ID NO: 6; and the VH CDR3 sequence of the first VH region has at most one amino acid modification relative to SEQ ID NO: 10.

In some embodiments, the VH CDR1 sequence of the second VH region has at most two amino acid modifications relative to SEQ ID NO: 15 or SEQ ID NO: 16; the VH CDR2 sequence of the second VH region has at most two amino acid modifications relative to SEQ ID NO: 17 or SEQ ID NO: 18; and the VH CDR3 sequence of the second VH region has at most two amino acid modifications relative to SEQ ID NO: 20.

In some embodiments, the VH CDR1 sequence of the second VH region has at most two amino acid modifications relative to SEQ ID NO: 15; the VH CDR2 sequence of the second VH region has at most two amino acid modifications relative to SEQ ID NO: 19; and the VH CDR3 sequence of the second VH region has at most two amino acid modifications relative to SEQ ID NO: 21.

In some embodiments, the VH CDR1 sequence of the second VH region has at most one amino acid modification relative to any one of SEQ ID NOs: 15-16. In some embodiments, the VH CDR2 sequence of the second VH region has at most one amino acid modification relative to any one of SEQ ID NOs: 17-19. In some embodiments, the VH CDR3 sequence of the second VH region has at most one amino acid modification relative to any one of SEQ ID NOs: 20-21.

In some embodiments, the VH CDR1 sequence of the second VH region has at most one amino acid modification relative to SEQ ID NO: 15 or SEQ ID NO: 16; the VH CDR2 sequence of the second VH region has at most one amino acid modification relative to SEQ ID NO: 17 or SEQ ID NO: 18; and the VH CDR3 sequence of the second VH region has at most one amino acid modification relative to SEQ ID NO: 20.

In some embodiments, the VH CDR1 sequence of the second VH region has at most one amino acid modification relative to SEQ ID NO: 15; the VH CDR2 sequence of the second VH region has at most one amino acid modification relative to SEQ ID NO: 19; and the VH CDR3 sequence of the second VH region has at most one amino acid modification relative to SEQ ID NO: 21.

In some embodiments, the at most one amino acid modification is an amino acid substitution. In some embodiments, the at most one amino acid modification is a conservative amino acid substitution. In some embodiments, the at most one amino acid modification is an amino acid deletion. In some embodiments, the at most one amino acid modification is an amino acid addition.

In some embodiments, each amino acid modification, if any, is a conservative amino acid substitution.

In some embodiments, the first VH region comprises a VH CDR1 comprising a sequence chosen from SEQ ID NOs: 1-3. In some embodiments, the first VH region comprises a VH CDR2 comprising a sequence chosen from SEQ ID NOs: 4-6. In some embodiments, the first VH region comprises a VH CDR3 comprising a sequence chosen from SEQ ID NOs: 7-10.

In some embodiments, the first VH region comprises:
a VH CDR1 comprising a sequence chosen from SEQ ID NOs: 1-3; and/or
a VH CDR2 comprising a sequence chosen from SEQ ID NOs: 4-6; and/or
a VH CDR3 comprising a sequence chosen from SEQ ID NOs: 7 10.

In some embodiments, the first VH region comprises:
a VH CDR1 comprising a sequence chosen from SEQ ID NOs: 1-3; and
a VH CDR2 comprising a sequence chosen from SEQ ID NOs: 4-6; and
a VH CDR3 comprising a sequence chosen from SEQ ID NOs: 7 10.

In some embodiments, the first VH region comprises a VH CDR1 comprising a sequence of SEQ ID NO: 1 or SEQ ID NO: 2; a VH CDR2 comprising a sequence of SEQ ID NO: 4 or SEQ ID NO: 5; and a VH CDR3 comprising a sequence chosen from SEQ ID NOs: 7-9.

In some embodiments, the first VH region comprises a VH CDR1, a VH CDR2, and a VH CDR3 comprising the sequences of SEQ ID NOs: 1, 4, and 7, respectively.

In some embodiments, the first VH region comprises a VH CDR1, a VH CDR2, and a VH CDR3 comprising the sequences of SEQ ID NOs: 1, 4, and 8, respectively.

In some embodiments, the first VH region comprises a VH CDR1, a VH CDR2, and a VH CDR3 comprising the sequences of SEQ ID NOs: 2, 5, and 9, respectively.

In some embodiments, the first VH region comprises a VH CDR1, a VH CDR2, and a VH CDR3 comprising the sequences of SEQ ID NOs: 3, 6, and 10, respectively.

In some embodiments, the second VH region comprises a VH CDR1 comprising a sequence chosen from SEQ ID NOs: 15-16. In some embodiments, the second VH region comprises a VH CDR2 comprising a sequence chosen from SEQ ID NOs: 17-19. In some embodiments, the second VH region comprises a VH CDR3 comprising a sequence chosen from SEQ ID NOs: 20-21.

In some embodiments, the second VH region comprises:
a VH CDR1 comprising a sequence chosen from SEQ ID NOs: 15-16; and/or
a VH CDR2 comprising a sequence chosen from SEQ ID NOs: 17-19; and/or
a VH CDR3 comprising a sequence chosen from SEQ ID NOs: 20-21.

In some embodiments, the second VH region comprises:
a VH CDR1 comprising a sequence chosen from SEQ ID NOs: 15-16; and
a VH CDR2 comprising a sequence chosen from SEQ ID NOs: 17-19; and
a VH CDR3 comprising a sequence chosen from SEQ ID NOs: 20-21.

In some embodiments, the second VH region comprises a VH CDR1 comprising a sequence of SEQ ID NO: 15 or SEQ ID NO: 16; a VH CDR2 comprising a sequence of SEQ ID NO: 17 or SEQ ID NO: 18; and a VH CDR3 comprising a sequence of SEQ ID NO: 20.

In some embodiments, the second VH region comprises a VH CDR1, a VH CDR2, and a VH CDR3 comprising the sequences of SEQ ID NOs: 15, 17, and 20, respectively.

In some embodiments, the second VH region comprises a VH CDR1, a VH CDR2, and a VH CDR3 comprising the sequences of SEQ ID NOs: 15, 18, and 20, respectively.

In some embodiments, the second VH region comprises a VH CDR1, a VH CDR2, and a VH CDR3 comprising the sequences of SEQ ID NOs: 16, 18, and 20, respectively.

In some embodiments, the second VH region comprises a VH CDR1, a VH CDR2, and a VH CDR3 comprising the sequences of SEQ ID NOs: 15, 19, and 21, respectively.

In some embodiments, the first VH region comprises a VH CDR1, a VH CDR2, and a VH CDR3 comprising the sequences of SEQ ID NOs: 1, 4, and 7, respectively; and the second VH region comprises a VH CDR1, a VH CDR2, and a VH CDR3 comprising the sequences of SEQ ID NOs: 15, 17, and 20, respectively.

In some embodiments, the first VH region comprises a VH CDR1, a VH CDR2, and a VH CDR3 comprising the sequences of SEQ ID NOs: 1, 4, and 8, respectively; and the second VH region comprises a VH CDR1, a VH CDR2, and a VH CDR3 comprising the sequences of SEQ ID NOs: 15, 18, and 20, respectively.

In some embodiments, the first VH region comprises a VH CDR1, a VH CDR2, and a VH CDR3 comprising the sequences of SEQ ID NOs: 1, 4, and 8, respectively; and the second VH region comprises a VH CDR1, a VH CDR2, and a VH CDR3 comprising the sequences of SEQ ID NOs: 16, 18, and 20, respectively.

In some embodiments, the first VH region comprises a VH CDR1, a VH CDR2, and a VH CDR3 comprising the sequences of SEQ ID NOs: 1, 4, and 8, respectively; and the second VH region comprises a VH CDR1, a VH CDR2, and a VH CDR3 comprising the sequences of SEQ ID NOs: 15, 19, and 21, respectively.

In some embodiments, the first VH region comprises a VH CDR1, a VH CDR2, and a VH CDR3 comprising the sequences of SEQ ID NOs: 2, 5, and 9, respectively; and the second VH region comprises a VH CDR1, a VH CDR2, and a VH CDR3 comprising the sequences of SEQ ID NOs: 15, 18, and 20, respectively.

In some embodiments, the first VH region comprises a VH CDR1, a VH CDR2, and a VH CDR3 comprising the sequences of SEQ ID NOs: 3, 6, and 10, respectively; and the second VH region comprises a VH CDR1, a VH CDR2, and a VH CDR3 comprising the sequences of SEQ ID NOs: 15, 17, and 20, respectively.

Also disclosed herein is a method of treating cancer in a subject in need thereof, comprising administering to the subject an anti-IL-2Rβγ heavy chain-only antibody or antigen binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   (1) (a) a VH complementarity determining region one (CDR1) comprising the sequence:

(SEQ ID NO: 29)
G F T F S X₁ Y G

wherein X₁ is S or T; and
   (b) a VH CDR2 comprising the sequence:

(SEQ ID NO: 31)
A R D L D Y D X₃ L T G D P V G G F D I

wherein X₂ is K or R; and

(SEQ ID NO: 30)
I S Y D G S N X₂

(c) a VH CDR3 comprising the sequence:

(SEQ ID NO: 26)
G G S I S S S X₁ W

wherein X₁ is D or N;
   (b) a VH CDR2 comprising the sequence:

(SEQ ID NO: 27)
I X₂ H S G S T

wherein X₂ is D or S; and
   (c) a VH CDR3 comprising the sequence:

(SEQ ID NO: 28)
X₃ R G X₄ W E L X₅ D A F D I

wherein X₃ is G or A; X₄ is S or Q; and X₅ is S or T; and
a second VH region that specifically binds to IL-2Rγ, comprising:
   (1) (a) a VH CDR1 comprising the sequence:

(SEQ ID NO: 32)
G F X₁ X₂ X₃ X₄ Y Y

wherein X₁ is T or I; X₂ is F or V; X₃ is S, N, or G; and X₄ is D or N; and
(b) a VH CDR2 comprising the sequence:

(SEQ ID NO: 33)
I S X₅ S G X₆ X₇ I

wherein X₅ is S or N; X₆ is D, S, G, or N; and X₇ is T or I; and
   (c) a VH CDR3 comprising the sequence ARGDAVSITGDY (SEQ ID NO: 20); or
   (2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is a method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   (1) (a) a VH complementarity determining region one (CDR1) comprising the sequence:

(SEQ ID NO: 29)
G F T F S X₁ Y G

wherein X₁ is S or T; and
   (b) a VH CDR2 comprising the sequence:

(SEQ ID NO: 30)
I S Y D G S N X₂

wherein X₂ is K or R; and
   (c) a VH CDR3 comprising the sequence:

(SEQ ID NO: 31)
A R D L D Y D X₃ L T G D P V G G F D I

wherein X₃ is V or I; or
(2) (a) a VH CDR1 comprising the sequence:

(SEQ ID NO: 26)
G G S I S S S X₁ W

wherein X₁ is D or N;
   (b) a VH CDR2 comprising the sequence:

(SEQ ID NO: 27)
I X₂ H S G S T

wherein X₂ is D or S; and
   (c) a VH CDR3 comprising the sequence:

(SEQ ID NO: 28)
X₃ R G X₄ W E L X₅ D AF D I

wherein X₃ is G or A; X₄ is S or Q; and X₅ is S or T; and
a second VH region that specifically binds to IL-2Rγ, comprising:
   (1) (a) a VH CDR1 comprising the sequence:

(SEQ ID NO: 32)
G F X₁ X₂ X₃ X₄ Y Y

wherein X₁ is T or I; X₂ is F or V; X₃ is S, N, or G; and X₄ is D or N; and
   (b) a VH CDR2 comprising the sequence:

(SEQ ID NO: 33)
I S X₅ S G X₆ X₇ I

wherein X₅ is S or N; X₆ is D, S, G, or N; and X₇ is T or I; and
   (c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the first VH region comprises a VH CDR1 comprising the sequence of SEQ ID NO: 29, a VH CDR2 comprising the sequence of SEQ ID NO: 30, and a VH CDR3 comprising the sequence of SEQ ID NO: 31. In other embodiments, the first VH region comprises a VH CDR1 comprising the sequence of SEQ ID NO: 26, a VH CDR2 comprising the sequence of SEQ ID NO: 27, and a VH CDR3 comprising the sequence of SEQ ID NO: 28.

In some embodiments, the second VH region comprises a VH CDR1 comprising the sequence of SEQ ID NO: 32, a VH CDR2 comprising the sequence of SEQ ID NO: 33, and a VH CDR3 comprising the sequence of SEQ ID NO: 20. In other embodiments, the second VH region comprises a VH CDR1 comprising the sequence of SEQ ID NO: 15, a VH CDR2 comprising the sequence of SEQ ID NO: 19, and a VH CDR3 comprising the sequence of SEQ ID NO: 21.

In some embodiments, the first VH region comprises a VH CDR1 comprising the sequence of SEQ ID NO: 29, a VH CDR2 comprising the sequence of SEQ ID NO: 30, and a VH CDR3 comprising the sequence of SEQ ID NO: 31; and the second VH region comprises a VH CDR1 comprising the sequence of SEQ ID NO: 32, a VH CDR2 comprising the sequence of SEQ ID NO: 33, and a VH CDR3 comprising the sequence of SEQ ID NO: 20.

In some embodiments, the first VH region comprises a VH CDR1 comprising the sequence of SEQ ID NO: 29, a VH CDR2 comprising the sequence of SEQ ID NO: 30, and a VH CDR3 comprising the sequence of SEQ ID NO: 31; and the second VH region comprises a VH CDR1 comprising the sequence of SEQ ID NO: 15, a VH CDR2 comprising the sequence of SEQ ID NO: 19, and a VH CDR3 comprising the sequence of SEQ ID NO: 21.

In some embodiments, the first VH region comprises a VH CDR1 comprising the sequence of SEQ ID NO: 26, a VH CDR2 comprising the sequence of SEQ ID NO: 27, and a VH CDR3 comprising the sequence of SEQ ID NO: 28; and the second VH region comprises a VH CDR1 comprising the sequence of SEQ ID NO: 32, a VH CDR2 comprising the sequence of SEQ ID NO: 33, and a VH CDR3 comprising the sequence of SEQ ID NO: 20.

In some embodiments, the first VH region comprises a VH CDR1 comprising the sequence of SEQ ID NO: 26, a VH CDR2 comprising the sequence of SEQ ID NO: 27, and a VH CDR3 comprising the sequence of SEQ ID NO: 28; and the second VH region comprises a VH CDR1 comprising the sequence of SEQ ID NO: 15, a VH CDR2 comprising the sequence of SEQ ID NO: 19, and a VH CDR3 comprising the sequence of SEQ ID NO: 21.

Also disclosed herein is a method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is a method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the full set of VH CDRs 1, 2, and 3 (combined) in the first VH region has at least 85% sequence identity to the VH CDRs 1, 2, and 3 of any one of SEQ ID NOs: 11-14. In some embodiments, the full set of VH CDRs 1, 2, and 3 (combined) in the second VH region has at least 85% sequence identity to the VH CDRs 1, 2, and 3 of any one of SEQ ID NOs: 22-25. In some embodiments, the full set of VH CDRs 1, 2, and 3 (combined) in the first VH region has at least 85% sequence identity to the VH CDRs 1, 2, and 3 of any one of SEQ ID NOs: 11-14; and the full set of VH CDRs 1, 2, and 3 (combined) in the second VH region has at least 85% sequence identity to the VH CDRs 1, 2, and 3 of any one of SEQ ID NOs: 22-25.

In some embodiments, the full set of VH CDRs 1, 2, and 3 (combined) in the first VH region has at least 90% sequence identity to the VH CDRs 1, 2, and 3 of any one of SEQ ID NOs: 11-14. In some embodiments, the full set of VH CDRs 1, 2, and 3 (combined) in the second VH region has at least 90% sequence identity to the VH CDRs 1, 2, and 3 of any one of SEQ ID NOs: 22-25. In some embodiments, the full set of VH CDRs 1, 2, and 3 (combined) in the first VH region has at least 90% sequence identity to the VH CDRs 1, 2, and 3 of any one of SEQ ID NOs: 11-14; and the full set of VH CDRs 1, 2, and 3 (combined) in the second VH region has at least 90% sequence identity to the VH CDRs 1, 2, and 3 of any one of SEQ ID NOs: 22-25.

In some embodiments, the full set of VH CDRs 1, 2, and 3 (combined) in the first VH region has at least 95% sequence identity to the VH CDRs 1, 2, and 3 of any one of SEQ ID NOs: 11-14. In some embodiments, the full set of VH CDRs 1, 2, and 3 (combined) in the second VH region has at least 95% sequence identity to the VH CDRs 1, 2, and 3 of any one of SEQ ID NOs: 22-25. In some embodiments, the full set of VH CDRs 1, 2, and 3 (combined) in the first VH region has at least 95% sequence identity to the VH CDRs 1, 2, and 3 of any one of SEQ ID NOs: 11-14; and the full set of VH CDRs 1, 2, and 3 (combined) in the second VH region has at least 95% sequence identity to the VH CDRs 1, 2, and 3 of any one of SEQ ID NOs: 22-25.

In some embodiments, the first VH region comprises the VH CDR1, VH CDR2, and VH CDR3 of any one of SEQ ID NOs: 11-14. In some embodiments, the second VH region comprises the VH CDR1, VH CDR2, and VH CDR3 of any one of SEQ ID NOs: 22-25. In some embodiments, the first VH region comprises the VH CDR1, VH CDR2, and VH CDR3 of any one of SEQ ID NOs: 11-14; and the second VH region comprises the VH CDR1, VH CDR2, and VH CDR3 of any one of SEQ ID NOs: 22-25.

In some embodiments, the VH CDR1, VH CDR2, and VH CDR3 sequences of the first VH region are present in a human VH framework. In some embodiments, the VH CDR1, VH CDR2, and VH CDR3 sequences of the second VH region are present in a human VH framework.

Also disclosed herein is a method of treating a cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 80% (e.g., at least 85%, at least 90%, at least 95%; 80%, 85%, 90%, 95%) sequence identity to any one of SEQ ID NOs: 11-14; and
a second VH region that specifically binds to IL-2Rγ, having at least 80% (e.g., at least 85%, at least 90%, at least 95%; 80%, 85%, 90%, 95%) sequence identity to any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is a method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 80% (e.g., at least 85%, at least 90%, at least 95%; 80%, 85%, 90%, 95%) sequence identity to any one of SEQ ID NOs: 11-14; and
a second VH region that specifically binds to IL-2Rγ, having at least 80% (e.g., at least 85%, at least 90%, at least 95%; 80%, 85%, 90%, 95%) sequence identity to any one of SEQ ID NOs: 22-25.

In some embodiments, the first VH region has at least 85% sequence identity to the sequence of any one of SEQ ID NOs: 11-14. In some embodiments, the second VH region has at least 85% sequence identity to the sequence of any one of SEQ ID NOs: 22-25. In some embodiments, the first VH region has at least 85% sequence identity to the sequence of any one of SEQ ID NOs: 11-14; and the second VH region has at least 85% sequence identity to the sequence of any one of SEQ ID NOs: 22-25.

In some embodiments, the first VH region has at least 90% sequence identity to the sequence of any one of SEQ ID NOs: 11-14. In some embodiments, the second VH region has at least 90% sequence identity to the sequence of any one of SEQ ID NOs: 22-25. In some embodiments, the first VH region has at least 90% sequence identity to the sequence of any one of SEQ ID NOs: 11-14; and the second VH region has at least 90% sequence identity to the sequence of any one of SEQ ID NOs: 22-25.

In some embodiments, the first VH region has at least 95% sequence identity to the sequence of any one of SEQ ID NOs: 11-14. In some embodiments, the second VH region has at least 95% sequence identity to the sequence of any one of SEQ ID NOs: 22-25. In some embodiments, the first VH region has at least 95% sequence identity to the sequence of any one of SEQ ID NOs: 11-14; and the second VH region has at least 95% sequence identity to the sequence of any one of SEQ ID NOs: 22-25.

In some embodiments, the first VH region comprises a sequence chosen from any one of SEQ ID NOs: 11-14. In some embodiments, the second VH region comprises a sequence chosen from any one of SEQ ID NOs: 22-25. In some embodiments, the first VH region comprises a sequence chosen from any one of SEQ ID NOs: 11-14; and the second VH region comprises a sequence chosen from any one of SEQ ID NOs: 22-25.

In some embodiments, the first VH region comprises a sequence having at least 80% (e.g., at least 85%, at least 90%, at least 95%; 80%, 85%, 90%, 95%) sequence identity to the sequence of SEQ ID NO: 11; and the second VH region comprises a sequence having at least 80% (e.g., at least 85%, at least 90%, at least 95%; 80%, 85%, 90%, 95%) sequence identity to the sequence of SEQ ID NO: 22. In some embodiments, the first VH region comprises the sequence of SEQ ID NO: 11; and the second VH region comprises the sequence of SEQ ID NO: 22.

In some embodiments, the first VH region comprises a sequence having at least 80% (e.g., at least 85%, at least 90%, at least 95%; 80%, 85%, 90%, 95%) sequence identity to the sequence of SEQ ID NO: 12; and the second VH region comprises a sequence having at least 80% (e.g., at least 85%, at least 90%, at least 95%; 80%, 85%, 90%, 95%) sequence identity to the sequence of SEQ ID NO: 23. In some embodiments, the first VH region comprises the sequence of SEQ ID NO: 12; and the second VH region comprises the sequence of SEQ ID NO: 23.

In some embodiments, the first VH region comprises a sequence having at least 80% (e.g., at least 85%, at least 90%, at least 95%; 80%, 85%, 90%, 95%) sequence identity to the sequence of SEQ ID NO: 12; and the second VH region comprises a sequence having at least 80% (e.g., at least 85%, at least 90%, at least 95%; 80%, 85%, 90%, 95%) sequence identity to the sequence of SEQ ID NO: 24. In some embodiments, the first VH region comprises the sequence of SEQ ID NO: 12; and the second VH region comprises the sequence of SEQ ID NO: 24.

In some embodiments, the first VH region comprises a sequence having at least 80% (e.g., at least 85%, at least 90%, at least 95%; 80%, 85%, 90%, 95%) sequence identity to the sequence of SEQ ID NO: 12; and the second VH region comprises a sequence having at least 80% (e.g., at least 85%, at least 90%, at least 95%; 80%, 85%, 90%, 95%) sequence identity to the sequence of SEQ ID NO: 25. In some embodiments, the first VH region comprises the sequence of SEQ ID NO: 12; and the second VH region comprises the sequence of SEQ ID NO: 25.

In some embodiments, the first VH region comprises a sequence having at least 80% (e.g., at least 85%, at least 90%, at least 95%; 80%, 85%, 90%, 95%) sequence identity to the sequence of SEQ ID NO: 13; and the second VH region comprises a sequence having at least 80% (e.g., at least 85%, at least 90%, at least 95%; 80%, 85%, 90%, 95%) sequence identity to the sequence of SEQ ID NO: 23. In some embodiments, the first VH region comprises the sequence of SEQ ID NO: 13; and the second VH region comprises the sequence of SEQ ID NO: 23.

In some embodiments, the first VH region comprises a sequence having at least 80% (e.g., at least 85%, at least 90%, at least 95%; 80%, 85%, 90%, 95%) sequence identity to the sequence of SEQ ID NO: 14; and the second VH region comprises a sequence having at least 80% (e.g., at least 85%, at least 90%, at least 95%; 80%, 85%, 90%, 95%) sequence identity to the sequence of SEQ ID NO: 22. In some embodiments, the first VH region comprises the sequence of SEQ ID NO: 14; and the second VH region comprises the sequence of SEQ ID NO: 22.

In some embodiments, the anti-IL-2Rβγ heavy chain-only antibody or antigen-binding fragment thereof further comprises a heavy chain constant region sequence in the absence of a CH1 sequence.

In some embodiments, the anti-IL-2Rβγ heavy chain-only antibody or antigen-binding fragment thereof further comprises a heavy chain constant region comprising a hinge region, a CH2 domain, and a CH3 domain.

In some embodiments, the hinge region comprises a wild type human IgG4 hinge region sequence (SEQ ID NO: 54). In some embodiments, the hinge region comprises a variant human IgG4 hinge region sequence comprising an S228P mutation (SEQ ID NO: 55).

In some embodiments, the CH2 domain comprises a wild type human IgG4 CH2 domain sequence (SEQ ID NO: 56). In some embodiments, the CH2 domain comprises a variant human IgG4 CH2 domain comprising an F234A mutation, an L235A mutation, or both an F234A mutation and an L235A mutation.

In some embodiments, the CH3 domain comprises a wild type human IgG4 CH3 domain sequence (SEQ ID NO: 58). In some embodiments, the CH3 domain comprises a variant human IgG4 CH3 domain sequence comprising a T366W mutation. In some embodiments, the CH3 domain comprises a variant human IgG4 CH3 domain sequence comprising a T366S, an L368A mutation, and a Y407V mutation.

In some embodiments, the anti-IL-2Rβγ heavy chain-only antibody or antigen-binding fragment thereof further comprises a Fc region. In some embodiments, the anti-IL-2Rβγ heavy chain-only antibody or antigen-binding fragment thereof further comprises a variant Fc region. In some embodiments, the variant Fc region comprises heterodimerizing alterations. In some embodiments, the Fc region is a silenced Fc region.

In some embodiments, the anti-IL-2Rβγ heavy chain-only antibody is an IgG1 antibody. In some embodiments, the anti-IL-2Rβγ heavy chain-only antibody is an IgG4 antibody.

In some embodiments, the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 53; and
a second polypeptide comprising a sequence having at least 80% (e.g., at least 85%, at least 90%, at least 95%; 80%, 85%, 90%, 95%) sequence identity to the sequence of SEQ ID NO: 61

In some embodiments, the first polypeptide comprises the sequence of SEQ ID NO: 53; and the second polypeptide comprises the sequence of SEQ ID NO: 61.

In some embodiments, the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 62; and
a second polypeptide comprising a sequence having at least 80% (e.g., at least 85%, at least 90%, at least 95%; 80%, 85%, 90%, 95%) sequence identity to the sequence of SEQ ID NO: 63.

In some embodiments, the first polypeptide comprises the sequence of SEQ ID NO: 62; and the second polypeptide comprises the sequence of SEQ ID NO: 63.

In some embodiments, the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 64; and
a second polypeptide comprising a sequence having at least 80% (e.g., at least 85%, at least 90%, at least 95%; 80%, 85%, 90%, 95%) sequence identity to the sequence of SEQ ID NO: 65.

In some embodiments, the first polypeptide comprises the sequence of SEQ ID NO: 64; and the second polypeptide comprises the sequence of SEQ ID NO: 65.

In some embodiments, the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 66; and
a second polypeptide comprising a sequence having at least 80% (e.g., at least 85%, at least 90%, at least 95%; 80%, 85%, 90%, 95%) sequence identity to the sequence of SEQ ID NO: 67.

In some embodiments, the first polypeptide comprises the sequence of SEQ ID NO: 66; and the second polypeptide comprises the sequence of SEQ ID NO: 67.

In some embodiments, the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 34; and
a second polypeptide comprising a sequence having at least 80% (e.g., at least 85%, at least 90%, at least 95%; 80%, 85%, 90%, 95%) sequence identity to the sequence of SEQ ID NO: 35.

In some embodiments, the first polypeptide comprises the sequence of SEQ ID NO: 34; and the second polypeptide comprises the sequence of SEQ ID NO: 35.

In some embodiments, the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 36; and
a second polypeptide comprising a sequence having at least 80% (e.g., at least 85%, at least 90%, at least 95%; 80%, 85%, 90%, 95%) sequence identity to the sequence of SEQ ID NO: 37.

In some embodiments, the first polypeptide comprises the sequence of SEQ ID NO: 36; and the second polypeptide comprises the sequence of SEQ ID NO: 37.

In some embodiments, the anti-IL-2Rβγ heavy chain-only antibody or antigen-binding fragment has an affinity for IL2R with a Kd of from about 10⁻¹¹ M to about 10⁻⁶ M.

In some embodiments, the anti-IL-2Rβγ heavy chain-only antibody or antigen-binding fragment has an affinity for IL2RP with a Kd of from about 10⁻⁸ M to about 2.5×10⁻⁷ M.

In some embodiments, the anti-IL-2Rβγ heavy chain-only antibody or antigen-binding fragment has an affinity for IL2RT with a Kd of from about 10⁻⁹ M to about 2.5×10⁻⁸ M.

In some embodiments, the anti-IL-2Rβγ heavy chain-only antibody or antigen-binding fragment functions as an IL-2Rβγ agonist.

In some embodiments, a method of the present disclosure further comprises administering a premedication to the subject prior to the administration of a first dose of the anti-IL-2Rβγ heavy chain-only antibody or antigen-binding fragment or a first dose of the T-cell redirecting therapy. In some embodiments, the premedication is chosen from antihistamines, glucocorticoids, IL-6 receptor antagonists, and tumor necrosis factor alpha (TNF-α) antagonists.

In some embodiments of a method disclosed herein, the T-cell redirecting therapy is a bispecific T-cell engaging molecule. In some embodiments, the bispecific T-cell engaging molecule comprises a first domain that specifically binds to a target cancer cell antigen and a second domain that specifically binds to human CD3. In some embodiments, the target cancer cell antigen is chosen from CEA, CD19, CD33, CD70, EGFRvIII, FLT3, GPRC5D, DLL3, BCMA, PSMA, STEAP1, STEAP2, MUC16, MUC17, and CLDN18.2.

In some embodiments, the bispecific T-cell engaging molecule further comprises a half-life extension domain. In some embodiments, the half-life extension domain provides a half-life for the bispecific T-cell engaging molecule of greater than about 24 hours. In some embodiments, the half-life extension domain is chosen from immunoglobulin Fc domains, domains derived from serum albumin (e.g., human serum albumin), albumin-binding domains (e.g., comprising human albumin binding peptides or an antibody fragment that specifically binds to serum albumin), peptides that bind to the neonatal Fc receptor (FcRn), and polyethylene glycol polymers.

In some embodiments, the bispecific T-cell engaging molecule is a three-chain antibody like molecule.

In alternative embodiments of a method disclosed herein, the T-cell redirecting therapy is a chimeric antigen receptor (CAR)-expressing T-cell. In some embodiments, the CAR-expressing T-cell comprises a first domain that specifically binds to a target cancer cell antigen, a transmembrane domain, and an intracellular signalling domain. In some embodiments, the target cancer cell antigen is chosen from CEA, CD19, CD33, CD70, EGFRvIII, FLT3, GPRC5D, DLL3, BCMA, PSMA, STEAP1, STEAP2, MUC16, MUC17, and CLDN18.2.

In some embodiments of a method disclosed herein, at least one dose of the anti-IL-2Rβγ heavy chain-only antibody or antigen-binding fragment thereof is administered to the subject prior to a first dose of the T-cell redirecting therapy.

In some embodiments, the method comprises administering the anti-IL-2Rβγ heavy chain-only antibody or antigen-binding fragment thereof in combination with the T-cell redirecting therapy in one or more treatment cycles. In some embodiments, each of the one or more treatment cycles comprises a single dose of the anti-IL-2Rβγ heavy chain-only antibody or antigen-binding fragment thereof and a single dose of the T-cell redirecting therapy. In some embodiments, each of the one or more treatment cycles comprises multiple doses of the anti-IL-2Rβγ heavy chain-only antibody or antigen-binding fragment thereof and a single dose of the T-cell redirecting therapy. In some embodiments, each of the one or more treatment cycles comprises a single dose of the anti-IL-2Rβγ heavy chain-only antibody or antigen-binding fragment thereof and multiple doses of the T-cell redirecting therapy. In some embodiments, each of the one or more treatment cycles comprises multiple doses of the anti-IL-2Rβγ heavy chain-only antibody or antigen-binding fragment thereof and multiple doses of the T-cell redirecting therapy.

In some embodiments of a method disclosed herein, the subject is diagnosed with a hematologic cancer. In some embodiments, the hematologic cancer is chosen from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.

In some embodiments of a method disclosed herein, the subject is diagnosed with a cancer chosen from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma.

In some embodiments, the subject has at least one tumor with low immune infiltration (e.g., low or no T-cell infiltration) prior to the co-administration. In some embodiments, the co-administration increases tumor T-cell infiltration. In some embodiments, the co-administration is associated with at least one anti-tumor effect. In some embodiments, the at least one anti-cancer effect is chosen from a decrease in the number of cancer cells, a decrease in the number of metastases, an increase in life expectancy, a decrease in cancer cell proliferation, a decrease in cancer cell survival, and an amelioration of various physiological symptoms associated with the cancerous condition.

In some embodiments of a method disclosed herein, the anti-IL-2Rβγ heavy chain-only antibody or antigen-binding fragment thereof is administered in a pharmaceutical composition adapted for intravenous or subcutaneous delivery.

In some embodiments of a method disclosed herein, the T-cell redirecting therapy is administered in a pharmaceutical composition adapted for intravenous or subcutaneous delivery. In some embodiments, the T-cell redirecting therapy is a bispecific T-cell engaging molecule; and the pharmaceutical composition comprises the bispecific T-cell engaging molecule, a buffer, a surfactant, and a stabilizing agent. In some embodiments, the T-cell redirecting therapy is a bispecific T-cell engaging molecule; and the pharmaceutical composition comprises the bispecific T-cell engaging molecule, a glutamate buffer, polysorbate 20 or polysorbate 80, and sucrose, at a pH of about 4.0 to about 4.4.

In some embodiments of a method disclosed herein, the anti-IL-2Rβγ heavy chain-only antibody or antigen-binding fragment thereof and the T-cell redirecting therapy are administered in separate pharmaceutical compositions. In some embodiments, the separate pharmaceutical compositions may be lyophilized and reconstituted prior to administration to a patient.

In some embodiments of a method disclosed herein, the anti-IL-2Rβγ heavy chain-only antibody or antigen-binding fragment thereof and the T-cell redirecting therapy are administered concurrently. In alternative embodiments of a method disclosed herein, the anti-IL-2Rβγ heavy chain-only antibody or antigen-binding fragment thereof and the T-cell redirecting therapy are administered sequentially.

In some embodiments of a method disclosed herein, the subject was previously administered a first line therapy for the cancer. In some embodiments, the subject was previously administered a first line therapy and a second line therapy for the cancer.

Also disclosed herein is use of an anti-IL-2Rβγ heavy chain-only antibody or antigen-binding fragment thereof in the manufacture of a medicament adapted for use in a method disclosed herein. In some embodiments, the method possesses one or more of the features of a method described above. In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is use of a T-cell redirecting therapy in the manufacture of a medicament adapted for use in a method disclosed herein. In some embodiments, the method possesses one or more of the features of a method described above.

Also disclosed herein is use of an anti-IL-2Rβγ heavy chain-only antibody or antigen-binding fragment thereof and a T-cell redirecting therapy in the manufacture of a medicament adapted for use in a method disclosed herein. In some embodiments, the method possesses one or more of the features of a method described above. In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is a pharmaceutical composition comprising an anti-IL-2Rβγ heavy chain-only antibody or antigen-binding fragment thereof for use in a method disclosed herein. In some embodiments, the method possesses one or more of the features of a method described above. In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is a pharmaceutical composition comprising a T-cell redirecting therapy for use in a method disclosed herein. In some embodiments, the method possesses one or more of the features of a method described above. In some embodiments, the T-cell redirecting therapy is a T-cell engaging molecule. In some embodiments, the T-cell redirecting therapy is a bispecific T-cell engaging molecule.

Also disclosed herein is a pharmaceutical composition comprising an anti-IL-2Rβγ heavy chain-only antibody or antigen-binding fragment thereof and a T-cell redirecting therapy for use in a method disclosed herein. In some embodiments, the method possesses one or more of the features of a method described above. In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody. In some embodiments, the T-cell redirecting therapy is a T-cell engaging molecule. In some embodiments, the T-cell redirecting therapy is a bispecific T-cell engaging molecule.

Also disclosed herein is a combination product comprising an anti-IL-2Rβγ heavy chain-only antibody or antigen-binding fragment thereof and a T-cell redirecting therapy. In some embodiments, the combination product is adapted for use in a method disclosed herein. In some embodiments, the method possesses one or more of the features of a method described above. In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody. In some embodiments, the T-cell redirecting therapy is a T-cell engaging molecule. In some embodiments, the T-cell redirecting therapy is a bispecific T-cell engaging molecule.

Also disclosed herein is a combination product for use in a method of treating cancer in a subject in need thereof, comprising an anti-IL-2Rβγ heavy chain-only antibody or antigen-binding fragment thereof and a T-cell redirecting therapy, wherein the anti-IL-2Rβγ heavy chain-only antibody enhances an anti-cancer effect associated with administration of the T-cell redirecting therapy. In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody. In some embodiments, the T-cell redirecting therapy is a T-cell engaging molecule. In some embodiments, the T-cell redirecting therapy is a bispecific T-cell engaging molecule.

Also disclosed herein is an anti-IL-2Rβγ heavy chain-only antibody or antigen-binding fragment thereof for use in the treatment of cancer in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   a VH complementarity determining region one (CDR1) sequence having at most two amino acid modifications relative to any one of SEQ ID NOs: 1-3; and
   a VH CDR2 sequence having at most two amino acid modifications relative to any one of SEQ ID NOs: 4-6; and
   a VH CDR3 sequence having at most two amino acid modifications relative to any one of SEQ ID NOs: 7-10; and
a second VH region that specifically binds to IL-2Rγ, comprising:
   a VH CDR1 sequence having at most two amino acid modifications relative to any one of SEQ ID NOs: 15-16; and
   a VH CDR2 sequence having at most two amino acid modifications relative to any one of SEQ ID NOs: 17-19; and
   a VH CDR3 sequence having at most two amino acid modifications relative to any one of SEQ ID NOs: 20-21.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the T-cell redirecting therapy is a T-cell engaging molecule. In some embodiments, the T-cell redirecting therapy is a bispecific T-cell engaging molecule.

Also disclosed herein is an anti-IL-2Rβγ heavy chain-only antibody or antigen-binding fragment thereof for use in the treatment of cancer in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   (1) (a) a VH complementarity determining region one (CDR1) comprising the sequence:

(SEQ ID NO: 29)
G F T F S X₁ Y G

wherein X₁ is S or T; and
   (b) a VH CDR2 comprising the sequence:

(SEQ ID NO: 30)
I S Y D G S N X₂

wherein X₂ is K or R; and
   (c) a VH CDR3 comprising the sequence:

(SEQ ID NO: 31)
A R D L D Y D X₃ L T G D P V G G F D I

wherein X₃ is V or I; or
(2) (a) a VH CDR1 comprising the sequence:

(SEQ ID NO: 26)
G G S I S S S X₁ W

wherein X₁ is D or N;
   (b) a VH CDR2 comprising the sequence:

(SEQ ID NO: 27)
I X₂ H S G S T

wherein X₂ is D or S; and
   (c) a VH CDR3 comprising the sequence:

(SEQ ID NO: 28)
X₃ R G X₄ W E L X₅ D A F D I

wherein X₃ is G or A; X₄ is S or Q; and X₅ is S or T; and
a second VH region that specifically binds to IL-2Rγ, comprising:
   (1) (a) a VH CDR1 comprising the sequence: PGP43,DNA

(SEQ ID NO: 32)
G F X₁ X₂ X₃ X₄ Y Y

wherein X₁ is T or I; X₂ is F or V; X₃ is S, N, or G; and X₄ is D or N; and
   (b) a VH CDR2 comprising the sequence:

(SEQ ID NO: 33)
I S X₅ S G X₆ X₇ I

wherein X₅ is S or N; X₆ is D, S, G, or N; and X₇ is T or I; and
   (c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the T-cell redirecting therapy is a T-cell engaging molecule. In some embodiments, the T-cell redirecting therapy is a bispecific T-cell engaging molecule.

Also disclosed herein is an anti-IL-2Rβγ heavy chain-only antibody or antigen-binding fragment thereof for use in the treatment of cancer in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 95% sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 95% sequence identity to the VH CDRs 1, 2, and 3 of any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the T-cell redirecting therapy is a T-cell engaging molecule. In some embodiments, the T-cell redirecting therapy is a bispecific T-cell engaging molecule.

Also disclosed herein is an anti-IL-2Rβγ heavy chain-only antibody or antigen-binding fragment thereof for use in the treatment of cancer in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 95% sequence identity to the sequence of any one of SEQ ID NOs: 11-14; and
   a second VH region that specifically binds to IL-2Rγ, having at least 95% sequence identity to the sequence of any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In some embodiments, the T-cell redirecting therapy is a T-cell engaging molecule. In some embodiments, the T-cell redirecting therapy is a bispecific T-cell engaging molecule.

Also disclosed herein is an anti-IL-2Rβγ heavy chain-only antibody or antigen-binding fragment thereof for use in enhancing an anti-cancer effect associated with administration of a T-cell redirecting therapy in a subject diagnosed with cancer, wherein the anti-IL-2Rβγ heavy chain-only antibody or antigen-binding fragment thereof comprises:
a first heavy chain variable (VH) region that specifically binds to IL-2Rβ, comprising:
   a VH complementarity determining region one (CDR1) sequence having at most two amino acid modifications relative to any one of SEQ ID NOs: 1-3; and
   a VH CDR2 sequence having at most two amino acid modifications relative to any one of SEQ ID NOs: 4-6; and
   a VH CDR3 sequence having at most two amino acid modifications relative to any one of SEQ ID NOs: 7-10; and
a second VH region that specifically binds to IL-2Rγ, comprising:
   a VH CDR1 sequence having at most two amino acid modifications relative to any one of SEQ ID NOs: 15-16; and/or
   a VH CDR2 sequence having at most two amino acid modifications relative to any one of SEQ ID NOs: 17-19; and/or
   a VH CDR3 sequence having at most two amino acid modifications relative to any one of SEQ ID NOs: 20-21.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is an anti-IL-2Rβγ heavy chain-only antibody or antigen-binding fragment thereof for use in enhancing an anti-cancer effect associated with administration of a T-cell redirecting therapy in a subject diagnosed with cancer, wherein the anti-IL-2Rβγ heavy chain-only antibody or antigen-binding fragment thereof comprises:
a first heavy chain variable (VH) region that specifically binds to IL-2Rβ, comprising:
   (1) (a) a VH complementarity determining region one (CDR1) comprising the sequence:

(SEQ ID NO: 29)
G F T F S X₁ Y G

wherein X₁ is S or T; and
   (b) a VH CDR2 comprising the sequence:

(SEQ ID NO: 30)
I S Y D G S N X₂

wherein X₂ is K or R; and
   (c) a VH CDR3 comprising the sequence:

(SEQ ID NO: 31)
A R D L D Y D X₃ L T G D P V G G F D I

wherein X₃ is V or I; or
(2) (a) a VH CDR1 comprising the sequence:

(SEQ ID NO: 26)
G G S I S S S X₁ W

wherein X₁ is D or N;
   (b) a VH CDR2 comprising the sequence:

(SEQ ID NO: 27)
I X₂ H S G S T

wherein X₂ is D or S; and
   (c) a VH CDR3 comprising the sequence:

(SEQ ID NO: 28)
X₃ R G X₄ W E L X₅ D A F D I

wherein X₃ is G or A; X₄ is S or Q; and X₅ is S or T; and

(SEQ ID NO: 32)
G F X₁ X₂ X₃ X₄ Y Y

wherein X₁ is T or I; X₂ is F or V; X₃ is S, N, or G; and X₄ is D or N; and
   (b) a VH CDR2 comprising the sequence:

(SEQ ID NO: 33)
I S X₅ S G X₆ X₇ I

wherein X₅ is S or N; X₆ is D, S, G, or N; and X₇ is T or I; and
   (c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is an anti-IL-2Rβγ heavy chain-only antibody or antigen-binding fragment thereof for use in enhancing an anti-cancer effect associated with administration of a T-cell redirecting therapy in a subject diagnosed with cancer, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 95% sequence identity to the VH CDRs 1, 2, and 3 of any one of SEQ ID NOs: 11-14; and
   a second VH region that specifically binds to IL-2Rγ, in which the full set of VH complementarity determining regions (CDRs) 1, 2, and 3 (combined) has at least 95% sequence identity to the VH CDRs 1, 2, and 3 of any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

Also disclosed herein is an anti-IL-2Rβγ heavy chain-only antibody or antigen-binding fragment thereof for use in enhancing an anti-cancer effect associated with administration of a T-cell redirecting therapy in a subject diagnosed with cancer, wherein the anti-IL-2Rβγ heavy chain-only antibody or antigen-binding fragment thereof comprises:
a first heavy chain variable (VH) region that specifically binds to IL-2Rβ, having at least 95% sequence identity to the sequence of any one of SEQ ID NOs: 11-14; and
a second VH region that specifically binds to IL-2Rγ, having at least 95% sequence identity to the sequence of any one of SEQ ID NOs: 22-25.

In some embodiments, the anti-IL-2Rβγ heavy chain-only antibody or antigen-binding fragment thereof is an anti-IL-2Rβγ heavy chain-only antibody. In some embodiments, the anti-IL-2Rβγ heavy chain-only antibody or antigen-binding fragment thereof is an antigen-binding fragment of an anti-IL-2Rβγ heavy chain-only antibody.

In alternative aspects of the present disclosure, an NK-cell redirecting therapy, such as an NK cell engaging molecule or a CAR-NK cell, may be used in place of a T-cell redirecting therapy in a method, use, pharmaceutical composition, or combination product described herein.

Without limitation, some example embodiments of the disclosure include:

E1. A method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   a VH complementarity determining region one (CDR1) sequence having at most two amino acid modifications relative to any one of SEQ ID NOs: 1-3; and
   a VH CDR2 sequence having at most two amino acid modifications relative to any one of SEQ ID NOs: 4-6; and
   a VH CDR3 sequence having at most two amino acid modifications relative to any one of SEQ ID NOs: 7-10; and
a second VH region that specifically binds to IL-2Rγ, comprising:
   a VH CDR1 sequence having at most two amino acid modifications relative to any one of SEQ ID NOs: 15-16; and
   a VH CDR2 sequence having at most two amino acid modifications relative to any one of SEQ ID NOs: 17-19; and
   a VH CDR3 sequence having at most two amino acid modifications relative to any one of SEQ ID NOs: 20-21.
      E2. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   a VH complementarity determining region one (CDR1) sequence having at most two amino acid modifications relative to any one of SEQ ID NOs: 1-3; and
   a VH CDR2 sequence having at most two amino acid modifications relative to any one of SEQ ID NOs: 4-6; and
   a VH CDR3 sequence having at most two amino acid modifications relative to any one of SEQ ID NOs: 7-10; and
a second VH region that specifically binds to IL-2Rγ, comprising:
   a VH CDR1 sequence having at most two amino acid modifications relative to any one of SEQ ID NOs: 15-16; and
   a VH CDR2 sequence having at most two amino acid modifications relative to any one of SEQ ID NOs: 17-19; and
   a VH CDR3 sequence having at most two amino acid modifications relative to any one of SEQ ID NOs: 20-21.
      E3. The method according to E1 or E2, wherein the VH CDR1 sequence of the first VH region has at most one amino acid modification relative to any one of SEQ ID NOs: 1-3.
      E4. The method according to any one of E1-E3, wherein the VH CDR2 sequence of the first VH region has at most one amino acid modification relative to any one of SEQ ID NOs: 4-6.
      E5. The method according to any one of E1-E4, wherein the VH CDR3 sequence of the first VH region has at most one amino acid modification relative to any one of SEQ ID NOs: 7-10.
      E6. The method according to any one of E1-E5, wherein the VH CDR1 sequence of the second VH region has at most one amino acid modification relative to any one of SEQ ID NOs: 15-16.
      E7. The method according to any one of E1-E6, wherein the VH CDR2 sequence of the second VH region has at most one amino acid modification relative to any one of SEQ ID NOs: 17-19.
      E8. The method according to any one of E1-E7, wherein the VH CDR3 sequence of the second VH region has at most one amino acid modification relative to any one of SEQ ID NOs: 20-21.
      E9. The method according to any one of E3-E8, wherein the at most one amino acid modification is an amino acid substitution.
      E10. The method according to any one of E3-E9, wherein the at most one amino acid modification is a conservative amino acid substitution.
      E11. The method according to any one of E3-E8, wherein the at most one amino acid modification is an amino acid deletion.
      E12. The method according to any one of E3-E8, wherein the at most one amino acid modification is an amino acid addition.
      E13. The method according to any one of E1-E8, wherein each amino acid modification, if any, is a conservative amino acid substitution.
      E14. The method according to any one of E1-13, wherein the first VH region comprises a VH CDR1 comprising a sequence chosen from SEQ ID NOs: 1-3.
      E15. The method according to any one of E1-14, wherein the first VH region comprises a VH CDR2 comprising a sequence chosen from SEQ ID NOs: 4-6.
      E16. The method according to any one of E1-15, wherein the first VH region comprises a VH CDR3 comprising a sequence chosen from SEQ ID NOs: 7-10.
      E17. The method according to any one of E1-16, wherein the first VH region comprises:
a VH CDR1 comprising a sequence chosen from SEQ ID NOs: 1-3; and
a VH CDR2 comprising a sequence chosen from SEQ ID NOs: 4-6; and
a VH CDR3 comprising a sequence chosen from SEQ ID NOs: 7-10.
   E18. The method according to any one of E1-17, wherein the first VH region comprises:
(a) a VH CDR1, a VH CDR2, and a VH CDR3 comprising the sequences of SEQ ID NOs: 1, 4, and 7, respectively; or
(b) a VH CDR1, a VH CDR2, and a VH CDR3 comprising the sequences of SEQ ID NOs: 1, 4, and 8, respectively; or
(c) a VH CDR1, a VH CDR2, and a VH CDR3 comprising the sequences of SEQ ID NOs: 2, 5, and 9, respectively; or
(d) a VH CDR1, a VH CDR2, and a VH CDR3 comprising the sequences of SEQ ID NOs: 3, 6, and 10, respectively.
   E19. The method according to any one of E1-E18, wherein the second VH region comprises a VH CDR1 comprising a sequence chosen from SEQ ID NOs: 15-16.
   E20. The method according to any one of E1-E19, wherein the second VH region comprises a VH CDR2 comprising a sequence chosen from SEQ ID NOs: 17-19.
   E21. The method according to any one of E1-E20, wherein the second VH region comprises a VH CDR3 comprising a sequence chosen from SEQ ID NOs: 20-21.
   E22. The method according to any one of E1-E21, wherein the second VH region comprises:
a VH CDR1 comprising a sequence chosen from SEQ ID NOs: 15-16; and
a VH CDR2 comprising a sequence chosen from SEQ ID NOs: 17-19; and
a VH CDR3 comprising a sequence chosen from SEQ ID NOs: 20-21.
   E23. The method according to any one of E1-E22, wherein the second VH region comprises:
(a) a VH CDR1, a VH CDR2, and a VH CDR3 comprising the sequences of SEQ ID NOs: 15, 17, and 20, respectively; or
(b) a VH CDR1, a VH CDR2, and a VH CDR3 comprising the sequences of SEQ ID NOs: 15, 18, and 20, respectively; or
(c) a VH CDR1, a VH CDR2, and a VH CDR3 comprising the sequences of SEQ ID NOs: 16, 18, and 20, respectively; or
(d) a VH CDR1, a VH CDR2, and a VH CDR3 comprising the sequences of SEQ ID NOs: 15, 19, and 21, respectively.
   E24. The method according to any one of E1-E23, wherein:
the first VH region comprises a VH CDR1, a VH CDR2, and a VH CDR3 comprising the sequences of SEQ ID NOs: 1, 4, and 7, respectively; and
the second VH region comprises a VH CDR1, a VH CDR2, and a VH CDR3 comprising the sequences of SEQ ID NOs: 15, 17, and 20, respectively.
   E25. The method according to any one of E1-E23, wherein:
the first VH region comprises a VH CDR1, a VH CDR2, and a VH CDR3 comprising the sequences of SEQ ID NOs: 1, 4, and 8, respectively; and
the second VH region comprises a VH CDR1, a VH CDR2, and a VH CDR3 comprising the sequences of SEQ ID NOs: 15, 18, and 20, respectively.
   E26. The method according to any one of E1-E23, wherein:
the first VH region comprises a VH CDR1, a VH CDR2, and a VH CDR3 comprising the sequences of SEQ ID NOs: 1, 4, and 8, respectively; and
the second VH region comprises a VH CDR1, a VH CDR2, and a VH CDR3 comprising the sequences of SEQ ID NOs: 16, 18, and 20, respectively.
   E27. The method according to any one of E1-E23, wherein:
the first VH region comprises a VH CDR1, a VH CDR2, and a VH CDR3 comprising the sequences of SEQ ID NOs: 1, 4, and 8, respectively; and
the second VH region comprises a VH CDR1, a VH CDR2, and a VH CDR3 comprising the sequences of SEQ ID NOs: 15, 19, and 21, respectively.
   E28. The method according to any one of E1-E23, wherein:
the first VH region comprises a VH CDR1, a VH CDR2, and a VH CDR3 comprising the sequences of SEQ ID NOs: 2, 5, and 9, respectively; and
the second VH region comprises a VH CDR1, a VH CDR2, and a VH CDR3 comprising the sequences of SEQ ID NOs: 15, 18, and 20, respectively.
   E29. The method according to any one of E1-E23, wherein:
the first VH region comprises a VH CDR1, a VH CDR2, and a VH CDR3 comprising the sequences of SEQ ID NOs: 3, 6, and 10, respectively; and
the second VH region comprises a VH CDR1, a VH CDR2, and a VH CDR3 comprising the sequences of SEQ ID NOs: 15, 17, and 20, respectively.
   E30. A method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
   (1) (a) a VH complementarity determining region one (CDR1) comprising the sequence:

(SEQ ID NO: 29)
G F T F S X₁ Y G

wherein X₁ is S or T; and
   (b) a VH CDR2 comprising the sequence:

(SEQ ID NO: 30)
I S Y D G S N X₂

wherein X₂ is K or R; and
   (c) a VH CDR3 comprising the sequence:

(SEQ ID NO: 31)
A R D L D Y D X₃ L T G D P V G G F D I

wherein X₃ is V or I; or
(2) (a) a VH CDR1 comprising the sequence:

(SEQ ID NO: 26)
G G S I S S S X₁ W

wherein X₁ is D or N;
   (b) a VH CDR2 comprising the sequence:

(SEQ ID NO: 27)
I X₂ H S G S T

wherein X₂ is D or S; and
   (c) a VH CDR3 comprising the sequence:

(SEQ ID NO: 28)
X₃ R G X₄ W E L X₅ D A F D I

wherein X₃ is G or A; X₄ is S or Q; and Xₛ is S or T; and
a second VH region that specifically binds to IL-2Rγ, comprising:
   (1) (a) a VH CDR1 comprising the sequence:

(SEQ ID NO: 32)
G F X₁ X₂ X₃ X₄ Y Y

wherein X₁ is T or I; X₂ is F or V; X₃ is S, N, or G; and X₄ is D or N; and
   (b) a VH CDR2 comprising the sequence:

(SEQ ID NO: 33)
I S X₅ S G X₆ X₇ I

wherein X₅ is S or N; X₆ is D, S, G, or N; and X₇ is T or I; and
   (c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).
   E31. A method of enhancing an anti-cancer effect associated with administration of a T-cell redirecting therapy in a subject diagnosed with cancer, comprising administering to the subject an anti-IL-2R37 heavy chain-only antibody or antigen-binding fragment thereof in combination with the T-cell redirecting therapy, wherein the anti-IL-2R37 heavy chain-only antibody or antigen-binding fragment thereof comprises:
a first heavy chain variable (VH) region that specifically binds to IL-2R3, comprising:
   (1) (a) a VH complementarity determining region one (CDR1) comprising the sequence:

(SEQ ID NO: 29)
G F T F S X₁ Y G

wherein X₁ is S or T; and
   (b) a VH CDR2 comprising the sequence:

(SEQ ID NO: 30)
I S Y D G S N X₂

wherein X₂ is K or R; and
   (c) a VH CDR3 comprising the sequence:

(SEQ ID NO: 31)
A R D L D Y D X₃ L T G D P V G G F D I

wherein X₃ is V or I; or
(2) (a) a VH CDR1 comprising the sequence:

(SEQ ID NO: 26)
G G S I S S S X₁ W

wherein X₁ is D or N;
   (b) a VH CDR2 comprising the sequence:

(SEQ ID NO: 27)
I X₂ H S G S T

wherein X₂ is D or S; and
   (c) a VH CDR3 comprising the sequence:

(SEQ ID NO: 28)
X₃ R G X₄ W E L X₅ D A F D I

wherein X₃ is G or A; X₄ is S or Q; and X₅ is S or T; and
a second VH region that specifically binds to IL-2Rγ, comprising:
   (1) (a) a VH CDR1 comprising the sequence:

(SEQ ID NO: 32)
G F X₁ X₂ X₃ X₄ Y Y

wherein X₁ is T or I; X₂ is F or V; X₃ is S, N, or G; and X₄ is D or N; and
   (b) a VH CDR2 comprising the sequence:

(SEQ ID NO: 33)
I S X₅ S G X₆ X₇ I

wherein X₅ is S or N; X₆ is D, S, G, or N; and X₇ is T or I; and
   (c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).
   E32. A method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 22-25.
   E33. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 80% (e.g., at least 85%, at least 90%, at least 95%; 80%, 85%, 90%, 95%) sequence identity to the VH CDRs 1, 2, and 3 of any one of SEQ ID NOs: 22-25.
   E34. The method according to E32 or E33, wherein:
the full set of VH CDRs 1, 2, and 3 (combined) in the first VH region has at least 85% sequence identity to the VH CDRs 1, 2, and 3 of any one of SEQ ID NOs: 11-14; and/or
the full set of VH CDRs 1, 2, and 3 (combined) in the second VH region has at least 85% sequence identity to the VH CDRs 1, 2, and 3 of any one of SEQ ID NOs: 22-25.
   E35. The method according to any one of E32-E34, wherein:
the full set of VH CDRs 1, 2, and 3 (combined) in the first VH region has at least 90% sequence identity to the VH CDRs 1, 2, and 3 of any one of SEQ ID NOs: 11-14; and/or
the full set of VH CDRs 1, 2, and 3 (combined) in the second VH region has at least 90% sequence identity to the VH CDRs 1, 2, and 3 of any one of SEQ ID NOs: 22-25.
   E36. The method according to any one of E32-E35, wherein:
the full set of VH CDRs 1, 2, and 3 (combined) in the first VH region has at least 95% sequence identity to the VH CDRs 1, 2, and 3 of any one of SEQ ID NOs: 11-14; and/or
the full set of VH CDRs 1, 2, and 3 (combined) in the second VH region has at least 95% sequence identity to the VH CDRs 1, 2, and 3 of any one of SEQ ID NOs: 22-25.
   E37. The method according to any one of E32-E36, wherein:
the full set of VH CDRs 1, 2, and 3 (combined) in the first VH region has at least 98% sequence identity to the VH CDRs 1, 2, and 3 of any one of SEQ ID NOs: 11-14; and/or
the full set of VH CDRs 1, 2, and 3 (combined) in the second VH region has at least 98% sequence identity to the VH CDRs 1, 2, and 3 of any one of SEQ ID NOs: 22-25.
   E38. The method according to any one of E32-37, wherein the first VH region comprises the VH CDR1, VH CDR2, and VH CDR3 of any one of SEQ ID NOs: 11-14.
   E39. The method according to any one of E32-38, wherein the second VH region comprises the VH CDR1, VH CDR2, and VH CDR3 of any one of SEQ ID NOs: 22-25.
   E40. The method according to any one of E32-E37, wherein:
the first VH region comprises the VH CDR1, VH CDR2, and VH CDR3 of any one of SEQ ID NOs: 11-14; and
the second VH region comprises the VH CDR1, VH CDR2, and VH CDR3 of any one of SEQ ID NOs: 22-25.
   E41. The method according to any one of E1-E40, wherein the VH CDR1, VH CDR2, and VH CDR3 sequences of the first VH region are present in a human VH framework.
   E42. The method according to any one of E1-E41, wherein the VH CDR1, VH CDR2, and VH CDR3 sequences of the second VH region are present in a human VH framework.
   E43. A method of treating a cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 80% (e.g., at least 85%, at least 90%, at least 95%; 80%, 85%, 90%, 95%) sequence identity to any one of SEQ ID NOs: 11-14; and
a second VH region that specifically binds to IL-2Rγ, having at least 80% (e.g., at least 85%, at least 90%, at least 95%; 80%, 85%, 90%, 95%) sequence identity to any one of SEQ ID NOs: 22-25.
   E44. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 80% (e.g., at least 85%, at least 90%, at least 95%; 80%, 85%, 90%, 95%) sequence identity to the sequence of any one of SEQ ID NOs: 11-14; and
a second VH region that specifically binds to IL-2Rγ, having at least 80% (e.g., at least 85%, at least 90%, at least 95%; 80%, 85%, 90%, 95%) sequence identity to the sequence of any one of SEQ ID NOs: 22-25.
   E45. The method according to E43 or E44, wherein:
the first VH region has at least 85% sequence identity to the sequence of any one of SEQ ID NOs: 11-14; and/or
the second VH region has at least 85% sequence identity to the sequence of any one of SEQ ID NOs: 22-25.
   E46. The method according to any one of E43-E45, wherein:
the first VH region has at least 90% sequence identity to the sequence of any one of SEQ ID NOs: 11-14; and/or
the second VH region has at least 90% sequence identity to the sequence of any one of SEQ ID NOs: 22-25.
   E47. The method according to any one of E43-E46, wherein:
the first VH region has at least 95% sequence identity to the sequence of any one of SEQ ID NOs: 11-14; and/or
the second VH region has at least 95% sequence identity to the sequence of any one of SEQ ID NOs: 22-25.
   E48. The method according to any one of E43-E47, wherein the first VH region comprises a sequence chosen from any one of SEQ ID NOs: 11-14.
   E49. The method according to any one of E43-E48, wherein the second VH region comprises a sequence chosen from any one of SEQ ID NOs: 22-25.
   E50. The method according to any one of E43-E47, wherein:
the first VH region comprises a sequence chosen from any one of SEQ ID NOs: 11-14; and
the second VH region comprises a sequence chosen from any one of SEQ ID NOs: 22-25.
   E51. The method according to any one of E43-E50, wherein:
the first VH region comprises a sequence having at least 80% (e.g., at least 85%, at least 90%, at least 95%; 80%, 85%, 90%, 95%) sequence identity to the sequence of SEQ ID NO: 11; and
the second VH region comprises a sequence having at least 80% (e.g., at least 85%, at least 90%, at least 95%; 80%, 85%, 90%, 95%) sequence identity to the sequence of SEQ ID NO: 22.
   E52. The method according to any one of E43-E50, wherein:
the first VH region comprises the sequence of SEQ ID NO: 11; and
the second VH region comprises the sequence of SEQ ID NO: 22.
   E53. The method according to any one of E43-E50, wherein:
the first VH region comprises a sequence having at least 80% (e.g., at least 85%, at least 90%, at least 95%; 80%, 85%, 90%, 95%) sequence identity to the sequence of SEQ ID NO: 12; and
the second VH region comprises a sequence having at least 80% (e.g., at least 85%, at least 90%, at least 95%; 80%, 85%, 90%, 95%) sequence identity to the sequence of SEQ ID NO: 23.
   E54. The method according to any one of E43-E50, wherein:
the first VH region comprises the sequence of SEQ ID NO: 12; and the second VH region comprises the sequence of SEQ ID NO: 23.
   E55. The method according to any one of E43-E50, wherein:
the first VH region comprises a sequence having at least 80% (e.g., at least 85%, at least 90%, at least 95%; 80%, 85%, 90%, 95%) sequence identity to the sequence of SEQ ID NO: 12; and
the second VH region comprises a sequence having at least 80% (e.g., at least 85%, at least 90%, at least 95%; 80%, 85%, 90%, 95%) sequence identity to the sequence of SEQ ID NO: 24.
   E56. The method according to any one of E43-E50, wherein:
the first VH region comprises the sequence of SEQ ID NO: 12; and the second VH region comprises the sequence of SEQ ID NO: 24.
   E57. The method according to any one of E43-E50, wherein:
the first VH region comprises a sequence having at least 80% (e.g., at least 85%, at least 90%, at least 95%; 80%, 85%, 90%, 95%) sequence identity to the sequence of SEQ ID NO: 12; and
the second VH region comprises a sequence having at least 80% (e.g., at least 85%, at least 90%, at least 95%; 80%, 85%, 90%, 95%) sequence identity to the sequence of SEQ ID NO: 25.
   E58. The method according to any one of E43-E50, wherein:
the first VH region comprises the sequence of SEQ ID NO: 12; and the second VH region comprises the sequence of SEQ ID NO: 25.
   E59. The method according to any one of E43-E50, wherein:
the first VH region comprises a sequence having at least 80% (e.g., at least 85%, at least 90%, at least 95%; 80%, 85%, 90%, 95%) sequence identity to the sequence of SEQ ID NO: 13; and
the second VH region comprises a sequence having at least 80% (e.g., at least 85%, at least 90%, at least 95%; 80%, 85%, 90%, 95%) sequence identity to the sequence of SEQ ID NO: 23.
   E60. The method according to any one of E43-E50, wherein:
the first VH region comprises the sequence of SEQ ID NO: 13; and the second VH region comprises the sequence of SEQ ID NO: 23.
   E61. The method according to any one of E43-E50, wherein:
the first VH region comprises a sequence having at least 80% (e.g., at least 85%, at least 90%, at least 95%; 80%, 85%, 90%, 95%) sequence identity to the sequence of SEQ ID NO: 14; and
the second VH region comprises a sequence having at least 80% (e.g., at least 85%, at least 90%, at least 95%; 80%, 85%, 90%, 95%) sequence identity to the sequence of SEQ ID NO: 22.
   E62. The method according to any one of E43-E50, wherein:
the first VH region comprises the sequence of SEQ ID NO: 14; and the second VH region comprises the sequence of SEQ ID NO: 22.
   E63. The method according to any one of E1-E62, wherein the anti-IL-2Rβγ heavy chain-only antibody or antigen-binding fragment thereof further comprises a heavy chain constant region sequence in the absence of a CH1 sequence.
   E64. The method according to any one of E1-E63, wherein the anti-IL-2Rβγ heavy chain-only antibody or antigen-binding fragment thereof further comprises a heavy chain constant region comprising a hinge region, a CH2 domain, and a CH3 domain.
   E65. The method according to E64, wherein the hinge region comprises:
a wild type human IgG4 hinge region sequence (SEQ ID NO: 54); or
a variant human IgG4 hinge region sequence comprising an S228P mutation (SEQ ID NO: 55).
   E66. The method according to E64 or E65, wherein the CH2 domain comprises:
a wild type human IgG4 CH2 domain sequence (SEQ ID NO: 56); or
a variant human IgG4 CH2 domain comprising an F234A mutation, an L235A mutation, or both an F234A mutation and an L235A mutation.
   E67. The method according to any one of E64-E66, wherein the CH3 domain comprises:
a wild type human IgG4 CH3 domain sequence (SEQ ID NO: 58); or
a variant human IgG4 CH3 domain sequence comprising a T366W mutation; or
a variant human IgG4 CH3 domain sequence comprising a T366S, an L368A mutation, and a Y407V mutation.
   E68. The method according to any one of E1-E63, wherein the anti-IL-2Rβγ heavy chain-only antibody or antigen-binding fragment thereof further comprises a Fc region.
   E69. The method according to any one of E1-E63, wherein the anti-IL-2Rβγ heavy chain-only antibody or antigen-binding fragment thereof further comprises a variant Fc region.
   E70. The method according to E69, wherein the variant Fc region comprises heterodimerizing alterations.
   E71. The method according to any one of E68-E70, wherein the Fc region is a silenced Fc region.
   E72. The method according to any one of E1-E64, wherein the anti-IL-2Rβγ heavy chain-only antibody is an IgG1 antibody.
   E73. The method according to any one of E1-E64, wherein the anti-IL-2Rβγ heavy chain-only antibody is an IgG4 antibody.
   E74. The method according to any one of E1, E2, E30-E33, E43, or E44, wherein the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 53; and
a second polypeptide comprising a sequence having at least 80% (e.g., at least 85%, at least 90%, at least 95%; 80%, 85%, 90%, 95%) sequence identity to the sequence of SEQ ID NO: 61.
   E75. The method according to E74, wherein:
the first polypeptide comprises the sequence of SEQ ID NO: 53; and the second polypeptide comprises the sequence of SEQ ID NO: 61.
   E76. The method according to any one of E1, E2, E30-E33, E43, or E44, wherein the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 62; and
a second polypeptide comprising a sequence having at least 80% (e.g., at least 85%, at least 90%, at least 95%; 80%, 85%, 90%, 95%) sequence identity to the sequence of SEQ ID NO: 63.
   E77. The method according to E76, wherein:
the first polypeptide comprises the sequence of SEQ ID NO: 62; and
the second polypeptide comprises the sequence of SEQ ID NO: 63.
   E78. The method according to any one of E1, E2, E30-E33, E43, or E44, wherein the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 64; and
a second polypeptide comprising a sequence having at least 80% (e.g., at least 85%, at least 90%, at least 95%; 80%, 85%, 90%, 95%) sequence identity to the sequence of SEQ ID NO: 65.
   E79. The method according to E78, wherein:
the first polypeptide comprises the sequence of SEQ ID NO: 64; and
the second polypeptide comprises the sequence of SEQ ID NO: 65.
   E80. The method according to any one of E1, E2, E30-E33, E43, or E44, wherein the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 66; and
a second polypeptide comprising a sequence having at least 80% (e.g., at least 85%, at least 90%, at least 95%; 80%, 85%, 90%, 95%) sequence identity to the sequence of SEQ ID NO: 67.
   E81. The method according to E80, wherein:
the first polypeptide comprises the sequence of SEQ ID NO: 66; and
the second polypeptide comprises the sequence of SEQ ID NO: 67.
   E82. The method according to any one of E1, E2, E30-E33, E43, or E44, wherein the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 34; and
a second polypeptide comprising a sequence having at least 80% (e.g., at least 85%, at least 90%, at least 95%; 80%, 85%, 90%, 95%) sequence identity to the sequence of SEQ ID NO: 35.
   E83. The method according to E82, wherein:
the first polypeptide comprises the sequence of SEQ ID NO: 34; and
the second polypeptide comprises the sequence of SEQ ID NO: 35.
   E84. The method according to any one of E1, E2, E30-E33, E43, or E44, wherein the anti-IL-2Rβγ heavy chain-only antibody or antigen-binding fragment thereof comprises:
a first polypeptide comprising a sequence having at least 80% (e.g., at least 85%, at least 90%, at least 95%; 80%, 85%, 90%, 95%) sequence identity to the sequence of SEQ ID NO: 36; and
a second polypeptide comprising a sequence having at least 80% (e.g., at least 85%, at least 90%, at least 95%; 80%, 85%, 90%, 95%) sequence identity to the sequence of SEQ ID NO: 37.
   E85. The method according to E84, wherein:
the first polypeptide comprises the sequence of SEQ ID NO: 36; and
the second polypeptide comprises the sequence of SEQ ID NO: 37.
   E86. The method according to any one of E1-E85, wherein:
the anti-IL-2Rβγ heavy chain-only antibody or antigen-binding fragment has an affinity for IL2R with a Kd of from about 10⁻¹¹ M to about 10⁻⁶ M; and/or
the anti-IL-2Rβγ heavy chain-only antibody or antigen-binding fragment has an affinity for IL2RP with a Kd of from about 10⁻⁸ M to about 2.5×10⁻⁷ M; and/or
the anti-IL-2Rβγ heavy chain-only antibody or antigen-binding fragment has an affinity for IL2RT with a Kd of from about 10⁻⁹ M to about 2.5×10⁻⁸ M.
   E87. The method according to any one of E1-E86, wherein the anti-IL-2Rβγ heavy chain-only antibody or antigen-binding fragment functions as an IL-2Rβγ agonist.
   E88. The method according to any one of E1-E87, further comprising administering a premedication to the subject prior to the administration of a first dose of the anti-IL-2Rβγ heavy chain-only antibody or antigen-binding fragment or a first dose of the T-cell redirecting therapy.
   E89. The method according to E88, wherein the premedication is chosen from antihistamines, glucocorticoids, IL-6 receptor antagonists, and tumor necrosis factor alpha (TNF-α) antagonists.
   E90. The method according to any one of E1-E89, wherein the T-cell redirecting therapy is a bispecific T-cell engaging molecule.
   E91. The method according to E90, wherein the bispecific T-cell engaging molecule comprises a first domain that specifically binds to a target cancer cell antigen and a second domain that specifically binds to human CD3.
   E92. The method according to E91, wherein the target cancer cell antigen is chosen from EpCAM, CEA, CD19, CD33, CD70, EGFRvIII, FLT3, GPRC5D, DLL3, BCMA, PSMA, STEAP1, STEAP2, MUC16, MUC17, and CLDN18.2.
   E93. The method according to any one of E90-E92, wherein the bispecific T-cell engaging molecule further comprises a half-life extension domain.
   E94. The method according to E93, wherein the half-life extension domain provides a half-life for the bispecific T-cell engaging molecule of greater than about 24 hours.
   E95. The method according to E93 or E94, wherein the half-life extension domain is chosen from immunoglobulin Fc domains, domains derived from serum albumin (e.g., human serum albumin), albumin-binding domains (e.g., comprising human albumin binding peptides or an antibody fragment that specifically binds to serum albumin), peptides that bind to the neonatal Fc receptor (FcRn), and polyethylene glycol polymers.
   E96. The method according to any one of E90-E92, wherein the bispecific T-cell engaging molecule is a three-chain antibody like molecule.
   E97. The method according to any one of E1-E89, wherein the T-cell redirecting therapy is a chimeric antigen receptor (CAR)-expressing T-cell.
   E98. The method according to E97, wherein the CAR-expressing T-cell comprises a first domain that specifically binds to a target cancer cell antigen, a transmembrane domain, and an intracellular signalling domain.
   E99. The method according to E98, wherein the target cancer cell antigen is chosen from EpCAM, CEA, CD19, CD33, CD70, EGFRvIII, FLT3, GPRC5D, DLL3, BCMA, PSMA, STEAP1, STEAP2, MUC16, MUC17, and CLDN18.2.
   E100. The method according to any one of E1-E99, wherein at least one dose of the anti-IL-2Rβγ heavy chain-only antibody or antigen-binding fragment thereof is administered to the subject prior to a first dose of the T-cell redirecting therapy.
   E101. The method according to any one of E1-E100, wherein the method comprises administering the anti-IL-2Rβγ heavy chain-only antibody or antigen-binding fragment thereof in combination with the T-cell redirecting therapy in one or more treatment cycles.
   E102. The method according to E101, wherein each of the one or more treatment cycles comprises a single dose of the anti-IL-2Rβγ heavy chain-only antibody or antigen-binding fragment thereof and a single dose of the T-cell redirecting therapy.
   E103. The method according to E101, wherein each of the one or more treatment cycles comprises multiple doses of the anti-IL-2Rβγ heavy chain-only antibody or antigen-binding fragment thereof and a single dose of the T-cell redirecting therapy.
   E104. The method according to E101, wherein each of the one or more treatment cycles comprises a single dose of the anti-IL-2Rβγ heavy chain-only antibody or antigen-binding fragment thereof and multiple doses of the T-cell redirecting therapy.
   E105. The method according to E101, wherein each of the one or more treatment cycles comprises multiple doses of the anti-IL-2Rβγ heavy chain-only antibody or antigen-binding fragment thereof and multiple doses of the T-cell redirecting therapy.
   E106. The method according to any one of E1-105, wherein the cancer is a hematologic cancer.
   E107. The method according to any one of E1-106, wherein the cancer is chosen from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.
   E108. The method according to any one of E1-105, wherein the cancer is chosen from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma.
   E109. The method according to E108, wherein the subject has at least one tumor with low immune infiltration (e.g., low or no T-cell infiltration) prior to the co-administration.
   E110. The method according to E108 or E109, wherein the co-administration increases tumor T-cell infiltration.
   E111. The method according to any one of E108-E110, wherein the co-administration is associated with at least one anti-tumor effect.
   E112. The method according to E111, wherein the at least one anti-tumor effect is chosen from a decrease in tumor volume, a decrease in the number of tumor cells, a decrease in tumor cell proliferation, and a decrease in tumor cell survival.
   E113. The method according to any one of E1-E112, wherein the co-administration is associated with at least one anti-cancer effect.
   E114. The method according to E113, wherein the at least one anti-cancer effect is chosen from a decrease in the number of cancer cells, a decrease in the number of metastases, an increase in life expectancy, a decrease in cancer cell proliferation, a decrease in cancer cell survival, and an amelioration of various physiological symptoms associated with the cancerous condition.
   E115. The method according to any one of E1-E114, wherein the anti-IL-2Rβγ heavy chain-only antibody or antigen-binding fragment thereof is administered in a pharmaceutical composition adapted for intravenous or subcutaneous delivery.
   E116. The method according to any one of E1-E115, wherein the T-cell redirecting therapy is administered in a pharmaceutical composition adapted for intravenous or subcutaneous delivery.
   E117. The method according to E116, wherein the pharmaceutical composition comprises the bispecific T-cell engaging molecule, a buffer, a surfactant, and a stabilizing agent.
   E118. The method according to E116 or E117, wherein the pharmaceutical composition comprises the bispecific T-cell engaging molecule, a glutamate buffer, polysorbate 20 or polysorbate 80, and sucrose, at a pH of about 4.0 to about 4.4.
   E119. The method according to any one of E1-E118, wherein the anti-IL-2Rβγ heavy chain-only antibody or antigen-binding fragment thereof and the T-cell redirecting therapy are administered in separate pharmaceutical compositions.
   E120. The method according to E119, wherein the separate pharmaceutical compositions may be lyophilized and reconstituted prior to administration to a patient.
   E121. The method according to any one of E1-E120, wherein the anti-IL-2Rβγ heavy chain-only antibody or antigen-binding fragment thereof and the T-cell redirecting therapy are administered concurrently.
   E122. The method according to any one of E1-E120, wherein the anti-IL-2Rβγ heavy chain-only antibody or antigen-binding fragment thereof and the T-cell redirecting therapy are administered sequentially.
   E123. The method according to any one of E1-E122, wherein the subject was previously administered a first line therapy for the cancer.
   E124. The method according to any one of E1-123, wherein the subject was previously administered a first line therapy and a second line therapy for the cancer.

These and further aspects will be more fully explained in the rest of the disclosure, including in the Examples.

### BRIEF DESCRIPTION OF THE DRAWINGS

FIG. 1 is a table summarizing the binding kinetics of the bispecific heavy chain-only antibody constructs BsAb-1(IL2RB_F09C**IL2RG_F16A), BsAb-2 (IL2RB_F09G**IL2RG_F16B), BsAb-3 (TL2RB_F09G**IL2RG_F16C), BsAb-4 (IL2RB_F09G**IL2RG_F18A), BsAb-5 (TL2RB_F09K**IL2RG F16B), BsAb-6 (IL2RB_F18E**IL2RG_F16A) with respect to human and cynomolgus IL2RB and IL2RG.

FIG. 2, panels A-C, are a series of heatmap tables depicting fold-induction of phosphorylated STAT5 (pSTAT5) in CD8⁺ T-cells from human PBMCs treated with: anti-IL2β/γ bispecific heavy chain-only antibodies (panel A); anti-IL2Rβ and anti-L2Rγ monospecific heavy chain-only antibodies in a 1:1 mixture or as single agents (panel B); or IL-2 as a control (panel C) at 50 nM for 1 hour. pSTAT5 levels were determined by flow cytometry and reported as geometric mean fluorescent intensity (gMFI) over the gMFI of unstimulated cells.

FIG. 3, panels A-C, are a series of graphs showing cell binding of the indicated cell type as a function of concentration for the depicted bispecific heavy-chain only antibody constructs. Cell binding was determined by flow cytometry and reported as geometric mean fluorescent intensity (gMFI) over the gMFI of cells stained only with secondary detection antibody.

FIG. 4, panels A-E, are a series of graphs showing STAT5 phosphorylation dose curves in human and cyno PBMCs as a function of concentration for the depicted bispecific heavy chain-only antibody constructs and control molecules (IL-2 and IL-2 variant). pSTAT5 levels were determined by flow cytometry and reported as a percentage of the indicated cell type.

FIG. 5, panels A-D, are a series of graphs showing proliferation (Ki67 dose curves) in the indicated human cells as a function of concentration for the depicted bispecific heavy chain-only antibody constructs and control molecules (IL-2 and IL-2 variant). Ki67 levels were determined by flow cytometry and were reported as a percentage of the indicated cell type.

FIG. 6, panels A-D, are a series of graphs showing cytokine secretion in human whole blood as a function of concentration for the depicted bispecific antibody constructs and control molecules (IL-2).

FIG. 7, panels A-B, provide cellular internalization data for the indicated bispecific heavy chain-only antibody constructs. Panel A depicts internalization of the indicated anti-IL2β/γ heavy chain-only antibodies by CD8⁺ T-cells from human PBMCs, as a function of time. Panel B depicts this data in tabular format. Surface levels of heavy chain-only antibody were detected by flow cytometry and reported relative to cells which had not been allowed to internalize. The observed half-lives ranged from 0.27 hours to 0.81 hours. As observed here, internalization was potentially partially dependent on the specific anti-IL2RG arm of the bispecific heavy chain-only antibody, as molecules comprising the IL2RG_F16B binding sequence internalized faster, and to a greater degree, than molecules containing different anti-TL2RG binding sequences.

FIG. 8, panels A-B, provide mouse model PK data in graphical (panel A) and tabular (panel B) formats. BALB/c mice (n=3 per group per time point) were administered 1 mg/kg of the indicated anti-IL2β/γ heavy chain-only antibodies by tail-vein injection. Serum was collected at 6 time points over two weeks and tested together by ELISA for human IgG4. Results are shown as a function of time (panel A) or in tabular format (panel B).

FIG. 9 is a table summarizing several properties of the indicated bispecific heavy chain-only antibody constructs. All constructs were expressed in an ExpiCHO expression system and were 2-step purified. Stability was determined based on percent aggregation by SE-HPLC after thermal stress. Tm and Tagg were measured using the UNcle platform. For SE-HPLC experiments, 20 μg of protein was run on TSK gel G3000 5 μm column.

FIG. 10, panels A-C, provide summary data from a mouse model of GVHD. Irradiated NSG mice (5 per treatment group) were engrafted with 20 million human PBMCs each. Animals were then treated with either vehicle only (100 μL), 22 μg rhIL-2 daily, or one of the two indicated bispecific antibody constructs at 1 mg/kg in 100 μL twice a week until sacrifice (20% body weight loss). Panel A provides an overview of the mouse model of GVHD and subsequent dosing scheme. Panel B shows animal body weights as a function of time for the indicated experimental groups. Panel C depicts an analysis of cells from spleens of the mice in the study, harvested after day 5 of treatment. Proliferation of CD8⁺ T-cells and CD4⁺ T-cells was compared between the 4 treatment groups by measuring CSFE staining in the different lymphocyte populations. The two tested bispecific heavy chain-only antibody constructs (IL2RB_F09C**IL2RG_F16A (BsAb-1) and IL2RB_F09G**IL2RG_F16B (BsAb-2)) both showed significantly more proliferating CD8⁺ T-cells compared to rhIL-2 and the vehicle control. CD4⁺ T-cells were expanded to a lesser extent; however, a significant increase in proliferating CD4⁺ T-cells was seen in IL2RB_F09G**IL2RG_F16B (BsAb-2)-treated mice compared to the vehicle control (panel C). The data demonstrate that cytokine receptor agonists promote immune effector activation and proliferation in vivo and accelerate GVHD in huPBMC-engrafted NSG mice at a rate similar to cytokine controls.

FIG. 11, panels A-J, are a series of graphs summarizing in vivo pharmacodynamic (PD) data from a non-GLP cynomolgus monkey study. Panels A-E depict the percentages of the indicated cell types as a function of time post dose. Panels F-J depict the concentration of the indicated cell types per μL of blood (x10⁵) as a function of time post dose. Panel K shows the ratio of CD8+ T-cells to CD4+ T-cells as a function of time post dose.

FIG. 12, Panel A is a graph showing serum concentration as a function of time (days) for the indicated bispecific heavy chain-only antibody. Panel B is a table showing molecule, dose and half life (t_{1/2}) information.

FIG. 13 is a graph showing body weight (%) as a function of time (study day) for animals in an accelerated GVHD model.

FIG. 14 is a graph showing probability of survival as a function of time (study day) for animals in an accelerated GVHD model.

FIG. 15, panels A-C, are a collection of graphs showing cell proliferation of the indicated cell type, measured at 5 days post-treatment, and separated into treatment groups. Panel A shows results for CD8+ T-cells, Panel B shows results for CD4+ T-cells, and Panel C shows results for NK-cells.

FIG. 16, panels A-L, are a collection of graphs showing cell proliferation and absolute cell concentration for the indicated cell types under the indicated dosing conditions, as a function of time.

FIG. 17, panels A and B, show results for a TDCC assay (E:T=1:1, 72 hour assay time) using H82_Luc cells investigating cytotoxicity for a bispecific T-cell engaging molecule that specifically binds to human CD3 and human EpCAM (TCE-1) alone and in combination with an anti-IL2β/γ heavy chain-only antibody (BsAb-5).

FIG. 18, panels A-C, show results for a TDCC assay (E:T=1:1, 1:3, 1:10, 72 hour assay time) using SHP77 Luc cells investigating TDCC activity for a bispecific T-cell engaging molecule that specifically binds to human CD3 and human EpCAM (TCE-1) alone and in combination with an anti-IL2β/γ heavy chain-only antibody (BsAb-5).

FIG. 19 is a schematic showing the design of an in vivo combination study in a NSG mouse model.

FIG. 20 is a graph showing tumor volume as a function of time between 11 and 31 days post tumor implantation for mice in an in vivo combination study.

FIG. 21 is a graph showing relative body weight (%) as a function of time between 11 and 31 days post tumor implantation for mice in an in vivo combination study.

FIG. 22, panels A-C, show results from an in vivo tumor pharmacodynamics study.

### DETAILED DESCRIPTION

The practice of the present disclosure will employ, unless otherwise indicated, conventional techniques of molecular biology (including recombinant techniques), microbiology, cell biology, biochemistry, and immunology, which are within the skill of the ordinary artisan. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

Unless indicated otherwise, antibody residues herein are numbered according to the Kabat numbering system (e.g., Kabat et al., Sequences of Immunological Interest. 5th Ed. Public Health Service, National Institutes of Health, Bethesda, Md. (1991)).

In the following description, numerous specific details are set forth to provide a more thorough understanding of the present disclosure. However, it will be apparent to one of skill in the art that the present disclosure may be practiced without one or more of these specific details. In other instances, well-known features and procedures well known to those skilled in the art have not been described in order to avoid obscuring the disclosure.

All references cited throughout the disclosure, including patent applications and publications, are incorporated by reference herein in their entirety. Where there is any discrepancy in definition between the cited references and the definitions provided herein, the definitions provided herein control.

### Definitions

In some embodiments, “about,” when used in connection with a measurable numerical variable, refers to the indicated value of the variable and to all values of the variable that are within the experimental error of the indicated value (e.g., within the 95% confidence interval for the mean) or +10% of the indicated value, whichever is greater. In some embodiments, numeric ranges are inclusive of the numbers defining the range (i.e., the endpoints).

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges also encompassed within the disclosure, subject to any specifically excluded limit in the stated range. Where the stated range includes one or both of the limits, ranges excluding either or both of those included limits are also included in the disclosure.

As used herein, the terms “a” and “an” mean “one or more” unless specifically indicated otherwise. Additionally, “one or more” and “at least one” are used interchangeably herein. Furthermore, unless otherwise required by context, singular terms include pluralities and plural terms include the singular.

As used herein, the term “polypeptide” refers to a polymer of amino acids comprising at least 50 amino acids, such as, e.g., at least 100 amino acids.

As used herein, the term “antibody” generally refers to a tetrameric immunoglobulin protein comprising two light chain polypeptides (such as, e.g., light chain polypeptides that are about 25 kDa each) and two heavy chain polypeptides (such as, e.g., heavy chain polypeptides that are about 50-70 kDa each). The term “light chain” or “immunoglobulin light chain,” as used herein, refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (k) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgAQ1 and IgA2, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e., between the light and heavy chain) and between the hinge regions of the two antibody heavy chains. In some embodiments, antibodies of the present disclosure are human antibodies or humanized antibodies and can be of the IgG1-, IgG2-, IgG3-, or IgG4-type.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., MSLN or CD3). From N-terminus to C-terminus, naturally-occurring light and heavy chain variable regions both typically conform with the following order of these elements: FR1, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT© (the international ImMunoGeneTics information system; Lefranc et al., Dev. Comp. Immunol. 29:185-203; 2005) and AHo (Honegger and Pluckthun, J. Mol. Biol. 309(3):657-670; 2001).

As used herein, “CDR” means a complementarity-determining region of an antibody as defined in Lefranc, M P et al., IMGT, the International ImMunoGeneTics database, Nucleic Acids Res., 27:209-212 (1999). However, one of skill in the art will understand that a number of definitions of the CDRs are commonly in use, including the Kabat definition (see Zhao et al. “A germline knowledge based computational approach for determining antibody complementarity determining regions.” Mol Immunol. 2010; 47:694-700), which is based on sequence variability and is the most commonly used. The Chothia definition is based on the location of the structural loop regions (Chothia et al. “Conformations of immunoglobulin hypervariable regions.” Nature. 1989; 342:877-883). Alternative CDR definitions of interest include, without limitation, those disclosed by Honegger, “Yet another numbering scheme for immunoglobulin variable domains: an automatic modeling and analysis tool.” J Mol Biol. 2001; 309:657-670; Ofran et al. “Automated identification of complementarity determining regions (CDRs) reveals peculiar characteristics of CDRs and B-cell epitopes.” J Immunol. 2008; 181:6230-6235; Almagro “Identification of differences in the specificity-determining residues of antibodies that recognize antigens of different size: implications for the rational design of antibody repertoires.” J Mol Recognit. 2004; 17:132-143; and Padlanet al. “Identification of specificity-determining residues in antibodies.” Faseb J. 1995; 9:133-139., each of which is herein specifically incorporated by reference.

“Framework Region” or “FR” residues are those variable domain residues other than the hypervariable region/CDR residues as herein defined.

Antibody residues herein are numbered according to the Kabat numbering system and the EU numbering system. The Kabat numbering system is generally used when referring to a residue in the variable domain (approximately residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies mean residue numbering by the Kabat numbering system. Unless stated otherwise herein, references to residue numbers in the constant domain of antibodies, single domain antibodies, antibody fragments, and the like mean residue numbering by the EU numbering system.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. In contrast to polyclonal antibody preparations which typically include different antibodies directed against different determinants (epitopes), a monoclonal antibody is generally directed against a single determinant on the antigen. As non-limiting examples, monoclonal antibodies in accordance with the present disclosure can be made by the hybridoma method first described by Kohler et al. (1975) Nature 256:495, and can also be made via recombinant protein production methods (see, e.g., U.S. Pat. No. 4,816,567).

The term “human antibody,” as used herein, is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences that are derived from the germline of another mammalian species, such as, e.g., a mouse, have been grafted onto human framework sequences.

As used herein, the term “chimeric antibody” refers to an antibody comprising amino acid sequences from at least two different Ig loci, e.g., a transgenic antibody comprising a portion encoded by a human Ig locus and a portion encoded by a rat Ig locus. Chimeric antibodies include transgenic antibodies with non-human Fc-regions or artificial Fc-regions, and human idiotypes. Such immunoglobulins can be isolated from animals of the disclosure that have been engineered to produce such chimeric antibodies.

As used herein, the term “antibody construct” refers to a molecule in which the structure and/or function is/are based on the structure and/or function of an antibody, e.g., of a full-length immunoglobulin molecule. Hence, an antibody construct immunospecifically binds to its target or antigen, and/or it comprises the heavy chain variable region (VH) and/or the light chain variable region (VL) of an antibody, or comprises domains derived therefrom.

As used herein, an “antibody fragment” generally refers to a fragment of a full-length antibody or heavy chain-only antibody, such as, e.g., VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain) or a modified fragment of a full-length antibody, such as, e.g., triple-chain antibody-like molecule, heavy-chain only antibody, single-chain variable fragment (scFv), di-scFv or bi(s)-scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains.

As used herein, the term “heavy chain-only antibody” refers to an immunoglobulin protein consisting of two heavy chain polypeptides (such as, e.g., heavy chain polypeptides that are about 50-70 kDa each). A “heavy chain-only antibody” is an antibody fragment that lacks the two light chain polypeptides found in a conventional antibody. Heavy-chain antibodies constitute about-one fourth of the IgG antibodies produced by the camelids, e.g., camels and llamas (Hamers-Casterman C., et al. Nature. 363, 446-448 (1993)). These antibodies are formed by two heavy chains but are devoid of light chains. As a consequence, the variable antigen binding part is referred to as the VHH domain, and it represents the smallest naturally occurring, intact, antigen-binding site, being only around 120 amino acids in length (Desmyter, A., et al. J. Biol. Chem. 276, 26285-26290 (2001)). Heavy chain antibodies with a high specificity and affinity can be generated against a variety of antigens through immunization (van der Linden, R. H., et al. Biochim. Biophys. Acta. 1431, 37-46 (1999)), and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments (Ghahroudi, M. A. et al. FEBS Lett. 414, 521-526 (1997)). Sharks have also been shown to have a single VH-like domain in their antibodies, termed VNAR. (Nuttall et al. Eur. J. Biochem. 270, 3543-3554 (2003); Nuttall et al. Function and Bioinformatics 55, 187-197 (2004); Dooley et al., Molecular Immunology 40, 25-33 (2003).)

In some embodiments, a “heavy chain-only antibody” is a homodimeric antibody comprising a VH antigen-binding domain and the CH2 and CH3 constant domains, in the absence of the CH1 domain. In some embodiments, a heavy chain-only antibody is composed of a variable region antigen-binding domain composed of framework 1, CDR1, framework 2, CDR2, framework 3, CDR3, and framework 4. In some embodiments, a heavy chain-only antibody is composed of an antigen-binding domain, at least part of a hinge region, and CH2 and CH3 domains. In some embodiments, a heavy chain-only antibody is composed of an antigen-binding domain, at least part of a hinge region, and a CH2 domain. In some embodiments, a heavy chain-only antibody is composed of an antigen-binding domain, at least part of a hinge region, and a CH3 domain. Heavy chain-only antibodies in which the CH2 and/or CH3 domain is truncated are also included herein. The heavy chain-only antibodies described herein may belong to the IgG subclass, but heavy chain-only antibodies belonging to other subclasses, such as IgM, IgA, IgD and IgE subclass, are also included herein. In some embodiments, a heavy chain-only antibody may belong to the IgG1, IgG2, IgG3, or IgG4 subtype, e.g., the IgG1 or IgG4 subtype. In some embodiments, a heavy chain antibody-only is of the IgG1 or IgG4 subtype, wherein one or more of the CH domains is modified to alter an effector function of the antibody. In some embodiments, a heavy chain-only antibody is of the IgG4 subtype, wherein one or more of the CH domains is modified to alter an effector function of the antibody. In some embodiments, a heavy chain-only antibody is of the IgG1 subtype, wherein one or more of the CH domains is modified to alter an effector function of the antibody. Modifications of CH domains that alter effector function are further described herein. Non-limiting examples of heavy-chain-only antibodies are described, for example, in WO2018/039180, the disclosure of which is incorporated herein by reference herein in its entirety.

As used herein, a “single domain antibody” refers to a single polypeptide chain that contains all or part of the heavy chain variable domain or all or part of the light chain variable domain of an antibody. In some embodiments, the single domain antibody is a human single domain antibody.

As used herein, the term “three-chain antibody like molecule” or “TCA” refers to antibody-like molecules comprising, consisting essentially of, or consisting of three polypeptide subunits, two of which comprise, consist essentially of, or consist of one heavy and one light chain of a monoclonal antibody, or antigen-binding fragments of such antibody chains, comprising an antigen-binding region and at least one CH domain. This heavy chain/light chain pair has binding specificity for a first antigen. The third polypeptide subunit comprises, consists essentially of, or consists of a heavy-chain only antibody comprising an Fc portion comprising CH2 and/or CH3 and/or CH4 domains, in the absence of a CH1 domain, and one or more antigen binding domains (such as, e.g., two antigen binding domains) that binds an epitope of a second antigen or a different epitope of the first antigen, where such binding domain is derived from or has sequence identity with the variable region of an antibody heavy or light chain. Parts of such variable region may be encoded by V_{H} and/or V_{L} gene segments, D and JH gene segments, or J_{L} gene segments. The variable region may be encoded by rearranged V_{H}DJ_{H}, V_{L}DJ_{H}, V_{H}J_{L}, or V_{L}J_{L} gene segments.

As used herein, an “antigen-binding fragment” is a portion of an antibody or a heavy chain-only antibody that lacks at least some of the amino acids present in a full-length heavy chain (in the case of an antibody or heavy chain-only antibody) and/or light chain (in the case of an antibody), but which is still capable of specifically binding to an antigen. An antigen-binding fragment includes, but is not limited to, a single-chain variable fragment (scFv), a nanobody (e.g. V_{H} domain of camelid heavy chain antibodies; VHH fragment, see Cortez-Retamozo et al., Cancer Research, Vol. 64:2853-57, 2004), a Fab fragment, a Fab′ fragment, a F(ab′)₂ fragment, a Fv fragment, a Fd fragment, and a CDR fragment, and can be derived from any mammalian source, such as human, mouse, rat, rabbit, or camelid.

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CH1 domain and variable region (VH) of one immunoglobulin heavy chain. The heavy chain of a Fab molecule cannot form a disulfide bond with another heavy chain molecule. The “Fd fragment” comprises the VH and CH1 domains from an immunoglobulin heavy chain. The Fd fragment represents the heavy chain component of the Fab fragment.

The “Fc fragment” or “Fc region” of an immunoglobulin generally comprises two constant domains, a CH2 domain and a CH3 domain, and optionally comprises a CH4 domain. The Fc region may be an Fc region from an IgG1, IgG2, IgG3, or IgG4 immunoglobulin. In some embodiments, the Fc region comprises CH2 and CH3 domains from a human IgG1 or human IgG2 immunoglobulin. The Fc region may retain effector function, such as C1q binding, complement dependent cytotoxicity (CDC), Fc receptor binding, antibody-dependent cell-mediated cytotoxicity (ADCC), and phagocytosis. In other embodiments, the Fc region may be modified to reduce or eliminate effector function.

A “functional Fc region” possesses an “effector function” of a native-sequence Fc region. Non-limiting examples of effector functions include C1q binding, CDC; Fc-receptor binding, ADCC, ADCP, down-regulation of cell-surface receptors (e.g., B-cell receptor), etc. Such effector functions generally require the Fc region to interact with a receptor, such as, e.g., the FcγRI; FcγRIIA; FcγRIIB1; FcγRIIB2; FcγRIIIA; FcγRIIIB receptors, and the low affinity FcRn receptor; and can be assessed using various assays known in the art.

A “dead” or “silenced” Fc is one that has been mutated to retain activity with respect to, for example, prolonging serum half-life, but which does not activate a high affinity Fc receptor, or which has a reduced affinity to an Fc receptor.

A “native-sequence Fc region” comprises an amino acid sequence identical to the amino acid sequence of an Fc region found in nature. Native-sequence human Fc regions include, for example, a native-sequence human IgG1 Fc region (non-A and A allotypes); native-sequence human IgG2 Fc region; native-sequence human IgG3 Fc region; and native-sequence human IgG4 Fc region, as well as naturally occurring variants thereof.

A “variant Fc region” comprises an amino acid sequence that differs from that of a native-sequence Fc region by virtue of at least one amino acid modification, for example, one or more (e.g., two or more, three or more, four or more) amino acid substitution(s). Illustratively, in some embodiments, the variant Fc region has at least one amino acid substitution compared to a native-sequence Fc region or to the Fc region of a parent polypeptide, e.g., from about one to about ten amino acid substitutions, e.g., from about one to about five amino acid substitutions in a native-sequence Fc region or in the Fc region of the parent polypeptide. In some embodiments, the variant Fc region herein will possess at least about 80% homology with a native-sequence Fc region and/or with an Fc region of a parent polypeptide, e.g., at least about 85% homology therewith, e.g., at least about 90% homology therewith, e.g., at least about 95% homology therewith, e.g., at least about 99% homology therewith.

As used herein, “heterodimerizing alterations” refer to alterations in the A and B chains of an Fc region (i.e., the two chains comprising the Fc region, wherein one chain is referred to herein as the “A” chain and the other is referred to herein as the “B” chain) that facilitate the formation of heterodimeric Fc regions, that is, Fc regions in which the A chain and the B chain of the Fc region do not have identical amino acid sequences. In some embodiments, heterodimerizing alterations can be asymmetric, that is, an A chain having a certain alteration can pair with a B chain having a different alteration. These alterations facilitate heterodimerization and disfavor homodimerization. Whether hetero- or homo-dimers have formed can be assessed, for example, by size differences as determined by polyacrylamide gel electrophoresis in situations where one polypeptide chain is a dummy Fc and the other is an scFv-Fc. One non-limiting example of such paired heterodimerizing alterations are the so-called “knobs and holes” substitutions. See, e.g., U.S. Pat. No. 7,695,936 and U.S. Patent Application Publication No. 2003/0078385. As used herein, an Fc region that comprises one pair of knobs and holes substitutions, comprises one substitution in the A chain and another in the B chain. For example, the following knobs and holes substitutions in the A and B chains of an IgG1 Fc region have been found to increase heterodimer formation as compared with that found with unmodified A and B chains and may be employed in non-limiting embodiments of this disclosure: 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. Alternatively or in addition to such alterations, substitutions creating new disulfide bridges can facilitate heterodimer formation. See, e.g., U.S. Patent Application Publication No. 2003/0078385. Such alterations in an IgG1 Fc region include, but are not limited to, the following substitutions: Y349C in one Fc polypeptide chain and S354C in the other; Y349C in one Fc polypeptide chain and E356C in the other; Y349C in one Fc polypeptide chain and E357C in the other; L351C in one Fc polypeptide chain and S354C in the other; T394C in one Fc polypeptide chain and E397C in the other; or D399C in one Fc polypeptide chain and K392C in the other. Additionally or alternatively, substitutions changing the charge of a one or more residue(s), for example, in the CH3-CH3 interface, can enhance heterodimer formation, as described, for example, in WO 2009/089004, which is incorporated by reference herein. Such substitutions are referred to herein as “charge pair substitutions,” and an Fc region comprising one pair of charge pair substitutions comprises one substitution in the A chain and a different substitution in the B chain. Non-limiting examples of charge pair substitutions include the following: 1) K409D or K409E in one chain plus D399K or D399R in the other; 2) K392D or K392E in one chain plus D399K or D399R in the other; 3) K439D or K439E in one chain plus E356K or E356R in the other; and 4) K370D or K370E in one chain plus E357K or E357R in the other. In addition, the substitutions R355D, R355E, K360D, or K360R in both chains can stabilize heterodimers when used with other heterodimerizing alterations. Specific charge pair substitutions can be used either alone or with other charge pair substitutions. Specific examples of single pairs of charge pair substitutions and combinations thereof include the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. Any of these heterodimerizing alterations can be used in polypeptides comprising variant Fc regions as described herein.

In some non-limiting embodiments, variant Fc sequences may include three amino acid substitutions in the CH2 region to reduce FcγRI binding at EU index positions 234, 235, and 237 (see Duncan et al., (1988) Nature 332:563). Two amino acid substitutions in the complement C1q binding site at EU index positions 330 and 331 reduce complement fixation (see Tao et al., J. Exp. Med. 178:661 (1993) and Canfield and Morrison, J. Exp. Med. 173:1483 (1991)). Substitution into human IgG1 or IgG2 residues at positions 233-236 and IgG4 residues at positions 327, 330 and 331 greatly reduces ADCC and CDC (see, for example, Armour KL. et al., 1999 Eur J Immunol. 29(8):2613-24; and Shields R.L. et al., 2001. J Biol Chem. 276(9):6591-604). The human IgG4 Fc amino acid sequence (UniProtKB No. P01861) is provided herein as SEQ ID NO: 43. Silenced IgG1 is described, for example, in Boesch, A. W., et al., “Highly parallel characterization of IgG Fc binding interactions.” MAbs, 2014. 6(4): p. 915-27, the disclosure of which is incorporated herein by reference in its entirety.

Other Fc variants are possible, including, without limitation, one in which a region capable of forming a disulfide bond is deleted, or in which certain amino acid residues are eliminated at the N-terminal end of a native Fc, or a methionine residue is added thereto. Thus, in some embodiments, one or more Fc portions of an antibody can comprise one or more mutations in the hinge region to eliminate disulfide bonding. In yet another embodiment, the hinge region of an Fc can be removed entirely. In still another embodiment, an antibody can comprise an Fc variant.

Further, an Fc variant can be constructed to remove or substantially reduce effector functions by substituting (mutating), deleting, or adding amino acid residues to effect complement binding or Fc receptor binding. For example, and not by way of limitation, a deletion may occur in a complement-binding site, such as a Clq-binding site. Techniques for preparing such sequence derivatives of the immunoglobulin Fc fragment are disclosed in International Patent Publication Nos. WO 97/34631 and WO 96/32478. In addition, the Fc domain may be modified by phosphorylation, sulfation, acylation, glycosylation, methylation, farnesylation, acetylation, amidation, and the like.

Antibodies and antibody fragments with reduced effector function include, but are not limited to, those with substitution of one or more of Fc region residues 238, 265, 269, 270, 297, 327 and 329 according to EU numbering (see, e.g., U.S. Pat. No. 6,737,056). In some embodiments, variant Fc regions with reduced effector function comprise substitutions at two or more of amino acid positions 265, 269, 270, 297 and 327 according to EU numbering, including the so-called “DANA” Fc mutant with substitution of residues 265 and 297 to alanine according to EU numbering (i.e., D265A and N297A according to EU numbering) (see, e.g., U.S. Pat. No. 7,332,581). In some embodiments, the variant Fc region with reduced effector function comprises the following two amino acid substitutions: D265A and N297A.

In some embodiments, effector function is reduced through a mutation in a constant region that eliminates glycosylation, e.g., an “effector-less mutation.” In some embodiments, the effector-less mutation is an N297A or a DANA mutation (D265A+N297A) in the CH2 region. Shields et al., J. Biol. Chem. 276 (9): 6591-6604 (2001). In some embodiments, the effector-less mutation is an N297G or a DANG mutation (D265A+N297G) in the CH2 region. In some embodiments, the variant Fc region lacks glycosylation at N297, e.g., the variant Fc region is a variant Fc region lacking glycosylation at N297 as described in International Patent Publication No. WO 2014/153063, which is incorporated by reference herein. Alternatively, additional mutations resulting in reduced or eliminated effector function include: K322A and L234A/L235A (LALA). Alternatively, effector function can be reduced or eliminated through production techniques, such as expression in host cells that do not glycosylate (e.g., E. coli) or in host cells which result in an altered glycolsylation pattern that is ineffective or less effective at promoting effector function (e.g., Shinkawa et al., J. Biol. Chem. 278(5): 3466-3473 (2003)).

In some embodiments, the proline at position 329 (EU numbering) (P329) of a wild-type human Fc region is substituted with glycine or arginine or an amino acid residue large enough to destroy the proline sandwich within the Fc/Fc7 receptor interface, that is formed between the P329 of the Fc and tryptophan residues W87 and Wi 10 of FcgRIII (Sondermann et al., Nature 406, 267-273 (20 Jul. 2000)). In some further embodiments, at least one further amino acid substitution in the Fc variant region is S228P, E233P, L234A, L235A, L235E, N297A, N297D, or P331S. In some embodiments, the at least one further amino acid substitution is L234A and L235A of the human IgG1 Fc region or S228P and L235E of the human IgG4 Fc region, all according to EU numbering (see, e.g., U.S. Pat. No. 8,969,526, which is incorporated by reference in its entirety).

In some embodiments, the variant Fc region has P329 of the human IgG Fc region substituted with glycine, wherein the variant Fc region comprises at least two further amino acid substitutions at L234A and L235A of the human IgG1 Fc region or S228P and L235E of the human IgG4 Fc region, and wherein the residues are numbered according to the EU numbering (see, e.g., U.S. Pat. No. 8,969,526). In some embodiments, the variant Fc region comprising the P329G, L234A and L235A (EU numbering) substitutions exhibits a reduced affinity to the human FcγRIIIA and FcγRIIA.

In some embodiments, the variant Fc region comprises a triple mutation: an amino acid substitution at position P329, a L234A, and a L235A mutation according to EU numbering (P329/LALA) (see, e.g., U.S. Pat. No. 8,969,526). In some embodiments, the variant Fc region comprises the following amino acid substitutions: P329G, L234A, and L235A according to EU numbering.

In some embodiments, an antibody or antibody fragment comprises a variant human IgG4 CH3 domain sequence comprising a T366W mutation, which can optionally be referred to herein as an IgG4 CH3 knob sequence. In some embodiments, an antibody or antibody fragment comprises a variant human IgG4 CH3 domain sequence comprising a T366S mutation, an L368A mutation, and a Y407V mutation, which can optionally be referred to herein as an IgG4 CH3 hole sequence. The IgG4 CH3 mutations described herein can be utilized in any suitable manner so as to place a “knob” on a first heavy chain constant region of a first monomer in an antibody dimer, and a “hole” on a second heavy chain constant region of a second monomer in an antibody dimer, thereby facilitating proper pairing (heterodimerization) of the desired pair of heavy chain polypeptide subunits in the antibody.

In some embodiments, an antibody or antibody fragment comprises a heavy chain polypeptide subunit comprising a variant human IgG4 Fc region comprising an S228P mutation, an F234A mutation, an L235A mutation, and a T366W mutation (knob). In some embodiments, an antibody or antibody fragment comprises a heavy chain polypeptide subunit comprising a variant human IgG4 Fc region comprising an S228P mutation, an F234A mutation, an L235A mutation, a T366S mutation, an L368A mutation, and a Y407V mutation (hole).

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks, and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only having the structure H₂L₂ (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276:26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure.

Antibodies and antibody fragments (such as, e.g., heavy chain-only antibodies and three-chain antibody like molecules) of the present disclosure include multi-specific antibodies and antibody fragments, which are antibodies and antibody fragments having more than one binding specificity. As used herein, the term “multi-specific” includes “bispecific” (i.e., two binding specificities) and “trispecific” (i.e., three binding specificities), as well as higher-order independent specific binding affinities, such as higher-order polyepitopic specificity.

As used herein, an “isolated” molecule (such as, e.g., an antibody, antibody fragment, single domain antibody) is a molecule which has been identified and separated and/or recovered from a component of its natural environment. Contaminant components of its natural environment are materials which may interfere with diagnostic or therapeutic uses for the molecule, such as, e.g., enzymes, hormones, and other proteinaceous or nonproteinaceous solutes. In some embodiments, the isolated molecule will be purified (1) to greater than 95% by weight of the molecule as determined by the Lowry method, such as, e.g., more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, e.g., silver stain. In some embodiments, an isolated molecule will be prepared by a process comprising at least one purification step.

Aspects of the present disclosure include antibodies and antibody fragments (e.g., heavy chain-only antibodies) comprising a heavy chain-only variable region in a monovalent or bivalent configuration. As used herein, the term “monovalent configuration”, as used in reference to a heavy chain-only variable region domain, means that only one heavy chain-only variable region domain is present, having a single binding site. In contrast, the term “bivalent configuration” as used in reference to a heavy chain-only variable region domain means that two heavy chain-only variable region domains are present (each having a single binding site), and are connected by a linker sequence. Non-limiting examples of linker sequences are discussed further herein, and include, without limitation, GS linker sequences of various lengths. When a heavy chain-only variable region is in a bivalent configuration, each of the two heavy chain-only variable region domains can bind to the same antigen, or to different antigens (e.g., to different epitopes on the same protein; to two different proteins, etc.). However, unless specifically noted otherwise, a heavy chain-only variable region denoted as being in a “bivalent configuration” is understood to contain two identical heavy chain-only variable region domains, connected by a linker sequence, wherein each of the two identical heavy chain-only variable region domains binds to the same target antigen.

Aspects of the present disclosure also include antibodies and antibody fragments (e.g., heavy chain-only antibodies) having multi-specific configurations, which include, without limitation, bispecific, trispecific, etc. configurations. A large variety of methods and protein configurations are known and used in bispecific monoclonal antibodies (BsMAB), tri-specific antibodies, etc.

Various methods for the production of multivalent artificial antibodies have been developed by recombinantly fusing variable domains of two or more antibodies. In some embodiments, a first and a second antigen-binding domain on a polypeptide are connected by a polypeptide linker. One non-limiting example of such a polypeptide linker is a GS linker, having an amino acid sequence of four glycine residues, followed by one serine residue, and wherein the sequence is repeated n times, where n is an integer ranging from 1 to about 10 (SEQ ID NO: 68), such as 2, 3, 4, 5, 6, 7, 8, or 9. Non-limiting examples of such linkers include GGGGS (SEQ ID NO: 49) (n=1) and GGGGSGGGGS (SEQ ID NO: 50) (n=2). Other suitable linkers can also be used, and are described, for example, in Chen et al., Adv Drug Deliv Rev. 2013 Oct. 15; 65(10): 1357-69, the disclosure of which is incorporated herein by reference in its entirety.

As used herein, the term “amino acid” or “amino acid residue” refers to an amino acid having its art recognized definition, such as, e.g., an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

As used herein, “amino acid modifications” include, but are not limited to, deletions from, and/or insertions into, and/or substitutions of, residues within an amino acid sequence. Any combination of deletion, insertion, and substitution may be made to arrive at a final construct, provided that the final construct possesses the desired characteristics. The amino acid changes also may alter post-translational processes of the antibody constructs, such as changing the number or position of glycosylation sites. Non-limiting example substitutions (or replacements) are conservative substitutions. However, any substitution (including non-conservative substitutions) is envisaged as long as the final construct retains its capability to bind to the target antigen.

One of skill in the art will realize that conservative variants of the antibodies, antibody fragments, and antigen-binding fragments thereof described herein can be produced. Such conservative variants employed in antibody fragments, such as dsFv fragments or in scFv fragments, will retain critical amino acid residues necessary for correct folding and stabilizing between the V_{H} and the V_{L} regions, and will retain the charge characteristics of the residues in order to preserve the low pI and low toxicity of the molecules. In some embodiments, amino acid substitutions (such as, e.g., at most one, at most two, at most three, at most four, or at most five amino acid substitutions) can be made in the V_{H} and/or the V_{L} regions to increase yield. Conservative amino acid substitution tables providing functionally similar amino acids are well known to one of ordinary skill in the art, such as, e.g., those described in Table A1.

TABLE A1
Example Conservative Substitutions

| | | Specific Example |
|---|---|---|
| Original | Example Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu, Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe | Leu |
| Leu (L) | Norleucine, Ile, Val, Met, Ala | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala | Leu |

As used herein, “percent (%) amino acid sequence identity” or “percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2.

As used herein, the term “cancer” refers to various conditions caused by the abnormal, uncontrolled growth of cells and includes neoplasms, primary tumors, secondary tumors and other metastatic lesions. Cancer can be detected in a number of ways including, but not limited to, the presence of a tumor in a tissue as detected by clinical or radiological means, detection of cancerous or abnormal cells in a biological sample (e.g., tissue biopsy), detection of a biomarker indicative of a cancer or a pre-cancerous condition, or detection of a genotype indicative of cancer or the risk of developing cancer. The term “cancer” encompasses various cancerous conditions regardless of stage, grade, invasiveness, aggressiveness, or tissue type. Cancers that may be treated according to the methods of the present disclosure include, but are not limited to, leukemia (e.g., myeloid leukemia, chronic lymphocytic leukemia, chronic myelogenous leukemia, acute lymphoblastic leukemia), lymphoma (e.g., diffuse large B-cell lymphoma, Burkitt lymphoma, Non-Hodgkin lymphoma, follicular lymphoma), multiple myeloma, lung cancer (e.g., small-cell lung cancer (SCLC), non-small cell lung cancer (NSCLC)), glioma, glioblastoma, melanoma, prostate cancer (e.g., castration-resistant prostate cancer, neuroendocrine prostate cancer), pancreatic cancer, breast cancer, bone cancer, cervical cancer, colon cancer, colorectal cancer, endometrial cancer, head and neck cancer, liver cancer, ovarian cancer, gastric cancer, gastroesophageal junction cancer, testicular cancer, thyroid cancer, adrenal cancer, renal cancer, bladder cancer, uterine cancer, esophageal cancer, urothelial cancer, carcinoma, and sarcoma, and metastatic cancer derived from any of the foregoing.

As used herein, the term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. As used herein, the term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The terms “IL2” and “IL-2” as used interchangeably herein refer to interleukin-2, which is a 15.5 to 16 kDa cytokine signaling protein molecule that regulates the activity of certain immune cells by binding to IL2 receptor complexes expressed by lymphocytes. The term “IL2” includes an IL2 protein of any human and non-human animal species, and specifically includes human IL2 as well as IL2 of non-human mammals. The human IL-2 sequence (UniProtKB No. P60568) is provided herein as SEQ ID NO: 41. The term “human IL2” as used herein includes any variants, isoforms, and species homologs of human IL2, regardless of its source or mode of preparation. Thus, “human IL2” includes human IL2 naturally expressed by cells and IL2 expressed on cells transfected with the human IL2 gene.

The terms “IL2R”, “IL-2R”, “IL2 receptor”, and “IL-2 receptor”, as used interchangeably herein refer generally to the IL2 receptor complex, which is composed of three polypeptide subunits, or chains, referred to as the alpha, A, or a chain, the beta, B, or R chain, and the gamma, G, or γ chain. IL-2R is a heterodimeric protein expressed on the surface of various immune cells, which serves as a cognate ligand for interleukin 2 (IL-2). The term “IL2R” includes any IL2R protein or any subunit of the IL2 receptor complex, of any human and non-human animal species, and specifically includes human IL2R as well as IL2R of non-human mammals. The term “human IL2R” as used herein includes any variants, isoforms, and species homologs of human IL2R, regardless of its source or mode of preparation. Thus, “human IL2R” includes human IL2R naturally expressed by cells and IL2R expressed on cells transfected with the human IL2R gene.

The term “IL2RA” or “IL2Ra” is also referred to as CD25, and the human IL2RA sequence (UniProtKB No. P01589) is provided herein as SEQ ID NO: 38.

The term “IL2RB” or “IL2R “is also referred to as CD122, and the human IL2RB sequence (UniProtKB No. P14784) is provided herein as SEQ ID NO: 39.

The term “IL2RG” or “IL2Ry” is also referred to as CD132, and the human IL2RG sequence (UniProtKB No. P31785) is provided herein as SEQ ID NO: 40.

The terms “anti-IL2R heavy chain-only antibody,” “IL2R heavy chain-only antibody,” “anti-IL2R heavy chain antibody,” and “IL2R heavy chain antibody” are used herein interchangeably to refer to a heavy chain-only antibody as hereinabove defined, that specifically binds to IL2R, including human IL2R, as hereinabove defined. The definition includes, without limitation, human heavy chain antibodies produced by transgenic animals, such as transgenic rats or transgenic mice expressing human immunoglobulin, including UniRats^{M} producing human anti-IL2R UniAb™ antibodies, as hereinabove defined. Similarly, as used herein, the term “anti-IL2R heavy chain-only antibody” refers to a heavy chain-only antibody that specifically binds to IL2RP and IL2Ry.

As used herein, the term “agonist” refers to a molecule that causes an increase in a function or activity as compared to the same function or activity in the absence of the molecule. An “agonist” of a signaling pathway is therefore a molecule whose presence causes an increase in a function or activity of the signaling pathway. The term “agonize,” as used herein, refers to causing an increase in a function or activity. In some embodiments, the agonist function of an antibody, antibody fragment, or antigen-binding fragment thereof may be determined using an assay described herein.

An “epitope” is the site on the surface of an antigen molecule to which a single antibody molecule binds. Generally, an antigen has several or many different epitopes and reacts with many different antibodies. The term specifically includes linear epitopes and conformational epitopes.

The term “valent” as used herein refers to a specified number of binding sites in an antibody molecule.

A “monovalent” antibody has one binding site. Thus, a monovalent antibody is also monospecific.

A “multi-valent” antibody has two or more binding sites. Thus, the terms “bivalent,” “trivalent,” and “tetravalent” refer to the presence of two binding sites, three binding sites, and four binding sites, respectively. Thus, a bispecific antibody according to the disclosure is at least bivalent and may be trivalent, tetravalent, or otherwise multi-valent. A bivalent antibody in accordance with embodiments of the disclosure may have two binding sites to the same epitope (i.e., bivalent, monoparatopic), or to two different epitopes (i.e., bivalent, biparatopic).

A large variety of methods and protein configurations are known and used for the preparation of bispecific monoclonal antibodies (BsMAB), tri-specific antibodies, and the like.

As used herein, the term “effector cell” refers to an immune cell which is involved in the effector phase of an immune response, as opposed to the cognitive and activation phases of an immune response. Some effector cells express specific Fc receptors and carry out specific immune functions. In some embodiments, an effector cell such as a natural killer cell is capable of inducing antibody-dependent cellular cytotoxicity (ADCC). For example, monocytes and macrophages, which express FcR, are involved in specific killing of target cells and presenting antigens to other components of the immune system, or binding to cells that present antigens. In some embodiments, an effector cell may phagocytose a target antigen or target cell.

“Human effector cells” are leukocytes which express receptors such as T-cell receptors or FcRs and perform effector functions. For example, in some embodiments, the cells express at least FcγRIII and perform ADCC effector function. Non-limiting examples of human leukocytes which mediate ADCC include natural killer (NK) cells, monocytes, cytotoxic T-cells, and neutrophils. The effector cells may be isolated from a native source thereof, e.g., from blood or PBMCs as described herein.

The term “immune cell” is used herein in the broadest sense, including, without limitation, cells of myeloid or lymphoid origin, for instance lymphocytes (such as B-cells and T-cells including cytolytic T-cells (CTLs)), killer cells, natural killer (NK) cells, macrophages, monocytes, eosinophils, polymorphonuclear cells, such as neutrophils, granulocytes, mast cells, and basophils.

Antibody “effector functions” refer to those biological activities attributable to the Fc region (a native sequence Fc region or amino acid sequence variant Fc region) of an antibody. Examples of antibody effector functions include C1q binding; complement dependent cytotoxicity (CDC); Fc receptor binding; antibody-dependent cell-mediated cytotoxicity (ADCC); phagocytosis; down regulation of cell surface receptors (e.g., B-cell receptor; BCR), etc.

“Antibody-dependent cell-mediated cytotoxicity” and “ADCC” refer to a cell-mediated reaction in which nonspecific cytotoxic cells that express Fc receptors (FcRs) (e.g., Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods 202:163 (1996), may be performed.

“Binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art. Low-affinity antibodies generally bind antigen slowly and tend to dissociate readily, whereas high-affinity antibodies generally bind antigen faster and tend to remain bound.

As used herein, the “Kd” or “Kd value” refers to a dissociation constant determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (kon). Antibody dissociation rates (koff) are measured in the final step, where the sensors are dipped into wells containing buffer only. The Kd is the ratio of koff/kon. (For further details see, Concepcion, J, et al., Comb Chem High Throughput Screen, 12(8), 791-800, 2009).

The term “KD” (M), as used herein, refers to the equilibrium dissociation constant of a particular antigen binding interaction as determined by BioLayer Interferometry, using an Octet QK384 instrument (Fortebio Inc., Menlo Park, CA) in kinetics mode. For example, anti-mouse Fc sensors are loaded with mouse-Fc fused antigen and then dipped into antibody-containing wells to measure concentration dependent association rates (ₖₒₙ). Antibody dissociation rates (k_{off}) are measured in the final step, where the sensors are dipped into wells containing buffer only. The KD is the ratio of k_{off}/kon. (For further details see, Concepcion, J, et al., Comb Chem High Throughput Screen, 12(8), 791-800, 2009).

As used herein, a molecule (such as, e.g., a molecule, antibody, antibody fragment, or antigen-binding fragment) “specifically binds” to a target antigen when it has a significantly higher binding affinity for, and consequently is capable of distinguishing, that antigen compared to its affinity for other unrelated proteins, under similar binding assay conditions. Molecules that specifically bind an antigen may bind to that antigen with an equilibrium dissociation constant (K_{D})≤1×10⁻⁶ M. Molecules specifically bind antigen with “high affinity” when the K_{D} is 1×10⁻⁸ M. In some embodiments, molecules described herein bind to a target antigen with a K_{D} Of 5×10⁻⁷ M. In some embodiments, molecules described herein bind to a target antigen with a K_{D} of 1×10⁻⁷ M. In some embodiments, molecules described herein bind to a target antigen with a K_{D} of 5×10⁻⁸ M. In some embodiments, molecules described herein bind to a target antigen with a K_{D} of 2×10⁻⁸ M. In some embodiments, molecules described herein bind to a target antigen with a K_{D} Of 1×10⁻⁸ M. In some embodiments, molecules described herein bind to a target antigen with a K_{D} of 1×10⁻⁹ M.

Affinity may be determined using a variety of techniques, a non-limiting example of which is an affinity ELISA assay. In some embodiments, affinity is determined by a surface plasmon resonance assay (e.g., BIAcore®-based assay). Using this methodology, the association rate constant (kₐ in M^{31 1}s⁻¹) and the dissociation rate constant (kₐ in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (kₐ in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and kₐ) and affinity (K_{D}) constants can be calculated in real-time using the bio-layer interferometry method.

As used herein, the term “administer” and its cognates (e.g., “administering”) includes both self-administration and administration to the patient by another person (e.g., a medical professional or caretaker).

As used herein, the term“in combination with,” in the context of administration, as well as “co-administer,” “combined administration,” and their cognates (e.g., “co-administering”), means administration of two or more therapeutic agents in a coordinated fashion to a single subject and includes, but is not limited to, concurrent administration. Specifically, “co-administration” encompasses administration of a co-formulation or simultaneous administration of separate therapeutic compositions, as well as serial or sequential administration, provided that administration of one therapeutic agent is conditioned in some way on administration of another therapeutic agent. The therapeutic agents are not necessarily administered at the same time and/or by the same route of administration. Illustratively, one therapeutic agent may be administered only after a different therapeutic agent has been administered and allowed to act for a prescribed period of time. Additionally, in some embodiments, co-administered therapeutic agents are present in the subject (PK), or otherwise induce an effect (PD), at similar, identical, or partially overlapping periods of time.

It is envisaged that “prior to”, in the context of a first therapeutic agent being administered prior to a second therapeutic agent, means within about 72 hours, about 48 hours, about 36 hours, about 24 hours, about 18 hours, about 16 hours, about 12 hours, about 6 hours, about 5 hours, about 4 hours, or about 3 hours, e.g., within about 120 minutes, about 90 minutes, about 60 minutes, or about 30 minutes before the start of administration of the second therapeutic agent.

In some embodiments, the combination partners may be administered entirely separately or be entirely separate pharmaceutical dosage forms. Additionally, in some embodiments, the combination partners may be pharmaceutical compositions that are also sold independently of each other, where instructions for their combined use are provided in the package equipment, e.g., leaflet or the like, or in other information, e.g., provided to physicians and medical staff (e.g., oral communications, communications in writing, or the like).

In some embodiments, the combination partners may be brought together into a combination therapy: (i) prior to release of the combination product to physicians (e.g., in the case of a kit comprising the combination partners); (ii) by the physician themselves (or under the guidance of a physician) shortly before administration; (iii) in the patient themselves, e.g. during sequential administration of the combination partners.

As used herein, a “combination product” refers to a pharmaceutical product that results from the mixing or combining of more than one active ingredient and includes both fixed and non-fixed combinations of the active ingredients (which may also be combined). A combination product includes a kit of components for combined administration.

As used herein, the term “non-fixed combination” refers to therapeutic agents that are administered to a patient as separate entities either simultaneously, concurrently, or sequentially with no specific time limits, wherein such administration provides therapeutically effective levels of both therapeutic agents. The latter also applies to cocktail therapy, e.g., the administration of three or more active ingredients. In a non-fixed combination, the combination partners may be dosed independently of each other or by use of different fixed combinations with distinguished amounts of the combination partners.

As used herein, the term “fixed combination” refers to at least two therapeutic agents that are both administered to a patient simultaneously in the form of a single entity or dosage (i.e., the therapeutic agents are present in one dosage form).

As used herein, the term “treatment” and its cognates (e.g., “treating”) encompass any improvement of a disease in the subject, including the slowing or stopping of the progression of a disease in the subject, a decrease in the number or severity of the symptoms of the disease, or an increase in frequency or duration of periods where the patient is free from the symptoms of the disease.

As used herein, a “therapeutically effective amount” refers to an amount of active agent that imparts therapeutic benefit to a subject. For example, a “therapeutically effective amount” is an amount which induces, ameliorates, or otherwise causes an improvement in the pathological symptoms, disease progression, or physiological conditions associated with a disease or which improves resistance to a disorder.

The terms “subject,” “individual,” and “patient” are used interchangeably herein to refer to a mammal being assessed for treatment and/or being treated. Subjects may be human, but also include other mammals, particularly those mammals useful as laboratory models for human disease, e.g., mouse, rat, etc. In some embodiments, the mammal is a human.

The term “pharmaceutical composition” refers to a preparation which is in such form as to permit the biological activity of the active ingredient to be effective, and which contains no additional components which are unacceptably toxic to a subject to which the formulation would be administered. Such compositions are sterile. “Pharmaceutically acceptable” excipients (e.g., vehicles, additives) are those which can reasonably be administered to a subject mammal to provide an effective dose of the active ingredient employed.

As used herein, a “sterile” composition is aseptic or free or essentially free from all living microorganisms and their spores. As used herein, a “frozen” composition is one at a temperature below 0° C.

As used herein, a “stable” composition is one in which the protein therein essentially retains its physical stability and/or chemical stability and/or biological activity upon storage. In some embodiments, the composition essentially retains its physical and chemical stability, as well as its biological activity upon storage. The storage period is generally selected based on the intended shelf-life of the composition. Various analytical techniques for measuring protein stability are available in the art and are reviewed in Peptide and Protein Drug Delivery, 247-301. Vincent Lee Ed., Marcel Dekker, Inc., New York, N.Y., Pubs. (1991) and Jones. A. Adv. Drug Delivery Rev. 10: 29-90) (1993), for example. Stability can be measured at a selected temperature for a selected time period. Stability can be evaluated qualitatively and/or quantitatively in a variety of different ways, including evaluation of aggregate formation (for example, using size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis; SDS-PAGE analysis to compare reduced and intact antibody; peptide map (for example tryptic or LYS-C) analysis; evaluating biological activity or antigen binding function of the antibody; etc. Instability may involve any one or more of. aggregation, deamidation (e.g., Asn deamidation), oxidation (e.g., Met oxidation), isomerization (e.g., Asp isomerization), clipping/hydrolysis/fragmentation (e.g., hinge region fragmentation), succinimide formation, unpaired cysteine(s), N-terminal extension, C-terminal processing, glycosylation differences, etc.

As used herein, the term “vector” refers to a nucleic acid molecule capable of transporting another nucleic acid to which it has been linked. One type of vector is a “plasmid,” which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (such as, e.g., bacterial vectors having a bacterial origin of replication and episomal mammalian vectors). Other vectors (such as, e.g., non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors.” In some embodiments, expression vectors for use in recombinant DNA techniques are in the form of plasmids.

As used herein, the term “host cell” refers to a cell into which an expression vector has been introduced. It should be understood that “host cell” is intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Example recombinant host cells include, but are not limited to, transfectomas, such as CHO cells, HEK293 cells, NS/0 cells, and lymphocytic cells.

As used herein, the term “autologous” refers to any material derived from an individual to whom the material is intended to be re-introduced.

As used herein, the term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

As used herein, the term “Chimeric Antigen Receptor,” or alternatively a “CAR,” refers to a recombinant polypeptide construct comprising at least an extracellular antigen binding domain, a transmembrane domain, and a cytoplasmic signaling domain (also referred to herein as “an intracellular signaling domain”) comprising a functional signaling domain derived from a stimulatory molecule. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains.

In some embodiments, the stimulatory molecule is the zeta chain associated with the T-cell receptor complex. In some embodiments, the cytoplasmic signaling domain comprises a primary signaling domain (e.g., a primary signaling domain of CD3-zeta). In some embodiments, the cytoplasmic signaling domain further comprises one or more functional signaling domains derived from at least one costimulatory molecule. In some embodiments, the costimulatory molecule is chosen from 4-1BB (i.e., CD137), CD27, ICOS, and CD28. In some embodiments, the CAR comprises a chimeric fusion protein comprising an extracellular antigen binding domain, a transmembrane domain, and an intracellular signaling domain comprising a functional signaling domain derived from a stimulatory molecule. In some embodiments, the CAR comprises a chimeric fusion protein comprising an extracellular antigen binding domain, a transmembrane domain, and an intracellular signaling domain comprising a functional signaling domain derived from a co-stimulatory molecule and a functional signaling domain derived from a stimulatory molecule. In some embodiments, the CAR comprises a chimeric fusion protein comprising an extracellular antigen binding domain, a transmembrane domain, and an intracellular signaling domain comprising two functional signaling domains derived from one or more co-stimulatory molecule(s) and a functional signaling domain derived from a stimulatory molecule. In some embodiments, the CAR comprises a chimeric fusion protein comprising an extracellular antigen binding domain, a transmembrane domain, and an intracellular signaling domain comprising at least two functional signaling domains derived from one or more co-stimulatory molecule(s) and a functional signaling domain derived from a stimulatory molecule. In some embodiments, the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen recognition domain (e.g., a scFv) during cellular processing and localization of the CAR to the cellular membrane.

As used herein, the term “signaling domain” refers to the functional portion of a protein which acts by transmitting information within the cell to regulate cellular activity via defined signaling pathways by generating second messengers or functioning as effectors by responding to such messengers. In some embodiments, the signaling domain of a CAR described herein is derived from a stimulatory molecule or co-stimulatory molecule, or is a synthesized or engineered signaling domain.

As used herein, an “intracellular signaling domain” refers to an intracellular portion of a molecule. The intracellular signaling domain generates a signal that promotes an immune effector function of the CAR-expressing cell, e.g., a CAR-T cell or CAR-expressing NK cell. Non-limiting examples of immune effector function, e.g., in a CAR-T cell or CAR-expressing NK cell, include cytolytic activity and helper activity, including the secretion of cytokines. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain may comprise a primary intracellular signaling domain. Example primary intracellular signaling domains include, but are not limited to, those derived from the molecules responsible for primary stimulation, or antigen dependent simulation. In some embodiments, the intracellular signaling domain comprises a costimulatory intracellular domain. Example costimulatory intracellular signaling domains include, but are not limited to, those derived from molecules responsible for costimulatory signals, or antigen independent stimulation. In some embodiments, the intracellular signaling domain is synthesized or engineered. For example, in the case of a CAR-expressing immune effector cell, e.g., CAR-T cell or CAR-expressing NK cell, a primary intracellular signaling domain may comprise a cytoplasmic sequence of a T cell receptor, a primary intracellular signaling domain may comprise a cytoplasmic sequence of a T cell receptor, and a costimulatory intracellular signaling domain may comprise cytoplasmic sequence from co-receptor or costimulatory molecule.

In some embodiments, a primary intracellular signaling domain comprises a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta, CD3 gamma, CD3 delta, CD3 epsilon, CDS, CD22, CD79a, CD79b, CD278 (“ICOS”), F∃cRI CD66d, DAP10 and DAP12.

As used herein, the term “costimulatory molecule” refers to the cognate binding partner on a T cell that specifically binds with a costimulatory ligand, thereby mediating a costimulatory response by the T cell, including, but not limited to, proliferation. Costimulatory molecules are cell surface molecules other than antigen receptors or their ligands that are required for an efficient immune response. Costimulatory molecules include, but are not limited to an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, a costimulatory intracellular signaling domain can be the intracellular portion of a costimulatory molecule. The intracellular signaling domain can comprise the entire intracellular portion, or the entire native intracellular signaling domain, of the molecule from which it is derived, or a functional fragment thereof

As used herein, the term “zeta,” or alternatively “zeta chain” or “CD3-zeta,” is defined as the protein provided as GenBan Acc. No. BAG36664.1, or the equivalent residues from a non-human species, e.g., mouse, rodent, monkey, ape, and the like, and a “zeta stimulatory domain,” or alternatively a “CD3-zeta stimulatory domain,” is defined as the amino acid residues from the cytoplasmic domain of the zeta chain that are sufficient to functionally transmit an initial signal necessary for T cell activation. In some embodiments, the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

As used herein, the term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

As used herein, the term “T-cell redirecting therapy” refers to a therapeutic agent, such as a T-cell engaging molecule or a CAR T-cell, capable of recruiting T-cells to a target cell or tissue.

As used herein, the term “T-cell engaging molecule” refers to a molecule that comprises at least one domain in which the structure is derived from or comprises the minimum structural features of an antibody, e.g., of a full-length immunoglobulin molecule, that allow for specific binding to an antigen on the surface of a T cell, such as CD3. Thus, a T-cell engaging molecule according to the present disclosure generally comprises one or more binding domains, each of which will typically comprise the minimum structural requirements of an antibody that allow for specific target binding. This minimum requirement may, for example, be defined by the presence of at least three light chain “complementarity determining regions” or CDRs (i.e., CDRL1, CDRL2 and CDRL3 of a VL region) and/or three heavy chain CDRs (i.e. CDRH1, CDRH2 and CDRH3 of a VH region), such as, e.g., all six CDRs from both the light and heavy chain variable regions. The T-cell engaging molecules according to the present disclosure may comprise domains or regions (e.g. CDRs or variable regions) from monoclonal, chimeric, humanized and human antibodies. In some embodiments, the T-cell engaging molecules used in the methods of the present disclosure are proteins and comprise one or more polypeptide chains. In some embodiments, the T-cell engaging molecules administered according to the methods of the present disclosure are single-chain polypeptides. In other embodiments, the T-cell engaging molecules administered according to the methods of the present disclosure comprise two or more polypeptide chains—e.g., are polypeptide dimers or multimers. In certain embodiments, the T-cell engaging molecules administered according to the methods of the present disclosure comprise four polypeptide chains, and may, e.g., have the format of an antibody or an immunoglobulin protein.

As used herein, the term “bispecific T-cell engaging molecule” refers to a molecule capable of specifically binding to two different antigens. In the context of the present disclosure, bispecific T-cell engaging molecules specifically bind to a cancer cell antigen (e.g., human cancer cell antigen) on the cell surface of target cells and CD3 (e.g., human CD3) on the cell surface of T cells. In some embodiments, the T-cell engaging molecules may bind to more than one cancer cell antigen (e.g., human cancer cell antigen) on the cell surface of target cells as well as to CD3 (e.g., human CD3) on the cell surface of T cells. Thus, in such embodiments, the T-cell engaging molecules are “multitargeting” in that they are capable of specifically binding to two or more different cancer cell antigens and redirecting T cells to more than one type of cancer cell or cancer cells expressing the two or more antigens.

A T-cell engaging molecule or binding domain thereof “specifically binds” to a target antigen when it has a significantly higher binding affinity for, and consequently is capable of distinguishing, that antigen compared to its affinity for other unrelated proteins, under similar binding assay conditions. T-cell engaging molecules or binding domains thereof that specifically bind an antigen may bind to that antigen with an equilibrium dissociation constant (K_{D})≤1 ×10⁻⁶ M. In one embodiment, the T-cell engaging molecules or binding domains thereof used in the methods of the present disclosure bind to a human cancer cell antigen and/or human CD3 with a K_{D} of ≤5×10⁻⁷ M. In another embodiment, the T-cell engaging molecules or binding domains thereof used in the methods of the present disclosure bind to a human cancer cell antigen and/or human CD3 with a K_{D} of <1×10⁻⁷ M. In yet another embodiment, the T-cell engaging molecules or binding domains thereof used in the methods of the present disclosure bind to a human cancer cell antigen and/or human CD3 with a K_{D} of 5×10⁻⁸ M. In another embodiment, the T-cell engaging molecules or binding domains thereof used in the methods of the present disclosure bind to a human cancer cell antigen and/or human CD3 with a K_{D} of 2 ×10⁻⁸ M. In certain embodiments, the T-cell engaging molecules or binding domains thereof used in the methods of the present disclosure bind to a human cancer cell antigen and/or human CD3 with a K_{D} of <1×10⁻⁸ M. In other embodiments, the T-cell engaging molecules or binding domains thereof used in the methods of the present disclosure bind to a human cancer cell antigen and/or human CD3 with a K_{D} Of≤1×10⁻⁹ M.

In some embodiments, the T-cell engaging molecules or binding domains thereof described herein exhibit desirable characteristics such as binding avidity as measured by kₐ (dissociation rate constant) for a human cancer cell antigen and/or human CD3 of about 10⁻², 10⁻³, 10⁻⁴, 10⁻⁵, 10⁻⁶, 10^{31 7}, 10⁻⁸, 10⁻⁹, 10⁻¹⁰ s⁻¹ or lower (lower values indicating higher binding avidity), and/or binding affinity as measured by K_{D} (equilibrium dissociation constant) for a human cancer cell antigen and/or human CD3 of about 10⁻⁷, 10⁻⁸, 10⁻⁹, 10⁻¹⁰, 10⁻¹¹ M or lower (lower values indicating higher binding affinity).

In some embodiments, bispecific T-cell engaging molecules used in the methods of the present disclosure may be antibodies and have the general structure of a full-length immunoglobulin. For example, the bispecific T-cell engaging molecules may comprise two full-length antibody heavy chains and two full-length antibody light chains. In particular embodiments, the bispecific T-cell engaging molecules are heterodimeric antibodies (used interchangeably herein with “hetero immunoglobulins” or “hetero Igs”), which refer to antibodies comprising two different light chains and two different heavy chains. For instance, in some embodiments, the heterodimeric antibody comprises a light chain and heavy chain from an antibody that specifically binds to a cancer cell antigen, such as the cancer cell antigens described further herein, and a light chain and heavy chain from an antibody that specifically binds to CD3.

The bispecific T-cell engaging molecules employed in the methods of the present disclosure may also comprise fragments of full-length antibodies, such as VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain). Bispecific T-cell engaging molecules according to the present disclosure may also comprise modified fragments of antibodies. Examples of such modified fragments include, but are not limited to, single-chain variable fragment (scFv), di-scFv or bi(s)-scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains.

In certain embodiments, the bispecific T-cell engaging molecules used in the methods of the present disclosure are multivalent. The valency of the T-cell engaging molecule denotes the number of individual antigen-binding domains within the T-cell engaging molecule. For example, the terms “monovalent,” “bivalent,” and “tetravalent” with reference to the T-cell engaging molecules in the context of the present disclosure refer to T-cell engaging molecules with one, two, and four antigen-binding domains, respectively. Thus, a multivalent T-cell engaging molecule comprises two or more antigen-binding domains. A T-cell engaging molecule can have more antigen-binding domains (e.g. a higher valency) than specificities. For example, a T-cell engaging molecule having two antigen-binding domains for a first target (e.g. cancer cell antigen) and one antigen-binding domain for a second target (CD3) —or vice versa —is considered to be trivalent (three antigen-binding domains) and bispecific (binds to two antigens). In certain embodiments, the bispecific T-cell engaging molecules used in the methods of the present disclosure are bivalent. Thus, such bispecific, bivalent T-cell engaging molecules contain two antigen binding domains: one antigen-binding domain for a cancer cell antigen (e.g. a human cancer cell antigen) and one antigen-binding domain for CD3 (e.g. human CD3). In other embodiments, the T-cell engaging molecules used in the methods of the present disclosure are trivalent, trispecific T-cell engaging molecules and comprise three antigen binding domains: one antigen binding domain for a first cancer cell antigen, another antigen binding domain for a second cancer cell antigen, and a third binding domain for CD3. In still other embodiments, the T-cell engaging molecules used in the methods of the present disclosure are tetravalent, trispecific T-cell engaging molecules and comprise four antigen binding domains: one antigen binding domain for a first cancer cell antigen, another antigen binding domain for a second cancer cell antigen, and two antigen binding domains for CD3.

In some embodiments, the bispecific T-cell engaging molecules employed in the methods of the present disclosure comprise a first binding domain that specifically binds to a target cancer cell antigen (e.g. a human target cancer cell antigen) and a second binding domain that specifically binds to CD3 (e.g. human CD3). As used herein, the term “antigen-binding domain,” which is used interchangeably with “binding domain,” refers to the region of the T-cell engaging molecule that contains the amino acid residues that interact with the antigen and confer on the T-cell engaging molecule its specificity and affinity for the antigen. In certain embodiments, one or more binding domains of the T-cell engaging molecules may be derived from an antibody or antigen-binding fragment thereof. For instance, the binding domains of the bispecific T-cell engaging molecules used in the methods of the present disclosure may comprise one or more CDRs from the light and heavy chain variable regions of antibodies that specifically bind to a human target cancer cell antigen and/or human CD3. In some embodiments, the anti-cancer cell antigen binding domain of the bispecific T-cell engaging molecules comprises all six CDRs of the heavy and light chain variable regions of an antibody that specifically binds to that human target cancer cell antigen and the anti-CD3 binding domain of the bispecific T-cell engaging molecules comprises all six CDRs of the heavy and light chain variable regions of an anti-CD3 antibody. In some embodiments, the binding domains (the anti-cancer cell antigen binding domain, the anti-CD3 binding domain or both) of the bispecific T-cell engaging molecules used in the methods of the present disclosure comprise a Fab, a Fab′, a F(ab′)₂, a Fv, a single-chain variable fragment (scFv), or a nanobody. In one embodiment, both binding domains of the bispecific T-cell engaging molecule are Fab fragments. In another embodiment, one binding domain of the bispecific T-cell engaging molecule is a Fab fragment and the other binding domain is a scFv. In yet another embodiment, both binding domains of the bispecific T-cell engaging molecule are scFvs.

### Anti-IL-2Rβγ Heavy Chain Only Antibodies and Antigen Binding Fragments Thereof

In a conventional IgG antibody, the association of the heavy chain and the light chain is due in part to a hydrophobic interaction between the light chain constant region and the CH1 constant domain of the heavy chain. There are additional residues in the heavy chain framework 2 (FR2) and framework 4 (FR4) regions that also contribute to this hydrophobic interaction between the heavy and light chains.

It is known, however, that sera of camelids (sub-order Tylopoda which includes camels, dromedaries, and llamas) contain a major type of antibodies composed solely of paired H-chains (heavy-chain only antibodies or HCAbs). The heavy chain-only antibodies of Camelidae (Camelus dromedarius, Camelus bactrianus, Lama glama, Lama guanaco, Lama alpaca and Lama vicugna) have a unique structure consisting of a single variable domain (VHH), a hinge region, and two constant domains (CH2 and CH3), which are highly homologous to the CH2 and CH3 domains of classical antibodies. These heavy chain-only antibodies lack the first domain of the constant region (CH1), which is present in the genome but is spliced out during mRNA processing. The absence of the CH1 domain explains the absence of the light chain in the heavy-chain only antibodies since this domain is the anchoring place for the constant domain of the light chain. Such heavy-chain only antibodies naturally evolved to confer antigen-binding specificity and high affinity by three CDRs from conventional antibodies or fragments thereof. Muyldermans, 2001; J Biotechnol 74:277-302; Revets et al., 2005; Expert Opin Biol Ther 5:111-124. Cartilaginous fish, such as sharks, have also evolved a distinctive type of immunoglobulin, designated as IgNAR, which lacks the light polypeptide chains and is composed entirely by heavy chains. IgNAR molecules can be manipulated by molecular engineering to produce the variable domain of a single heavy chain polypeptide (vNARs). Nuttall et al. Eur. J Biochem. 270, 3543-3554 (2003); Nuttall et al. Function and Bioinformatics 55, 187-197 (2004); Dooley et al., Molecular Immunology 40, 25-33 (2003).

The ability of heavy chain only-antibodies devoid of light chain to bind antigen was established in the 1960s (Jaton et al. (1968) Biochemistry, 7, 4185-4195). Heavy chain immunoglobulin physically separated from light chain retained 80% of antigen-binding activity relative to the tetrameric antibody. Sitia et al. (1990) Cell, 60, 781-790 demonstrated that removal of the CH1 domain from a rearranged mouse gene results in the production of a heavy chain-only antibody, devoid of light chain, in mammalian cell culture. The antibodies produced retained VH binding specificity and effector functions.

Heavy chain antibodies with high specificity and affinity can be generated against a variety of antigens through immunization (van der Linden, R. H., et al. Biochim. Biophys. Acta. 1431, 37-46 (1999)), and the VHH portion can be readily cloned and expressed in yeast (Frenken, L. G. J., et al. J. Biotechnol. 78, 11-21 (2000)). Their levels of expression, solubility and stability are significantly higher than those of classical F(ab) or Fv fragments. Ghahroudi, M. A. et al. FEBS Lett. 414, 521-526 (1997).

Mice in which the λ (lambda) light (L) chain locus and/or the λ and κ (kappa) L chain loci have been functionally silenced and antibodies produced by such mice are described in U.S. Pat. Nos. 7,541,513 and 8,367,888. Recombinant production of heavy chain-only antibodies in mice and rats has been reported, for example, in WO2006008548; U.S. Application Publication No. 20100122358; Nguyen et al., 2003, Immunology; 109(1), 93-101; Bruggemann et al., Crit. Rev. Immunol.; 2006, 26(5):377-90; and Zou et al., 2007, J Exp Med; 204(13): 3271-3283. The production of knockout rats via embryo microinjections of zinc-finger nucleases is described in Geurts et al., 2009, Science, 325(5939):433. Soluble heavy chain only-antibodies and transgenic rodents comprising a heterologous heavy chain locus producing such antibodies are described in U.S. Pat. Nos. 8,883,150 and 9,365,655. CAR-T structures comprising single-domain antibodies as binding (targeting) domains are described, for example, in Iri-Sofla et al., 2011, Experimental Cell Research 317:2630-2641, and Jamnani et al., 2014, Biochim Biophys Acta, 1840:378-386.

The present disclosure provides various families of heavy chain-only antibodies and antigen-binding fragments thereof that bind to human IL2Rβγ for use in a method disclosed herein. In some embodiments, an anti-IL-2Rβγ heavy chain-only antibody is used in a method disclosed herein. In some embodiments, an antigen-binding fragment of an anti-IL-2Rβγ heavy chain-only antibody is used in a method disclosed herein.

In some embodiments, an anti-IL-2Rβγ heavy chain-only antibody or antigen-binding fragment thereof comprises CDR sequences having the following sequence formulae. An X indicates a variable amino acid, which may, in some embodiments, be the specific amino acid listed below:

CDR1 (IL2RB_F09)
(SEQ ID NO: 26)
G G S I S S S X₁ W

wherein X₁ is D or N;

CDR2 (IL2RB_F09)
(SEQ ID NO: 27)
I X₂ H S G S T

wherein X₂ is D or S; and

CDR3 (IL2RB_F09)
(SEQ ID NO: 28)
X₃ R G X₄ W E L X₅ D A F D I

wherein:
   X₃ is G or A;
   X₄ is S or Q; and
   X₅ is S or T.

In some embodiments, an anti-IL-2Rβγ heavy chain-only antibody or antigen-binding fragment thereof comprises any combination of CDR1, CDR2, and CDR3 sequences comprising the sequence formulae of SEQ ID NOs: 26, 27, and 28, respectively. Heavy chain-only antibodies of this family can be referred to herein as IL2RB_F09 antibodies.

In some embodiments, an anti-IL-2Rβγ heavy chain-only antibody or antigen-binding fragment thereof comprises CDR sequences having the following sequence formulae. An X indicates a variable amino acid, which may, in some embodiments, be the specific amino acid listed below:

CDR1 (IL2RB_F18)
(SEQ ID NO: 29)
G F T F S X₁ Y G

where X₁ is S or T;

CDR2 (IL2RB_F18)
(SEQ ID NO: 30)
I S Y D G S N X₂

where X₂ is K or R; and

CDR3 (IL2RB_F18)
(SEQ ID NO: 31)
ARDLDYDX₃LTGDPVGGFDI

where X₃ is V or I.

In some embodiments, an anti-IL-2Rβγ heavy chain-only antibody or antigen-binding fragment thereof comprises any combination of CDR1, CDR2, and CDR3 sequences comprising the sequence formulae of SEQ ID NOs: 29, 30, and 31, respectively. Heavy chain-only antibodies of this family can be referred to herein as IL2RB_F18 antibodies.

Heavy chain-only antibodies and antigen-binding fragments thereof in accordance with embodiments of the disclosure that bind to IL2RB can comprise a set of CDR sequences as defined herein and exemplified by the provided heavy chain CDR1, CDR2, and CDR3 sequences set forth in Table 1, and the heavy chain variable region (VH) sequences set forth in Table 2. These heavy chain-only antibodies and antigen-binding fragments thereof provide a number of benefits that contribute to utility as clinically therapeutic agent(s). The heavy chain-only antibodies and antigen-binding fragments include members with a range of binding affinities, allowing the selection of a specific sequence with a desired binding affinity.

TABLE 1
Anti-IL2RB Heavy Chain Antibody Unique CDR Amino Acid Sequences

| Clone | Family | | | |
|---|---|---|---|---|
| ID No. | ID No. | CDR1 | CDR2 | CDR3 |
| 387205 | IL2RB_F09C | GGSISSSDW | IDHSGST | GRGSWELSDAFDI |
| | | (SEQ ID NO: 1) | (SEQ ID NO: 4) | (SEQ ID NO: 7) |
| 387172 | IL2RB_F09G | GGSISSSDW | IDHSGST | ARGSWELTDAFDI |
| | | (SEQ ID NO: 1) | (SEQ ID NO: 4) | (SEQ ID NO: 8) |
| 387111 | IL2RB_F09K | GGSISSSNW | ISHSGST | GRGSWELTDAFDI |
| | | (SEQ ID NO: 2) | (SEQ ID NO: 5) | (SEQ ID NO: 9) |
| 388252 | IL2RB_F18E | GFTFSSYG | ISYDGSNK | ARDLDYDVLTGDPVGGF |
| | | (SEQ ID NO: 3) | (SEQ ID NO: 6) | DI |
| | | | | (SEQ ID NO: 10) |

TABLE 2
Anti-IL2RB Heavy Chain Antibody Variable Region Amino Acid Sequences

| Clone | Family | |
|---|---|---|
| ID No. | ID No. | VH sequence |
| 387205 | IL2RB_F09C | QVQLQESGPGLVKPSGTLSLTCAVSGGSISSSDWWSWVR |
| | | QPPGKGLEWIGEIDHSGSTNYNPSLMSRVTISVDKSKNQF |
| | | SLKLSSVTAADTAVYFCGRGSWELSDAFDIRGQGTLVTV |
| | | SS (SEQ ID NO: 11) |
| 387172 | IL2RB_F09G | QVQLQESGPGLVKSSETLSLTCTVSGGSISSSDWWSWVR |
| | | QPPGKGLEWIGEIDHSGSTNYNPSLMSRVTISVDKSKNQF |
| | | SLKLSSVTAADTAVYFCARGSWELTDAFDIRGQGTLVTV |
| | | SS (SEQ ID NO: 12) |
| 387111 | IL2RB_F09K | QVQLQESSPGLVKPSETLSLTCTVSGGSISSSNWWSWVR |
| | | QPPGKGLEWIGEISHSGSTNYNPSLKSRVTISVDKSKNQF |
| | | SLRLSSVTAADTAVYFCGRGSWELTDAFDIRGQGTLVTV |
| | | SS (SEQ ID NO: 13) |
| 388252 | IL2RB_F18E | QVQLVESGGGVVQPGRSLRLSCAASGFTFSSYGMHWVR |
| | | QAPGKEREWVAVISYDGSNKYYTDSVKGRFTISRDNSK |
| | | NTLYLEMNSLRAEDTAVYYCARDLDYDVLTGDPVGGF |
| | | DIWGQGTLVTVSS (SEQ ID NO: 14) |

In some embodiments, an anti-IL-2Rβγ heavy chain-only antibody or antigen-binding fragment thereof comprises CDR sequences having the following sequence formulae. An X indicates a variable amino acid, which may, in some embodiments, be the specific amino acid listed below:

CDR1 (IL2RG_F16)
(SEQ ID NO: 32)
GFX₁X₂X₃X₄YY

wherein:
   X₁ is T or I;
   X₂ is F or V;
   X₃ is S, N, or G; and
   X₄ is D or N;

CDR2 (IL2RG_F16)
(SEQ ID NO: 33)
ISX₅SGX₆X₇I

wherein:
   X₅ is S or N;
   X₆ is D, S, G, or N; and
   X₇ is T or I; and

CDR3 (IL2RG_F16)
(SEQ ID NO: 20)
ARGDAVSITGDY

In some embodiments, an anti-IL-2Rβγ heavy chain-only antibody or antigen-binding fragment thereof comprises any combination of CDR1, CDR2, and CDR3 sequences comprising the sequence formulae of SEQ ID NOs: 32, 33, and 34, respectively. Heavy chain-only antibodies of this family can be referred to herein as IL2RG_F16 antibodies.

In some embodiments, an anti-IL-2βγ heavy chain-only antibody or antigen-binding fragment thereof comprises a CDR1 sequence comprising GFTFSDYY (SEQ ID NO: 15), a CDR2 (IL2RG F18) sequence comprising ISSSGTTT (SEQ ID NO: 19), and a CDR3 (IL2RGF18) sequence comprising ARGAAVAPGFDS (SEQ ID NO: 21). Heavy chain-only antibodies of this family can be referred to herein as IL2RG_F18 antibodies.

Heavy chain-only antibodies and antigen-binding fragments thereof in accordance with embodiments of the disclosure that bind to IL2RG comprise a set of CDR sequences as defined herein and exemplified by the provided heavy chain CDR1, CDR2, and CDR3 sequences set forth in Table 3, and the heavy chain variable region (VH) sequences set forth in Table 4. This family of heavy chain-only antibodies and antigen-binding fragments thereof provides a number of benefits that contribute to utility as clinically therapeutic agent(s). The heavy chain-only antibodies and antigen-binding fragments thereof include members with a range of binding affinities, allowing the selection of a specific sequence with a desired binding affinity.

TABLE 3
Anti-IL2RG Heavy Chain Antibody CDR1, CDR2 and CDR3 Amino Acid Sequences

| Clone | Family | | | |
|---|---|---|---|---|
| ID No. | ID No. | CDR1 | CDR2 | CDR3 |
| 363256 | IL2RG_F16A | GFTFSDYY | ISSSGDTI | ARGDAVSITGDY |
| | | (SEQ ID NO: 15) | (SEQ ID NO: 17) | (SEQ ID NO: 20) |
| 363544 | IL2RG_F16B | GFTFSDYY | ISSSGSTI | ARGDAVSITGDY |
| | | (SEQ ID NO: 15) | (SEQ ID NO: 18) | (SEQ ID NO: 20) |
| 388582 | IL2RG_F16C | GFTFNDYY | ISSSGSTI | ARGDAVSITGDY |
| | | (SEQ ID NO: 16) | (SEQ ID NO: 18) | (SEQ ID NO: 20) |
| 363435 | IL2RG_F18A | GFTFSDYY | ISSSGTTT | ARGAAVAPGFDS |
| | | (SEQ ID NO: 15) | (SEQ ID NO: 19) | (SEQ ID NO: 21) |

TABLE 4
Anti-IL2RG Heavy Chain Antibody Variable Region Amino Acid Sequences

| Clone | Family | |
|---|---|---|
| ID No. | ID No. | VH Sequence |
| 363256 | IL2RG_F16A | QVQLVESGGGLVKPGGSLRLSCAASGFTFSDYYMSWIRQ |
| | | APGKGLEWVSSISSSGDTIYYADSVQGRFTLSRDNAENSLF |
| | | LQMNSLRAEDTAVYYCARGDAVSITGDYRGQGTLVTVSS |
| | | (SEQ ID NO: 22) |
| 363544 | IL2RG_F16B | QVQLVESGGGLVKPGGSLRLSCAASGFTFSDYYMSWIRQ |
| | | APGKGLEWVSYISSSGSTIYYADSVKGRFTISRDNAKNSLY |
| | | LQMNSLRAEDTAVYYCARGDAVSITGDYRGQGTLVTVSS |
| | | (SEQ ID NO: 23) |
| 388582 | IL2RG_F16C | QVQLVESGGGLVKPGGSLRLSCAASGFTFNDYYMSWIRQ |
| | | APGKGLEWVSHISSSGSTIYYADSVKGRFTVSRDNANNSL |
| | | YLQMHSLRAEDTAVYYCARGDAVSITGDYRGQGTLVTVS |
| | | S (SEQ ID NO: 24) |
| 363435 | IL2RG_F18A | QVQLVESGGDLVKPGGSLRLSCAASGFTFSDYYMSWLRQ |
| | | APGKELEWVSHISSSGTTTYYADSVEGRFTITRDNAKNSL |
| | | YLQMNSLRAEDTAVYYCARGAAVAPGFDSRGQGTLVTV |
| | | SS (SEQ ID NO: 25) |

Determination of affinity for an anti-IL-2Rβγ heavy chain-only antibody or antigen-binding fragment thereof can be performed using methods known in the art, such as, e.g., using Biacore measurements. Members of the heavy chain-only antibody and antigen-binding fragment families described herein may have an affinity for IL2R with a Kd of from about 10⁻⁶ to about 10⁻¹¹, including without limitation: from about 10⁻⁶ to about 10⁻¹⁰; from about 10⁻⁶ to about 10⁻⁹; from about 10⁻⁶ to about 10⁻¹⁰; from about 10⁻¹ to about 10⁻¹¹; from about 10⁻⁸ to about 10⁻¹⁰; from about 10⁻⁸ to about 10⁻⁹; from about 10⁻⁹ to about 10⁻¹¹; from about 10⁻⁹ to about 10⁻¹⁰; or any value within these ranges. The affinity selection may be confirmed with a biological assessment for modulating, e.g., agonizing, an IL2R biological activity, including in vitro assays, pre-clinical models, and clinical trials, as well as assessment of potential toxicity.

Members of the heavy chain-only antibody and antigen-binding fragment families described herein are cross-reactive with the IL2R protein of Cynomolgus macaque, which facilitates the use of Cynomolgus macaque as an animal model for validating, e.g., mechanism of action, pharmacokinetics, toxicology, and other attributes of the heavy chain-only antibodies and antigen-binding fragments described herein.

In some embodiments, the IL2R-specific heavy chain-only antibodies and antigen-binding fragments described herein comprise a VH domain, comprising CDR1, CDR2, and CDR3 sequences in a human VH framework. The CDR sequences may be situated, as an example, in the region of around amino acid residues 26-33; 51-58; and 97-116 for CDR1, CDR2, and CDR3, respectively, of the provided exemplary variable region sequences set forth in SEQ ID NOs: 11-14 and 22-25. It will be understood by one of ordinary skill in the art that the CDR sequences may be in different positions if a different framework sequence is selected, although generally the order of the sequences will remain the same.

In a particular embodiment, an anti-IL-2Rβγ heavy chain-only antibody or antigen-binding fragment thereof comprises a CDR1 sequence of any one of SEQ ID NOs: 1-3. In a particular embodiment, the CDR1 sequence comprises SEQ ID NO: 1. In a particular embodiment, the CDR1 sequence comprises SEQ ID NO: 2. In a particular embodiment, the CDR1 sequence comprises SEQ ID NO: 3.

In a particular embodiment, an anti-IL-2Rβγ heavy chain-only antibody or antigen-binding fragment thereof comprises a CDR2 sequence of any one of SEQ ID NOs: 4-6. In a particular embodiment, the CDR2 sequence comprises SEQ ID NO: 4. In a particular embodiment, the CDR2 sequence comprises SEQ ID NO: 5. In a particular embodiment, the CDR2 sequence comprises SEQ ID NO: 6.

In a particular embodiment, an anti-IL-2Rβγ heavy chain-only antibody or antigen-binding fragment thereof comprises a CDR3 sequence of any one of SEQ ID NOs: 7-10. In a particular embodiment, the CDR3 sequence comprises SEQ ID NO: 7. In a particular embodiment, the CDR2 sequence comprises SEQ ID NO: 8. In a particular embodiment, the CDR2 sequence comprises SEQ ID NO: 9. In a particular embodiment, the CDR2 sequence comprises SEQ ID NO: 10.

In a further embodiment, an anti-IL-2Rβγ heavy chain-only antibody or antigen-binding fragment thereof comprises the CDR1 sequence of SEQ ID NO: 1; the CDR2 sequence of SEQ ID NO: 4; and the CDR3 sequence of SEQ ID NO: 7.

In a further embodiment, an anti-IL-2Rβγ heavy chain-only antibody or antigen-binding fragment thereof comprises the CDR1 sequence of SEQ ID NO: 1; the CDR2 sequence of SEQ ID NO: 4; and the CDR3 sequence of SEQ ID NO: 8.

In a further embodiment, an anti-IL-2Rβγ heavy chain-only antibody or antigen-binding fragment thereof comprises the CDR1 sequence of SEQ ID NO: 2; the CDR2 sequence of SEQ ID NO: 5; and the CDR3 sequence of SEQ ID NO: 9.

In a further embodiment, an anti-IL-2Rβγ heavy chain-only antibody or antigen-binding fragment thereof comprises the CDR1 sequence of SEQ ID NO: 3; the CDR2 sequence of SEQ ID NO: 6; and the CDR3 sequence of SEQ ID NO: 10.

In a further embodiment, an anti-IL-2Rβγ heavy chain-only antibody or antigen-binding fragment thereof comprises any of the heavy chain variable region amino acid sequences of SEQ ID NOs: 11-14 (Table 2).

In a still further embodiment, an anti-IL-2Rβγ heavy chain-only antibody or antigen-binding fragment thereof comprises the heavy chain variable region sequence of SEQ ID NO: 11. In a still further embodiment, an anti-IL-2Rβγ heavy chain-only antibody or antigen-binding fragment thereof comprises the heavy chain variable region sequence of SEQ ID NO: 12. In a still further embodiment, an anti-IL-2Rβγ heavy chain-only antibody or antigen-binding fragment thereof comprises the heavy chain variable region sequence of SEQ ID NO: 13. In a still further embodiment, an anti-IL-2Rβγ heavy chain-only antibody or antigen-binding fragment thereof comprises the heavy chain variable region sequence of SEQ ID NO: 14.

In some embodiments, a CDR sequence in an anti-IL-2Rβγ heavy chain-only antibody or antigen-binding fragment thereof of the disclosure comprises one or two amino acid substitutions relative to a CDR1, CDR2, and/or CDR3 sequence or set of CDR1, CDR2, and CDR3 sequences in any one of SEQ ID NOs: 1-10 (Table 1).

In some embodiments, an anti-IL-2Rβγ heavy chain-only antibody or antigen-binding fragment thereof comprises a heavy chain variable domain (VH) in which the CDR3 sequence has greater than or equal to 80%, such as at least 85%, at least 90%, at least 95%, or at least 99% sequence identity at the amino acid level to a CDR3 sequence of any one of the antibodies whose CDR3 sequences are provided in Table 1, and specifically binds to IL2RB.

In some embodiments, an anti-IL-2Rβγ heavy chain-only antibody or antigen-binding fragment thereof comprises a heavy chain variable domain (V_{H}) in which the full set of CDRs 1, 2, and 3 (combined) has greater than or equal to eighty-five percent (85%) (e.g., ≥90%, ≥95%, ≥98%, ≥99%) sequence identity at the amino acid level to the CDRs 1, 2, and 3 (combined) of the antibodies whose CDR sequences are provided in Table 1, and specifically binds to IL2RB.

In some embodiments, an anti-IL-2Rβγ heavy chain-only antibody or antigen-binding fragment thereof comprises a heavy chain variable region sequence with at least about 80% identity, at least 85% identity, at least 90% identity, at least 95% identity, at least 98% identity, or at least 99% identity to any of the heavy chain variable region sequences of SEQ ID NOs: 11-14 (shown in Table 2), and specifically binds to IL2RB.

In some alternative embodiments, an anti-IL-2Rβγ antibody or antigen-binding fragment thereof is used in place of an anti-IL-2Rβγ heavy chain-only antibody or antigen-binding fragment thereof. In some embodiments, the anti-IL-2Rβγ antibody or antigen-binding fragment thereof comprises a heavy chain variable region sequence as described herein, paired with a fixed light chain sequence. In some embodiments, the fixed light chain comprises a CDR1 sequence of SEQ ID NO: 44, a CDR2 sequence of SEQ ID NO: 45, and a CDR3 sequence of SEQ ID NO: 46, in a human VL framework. Together, the anti-IL2RB VH region and the fixed light chain variable region have binding affinity for IL2RB. In some embodiments, a fixed light chain comprises a light chain variable region sequence of SEQ ID NO: 47. In some embodiments, a fixed light chain comprises a sequence having at least about 80%, at least about 85%, at least about 90%, at least about 95%, or at least about 99% percent identity to the heavy chain variable region sequence of SEQ ID NO: 47. In some embodiments, a fixed light chain further comprises a light chain constant region sequence (CL). In some embodiments, a fixed light chain comprises the sequence of SEQ ID NO: 48.

In a particular embodiment, an anti-IL-2Rβγ heavy chain-only antibody or antigen-binding fragment thereof comprises a CDR1 sequence of any one of SEQ ID NOs: 15-16. In a particular embodiment, the CDR1 sequence comprises SEQ ID NO: 15. In a particular embodiment, the CDR1 sequence comprises SEQ ID NO: 16.

In a particular embodiment, an anti-IL-2Rβγ heavy chain-only antibody or antigen-binding fragment thereof comprises a CDR2 sequence of any one of SEQ ID NOs: 17-19. In a particular embodiment, the CDR2 sequence comprises SEQ ID NO: 17. In a particular embodiment, the CDR2 sequence comprises SEQ ID NO: 18. In a particular embodiment, the CDR2 sequence comprises SEQ ID NO: 19.

In a particular embodiment, an anti-IL-2Rβγ heavy chain-only antibody or antigen-binding fragment thereof comprises a CDR3 sequence of any one of SEQ ID NOs: 20-21. In a particular embodiment, the CDR3 sequence comprises SEQ ID NO: 20. In a particular embodiment, the CDR2 sequence comprises SEQ ID NO: 21.

In a further embodiment, an anti-IL-2Rβγ heavy chain-only antibody or antigen-binding fragment thereof comprises the CDR1 sequence of SEQ ID NO: 15; the CDR2 sequence of SEQ ID NO: 17; and the CDR3 sequence of SEQ ID NO: 20.

In a further embodiment, an anti-IL-2Rβγ heavy chain-only antibody or antigen-binding fragment thereof comprises the CDR1 sequence of SEQ ID NO: 15; the CDR2 sequence of SEQ ID NO: 18; and the CDR3 sequence of SEQ ID NO: 20.

In a further embodiment, an anti-IL-2Rβγ heavy chain-only antibody or antigen-binding fragment thereof comprises the CDR1 sequence of SEQ ID NO: 16; the CDR2 sequence of SEQ ID NO: 18; and the CDR3 sequence of SEQ ID NO: 20.

In a further embodiment, an anti-IL-2Rβγ heavy chain-only antibody or antigen-binding fragment thereof comprises the CDR1 sequence of SEQ ID NO: 15; the CDR2 sequence of SEQ ID NO: 19; and the CDR3 sequence of SEQ ID NO: 21.

In a further embodiment, an anti-IL-2Rβγ heavy chain-only antibody or antigen-binding fragment thereof comprises any of the heavy chain variable region amino acid sequences of SEQ ID NOs: 22-25 (Table 4).

In a still further embodiment, an anti-IL-2Rβγ heavy chain-only antibody or antigen-binding fragment thereof comprises the heavy chain variable region sequence of SEQ ID NO: 22. In a still further embodiment, an anti-IL-2Rβγ heavy chain-only antibody or antigen-binding fragment thereof comprises the heavy chain variable region sequence of SEQ ID NO: 23. In a still further embodiment, an anti-IL-2Rβγ heavy chain-only antibody or antigen-binding fragment thereof comprises the heavy chain variable region sequence of SEQ ID NO: 24. In a still further embodiment, an anti-IL-2Rβγ heavy chain-only antibody or antigen-binding fragment thereof comprises the heavy chain variable region sequence of SEQ ID NO: 25.

In some embodiments, a CDR sequence in an anti-IL-2Rβγ heavy chain-only antibody or antigen-binding fragment thereof of the disclosure comprises one or two amino acid substitutions relative to a CDR1, CDR2, and/or CDR3 sequence or set of CDR1, CDR2, and CDR3 sequences in any one of SEQ ID NOs: 15-21 (Table 3).

In some embodiments, an anti-IL-2Rβγ heavy chain-only antibody or antigen-binding fragment thereof comprises a heavy chain variable domain (VH) in which the CDR3 sequence has greater than or equal to 80%, such as at least 85%, at least 90%, at least 95%, or at least 99% sequence identity at the amino acid level to a CDR3 sequence of any one of the antibodies whose CDR3 sequences are provided in Table 3, and specifically binds to IL2RG.

In some embodiments, an anti-IL-2Rβγ heavy chain-only antibody or antigen-binding fragment thereof comprises a heavy chain variable domain (VH) in which the full set of CDRs 1, 2, and 3 (combined) has greater than or equal to eighty-five percent (85%) (e.g., ≥90%, ≥95%, ≥98%, ≥99%) sequence identity at the amino acid level to the CDRs 1, 2, and 3 (combined) of the antibodies whose CDR sequences are provided in Table 3, and specifically binds to IL2RG.

In some embodiments, an anti-IL-2Rβγ heavy chain-only antibody or antigen-binding fragment thereof comprises a heavy chain variable region sequence with at least about 80% identity, at least 85% identity, at least 90% identity, at least 95% identity, at least 98% identity, or at least 99% identity to any of the heavy chain variable region sequences of SEQ ID NOs: 22-25 (shown in Table 4), and specifically binds to IL2RG.

In some alternative embodiments, an anti-IL-2Rβγ antibody or antigen-binding fragment thereof is used in place of an anti-IL-2Rβγ heavy chain-only antibody or antigen-binding fragment thereof. In some embodiments, the anti-IL-2Rβγ antibody or antigen-binding fragment thereof comprises a heavy chain variable region sequence as described herein, paired with a fixed light chain sequence. In some embodiments, the fixed light chain comprises a CDR1 sequence of SEQ ID NO: 44, a CDR2 sequence of SEQ ID NO: 45, and a CDR3 sequence of SEQ ID NO: 46, in a human VL framework. Together, the anti-IL2RG VH region and the fixed light chain variable region have binding affinity for IL2RG. In some embodiments, a fixed light chain comprises a light chain variable region sequence of SEQ ID NO: 47. In some embodiments, a fixed light chain comprises a sequence having at least about 80%, at least about 85%, at least about 90%, at least about 95%, or at least about 99% percent identity to the heavy chain variable region sequence of SEQ ID NO: 47. In some embodiments, a fixed light chain further comprises a light chain constant region sequence (CL). In some embodiments, a fixed light chain comprises the sequence of SEQ ID NO: 48.

In some embodiments, an anti-IL-2Rβγ heavy chain-only antibody or antigen-binding fragment thereof used in a method described herein may have any of the configurations discussed herein, including, without limitation, a bispecific, bivalent heavy-chain antibody format comprising two non-identical heavy chain polypeptide subunits that are associated with one another via an asymmetric (e.g., knobs-in-holes (KiH)) interface. In certain embodiments, a bispecific, bivalent heavy chain antibody can comprise two non-identical heavy chain polypeptide subunits that are associated with one another via an asymmetric interface, and may optionally further include two identical fixed light chain polypeptide subunits, each of which associates with one of the two heavy chain polypeptide subunits.

In some embodiments, an anti-IL-2Rβγ heavy chain-only antibody or antigen-binding fragment thereof comprises at least one heavy chain variable region that specifically binds to IL2RB and at least one heavy chain variable region that specifically binds to IL2RG. In some embodiments, an anti-IL-2Rβγ heavy chain-only antibody or antigen-binding fragment further comprises an Fc portion comprising CH2 and/or CH3 and/or CH4 domains, in the absence of a CH1 domain.

In some embodiments, an anti-IL-2Rβγ heavy chain-only antibody or antigen-binding fragment thereof comprises a first variable region comprising a member of the IL2RB_F09 family, comprising a CDR1 sequence comprising SEQ ID NO: 26, a CDR2 sequence comprising SEQ ID NO: 27, and a CDR3 sequence comprising SEQ ID NO: 28, and a second variable region comprising a member of the IL2RG_F16 family, comprising a CDR1 sequence comprising SEQ ID NO: 32, a CDR2 sequence comprising SEQ ID NO: 33, and a CDR3 sequence comprising SEQ ID NO: 20.

In some embodiments, an anti-IL-2Rβγ heavy chain-only antibody or antigen-binding fragment thereof comprises a first variable region comprising a member of the IL2RB_F09 family, comprising a CDR1 sequence comprising SEQ ID NO: 26, a CDR2 sequence comprising SEQ ID NO: 27, and a CDR3 sequence comprising SEQ ID NO: 28, and a second variable region comprising a member of the IL2RG_F18 family, comprising a CDR1 sequence comprising SEQ ID NO: 15, a CDR2 sequence comprising SEQ ID NO: 19, and a CDR3 sequence comprising SEQ ID NO: 21.

In some embodiments, an anti-IL-2Rβγ heavy chain-only antibody or antigen-binding fragment thereof comprises a first variable region comprising a member of the IL2RB_F18 family, comprising a CDR1 sequence comprising SEQ ID NO: 29, a CDR2 sequence comprising SEQ ID NO: 30, and a CDR3 sequence comprising SEQ ID NO: 31, and a second variable region comprising a member of the IL2RG_F16 family, comprising a CDR1 sequence comprising SEQ ID NO: 32, a CDR2 sequence comprising SEQ ID NO: 33, and a CDR3 sequence comprising SEQ ID NO: 20.

In some embodiments, an anti-IL-2Rβγ heavy chain-only antibody or antigen-binding fragment thereof comprises a first variable region comprising a member of the IL2RB_F18 family, comprising a CDR1 sequence comprising SEQ ID NO: 29, a CDR2 sequence comprising SEQ ID NO: 30, and a CDR3 sequence comprising SEQ ID NO: 31, and a second variable region comprising a member of the IL2RG_F18 family, comprising a CDR1 sequence comprising SEQ ID NO: 15, a CDR2 sequence comprising SEQ ID NO: 19, and a CDR3 sequence comprising SEQ ID NO: 21.

In some embodiments, an anti-IL-2Rβγ heavy chain-only antibody or antigen-binding fragment thereof comprises a first variable region that specifically binds to IL2RB, comprising a CDR1 sequence of SEQ ID NO: 1, a CDR2 sequence of SEQ ID NO: 4, and a CDR3 sequence of SEQ ID NO: 7, and a second variable region that specifically binds to IL2RG, comprising a CDR1 sequence of SEQ ID NO: 15, a CDR2 sequence of SEQ ID NO: 17, and a CDR3 sequence of SEQ ID NO: 20.

In some embodiments, an anti-IL-2Rβγ heavy chain-only antibody or antigen-binding fragment thereof comprises a first variable region that specifically binds to IL2RB, comprising a CDR1 sequence of SEQ ID NO: 1, a CDR2 sequence of SEQ ID NO: 4, and a CDR3 sequence of SEQ ID NO: 8, and a second variable region that specifically binds to IL2RG, comprising a CDR1 sequence of SEQ ID NO: 15, a CDR2 sequence of SEQ ID NO: 18, and a CDR3 sequence of SEQ ID NO: 20.

In some embodiments, an anti-IL-2Rβγ heavy chain-only antibody or antigen-binding fragment thereof comprises a first variable region that specifically binds to IL2RB, comprising a CDR1 sequence of SEQ ID NO: 2, a CDR2 sequence of SEQ ID NO: 5, and a CDR3 sequence of SEQ ID NO: 9, and a second variable region that specifically binds to IL2RG, comprising a CDR1 sequence of SEQ ID NO: 15, a CDR2 sequence of SEQ ID NO: 18, and a CDR3 sequence of SEQ ID NO: 20.

In some embodiments, an anti-IL-2Rβγ heavy chain-only antibody or antigen-binding fragment thereof comprises a first variable region that specifically binds to IL2RB, comprising a CDR1 sequence of SEQ ID NO: 3, a CDR2 sequence of SEQ ID NO: 6, and a CDR3 sequence of SEQ ID NO: 10, and a second variable region that specifically binds to IL2RG, comprising a CDR1 sequence of SEQ ID NO: 15, a CDR2 sequence of SEQ ID NO: 17, and a CDR3 sequence of SEQ ID NO: 20.

In some embodiments, an anti-IL-2Rβγ heavy chain-only antibody or antigen-binding fragment thereof comprises a first variable region that specifically binds to IL2RB, comprising a CDR1 sequence of SEQ ID NO: 1, a CDR2 sequence of SEQ ID NO: 4, and a CDR3 sequence of SEQ ID NO: 8, and a second variable region that specifically binds to IL2RG, comprising a CDR1 sequence of SEQ ID NO: 16, a CDR2 sequence of SEQ ID NO: 18, and a CDR3 sequence of SEQ ID NO: 20.

In some embodiments, an anti-IL-2Rβγ heavy chain-only antibody or antigen-binding fragment thereof comprises a first variable region that specifically binds to IL2RB, comprising a CDR1 sequence of SEQ ID NO: 1, a CDR2 sequence of SEQ ID NO: 4, and a CDR3 sequence of SEQ ID NO: 8, and a second variable region that specifically binds to IL2RG, comprising a CDR1 sequence of SEQ ID NO: 15, a CDR2 sequence of SEQ ID NO: 19, and a CDR3 sequence of SEQ ID NO: 21.

Table 5 provides a summary of various CDR combinations of bispecific IL2RB x IL2RG heavy-chain only antibodies and antigen-binding fragments thereof in accordance with certain embodiments of the present disclosure.

TABLE 5
Bispecific IL2RB x IL2RG Heavy-Chain Only Antibodies and Antigen-Binding
Fragments, CDR Sequence Combinations

| Family ID No. | | | |
|---|---|---|---|
| Combination | CDR1 | CDR2 | CDR3 |
| IL2RB_F09C | GGSISSSDW | IDHSGST | GRGSWELSDAFDI |
| ** | (SEQ ID NO: 1) | (SEQ ID NO: 4) | (SEQ ID NO: 7) |
| IL2RG_F16A | GFTFSDYY | ISSSGDTI | ARGDAVSITGDY |
| | (SEQ ID NO: 15) | (SEQ ID NO: 17) | (SEQ ID NO: 20) |
| IL2RB_F09G | GGSISSSDW | IDHSGST | ARGSWELTDAFDI |
| ** | (SEQ ID NO: 1) | (SEQ ID NO: 4) | (SEQ ID NO: 8) |
| IL2RG_F16B | GFTFSDYY | ISSSGSTI | ARGDAVSITGDY |
| | (SEQ ID NO: 15) | (SEQ ID NO: 18) | (SEQ ID NO: 20) |
| IL2RB_F09K | GGSISSSNW | ISHSGST | GRGSWELTDAFDI |
| ** | (SEQ ID NO: 2) | (SEQ ID NO: 5) | (SEQ ID NO: 9) |
| IL2RG_F16B | GFTFSDYY | ISSSGSTI | ARGDAVSITGDY |
| | (SEQ ID NO: 15) | (SEQ ID NO: 18) | (SEQ ID NO: 20) |
| IL2RB_F18E | GFTFSSYG | ISYDGSNK | ARDLDYDVLTGDPVGGFDI |
| ** | (SEQ ID NO: 3) | (SEQ ID NO: 6) | (SEQ ID NO: 10) |
| IL2RG_F16A | GFTFSDYY | ISSSGDTI | ARGDAVSITGDY |
| | (SEQ ID NO: 15) | (SEQ ID NO: 17) | (SEQ ID NO: 20) |
| IL2RB_F09G | GGSISSSDW | IDHSGST | ARGSWELTDAFDI |
| ** | (SEQ ID NO: 1) | (SEQ ID NO: 4) | (SEQ ID NO: 8) |
| IL2RG_F16C | GFTFNDYY | ISSSGSTI | ARGDAVSITGDY |
| | (SEQ ID NO: 16) | (SEQ ID NO: 18) | (SEQ ID NO: 20) |
| IL2RB_F09G | GGSISSSDW | IDHSGST | ARGSWELTDAFDI |
| ** | (SEQ ID NO: 1) | (SEQ ID NO: 4) | (SEQ ID NO: 8) |
| IL2RG_F18A | GFTFSDYY | ISSSGTTT | ARGAAVAPGFDS |
| | (SEQ ID NO: 15) | (SEQ ID NO: 19) | (SEQ ID NO: 21) |

In some embodiments, an anti-IL-2Rβγ heavy chain-only antibody or antigen-binding fragment thereof comprises a first variable region that specifically binds to IL2RB, comprising a heavy chain variable region sequence of SEQ ID NO: 11, and a second variable region that specifically binds to IL2RG, comprising a heavy chain variable region sequence of SEQ ID NO: 22.

In some embodiments, an anti-IL-2Rβγ heavy chain-only antibody or antigen-binding fragment thereof comprises a first variable region that specifically binds to IL2RB, comprising a heavy chain variable region sequence of SEQ ID NO: 12, and a second variable region that specifically binds to IL2RG, comprising a heavy chain variable region sequence of SEQ ID NO: 23.

In some embodiments, an anti-IL-2Rβγ heavy chain-only antibody or antigen-binding fragment thereof comprises a first variable region that specifically binds to IL2RB, comprising a heavy chain variable region sequence of SEQ ID NO: 13, and a second variable region that specifically binds to IL2RG, comprising a heavy chain variable region sequence of SEQ ID NO: 23.

In some embodiments, an anti-IL-2Rβγ heavy chain-only antibody or antigen-binding fragment thereof comprises a first variable region that specifically binds to IL2RB, comprising a heavy chain variable region sequence of SEQ ID NO: 14, and a second variable region that specifically binds to IL2RG, comprising a heavy chain variable region sequence of SEQ ID NO: 22.

In some embodiments, an anti-IL-2Rβγ heavy chain-only antibody or antigen-binding fragment thereof comprises a first variable region that specifically binds to IL2RB, comprising a heavy chain variable region sequence of SEQ ID NO: 12, and a second variable region that specifically binds to IL2RG, comprising a heavy chain variable region sequence of SEQ ID NO: 24.

In some embodiments, an anti-L-2Rβγ heavy chain-only antibody or antigen-binding fragment thereof comprises a first variable region that specifically binds to IL2RB, comprising a heavy chain variable region sequence of SEQ TD NO: 12, and a second variable region that specifically binds to IL2RG, comprising a heavy chain variable region sequence of SEQ TD NO: 25.

Table 6 provides a summary of various heavy chain variable region combinations of bispecific IL2RB x IL2RG heavy-chain only antibodies in accordance with certain embodiments of the present disclosure.

TABLE 6
Bispecific IL2RB x IL2RG Heavy Chain-Only Antibodies, VH Sequence
Combinations

| Family ID No. | |
|---|---|
| Combination | VH Sequences |
| IL2RB_F09C | QVQLQESGPGLVKPSGTLSLTCAVSGGSISSSDWWSWVRQPPGKG |
| ** | LEWIGEIDHSGSTNYNPSLMSRVTISVDKSKNQFSLKLSSVTAADTA |
| IL2RG_F16A | VYFCGRGSWELSDAFDIRGQGTLVTVSS (SEQ ID NO: 11) |
| | QVQLVESGGGLVKPGGSLRLSCAASGFTFSDYYMSWIRQAPGKGL |
| | EWVSSISSSGDTIYYADSVQGRFTLSRDNAENSLFLQMNSLRAEDT |
| | AVYYCARGDAVSITGDYRGQGTLVTVSS (SEQ ID NO: 22) |
| IL2RB_F09G | QVQLQESGPGLVKSSETLSLTCTVSGGSISSSDWWSWVRQPPGKGL |
| ** | EWIGEIDHSGSTNYNPSLMSRVTISVDKSKNQFSLKLSSVTAADTA |
| IL2RG_F16B | VYFCARGSWELTDAFDIRGQGTLVTVSS (SEQ ID NO: 12) |
| | QVQLVESGGGLVKPGGSLRLSCAASGFTFSDYYMSWIRQAPGKGL |
| | EWVSYISSSGSTIYYADSVKGRFTISRDNAKNSLYLQMNSLRAEDT |
| | AVYYCARGDAVSITGDYRGQGTLVTVSS (SEQ ID NO: 23) |
| IL2RB_F09K | QVQLQESSPGLVKPSETLSLTCTVSGGSISSSNWWSWVRQPPGKGL |
| ** | EWIGEISHSGSTNYNPSLKSRVTISVDKSKNQFSLRLSSVTAADTAV |
| IL2RG_F16B | YFCGRGSWELTDAFDIRGQGTLVTVSS (SEQ ID NO: 13) |
| | QVQLVESGGGLVKPGGSLRLSCAASGFTFSDYYMSWIRQAPGKGL |
| | EWVSYISSSGSTIYYADSVKGRFTISRDNAKNSLYLQMNSLRAEDT |
| | AVYYCARGDAVSITGDYRGQGTLVTVSS (SEQ ID NO: 23) |
| IL2RB_F18E | QVQLVESGGGVVQPGRSLRLSCAASGFTFSSYGMHWVRQAPGKE |
| ** | REWVAVISYDGSNKYYTDSVKGRFTISRDNSKNTLYLEMNSLRAE |
| IL2RG_F16A | DTAVYYCARDLDYDVLTGDPVGGFDIWGQGTLVTVSS (SEQ ID |
| | NO: 14) |
| | QVQLVESGGGLVKPGGSLRLSCAASGFTFSDYYMSWIRQAPGKGL |
| | EWVSSISSSGDTIYYADSVQGRFTLSRDNAENSLFLQMNSLRAEDT |
| | AVYYCARGDAVSITGDYRGQGTLVTVSS (SEQ ID NO: 22) |
| IL2RB_F09G | QVQLQESGPGLVKSSETLSLTCTVSGGSISSSDWWSWVRQPPGKGL |
| ** | EWIGEIDHSGSTNYNPSLMSRVTISVDKSKNQFSLKLSSVTAADTA |
| IL2RG_F16C | VYFCARGSWELTDAFDIRGQGTLVTVSS (SEQ ID NO: 12) |
| | QVQLVESGGGLVKPGGSLRLSCAASGFTFNDYYMSWIRQAPGKGL |
| | EWVSHISSSGSTIYYADSVKGRFTVSRDNANNSLYLQMHSLRAEDT |
| | AVYYCARGDAVSITGDYRGQGTLVTVSS (SEQ ID NO: 24) |
| IL2RB_F09G | QVQLQESGPGLVKSSETLSLTCTVSGGSISSSDWWSWVRQPPGKGL |
| ** | EWIGEIDHSGSTNYNPSLMSRVTISVDKSKNQFSLKLSSVTAADTA |
| IL2RG_F18A | VYFCARGSWELTDAFDIRGQGTLVTVSS (SEQ ID NO: 12) |
| | QVQLVESGGDLVKPGGSLRLSCAASGFTFSDYYMSWLRQAPGKEL |
| | EWVSHISSSGTTTYYADSVEGRFTITRDNAKNSLYLQMNSLRAEDT |
| | AVYYCARGAAVAPGFDSRGQGTLVTVSS (SEQ ID NO: 25) |

In some embodiments, an anti-IL-2Rβγ heavy chain-only antibody or antigen-binding fragment thereof includes a first and a second polypeptide, i.e., a first and a second polypeptide subunit, wherein each polypeptide comprises an antigen-binding domain of a heavy chain-only antibody. In some embodiments, each of the first and second polypeptides further includes a hinge region, or at least a portion of a hinge region, which can facilitate formation of at least one disulfide bond between the first and second polypeptides. In some embodiments, each of the first and second polypeptides further includes at least one heavy chain constant region (CH) domain, such as a CH2 domain, and/or a CH3 domain, and/or a CH4 domain. In certain embodiments, the CH domain lacks a CH1 domain. The antigen-binding domain of each of the first and second polypeptides can incorporate any of the CDR sequences and/or variable region sequences described herein in order to impart antigen-binding capability on the anti-IL-2Rβγ heavy chain-only antibody or antigen-binding fragment. As such, in certain embodiments, each polypeptide subunit in an anti-IL-2Rβγ heavy chain-only antibody or antigen-binding fragment can include an antigen-binding domain that specifically binds to a different IL2R subunit, or chain (e.g., IL2RB and IL2RG).

In some embodiments, an anti-IL-2Rβγ heavy chain-only antibody or antigen-binding fragment thereof comprises a variant human IgG4 Fc domain comprising a first heavy chain constant region sequence comprising an S228P mutation, an F234A mutation, an L235A mutation, and a T366W mutation (knob), and a second heavy chain constant region sequence comprising an S228P mutation, an F234A mutation, an L235A mutation, a T366S mutation, an L368A mutation, and a Y407V mutation (hole). This variant, or modified, IgG4 Fc domain prevents unwanted Fab exchange, reduces effector function of the anti-IL-2Rβγ heavy chain-only antibody or antigen-binding fragment, and also facilitates heterodimerization of the heavy chain polypeptide subunits to form the multispecific (e.g., bispecific) antibody.

The components of the anti-IL-2Rβγ heavy chain-only antibodies and antigen-binding fragments described herein (i.e., CDR sequences, variable region sequences, and Fc domain sequences (e.g., hinge, CH2, and CH3 domain sequences)) can be combined in various ways to generate heavy chain-only antibodies and antigen-binding fragments that bind to IL2R, e.g., to IL2RB and IL2RB, and that have beneficial properties, e.g., reduced effector function activity, increased IL2R agonistic activity, etc.

Table 7 provides the sequences of human IgG1 and IgG4 Fc region sequences, as well as versions of these sequences that incorporate additional mutations (variants) that impart additional desired properties.

TABLE 7
Human IgG1 and IgG4 Fc Region Sequences and Variants Thereof

| Polypeptide Name | Amino Acid Sequence |
|---|---|
| Human IgG1 | ASTKGPSVFPLAPSSKSTSGGTAALGCLVKDYFPEPVTVS |
| (UniProt No. | WNSGALTSGVHTFPAVLQSSGLYSLSSVVTVPSSSLGTQT |
| P01857) | YICNVNHKPSNTKVDKKVEPKSCDKTHTCPPCPAPELLGG |
| | PSVFLFPPKPKDTLMISRTPEVTCVVVDVSHEDPEVKFNW |
| | YVDGVEVHNAKTKPREEQYNSTYRVVSVLTVLHQDWLNGK |
| | EYKCKVSNKALPAPIEKTISKAKGQPREPQVYTLPPSRDE |
| | LTKNQVSLTCLVKGFYPSDIAVEWESNGQPENNYKTTPPV |
| | LDSDGSFFLYSKLTVDKSRWQQGNVFSCSVMHEALHNHYT |
| | QKSLSLSPGK (SEQ ID NO: 42) |
| Human IgG4 | ASTKGPSVFPLAPCSRSTSESTAALGCLVKDYFPEPVTVS |
| (UniProt No. | WNSGALTSGVHTFPAVLQSSGLYSLSSVVTVPSSSLGTKT |
| P01861) | YTCNVDHKPSNTKVDKRVESKYGPPCPSCPAPEFLGGPSV |
| | FLFPPKPKDTLMISRTPEVTCVVVDVSQEDPEVQFNWYVD |
| | GVEVHNAKTKPREEQFNSTYRVVSVLTVLHQDWLNGKEYK |
| | CKVSNKGLPSSIEKTISKAKGQPREPQVYTLPPSQEEMTK |
| | NQVSLTCLVKGFYPSDIAVEWESNGQPENNYKTTPPVLDS |
| | DGSFFLYSRLTVDKSRWQEGNVFSCSVMHEALHNHYTQKS |
| | LSLSLGK (SEQ ID NO: 43) |
| Human IgG1 with | ASTKGPSVFPLAPSSKSTSGGTAALGCLVKDYFPEPVTVSWN |
| silencing mutations | SGALTSGVHTFPAVLQSSGLYSLSSVVTVPSSSLGTQTYICNV |
| (Fc region) | NHKPSNTKVDKKVEPKSCDKTHTCPPCPAPEAAGGPSVFLFP |
| | PKPKDTLMISRTPEVTCVVVDVSHEDPEVKFNWYVDGVEVH |
| | NAKTKPREEQYNSTYRVVSVLTVLHQDWLNGKEYKCKVSN |
| | KALPAPIEKTISKAKGQPREPQVYTLPPSREEMTKNQVSLTCL |
| | VKGFYPSDIAVEWESNGQPENNYKTTPPVLDSDGSFFLYSKL |
| | TVDKSRWQQGNVFSCSVMHEALHNHYTQKSLSLSPGK (SEQ |
| | ID NO: 51) |
| Human IgG4 with | ASTKGPSVFPLAPCSRSTSESTAALGCLVKDYFPEPVTVSWNS |
| silencing mutations | GALTSGVHTFPAVLQSSGLYSLSSVVTVPSSSLGTKTYTCNV |
| (Fc region) | DHKPSNTKVDKRVESKYGPPCPPCPAPEAAGGPSVFLFPPKP |
| | KDTLMISRTPEVTCVVVDVSQEDPEVQFNWYVDGVEVHNA |
| | KTKPREEQFNSTYRVVSVLTVLHQDWLNGKEYKCKVSNKGL |
| | PSSIEKTISKAKGQPREPQVYTLPPSQEEMTKNQVSLTCLVKG |
| | FYPSDIAVEWESNGQPENNYKTTPPVLDSDGSFFLYSRLTVD |
| | KSRWQEGNVFSCSVMHEALHNHYTQKSLSLSLGK |
| | (SEQ ID NO: 52) |
| Human IgG4 hinge | ESKYGPPCPSCPA (SEQ ID NO: 54) |
| region (wild type) | |
| Human IgG4 hinge | ESKYGPPCPPCPA (SEQ ID NO: 55) |
| region (S228P) | |
| Human IgG4 CH2 | APEFLGGPSVFLFPPKPKDTLMISRTPEVTCVVVDVSQEDPEV |
| domain sequence | QFNWYVDGVEVHNAKTKPREEQFNSTYRVVSVLTVLHQDW |
| (wild type) | LNGKEYKCKVSNKGLPSSIEKTISKAK (SEQ ID NO: 56) |
| Human IgG4 CH2 | APEAAGGPSVFLFPPKPKDTLMISRTPEVTCVVVDVSQEDPEV |
| domain sequence | QFNWYVDGVEVHNAKTKPREEQFNSTYRVVSVLTVLHQDW |
| F234A, L235A) | LNGKEYKCKVSNKGLPSSIEKTISKAK (SEQ ID NO: 57) |
| Human IgG4 CH3 | GQPREPQVYTLPPSQEEMTKNQVSLTCLVKGFYPSDIAVEWE |
| domain sequence | SNGQPENNYKTTPPVLDSDGSFFLYSRLTVDKSRWQEGNVFS |
| (wild type) | CSVMHEALHNHYTQKSLSLSLGK (SEQ ID NO: 58) |
| Human IgG4 CH3 | GQPREPQVYTLPPSQEEMTKNQVSLWCLVKGFYPSDIAVEW |
| domain sequence | ESNGQPENNYKTTPPVLDSDGSFFLYSRLTVDKSRWQEGNVF |
| (knob, T366W) | SCSVMHEALHNHYTQKSLSLSLGK (SEQ ID NO: 59) |
| Human IgG4 CH3 | GQPREPQVYTLPPSQEEMTKNQVSLSCAVKGFYPSDIAVEWE |
| domain sequence | SNGQPENNYKTTPPVLDSDGSFFLVSRLTVDKSRWQEGNVFS |
| (hole, T366S, L368A, | CSVMHEALHNHYTQKSLSLSLGK (SEQ ID NO: 60) |
| Y407V) | |

In some embodiments, an anti-IL-2Rβγ heavy chain-only antibody or antigen-binding fragment thereof comprises a first heavy chain polypeptide subunit comprising a variable region that specifically binds to IL2RB, wherein the first heavy chain polypeptide subunit comprises the sequence of SEQ ID NO: 53, and a second heavy chain polypeptide subunit comprising a variable region that specifically binds to IL2RG, wherein the second heavy chain polypeptide subunit comprises the sequence of SEQ ID NO: 61.

In some embodiments, an anti-IL-2Rβγ heavy chain-only antibody or antigen-binding fragment thereof comprises a first heavy chain polypeptide subunit comprising a variable region that specifically binds to IL2RB, wherein the first heavy chain polypeptide subunit comprises the sequence of SEQ ID NO: 62, and a second heavy chain polypeptide subunit comprising a variable region that specifically binds to IL2RG, wherein the second heavy chain polypeptide subunit comprises the sequence of SEQ ID NO: 63.

In some embodiments, an anti-IL-2Rβγ heavy chain-only antibody or antigen-binding fragment thereof comprises a first heavy chain polypeptide subunit comprising a variable region that specifically binds to IL2RB, wherein the first heavy chain polypeptide subunit comprises the sequence of SEQ ID NO: 64, and a second heavy chain polypeptide subunit comprising a variable region that specifically binds to IL2RG, wherein the second heavy chain polypeptide subunit comprises the sequence of SEQ ID NO: 65.

In some embodiments, an anti-IL-2Rβγ heavy chain-only antibody or antigen-binding fragment thereof comprises a first heavy chain polypeptide subunit comprising a variable region that specifically binds to IL2RB, wherein the first heavy chain polypeptide subunit comprises the sequence of SEQ ID NO: 66, and a second heavy chain polypeptide subunit comprising a variable region that specifically binds to IL2RG, wherein the second heavy chain polypeptide subunit comprises the sequence of SEQ ID NO: 67.

In some embodiments, an anti-IL-2Rβγ heavy chain-only antibody or antigen-binding fragment thereof comprises a first heavy chain polypeptide subunit comprising a variable region that specifically binds to IL2RB, wherein the first heavy chain polypeptide subunit comprises the sequence of SEQ ID NO: 34, and a second heavy chain polypeptide subunit comprising a variable region that specifically binds to IL2RG, wherein the second heavy chain polypeptide subunit comprises the sequence of SEQ ID NO: 35.

In some embodiments, an anti-IL-2Rβγ heavy chain-only antibody or antigen-binding fragment thereof comprises a first heavy chain polypeptide subunit comprising a variable region that specifically binds to IL2RB, wherein the first heavy chain polypeptide subunit comprises the sequence of SEQ ID NO: 36, and a second heavy chain polypeptide subunit comprising a variable region that specifically binds to IL2RG, wherein the second heavy chain polypeptide subunit comprises the sequence of SEQ ID NO: 37.

Table 8 provides a summary of various heavy chain polypeptide subunit sequence combinations of bispecific IL2RB x IL2RG heavy chain-only antibodies in accordance with certain embodiments of the present disclosure.

TABLE 8
Bispecific IL2RB x IL2RG Heavy Chain-Only Antibodies, Full Length Polypeptide
Sequence Combinations

| Family ID No. | Polypeptide | |
|---|---|---|
| Combination | Name | Full Length Sequences |
| IL2RB_F09C | IL2RB_F09C | QVQLQESGPGLVKPSGTLSLTCAVSGGSISSSDWWS |
| hole | IgG4 hole | WVRQPPGKGLEWIGEIDHSGSTNYNPSLMSRVTISV |
| ** | | DKSKNQFSLKLSSVTAADTAVYFCGRGSWELSDAF |
| IL2RG_F16A | | DIRGQGTLVTVSSESKYGPPCPPCPAPEAAGGPSVF |
| knob | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLSCAVKGFYPSDIAVE |
| | | WESNGQPENNYKTTPPVLDSDGSFFLVSRLTVDKS |
| | | RWQEGNVFSCSVMHEALHNHYTQKSLSLSLGK |
| | | (SEQ ID NO: 53) |
| | IL2RG_F16A | QVQLVESGGGLVKPGGSLRLSCAASGFTFSDYYMS |
| | IgG4 knob | WIRQAPGKGLEWVSSISSSGDTIYYADSVQGRFTLS |
| | | RDNAENSLFLQMNSLRAEDTAVYYCARGDAVSITG |
| | | DYRGQGTLVTVSSESKYGPPCPPCPAPEAAGGPSVF |
| | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 61) |
| IL2RB_F09G | IL2RB_F09G | QVQLQESGPGLVKSSETLSLTCTVSGGSISSSDWWS |
| hole | IgG4 hole | WVRQPPGKGLEWIGEIDHSGSTNYNPSLMSRVTISV |
| ** | | DKSKNQFSLKLSSVTAADTAVYFCARGSWELTDAF |
| IL2RG_F16B | | DIRGQGTLVTVSSESKYGPPCPPCPAPEAAGGPSVF |
| knob | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLSCAVKGFYPSDIAVE |
| | | WESNGQPENNYKTTPPVLDSDGSFFLVSRLTVDKS |
| | | RWQEGNVFSCSVMHEALHNHYTQKSLSLSLGK |
| | | (SEQ ID NO: 62) |
| | IL2RG_F16B | QVQLVESGGGLVKPGGSLRLSCAASGFTFSDYYMS |
| | IgG4 knob | WIRQAPGKGLEWVSYISSSGSTIYYADSVKGRFTIS |
| | | RDNAKNSLYLQMNSLRAEDTAVYYCARGDAVSIT |
| | | GDYRGQGTLVTVSSESKYGPPCPPCPAPEAAGGPSV |
| | | FLFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 63) |
| IL2RB_F09K | IL2RB_F09K | QVQLQESSPGLVKPSETLSLTCTVSGGSISSSNWWS |
| hole | IgG4 hole | WVRQPPGKGLEWIGEISHSGSTNYNPSLKSRVTISV |
| ** | | DKSKNQFSLRLSSVTAADTAVYFCGRGSWELTDAF |
| IL2RG_F16B | | DIRGQGTLVTVSSESKYGPPCPPCPAPEAAGGPSVF |
| knob | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLSCAVKGFYPSDIAVE |
| | | WESNGQPENNYKTTPPVLDSDGSFFLVSRLTVDKS |
| | | RWQEGNVFSCSVMHEALHNHYTQKSLSLSLGK |
| | | (SEQ ID NO: 64) |
| | IL2RG_F16B | QVQLVESGGGLVKPGGSLRLSCAASGFTFSDYYMS |
| | IgG4 knob | WIRQAPGKGLEWVSYISSSGSTIYYADSVKGRFTIS |
| | | RDNAKNSLYLQMNSLRAEDTAVYYCARGDAVSIT |
| | | GDYRGQGTLVTVSSESKYGPPCPPCPAPEAAGGPSV |
| | | FLFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 65) |
| IL2RB_F18E | IL2RB_F18E | QVQLVESGGGVVQPGRSLRLSCAASGFTFSSYGMH |
| hole | IgG4 hole | WVRQAPGKEREWVAVISYDGSNKYYTDSVKGRFT |
| ** | | ISRDNSKNTLYLEMNSLRAEDTAVYYCARDLDYDV |
| IL2RG_F16A | | LTGDPVGGFDIWGQGTLVTVSSESKYGPPCPPCPAP |
| knob | | EAAGGPSVFLFPPKPKDTLMISRTPEVTCVVVDVSQ |
| | | EDPEVQFNWYVDGVEVHNAKTKPREEQFNSTYRV |
| | | VSVLTVLHQDWLNGKEYKCKVSNKGLPSSIEKTIS |
| | | KAKGQPREPQVYTLPPSQEEMTKNQVSLSCAVKGF |
| | | YPSDIAVEWESNGQPENNYKTTPPVLDSDGSFFLVS |
| | | RLTVDKSRWQEGNVFSCSVMHEALHNHYTQKSLS |
| | | LSLGK (SEQ ID NO: 66) |
| | IL2RG_F16A | QVQLVESGGGLVKPGGSLRLSCAASGFTFSDYYMS |
| | IgG4 knob | WIRQAPGKGLEWVSSISSSGDTIYYADSVQGRFTLS |
| | | RDNAENSLFLQMNSLRAEDTAVYYCARGDAVSITG |
| | | DYRGQGTLVTVSSESKYGPPCPPCPAPEAAGGPSVF |
| | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 67) |
| IL2RB_F09G | IL2RB_F09G | QVQLQESGPGLVKSSETLSLTCTVSGGSISSSDWWS |
| hole | IgG4 hole | WVRQPPGKGLEWIGEIDHSGSTNYNPSLMSRVTISV |
| ** | | DKSKNQFSLKLSSVTAADTAVYFCARGSWELTDAF |
| IL2RG_F16C | | DIRGQGTLVTVSSESKYGPPCPPCPAPEAAGGPSVF |
| knob | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLSCAVKGFYPSDIAVE |
| | | WESNGQPENNYKTTPPVLDSDGSFFLVSRLTVDKS |
| | | RWQEGNVFSCSVMHEALHNHYTQKSLSLSLGK |
| | | (SEQ ID NO: 34) |
| | IL2RG_F16C | QVQLVESGGGLVKPGGSLRLSCAASGFTFNDYYMS |
| | IgG4 knob | WIRQAPGKGLEWVSHISSSGSTIYYADSVKGRFTVS |
| | | RDNANNSLYLQMHSLRAEDTAVYYCARGDAVSIT |
| | | GDYRGQGTLVTVSSESKYGPPCPPCPAPEAAGGPSV |
| | | FLFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 35) |
| IL2RB_F09G | IL2RB_F09G | QVQLQESGPGLVKSSETLSLTCTVSGGSISSSDWWS |
| hole | IgG4 hole | WVRQPPGKGLEWIGEIDHSGSTNYNPSLMSRVTISV |
| ** | | DKSKNQFSLKLSSVTAADTAVYFCARGSWELTDAF |
| IL2RG_F18A | | DIRGQGTLVTVSSESKYGPPCPPCPAPEAAGGPSVF |
| knob | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLSCAVKGFYPSDIAVE |
| | | WESNGQPENNYKTTPPVLDSDGSFFLVSRLTVDKS |
| | | RWQEGNVFSCSVMHEALHNHYTQKSLSLSLGK |
| | | (SEQ ID NO: 36) |
| | IL2RG_F18A | QVQLVESGGDLVKPGGSLRLSCAASGFTFSDYYMS |
| | IgG4 knob | WLRQAPGKELEWVSHISSSGTTTYYADSVEGRFTIT |
| | | RDNAKNSLYLQMNSLRAEDTAVYYCARGAAVAPG |
| | | FDSRGQGTLVTVSSESKYGPPCPPCPAPEAAGGPSV |
| | | FLFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPSQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| | | (SEQ ID NO: 37) |

Additional sequences referred to herein are provided in Tables 9 and 10 for reference.

TABLE 9
Additional Sequences

| Protein | UniProt | |
|---|---|---|
| Name | KB No. | Amino Acid Sequence |
| Human | P01589 | MDSYLLMWGLLTFIMVPGCQAELCDDDPPEIPHATFKA |
| IL2RA | | MAYKEGTMLNCECKRGFRRIKSGSLYMLCTGNSSHSS |
| | | WDNQCQCTSSATRNTTKQVTPQPEEQKERKTTEMQSP |
| | | MQPVDQASLPGHCREPPPWENEATERIYHFVVGQMVY |
| | | YQCVQGYRALHRGPAESVCKMTHGKTRWTQPQLICTG |
| | | EMETSQFPGEEKPQASPEGRPESETSCLVTTTDFQIQTE |
| | | MAATMETSIFTTEYQVAVAGCVFLLISVLLLSGLTWQR |
| | | RQRKSRRTI (SEQ ID NO: 38) |
| Human | P14784 | MAAPALSWRLPLLILLLPLATSWASAAVNGTSQFTCFY |
| IL2RB | | NSRANISCVWSQDGALQDTSCQVHAWPDRRRWNQTCE |
| | | LLPVSQASWACNLILGAPDSQKLTTVDIVTLRVLCREGV |
| | | RWRVMAIQDFKPFENLRLMAPISLQVVHVETHRCNISW |
| | | EISQASHYFERHLEFEARTLSPGHTWEEAPLLTLKQKQE |
| | | WICLETLTPDTQYEFQVRVKPLQGEFTTWSPWSQPLAF |
| | | RTKPAALGKDTIPWLGHLLVGLSGAFGFIILVYLLINCR |
| | | NTGPWLKKVLKCNTPDPSKFFSQLSSEHGGDVQKWLSS |
| | | PFPSSSFSPGGLAPEISPLEVLERDKVTQLLLQQDKVPEP |
| | | ASLSSNHSLTSCFTNQGYFFFHLPDALEIEACQVYFTYD |
| | | PYSEEDPDEGVAGAPTGSSPQPLQPLSGEDDAYCTFPSR |
| | | DDLLLFSPSLLGGPSPPSTAPGGSGAGEERMPPSLQERVP |
| | | RDWDPQPLGPPTPGVPDLVDFQPPPELVLREAGEEVPD |
| | | AGPREGVSFPWSRPPGQGEFRALNARLPLNTDAYLSLQ |
| | | ELQGQDPTHLV (SEQ ID NO: 39) |
| Human | P31785 | MLKPSLPFTSLLFLQLPLLGVGLNTTILTPNGNEDTTADF |
| IL2RG | | FLTTMPTDSLSVSTLPLPEVQCFVFNVEYMNCTWNSSSE |
| | | PQPTNLTLHYWYKNSDNDKVQKCSHYLFSEEITSGCQL |
| | | QKKEIHLYQTFVVQLQDPREPRRQATQMLKLQNLVIPW |
| | | APENLTLHKLSESQLELNWNNRFLNHCLEHLVQYRTD |
| | | WDHSWTEQSVDYRHKFSLPSVDGQKRYTFRVRSRFNP |
| | | LCGSAQHWSEWSHPIHWGSNTSKENPFLFALEAVVISV |
| | | GSMGLIISLLCVYFWLERTMPRIPTLKNLEDLVTEYHGN |
| | | FSAWSGVSKGLAESLQPDYSERLCLVSEIPPKGGALGEG |
| | | PGASPCNQHSPYWAPPCYTLKPET (SEQ ID NO: 40) |
| Human IL2 | P60568 | MYRMQLLSCIALSLALVTNSAPTSSSTKKTQLQLEHLLL |
| | | DLQMILNGINNYKNPKLTRMLTFKFYMPKKATELKHLQ |
| | | CLEEELKPLEEVLNLAQSKNFHLRPRDLISNINVIVLELK |
| | | GSETTFMCEYADETATIVEFLNRWITFCQSIISTLT (SEQ |
| | | ID NO: 41) |

TABLE 10
Additional Sequences

| Protein Name | Amino Acid Sequence |
|---|---|
| Fixed light chain, | QSVSSN (SEQ ID NO: 44) |
| CDR1 sequence | |
| Fixed light chain, | GAS (SEQ ID NO: 45) |
| CDR2 sequence | |
| Fixed light chain, | QQYNNWPWT (SEQ ID NO: 46) |
| CDR3 sequence | |
| Fixed light chain, VL | EIVMTQSPATLSVSPGERATLSCRASQSVSSNLAWYQQKPGQA |
| sequence | PRLLIYGASTRATGIPARFSGSGSGTEFTLTISSLQSEDFAVYYC |
| | QQYNNWPWTFGQGTKVEIK (SEQ ID NO: 47) |
| Fixed light chain, full | EIVMTQSPATLSVSPGERATLSCRASQSVSSNLAWYQQKPGQA |
| length sequence (VL + | PRLLIYGASTRATGIPARFSGSGSGTEFTLTISSLQSEDFAVYYC |
| CL) | QQYNNWPWTFGQGTKVEIKRTVAAPSVFIFPPSDEQLKSGTAS |
| | VVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 48) |

### Cellular Internalization

In some embodiments of the present disclosure, anti-IL-2Rβγ heavy chain-only antibodies and antigen-binding fragments thereof disclosed herein, once bound to a binding target (e.g., IL2R), internalize into cells, wherein internalization is at least about 10%, at least about 20%, at least about 30%, at least about 40%, at least about 50%, at least about 60%, at least about 70%, at least about 80%, or at least about 90%, at least about 100%, at least about 110%, at least about 120%, at least about 130%, at least about 140%, at least about 150%, at least about 160%, at least about 170%, at least about 180%, at least about 190%, or at least about 200% or more, in comparison to one or more control antibodies that do not internalize.

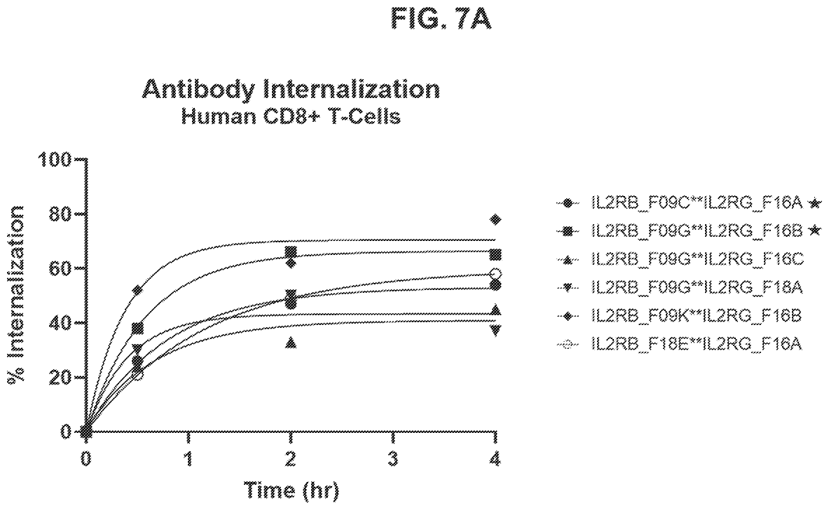

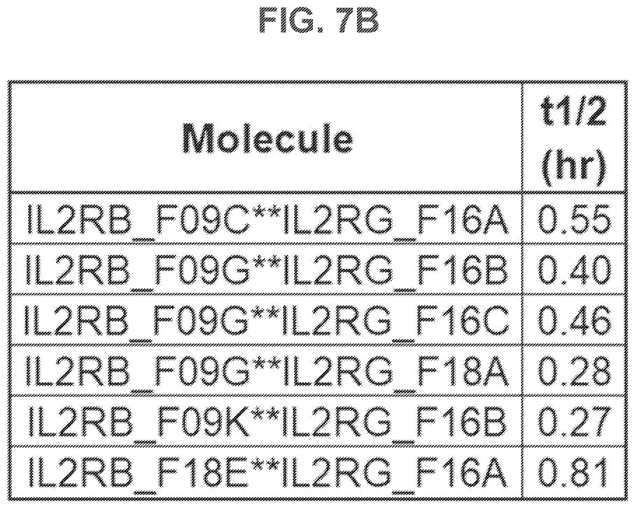

Cellular internalization results are provided in FIG. 7, panels A-B. Panel A depicts internalization of the indicated anti-IL2β/γ heavy chain-only antibodies by CD8⁺ T-cells from human PBMCs, as a function of time. Panel B provides depicts this data in tabular format. Surface levels of heavy chain-only antibody were detected by flow cytometry and reported relative to cells which had not been allowed to internalize. The observed half-lives ranged from 0.27 hours to 0.81 hours. As observed here, internalization was potentially partially dependent on the specific anti-IL2RG arm of the bispecific heavy chain-only antibody, as molecules comprising the IL2RG_F16B binding sequence internalized faster, and to a greater degree, than molecules containing different anti-IL2RG binding sequences.

### Preparation of Anti-IL2R Heavy Chain-Only Antibodies

The anti-IL2β/γ heavy chain-only antibodies and antigen-binding fragments thereof of the present disclosure can be prepared by methods known in the art. In some embodiments, the anti-IL2β/γ heavy chain-only antibodies and antigen-binding fragments thereof are produced by transgenic animals, including transgenic mice and rats, e.g., transgenic rats, in which the endogenous immunoglobulin genes are knocked out or disabled. In some embodiments, the anti-IL-2Rβγ heavy chain-only antibodies and antigen-binding fragments thereof described herein are produced in a UniRat™. UniRat™ have their endogenous immunoglobulin genes silenced and use a human immunoglobulin heavy-chain translocus to express a diverse, naturally optimized repertoire of fully human HCAbs. While endogenous immunoglobulin loci in rats can be knocked out or silenced using a variety of technologies, in UniRat™ the zinc-finger (endo)nuclease (ZNF) technology was used to inactivate the endogenous rat heavy chain J-locus, light chain Cx locus and light chain CX locus. ZNF constructs for microinjection into oocytes can produce IgH and IgL knock out (KO) lines. For details, see, e.g., Geurts et al., 2009, Science 325:433. Characterization of Ig heavy chain knockout rats has been reported by Menoret et al., 2010, Eur. J. Immunol. 40:2932-2941. Advantages of the ZNF technology are that non-homologous end joining to silence a gene or locus via deletions up to several kb can also provide a target site for homologous integration (Cui et al., 2011, Nat Biotechnol 29:64-67). Human heavy chain-only antibodies produced in UniRat™ are called UniAbs™ and can bind epitopes that cannot be attacked with conventional antibodies. Their high specificity, affinity, and small size make them ideal for mono- and poly-specific applications.

In addition to UniAbs™, specifically included herein are heavy chain-only antibodies and antigen-binding fragments thereof lacking the camelid VHH framework and mutations, and their functional VH regions. Such heavy chain-only antibodies and antigen-binding fragments thereof can, for example, be produced in transgenic rats or mice which comprise fully human heavy chain-only gene loci as described, e.g., in WO2006/008548, but other transgenic mammals, such as rabbit, guinea pig, and rat can also be used. Heavy chain-only antibodies and antigen-binding fragments, including their VHH or VH functional fragments, can also be produced by recombinant DNA technology, by expression of the encoding nucleic acid in a suitable eukaryotic or prokaryotic host, including, for example, mammalian cells (e.g., CHO cells), E. coli, or yeast.

Domains of heavy chain-only antibodies and antigen-binding fragments thereof combine advantages of antibodies and small molecule drugs: can be mono- or multi-valent; have low toxicity; and are cost-effective to manufacture. Due to their small size, these domains are easy to administer, including oral or topical administration, are characterized by high stability, including gastrointestinal stability; and their half-life can be tailored to the desired use or indication. In addition, VH and VHH domains of HCAbs can be manufactured in a cost-effective manner.

In a particular embodiment, the heavy chain-only antibodies and antigen-binding fragments thereof of the present disclosure, including UniAbs™, have the native amino acid residue at the first position of the FR4 region (amino acid position 101 according to the Kabat numbering system), substituted by another amino acid residue, which is capable of disrupting a surface-exposed hydrophobic patch comprising or associated with the native amino acid residue at that position. Such hydrophobic patches are normally buried in the interface with the antibody light chain constant region but become surface exposed in HCAbs and are, at least partially, for the unwanted aggregation and light chain association of HCAbs. In some embodiments, the substituted amino acid residue is charged. In some embodiments, the substituted amino acid residue is positively charged, such as lysine (Lys, K), arginine (Arg, R) or histidine (His, H), e.g., arginine (R). In some embodiments, the heavy chain-only antibodies and antigen-binding fragments thereof derived from the transgenic animals contain a Trp to Arg mutation at position 101. In some embodiments, the resultant heavy chain-only antibodies and antigen-binding fragments thereof have high antigen-binding affinity and solubility under physiological conditions in the absence of aggregation.

Human IgG anti-IL2R heavy chain-only antibodies and antigen-binding fragments thereof with unique sequences from UniRat™ animals (UniAb™) were identified that bind to human IL2R in ELISA protein and cell-binding assays. The identified heavy chain variable region (VH) sequences are positive for human IL2R protein binding and/or for binding to IL2R+ cells, and are all negative for binding to cells that do not express IL2R.

Heavy chain-only antibodies and antigen-binding fragments that specifically bind to non-overlapping epitopes on an IL2R protein, such as, e.g., UniAbs™, can be identified by competition binding assays, such as enzyme-linked immunoassays (ELISA assays) or flow cytometric competitive binding assays. For example, one can use competition between known antibodies binding to the target antigen and the heavy chain-only antibody or antigen-binding fragment of interest. By using this approach, one can divide a set of heavy chain-only antibodies and antigen-binding fragments into those that compete with the reference antibody and those that do not. The non-competing heavy chain-only antibodies and antigen-binding fragments are identified as binding to a distinct epitope that does not overlap with the epitope bound by the reference antibody. Often, one antibody is immobilized, the antigen is bound, and a second, labeled (e.g., biotinylated) antibody is tested in an ELISA assay for ability to bind the captured antigen. This can be performed also by using surface plasmon resonance (SPR) platforms, including ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). For further details, see the Examples herein.

Typically, a heavy chain-only antibody or antigen-binding fragment thereof “competes” with a reference antibody if it causes about 15-100% reduction in the binding of the reference antibody to the target antigen, as determined by standard techniques, such as by the competition binding assays described above. In some embodiments, competitive binding is measured using an enzyme-linked immunoassay (ELISA assay). In some embodiments, one antibody construct is immobilized, the antigen is bound, and a second, labeled (e.g., biotinylated) antibody is tested in an ELISA assay for ability to bind the captured antigen. This can be performed, for example, using a surface plasmon resonance (SPR) platform, such as, for example, ProteOn XPR36 (BioRad, Inc), Biacore 2000 and Biacore T200 (GE Healthcare Life Sciences), and MX96 SPR imager (Ibis technologies B.V.), as well as on biolayer interferometry platforms, such as Octet Red384 and Octet HTX (ForteBio, Pall Inc). In some embodiments, competitive binding is measured using a flow cytometric competitive binding assay.

In various embodiments, the relative inhibition is at least about 15%, at least about 20%, at least about 25%, at least about 30%, at least about 35%, at least about 40%, at least about 45%, at least about 50% at least about 55%, at least about 60%, at least about 65%, at least about 70%, at least about 75%, at least about 80%, at least about 85%, at least about 90%, at least about 95% or higher.

### T-Cell Redirecting Therapies

T-cell redirecting therapies, such as bispecific T-cell engaging molecules and CAR-expressing T-cells, represent a promising class of immunotherapies under development for the treatment of various solid and liquid cancers. While any therapeutic agent capable of recruiting T-cells to a target cell or tissue is envisioned for use in the methods described herein, specific embodiments related to bispecific T-cell engaging molecules and CAR-expressing T-cells are provided for the sake of illustration.

In some embodiments, the T-cell redirecting therapy used in a method described herein is a bispecific T-cell engaging molecule. The bispecific T-cell engaging molecules employed in the methods of the present disclosure generally comprise a first domain that specifically binds to a target cancer cell antigen (e.g. CEA, CD19, CD33, CD70, EGFRvIII, EpCAM, FLT3, GPRC5D, DLL3, BCMA, PSMA, STEAP1, STEAP2, MUC16, MUC17, or CLDN18.2), a second domain that specifically binds to human CD3, and a half-life extension domain that provides a half-life for the molecule of greater than 24 hours. The half-life extension domain can be an immunoglobulin Fc domain, a domain derived from serum albumin (e.g. human serum albumin), an albumin-binding domain (e.g. comprising human albumin binding peptides or an antibody fragment that specifically binds to serum albumin), peptides that bind to the neonatal Fc receptor (FcRn), and polyethylene glycol polymers. In certain embodiments, the bispecific T-cell engaging molecules used in the methods of the present disclosure comprise an immunoglobulin Fc domain. In some such embodiments, the bispecific T-cell engaging molecule can be a bispecific antibody and have the general structure of a full-length immunoglobulin. For instance, in some embodiments, the bispecific T-cell engaging molecule can be a heterodimeric antibody comprising a light chain and heavy chain from an antibody that specifically binds to a target cancer cell antigen, and a light chain and heavy chain from an antibody that specifically binds to human CD3. In other embodiments, the bispecific T-cell engaging molecule can be an antibody fragment (e.g., a TCA) comprising a heavy chain from a heavy chain-only antibody that specifically binds to a target cancer cell antigen, and a light chain and heavy chain from an antibody that specifically binds to human CD3. In still other embodiments, the bispecific T-cell engaging molecule employed in the methods of the present disclosure comprises, in an amino to carboxyl order: (i) a first domain that specifically binds to a target cancer cell antigen; (ii) a second domain that specifically binds to human CD3; and (iii) an Fc domain comprising two Fc monomers, each monomer comprising an immunoglobulin hinge region, a CH2 domain, and a CH3 domain, wherein said two monomers are fused to each other via a peptide linker. In such embodiments, the bispecific T-cell engaging molecule can be a single chain polypeptide where all three domains are linked together, optionally via peptide linkers, to form a single polypeptide chain.

In certain embodiments, the binding domains of the bispecific T-cell engaging molecules used in the methods of the present disclosure comprise an immunoglobulin heavy chain variable region (VH) and an immunoglobulin light chain variable region (VL) of an antibody or antibody fragment which specifically binds to the desired antigen. For instance, the anti-cancer cell antigen binding domain of the bispecific T-cell engaging molecules of the present disclosure comprises a VH region and VL region from an antibody that specifically binds to a target cancer cell antigen and the anti-CD3 binding domain comprises a VH region and VL region from an antibody that specifically binds to CD3. The binding domains that specifically bind to a human cancer cell antigen or human CD3 can be derived from known antibodies to these antigens or from new antibodies or antibody fragments obtained by de novo immunization methods using the antigen proteins or fragments thereof, by phage display, or other methods known in the art. The antibodies from which the binding domains for the bispecific T-cell engaging molecules are derived can be monoclonal antibodies, recombinant antibodies, chimeric antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The first binding domain of the bispecific T-cell engaging molecules used in the methods of the present disclosure specifically binds to a target cancer cell antigen, such as, e.g., a human target cancer cell antigen. This binding domain is referred to herein as an anti-cancer cell antigen binding domain. The term “target cancer cell antigen” refers to an antigen expressed on the surface of a malignant cell, tumor cell, or other type of cancerous cell. A target cancer cell antigen may be expressed exclusively in cancer cells or may be overexpressed in cancer cells relative to normal cells. A target cancer cell antigen may also include a mutated or aberrant form of a protein expressed in cancer cells but not normal cells. Examples of a target cancer cell antigen include, but are not limited to, 5T4, AFP, BCMA, beta-catenin, BRCA1, CD19, CD20, CD22, CD33, CD70, CD123, CDH3, CDH19, CDK4, CEA, CLDN18.2, DLL3, DLL4, EGFR, EGFRvIII, EpCAM, EphA2, FLT3, FOLR1, gpA33, GPRC5D, HER2, IGFR, MAGE-1, MAGE-2, MAGE-3, MAGE-4, MAGE-6, MAGE-12, MSLN, MUC1, MUC2, MUC3, MUC4, MUC5, MUC16, MUC17, PSCA, PSMA, RAGE proteins, STEAP1, STEAP2, TRP1, and TRP2. In certain embodiments, the first domain of the bispecific T-cell engaging molecules used in the methods of the present disclosure specifically binds to a target cancer cell antigen chosen from MUC17, CLDN18.2, CD19, CD33, FLT3, DLL3, BCMA and PSMA.

The second binding domain of the bispecific T-cell engaging molecules used in the methods of the present disclosure specifically binds to CD3, such as, e.g., human CD3. This binding domain is referred to herein as an anti-CD3 binding domain. “CD3” (cluster of differentiation 3) is a T-cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to form the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide, which in humans is encoded by the CD3E gene which resides on chromosome 11.

The redirected lysis of target cells via the recruitment of T-cells by a T-cell engaging molecule which binds to CD3 on the T cell and to a target protein (e.g. cancer cell antigen) on the target cell (e.g. tumor cell) generally involves cytolytic synapse formation and delivery of perforin and granzymes. The engaged T-cells are capable of serial target cell lysis and are not affected by immune escape mechanisms interfering with peptide antigen processing and presentation, or clonal T cell differentiation; see, for example, WO 2007/042261.

In certain embodiments, the second binding domain of the bispecific T-cell engaging molecules used in the methods of the present disclosure specifically binds to CD3 on the surface of a T cell, such as, e.g., to human CD3 on the surface of a T cell. In some embodiments, the second binding domain of the bispecific T-cell engaging molecules specifically binds to CD3 epsilon, such as, e.g., human CD3 epsilon, e.g. human CD3 epsilon on the surface of a T-cell. In some embodiments, the second binding domain of the bispecific T-cell engaging molecules specifically binds to CD3 delta/epsilon, such as, e.g., human CD3 delta/epsilon, e.g. human CD3 epsilon on the surface of a T-cell. Examples of anti-CD3 antibodies or anti-CD3 binding domains from which the second binding domain of the bispecific T-cell engaging molecules used in the methods of the present disclosure can be constructed or derived are described in WO 2007/042261, WO 2008/119567, WO 2017/053856, WO 2017/201493, WO 2017/223111, WO 2018/052503, and WO 2019/224717, all of which are hereby incorporated by reference in their entireties. In certain embodiments, the second domain of the bispecific T-cell engaging molecules used in the methods of the present disclosure specifically binds to an epitope in the extracellular domain of human CD3 epsilon.

The bispecific T-cell engaging molecules suitable for use in the methods of the present disclosure may comprise additional domains, which, e.g., can modulate the pharmacokinetic profile of the molecule. For instance, the bispecific T-cell engaging molecules may further comprise a domain or moiety that increases the elimination half-life of the molecule. The elimination half-life refers to the time it takes for the concentration of a drug in the plasma or the total amount in the body to be reduced by 50%. Thus, after one half-life, the concentration of the drug in the body will be half of the starting dose. For example, the bispecific T-cell engaging molecules may comprise a half-life extension moiety that provides a half-life for the molecule of greater than 24 hours, greater than 48 hours, greater than 72 hours, greater than 5 days, greater than 7 days, greater than 10 days, greater than 14 days, or greater than 21 days. Accordingly, the bispecific T-cell engaging molecules suitable for use in the methods of the present disclosure may have a half-life of about 2 days to about 21 days, about 3 days to about 14 days, about 5 days to about 15 days, about 3 days to about 7 days, or about 2 days to about 5 days. Examples of half-life extension moieties that can be incorporated into the bispecific T-cell engaging molecules used in the methods of the present disclosure can include, but are not limited to, an immunoglobulin Fc domain, a domain derived from serum albumin (e.g. human serum albumin), or an albumin-binding domain (e.g. comprising human albumin binding peptides), peptides that bind to the neonatal Fc receptor (FcRn), and polyethylene glycol polymers. Non-limiting examples of domains derived from human serum albumin or variants thereof that can be incorporated into the bispecific T-cell engaging molecules are described, for example, in WO 2011/051489, WO 2012/059486, WO 2013/075066, WO 2013/135896, and WO 2014/072481, all of which are hereby incorporated by reference in their entireties. In some embodiments, the half-life extension moiety incorporated into the bispecific T-cell engaging molecules used in the methods of the present disclosure is an albumin-binding domain, such as a domain comprising an albumin-binding peptide or an antibody fragment (e.g., single domain antibodies or scFv domains) that specifically binds to serum albumin. Non-limiting examples of albumin-binding domains that may be incorporated into the bispecific T-cell engaging molecules suitable for use in the methods of the present disclosure are described in, for example, WO 2013/128027, WO 2014/140358, and WO 2017/201488, all of which are hereby incorporated by reference in their entireties.

In certain embodiments, the bispecific T-cell engaging molecules used in the methods of the present disclosure comprise an immunoglobulin Fc domain. The immunoglobulin Fc domain may comprise one or more Fc monomers. Each “Fc monomer” typically comprises at least a CH2 domain and a CH3 domain from an immunoglobulin molecule. The Fc monomer may comprise the CH2 and CH3 domains from an IgG1, IgG2, IgG3, or IgG4 immunoglobulin. As an example, the CH2 domain comprises amino acids 231 to 340 of an IgG1 immunoglobulin and the CH3 domain comprises amino acids 341 to 446 of an IgG1 immunoglobulin, where the amino acid numbering is according to the EU numbering system described in Edelman et al., Proc. Natl. Acad. USA, Vol. 63: 78-85 (1969) and Kabat et al., Sequences of Proteins of Immunological Interest, 5th Ed. Public Health Service, National Institutes of Health Publication No. 91-3242, Bethesda, MD (1991). The boundaries of the CH2 and CH3 domains may vary slightly from one IgG isoform to another, but the CH2 and CH3 domains in IgG2, IgG3, and IgG4 can be ascertained by alignment with the CH2 and CH3 domains in IgG1.

In some embodiments, the Fc monomer may comprise an immunoglobulin hinge region or portion thereof. The immunoglobulin hinge region is typically the region defined by amino acids 216 to 231 (according to the EU numbering system) of IgG immunoglobulins. In certain embodiments, the Fc monomer comprises a hinge region from an IgG1 immunoglobulin or a portion thereof. In certain embodiments, the Fc monomer comprises, in amino to carboxyl order, an immunoglobulin hinge region, an immunoglobulin CH2 domain, and an immunoglobulin CH3 domain.

In certain embodiments, the bispecific T-cell engaging molecules comprise an Fc domain having one Fc monomer. In alternative embodiments, the bispecific T-cell engaging molecules comprise an Fc domain having two or more Fc monomers. For instance, in one embodiment, the bispecific T-cell engaging molecules used in the methods of the present disclosure comprise an Fc domain having two Fc monomers. The two Fc monomers can be present on separate polypeptide chains and associate to form a dimer, e.g. via non-covalent interactions and/or disulfide bonds (e.g. between cysteine residues in the hinge regions of Fc monomers). In another embodiment, the two Fc monomers are fused to each other via a peptide linker, such as, e.g., a linker sufficient in length to allow the Fc monomers to associate and form an intra-chain dimer. The fusion of two Fc monomers to form a single polypeptide chain is referred to herein as a single-chain Fc domain (scFc domain) and is described in more detail below.

The peptide linker, by which the Fc monomers are fused to each other to form a single-chain Fc domain, may comprise at least 25 amino acid residues (e.g. 25, 26, 27, 28, 29, 30 or more). For instance, in some embodiments, this peptide linker comprises at least 30 amino acid residues (e.g. 30, 31, 32, 33, 34, 35 or more). In some embodiments, the linker comprises up to 40 amino acid residues, such as, e.g., up to 35 amino acid residues, e.g., exactly 30 amino acid residues.

The Fc monomer may contain one or more amino acid substitutions relative to the native CH2 or CH3 immunoglobulin amino acid sequences, e.g. to modulate effector function, alter glycosylation, or enhance stability. For instance, in one embodiment, the glycosylation site in the CH2 domain at amino acid position 297 according to EU numbering is removed by substituting a different amino acid for the asparagine residue at this position. A N297G substitution is employed in some embodiments. Stability-enhancing mutations include the substitution of one or more amino acids in the CH2 and/or CH3 domains with cysteine residues to promote disulfide bond formation. In some embodiments, specific pairs of residues are substituted with cysteine such that they preferentially form a disulfide bond with each other, thus limiting or preventing disulfide bond scrambling. Example pairs include, but are not limited to, A287C and L306C, V259C and L306C, R292C and V302C, and V323C and I332C, with the amino acid positions numbered according to the EU numbering system. In one particular embodiment, the Fc monomer(s) incorporated into the Fc domain of the bispecific T-cell engaging molecules comprises N297G, R292C, and V302C substitutions, with the amino acid positions numbered according to the EU numbering system.

In certain embodiments, the bispecific T-cell engaging molecules used in the methods of the present disclosure comprise an Fc domain, which is a single-chain Fc domain. Accordingly, in certain such embodiments, the Fc domain comprises two Fc monomers, each monomer comprising an immunoglobulin hinge region, an immunoglobulin CH2 domain, and an immunoglobulin CH3 domain, wherein the two Fc monomers are fused to each other via a peptide linker as described herein.

In certain embodiments, the bispecific T-cell engaging molecules used in the methods of the present disclosure comprise, in an amino to carboxyl order:
(i) a first domain that specifically binds to a target cancer cell antigen (e.g., a human cancer cell antigen) comprising a first immunoglobulin heavy chain variable region (VH1) and a first immunoglobulin light chain variable region (VL1);
(ii) a second domain that specifically binds to CD3 (e.g., human CD3) comprising a second immunoglobulin heavy chain variable region (VH2) and a second immunoglobulin light chain variable region (VL2); and
(iii) an Fc domain comprising two Fc monomers.

In certain embodiments, the bispecific T-cell engaging molecules used in the methods of the present disclosure are single chain polypeptides or single chain fusion proteins. As used herein, a “single chain polypeptide” or “single chain fusion protein” refers to a molecule consisting of only one polypeptide chain, i.e. all of the domains in the bispecific T-cell engaging molecule are linked together, optionally via peptide linkers, to form a single polypeptide chain. One non-limiting example of such a single chain polypeptide or single chain fusion protein in the context of the present disclosure is a single chain polypeptide comprising, in an amino to carboxyl order, an anti-cancer cell antigen scFv domain, a first peptide linker, an anti-CD3 scFv domain, a second peptide linker, and an scFc domain.

In one embodiment, the subject to be treated according to the methods of the present disclosure is diagnosed with or has leukemia or lymphoma, such as diffuse large B-cell lymphoma, Burkitt lymphoma, follicular lymphoma, Non-Hodgkin lymphoma, or acute lymphoblastic leukemia, and the anti-cancer cell antigen binding domain of the bispecific T-cell engaging molecule specifically binds to CD19.

In another embodiment, the subject to be treated according to the methods of the present disclosure is diagnosed with myeloid leukemia, such as, e.g., acute myeloid leukemia, and the anti-cancer cell antigen binding domain of the bispecific T-cell engaging molecule specifically binds to CD33 or FLT3.

In yet another embodiment, the subject to be treated according to the methods of the present disclosure is diagnosed with or has a DLL3-expressing cancer, such as small-cell lung cancer, neuroendocrine prostate cancer, melanoma, or glioblastoma, and the anti-cancer cell antigen binding domain of the bispecific T-cell engaging molecule specifically binds to DLL3.

In certain embodiments, the subject to be treated according to the methods of the present disclosure is diagnosed with or has a BCMA-positive cancer, and the anti-cancer cell antigen binding domain of the bispecific T-cell engaging molecule specifically binds to BCMA. In some embodiments, the BCMA-positive cancer is multiple myeloma. The multiple myeloma may be refractory and/or relapsed multiple myeloma.

In certain other embodiments, the subject to be treated according to the methods of the present disclosure is diagnosed with or has a PSMA-expressing cancer, such as prostate cancer, non-small cell lung cancer, small cell lung cancer, renal cell carcinoma, hepatocellular carcinoma, bladder cancer, testicular cancer, colon cancer, glioblastoma, breast cancer, ovarian cancer, endometrial cancer, or melanoma, and the anti-cancer cell antigen binding domain of the bispecific T-cell engaging molecule specifically binds to PSMA. In some embodiments, the PSMA-expressing cancer is prostate cancer. The prostate cancer may be castration-resistant prostate cancer (prostate cancer that is resistant to androgen deprivation therapy). In these and other embodiments, the prostate cancer is metastatic prostate cancer, particularly metastatic castration-resistant prostate cancer.

In some embodiments, the subject to be treated according to the methods of the present disclosure is diagnosed with a CLDN18.2-expressing cancer, such as colorectal cancer, pancreatic cancer, ovarian cancer, lung cancer, and gastrointestinal cancer, such as, e.g., gastric cancer, esophageal cancer, and gastroesophageal junction cancer, and the anti-cancer cell antigen binding domain of the bispecific T-cell engaging molecule specifically binds to CLDN18.2.

In other embodiments, the subject to be treated according to the methods of the present disclosure is diagnosed with a MUC17-expressing cancer, such as colorectal cancer, pancreatic cancer, and gastrointestinal cancer, such as, e.g., gastric cancer and gastroesophageal junction cancer, and the anti-cancer cell antigen binding domain of the bispecific T-cell engaging molecule specifically binds to MUC17.

The bispecific T-cell engaging molecules for use in the methods of the present disclosure may be prepared by any of a number of conventional techniques. For example, the bispecific T-cell engaging molecules described herein may be produced by recombinant expression systems, using any technique known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

Bispecific T-cell engaging molecules or components thereof (e.g., Fv fragments, Fc monomers) can be expressed in hybridoma cell lines or in cell lines other than hybridomas. Expression vectors or constructs encoding the bispecific T-cell engaging molecules can be used to transform a mammalian, insect or microbial host cell. Examples of vectors include, but are not limited to, plasmids, viral vectors, non-episomal mammalian vectors and expression vectors, for example, recombinant expression vectors. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Recombinant expression vectors or constructs will typically comprise a nucleic acid molecule encoding a polypeptide comprising one or more of the following: one or more CDRs provided herein; a light chain constant region; a light chain variable region; a heavy chain constant region (e.g., CH1, CH2 and/or CH3); a heavy chain variable region; hinge region, Fc domain, and/or another scaffold portion of an antibody specifically binding to a cancer cell antigen or anti-CD3 antibody. These nucleic acid sequences are inserted into an appropriate expression vector using standard ligation techniques. In embodiments in which the bispecific T-cell engaging molecule is a single chain polypeptide or single chain fusion protein, the nucleic acid comprised in the recombinant expression vector will typically encode the full-length single chain polypeptide (e.g., full-length single chain fusion protein). The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964, which is hereby incorporated by reference). Suitable expression vectors can be purchased, for example, from Invitrogen Life Technologies or BD Biosciences (formerly “Clontech”). Other useful vectors for cloning and expressing the antibody constructs and fragments include those described in Bianchi and McGrew, 2003, Biotech. Biotechnol. Bioeng. 84:439-44, which is hereby incorporated by reference. Additional suitable expression vectors are discussed, for example, in Methods Enzymol., vol. 185 (D. V. Goeddel, ed.), 1990, New York: Academic Press.

Typically, expression vectors used in any of the host cells to produce a bispecific T-cell engaging molecule will contain sequences for cloning and expression of exogenous nucleotide sequences encoding the bispecific T-cell engaging molecule or components thereof. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, i.e., an oligonucleotide molecule located at the 5′ or 3′ end of the bispecific T-cell engaging molecule coding sequence; the oligonucleotide sequence encodes polyHis (such as hexaHis), or another “tag” such as FLAG® tag, HA (hemaglutinin influenza virus), or myc, for which commercially available antibodies exist. This tag is typically fused to the polypeptide upon expression of the polypeptide and can serve as a means for affinity purification or detection of the bispecific T-cell engaging molecule from the host cell. Affinity purification can be accomplished, for example, by column chromatography using antibodies against the tag as an affinity matrix. Optionally, the tag can subsequently be removed from the purified T-cell engaging molecule by various means such as using certain peptidases for cleavage.

Expression and cloning vectors will typically contain a promoter that is recognized by the host cell and operably linked to the nucleic acid molecule encoding a bispecific T-cell engaging molecule. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system. A large number of promoters, recognized by a variety of potential host cells, are well known to those of skill in the art. For example, suitable promoters for use with mammalian host cells include those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). A suitable promoter is operably linked to the polynucleotide encoding, e.g., a bispecific T-cell engaging molecule or component thereof, by removing the promoter from the source nucleic acid by restriction enzyme digestion and inserting the desired promoter sequence into the vector.

The expression vectors for recombinant production of the bispecific T-cell engaging molecules described herein may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well-known to one skilled in the art. The expression vectors can be introduced into host cells to thereby produce the bispecific T-cell engaging molecules encoded by the nucleic acids present in the vectors.

After the vector has been constructed and one or more nucleic acid molecules encoding the bispecific T-cell engaging molecule or component thereof has been inserted into the proper site(s) of the vector or vectors, the completed vector(s) may be inserted into a suitable host cell for amplification and/or polypeptide expression. The term includes the progeny of the parent cell, whether or not the progeny is identical in morphology or in genetic make-up to the original parent cell, so long as the gene of interest is present. A host cell that comprises an isolated polynucleotide or nucleic acid encoding a bispecific T-cell engaging molecule, which may be operably linked to at least one expression control sequence (e.g. promoter or enhancer), is a “recombinant host cell.”

The transformation of an expression vector for a polypeptide into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

A host cell, when cultured under appropriate conditions, synthesizes a bispecific T-cell engaging molecule that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. Suitable host cells include, but are not limited to, prokaryotic cells (e.g. E. coli, B. subtilis), yeast cells (Saccharmoyces cerevisiae, Pichia pastoris), and mammalian cells (e.g. Chinese hamster ovary (CHO), human embryonic kidney (HEK)). In some embodiments, CHO cells are employed as host cells for expressing the bispecific T-cell engaging molecules.

Host cells are transformed or transfected with the above-described expression vectors for production of the T-cell engaging molecules and are cultured in conventional nutrient media modified as appropriate for inducing promoters, selecting transformants, or amplifying the genes encoding the desired sequences. The host cells used to produce the antibody constructs may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58: 44, 1979; Barnes et al., Anal. Biochem. 102: 255, 1980; U.S. Pat. Nos. 4,767,704; 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinary skilled artisan.

Upon culturing the host cells, the T-cell engaging molecule can be produced intracellularly, in the periplasmic space, or directly secreted into the medium. If the T-cell engaging molecule is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the T-cell engaging molecule is secreted into the culture medium, the T-cell engaging molecule can be separated from host cells through centrifugation or microfiltration, and optionally, subsequently concentrated through ultrafiltration. The bispecific T-cell engaging molecules can be further purified or partially purified using, for example, one or more chromatography steps, such as affinity chromatography (e.g. protein A, protein L, or protein G affinity chromatography), cation exchange chromatography, anion exchange chromatography, hydroxyapatite chromatography, hydrophobic interaction chromatography, or mixed mode chromatography.

Alternatively, the T-cell redirecting therapy used in a method described herein may be a CAR-expressing T-cell. CAR-expressing T-cells employed in the methods of the present disclosure typically comprise a first domain that specifically binds to a target cancer cell antigen, a transmembrane domain, and an intracellular signalling domain. In some embodiments, the intracellular signaling domain comprises a costimulatory domain and/or a primary signaling domain.

In some embodiments, the first domain specifically binds to a target cancer cell antigen chosen from CEA, CD19, CD33, CD70, EGFRvIII, EpCAM, FLT3, GPRC5D, DLL3, BCMA, PSMA, STEAP1, STEAP2, MUC16, MUC17, and CLDN18.2.

In some embodiments, the CAR-expressing T-cells comprise a transmembrane domain that comprises a transmembrane domain of a protein, such as, e.g., a protein chosen from the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137, and CD154. In some embodiments, first domain (i.e., the antigen binding domain) is connected to the transmembrane domain by a hinge region.

In some embodiments, the costimulatory domain is a functional signaling domain from a protein, such as, e.g., a protein chosen from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11 a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11 d, ITGAE, CD103, ITGAL, CD11 a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some embodiments, the primary signaling domain comprises a functional signaling domain of CD3 zeta.

In some embodiments, the intracellular signaling domain comprises a functional signaling domain of 4-1BB and/or a functional signaling domain of CD3 zeta. In some embodiments, the intracellular signaling domain comprises a functional signaling domain of CD27 and/or a functional signaling domain of CD3 zeta. In some embodiments, the intracellular signaling domain comprises a functional signaling domain of CD28 and/or a functional signaling domain of CD3 zeta. In some embodiments, the intracellular signaling domain comprises a functional signaling domain of ICOS and/or a functional signaling domain of CD3 zeta.

In some embodiments, the CAR further comprises a leader sequence.

CAR-expressing T-cells employed in the methods of the present disclosure may be allogenic or autologous and may be prepared according to standard techniques of the art.

### Pharmaceutical Compositions

Anti-IL-2Rβγ heavy chain-only antibodies, antigen-binding fragment thereof, and T-cell redirecting therapies can be administered to the subject using any suitable pharmaceutical composition and via any suitable administration route. Pharmaceutical compositions for use in the methods of the present disclosure may comprise: (i) an anti-IL-2Rβγ heavy chain-only antibody or antigen-binding fragment thereof and/or a T-cell redirecting therapy; and a pharmaceutically acceptable carrier. Pharmaceutically acceptable carriers include, but are not limited to, adjuvants, solid carriers, water, buffers, other carriers used in the art to hold therapeutic components, and combinations thereof.

Pharmaceutical compositions employed in methods of the present disclosure may be prepared for storage by mixing therapeutic agents having the desired degree of purity with optional pharmaceutically acceptable carriers, excipients, or stabilizers (see, e.g. Remington's Pharmaceutical Sciences 16th edition, Osol, A. Ed. (1980)), such as in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as, e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™, or polyethylene glycol (PEG).

Pharmaceutical compositions can be provided in unit dosage form (i.e., the dosage for a single administration). The formulation depends on the route of administration chosen. The antibodies herein can be administered by intravenous injection or infusion or subcutaneously. Typically, pharmaceutical compositions for use in the disclosed methods are prepared as injectables, either as liquid solutions or suspensions; solid forms suitable for solution in, or suspension in, liquid vehicles prior to injection can also be prepared. Pharmaceutical compositions described herein may be suitable for intravenous or subcutaneous administration, directly or after reconstitution of solid (e.g., lyophilized) compositions. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The therapeutic agents of this disclosure can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

For injection administration, the antibodies herein can be formulated in aqueous solutions, e.g., in physiologically-compatible buffers to reduce discomfort at the site of injection. The solution can contain carriers, excipients, or stabilizers as discussed above. Alternatively, antibodies can be in lyophilized form for constitution with a suitable vehicle, e.g., sterile pyrogen-free water, before use.

In some embodiments, pharmaceutical compositions for parenteral administration are sterile and substantially isotonic and manufactured under Good Manufacturing Practice (GMP) conditions.

Antibody formulations are disclosed, for example, in U.S. Pat. No. 9,034,324. Similar formulations can be used for the heavy chain-only antibodies and antigen-binding fragments thereof, including UniAbs™, of the present disclosure. Subcutaneous antibody formulations are described, for example, in US20160355591 and US20160166689.

In some embodiments, a pharmaceutical composition comprising a bispecific T-cell engaging molecule further comprises a buffer, a surfactant, and a stabilizing agent. In some embodiments, the pharmaceutical composition comprises a bispecific T-cell engaging molecule, a glutamate buffer, polysorbate 20 or polysorbate 80, and sucrose, at a pH of about 4.0 to about 4.4. In some embodiments, the pharmaceutical compositions may be lyophilized and reconstituted prior to administration to a patient.

Non-limiting example pharmaceutical compositions comprising bispecific T-cell engaging molecules are described in WO 2018/141910, which is hereby incorporated by reference in its entirety. In certain embodiments, a pharmaceutical composition useful for the treatment of cancer according to the methods described herein comprises about 0.5 mg/ml to about 2 mg/ml of a bispecific T-cell engaging molecule, about 5 mM to about 20 mM L-glutamic acid, about 0.005% to about 0.015% weight/volume (w/v) polysorbate (e.g. polysorbate 20 or polysorbate 80), and about 7% to about 12% (w/v) sucrose. In other embodiments, the pharmaceutical composition comprises about 0.5 mg/ml to about 1.5 mg/ml of a bispecific T-cell engaging molecule, about 8 mM to about 12 mM L-glutamic acid, about 0.008% to about 0.012% (w/v) polysorbate (e.g. polysorbate 20 or polysorbate 80), and about 8% to about 10% (w/v) sucrose. The pH of these compositions is in the range of about 4.0 to about 4.4 (e.g., pH of about 4.0, about 4.1, about 4.2, about 4.3, or about 4.4).

Any of the pharmaceutical compositions comprising the bispecific T-cell engaging molecules and/or the anti-IL-2Rβγ heavy chain-only antibody or antigen-binding fragment thereof described herein can be lyophilized and reconstituted with, e.g. sterile water for injection, prior to administration to the patient. Reconstitution volumes will depend on the protein content following lyophilization and the desired concentration of the bispecific T-cell engaging molecule and/or the anti-IL-2Rβγ heavy chain-only antibody or antigen-binding fragment thereof in the reconstituted solution, but may be from about 0.5 ml to about 5 ml. The solution following reconstitution can be further diluted with a diluent (e.g. saline and/or intravenous solution stabilizer (IVSS)) prior to administration to the patient as appropriate in order to administer the doses described herein according to the methods of the present disclosure.

The concentration(s) of therapeutic agent(s) in the pharmaceutical compositions of the present disclosure can vary widely, and may be chosen based on fluid volumes, viscosities, body weight, and the like in accordance with the particular mode of administration selected and the patient's needs (see, e.g., Remington's Pharmaceutical Science (15th ed., 1980) and Goodman & Gillman, The Pharmacological Basis of Therapeutics (Hardman et al., eds., 1996)).

The use of bispecific T-cell engaging molecules, CAR-expressing T-cells, and anti-IL-2Rβγ heavy chain-only antibodies and antigen-binding fragments thereof for preparation of medicaments for administration according to any of the methods disclosed herein is specifically contemplated.

### Dosing

Effective doses of the therapeutic agents of the present disclosure for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g., companion animals such as dogs, cats, horses, etc., laboratory mammals such as rabbits, mice, rats, etc., and the like. Treatment dosages can be titrated to optimize safety and efficacy.

Dosage levels can be readily determined by the ordinarily skilled clinician, and can be modified as required, e.g., as required to modify a subject's response to therapy. The amount of therapeutic agent that can be combined with the carrier materials to produce a single dosage form varies depending upon the host treated and the particular mode of administration. Dosage unit forms generally contain between from about 1 mg to about 500 mg of an active ingredient.

In some embodiments, the therapeutic dosage of the anti-IL-2Rβγ heavy chain-only antibody or antigen-binding fragment thereof may range from about 0.0001 to 100 mg/kg, and more usually 0.01 to 5 mg/kg, of the host body weight. For example, dosages can be 1 mg/kg body weight or 10 mg/kg body weight or within the range of 1-10 mg/kg. An exemplary treatment regime entails administration once every two weeks or once a month or once every 3 to 6 months.

The therapeutic doses of bispecific T-cell engaging molecules administered according to the methods of the present disclosure may range from about 50 μg to about 200 mg or from about 200 μg to about 80 mg depending on the specific bispecific T-cell engaging molecule employed and the type, grade, or stage of cancer to be treated in the patient. Non-limiting example ranges of therapeutic doses of a bispecific T-cell engaging molecule for the treatment of cancer may include, but are not limited to, doses of about 50 μg to about 200 mg, from about 200 μg to about 80 mg, from about 90 μg to about 30 mg, from about 300 μg to about 15 mg, from about 150 μg to about 2 mg, from about 6 mg to about 25 mg, from about 1 mg to about 20 mg, from about 10 mg to about 100 mg, or from about 50 mg to about 150 mg.

When the T-cell redirecting therapy is a CAR-expressing T-cell, a dose of CAR-expressing T-cells cells may comprise about 10⁴ cells/kg to about 10⁹ cells/kg, such as, e.g., about 10⁴ cells/kg to about 10⁵ cells/kg, about 10⁵ cells/kg to about 10⁶ cells/kg, about 10⁶ cells/kg to about 10⁷ cells/kg, about 10⁷ cells/kg to about 10′ cells/kg, or about 10′ cells/kg to about 10⁹ cells/kg; or at least one of: about 1×10⁷ cells, about 1.5×10⁷ cells, about 2×10⁷ cells, about 2.5×10⁷ cells, about 3×10⁷ cells, about 3.5×10⁷ cells, about 4×10⁷ cells, about 5 ×107 cells, about 1×10⁸ cells, about 1.5×10⁸ cells, about 2×10⁸ cells, about 2.5×10⁸ cells, about 3×10⁸ cells, about 3.5×10⁸ cells, about 4×10⁸ cells, about 5×10⁸ cells, about 1×10⁹ cells, about 2×10⁹ cells, or about 5×10⁹ cells. In some embodiments, a dose of CAR-expressing T-cells comprises at least about 1-5×10⁷ cells to about 1-5×10⁸ cells.

In some embodiments, the CAR-expressing T-cells are administered to the subject according to a dosing regimen comprising a total dose of cells administered to the subject by dose fractionation, e.g., one, two, three, or more separate administration of a partial dose. In some embodiments, a first percentage of the total dose is administered on a first treatment day, a second percentage of the total dose is administered on a subsequent (e.g., second, third, fourth, fifth, sixth, or seventh or later) treatment day, and optionally, a third percentage (e.g., the remaining percentage) of the total dose is administered on a yet subsequent (e.g., third, fourth, fifth, sixth, seventh, eighth, ninth, tenth, or later) treatment day. In some embodiments, the CAR-expressing T-cell is administered at a dose of about 1-10×10⁸ cells per infusion, such as, e.g., about 5×10⁸ cells per infusion.

Therapeutic agents of the present disclosure may be administered on multiple occasions, sequentially or concurrently. Intervals between single dosages can be weekly, monthly, or yearly. Intervals can also be irregular as indicated by measuring blood levels of the therapeutic entity in the patient. Alternatively, therapeutic agents of the present disclosure can be administered as a sustained release formulation, in which case less frequent administration is required. Dosage and frequency vary depending on the half-life of the therapeutic agent in the subject.

In some embodiments, the methods of the present disclosure comprise administering a T-cell redirecting therapy (e.g., a bispecific T-cell engaging molecule, a CAR-expressing T-cell) and/or an anti-IL-2Rβγ heavy chain-only antibody or antigen-binding fragment thereof to the patient in at least one initiation cycle. As used herein, an “initiation cycle” is a treatment cycle in which the therapeutic agent is administered at two or more different doses at a dosing frequency and mode of administration designed to minimize adverse events, for example, such as adverse events associated with CRS, while enabling exposure of the patient to a therapeutically effective dose of the therapeutic agent in the shortest time possible. An initiation cycle is preferably administered to a patient as the first treatment cycle when the patient begins a course of treatment with the therapeutic agent. An initiation cycle may also be administered to a patient when the patient re-starts a course of treatment with the therapeutic agent, for example, following a treatment-free period, dosing interruption (e.g. when a patient didn't complete a previous treatment cycle), or a relapse or progression of a cancer in the patient. Although administration of one initiation cycle will typically be sufficient, in some embodiments of the methods of the present disclosure, administration of two or more initiation cycles is contemplated. In one particular embodiment, only one initiation cycle is administered to the patient.

Generally, the methods of the present disclosure comprise administering a T-cell redirecting therapy (e.g., a T-cell engaging molecule, a CAR-expressing T-cell) and an anti-IL-2Rβγ heavy chain-only antibody or antigen-binding fragment thereof to the subject in one or more treatment cycles. A “treatment cycle” or “cycle” refers to a period of administration of one or more therapeutic agent(s) at specific dosages and dosing intervals. According to the methods of the present disclosure, a subject can receive multiple treatment cycles (e.g. 2, 3, 4, 5, 6, 7, 8, 9, 10, 11, 12, 13, 14 or more cycles). The treatment cycles can be administered to the patient consecutively with no break or period without administration of one or more therapeutic agent(s) between the cycles. Alternatively, a period without administration of one or more therapeutic agent(s) (e.g. a “treatment-free period” or “break”) can be employed between the treatment cycles. The length of the treatment-free period can be adjusted based on the patient's characteristics and/or response to treatment. For example, the patient may receive treatment cycles of one or more therapeutic agent(s) until the patient achieves a desired level of response, such as a complete response or partial response.

Additionally, a patient may be treated according to the methods of the present disclosure for a set treatment period. A “treatment period” begins upon administration of a first dose of a therapeutic agent in an initiation cycle and ends upon administration of a final dose of a therapeutic agent in a maintenance cycle. The treatment period may be from about 3 months to about 36 months, from about 12 months to about 24 months, or from about 6 months to about 12 months. For instance, the treatment period may be about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, about 33 months, or about 36 months. In some embodiments, the treatment period is about 6 months. In other embodiments, the treatment period is about 9 months. In yet other embodiments, the treatment period is about 12 months. The treatment period can be adjusted for each patient depending on the patient's response to treatment. In one particular embodiment, the patient is treated according to the methods of the present disclosure until the patient achieves a complete response or until evidence of the particular cancer is otherwise undetectable in the patient.

### Premedication

In certain embodiments of the methods of the present disclosure, one or more premedications can be administered to the patient prior to the administration of a first and/or subsequent dose of a T-cell redirecting therapy (e.g., a bispecific T-cell engaging molecule, a CAR-expressing T-cell) or a first and/or subsequent dose of an anti-IL-2Rβγ heavy chain-only antibody or antigen-binding fragment thereof. Depending on the type of premedication used and the route by which it is administered, the premedication may e.g. be administered 30-120 or 30-60 minutes prior to start of administration of the T-cell redirecting therapy (e.g., bispecific T-cell engaging molecule, CAR-expressing T-cell) and/or the anti-IL-2Rβγ heavy chain-only antibody or antigen-binding fragment thereof. The premedication may be administered, e.g. to prevent or reduce severity of infusion-related reactions and/or to prevent or reduce severity of cytokine release syndrome or its symptoms.

In some embodiments in which premedication is administered, the premedication is an antihistamine. The antihistamine can be administered orally or intravenously and can be administered at a dose equivalent to diphenhydramine 50 mg i.v. Suitable antihistamines that can be administered as a premedication include, but are not limited to, antihistamines of oral, parenteral or rectal route such as: azatadine (maximum dose e.g. 4 mg/day), brompheniramine (maximum dose e.g. 30 mg/day), cetirizine (maximum dose e.g. 15 mg/day), chlorpheniramine (maximum dose e.g. 30 mg/day), clemastine (maximum dose e.g. 10 mg/day), cyproheptadine (maximum dose e.g. 15 mg/day), desloratadine (maximum dose e.g. 7 mg/day), dexchlorpheniramine (maximum dose e.g. 15 mg/day), diphenhydramine (maximum dose e.g. 350 mg/per day), doxylamine (maximum dose e.g. 180 mg/day), fexofenadine (maximum dose e.g. 200 mg/day), loratadine (maximum dose e.g. 15 mg/day), and phenindamine (maximum dose e.g. 180 mg/day).

In other embodiments in which premedication is administered, the premedication is a glucocorticoid. Glucocorticoids are a class of corticosteroids, which are a class of steroid hormones. Glucocorticoids are corticosteroids that bind to the glucocorticoid receptor. A less common synonym is glucocorticosteroid. Cortisol (known as hydrocortisone when used as a medication) is the most important human glucocorticoid. A variety of synthetic glucocorticoids, some far more potent than cortisol, have been created for therapeutic use. Cortisol is the standard of comparison for glucocorticoid potency. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. The glucocorticoid can be administered orally or intravenously and can be administered at a dose equivalent to 4-20 mg dexamethasone i.v. (the equivalence referring to the glucocorticoid potency). The dose of glucocorticoid can be the same at each administration (i.e. at each time the glucocorticoid premedication is administered). Alternatively, the dose of glucocorticoid can be reduced in subsequent administrations, e.g. by 50% of the previous dose, if there are no or minimal signs of infusion reactions and/or CRS symptoms following the previous administration of the bispecific T-cell engaging molecule. In certain embodiments, glucocorticoids are only administered as premedications during the initiation cycle and are not administered in subsequent treatment cycles (e.g. maintenance cycles).

Examples of glucocorticoids to be used as a premedication include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, triamcinolone acetonide, as well as combinations and/or pharmaceutically acceptable derivatives thereof. The different glucocorticoids may be used alone or in combination. In certain embodiments of the methods of the present disclosure, the glucocorticoid administered to the patient prior to administration of one or more (or all) doses of the T-cell redirecting therapy (e.g., bispecific T-cell engaging molecule, CAR-expressing T-cell) during the initiation cycle and/or maintenance cycle is dexamethasone. Dexamethasone can be administered at a dose of about 4-20 mg, 6-18 mg, 8-16 mg, about 16 mg, or about 8 mg at each administration.

In certain embodiments in which a premedication is administered, the premedication can be an IL-6 receptor antagonist, such as tocilizumab. Tocilizumab has been reported to effectively reduce or reverse symptoms of CRS induced by T cell-engaging therapies. See, e.g., Maude et al., Cancer J., Vol. 20:119-122, 2014. Tocilizumab can be administered at a dose of about 1 mg/kg to about 20 mg/kg body weight, about 8 mg/kg to about 12 mg/kg body weight, or about 4 mg/kg to about 8 mg/kg body weight. Tocilizumab can be administered about 1 hour to about 2 hours prior to each dose of the T-cell redirecting therapy (e.g., bispecific T-cell engaging molecule, CAR-expressing T-cell) in the initiation cycle and/or one or more maintenance cycles. Additionally or alternatively, tocilizumab can be administered immediately after each dose of the T-cell directing therapy (e.g., bispecific T-cell engaging molecule, CAR-expressing T-cell) in the initiation cycle and/or one or more maintenance cycles. Other antagonists of IL-6/IL-6 receptor signaling, such as siltuximab, olokizumab, clazakizumab, sarilumab, and sirukumab, can be used as a premedication according to the methods of the present disclosure to reduce the occurrence or severity of CRS.

In certain other embodiments in which a premedication is administered, the premedication is a tumor necrosis factor alpha (TNF-alpha) antagonist. CRS symptoms have been previously reported to be mediated in part by release of TNF-alpha (Lee et al., Blood, Vol. 124:188-195, 2014; Grupp et al., N Engl J Med., Vol. 368:1509-1518, 2013). Recent studies have suggested that treatment with TNF-alpha antagonists prior to administration of immunotherapy agents may mitigate CRS symptoms (Li et al., Sci Transl Med., Vol. 11(508), 2019; Lee et al., 2014, supra; Grupp et al., 2013, supra). Accordingly, in certain embodiments, the methods of the present disclosure further comprise administering to the patient a TNF-alpha antagonist prior to administration of each dose of the T-cell redirecting therapy (e.g., bispecific T-cell engaging molecule, CAR-expressing T-cell) during the initiation cycle and/or one or more maintenance cycles. Examples of TNF-alpha antagonists that can be used as a premedication include, but are not limited to, etanercept, infliximab, adalimumab, certolizumab pegol, and golimumab.

### Kits and Combination Products

Some embodiments of the present disclosure relate to combination products comprising an anti-IL-2Rβγ heavy chain-only antibody or antigen binding fragment thereof described herein and a T-cell redirecting therapy (e.g., a T-cell engaging molecule, CAR-expressing T-cell) described herein. In some embodiments, the combination product is adapted for use in a method disclosed herein. In some embodiments, the method possesses one or more of the features of a method described above.

Also disclosed herein is a combination product for use in a method of treating cancer in a subject in need thereof, comprising an anti-IL-2Rβγ heavy chain-only antibody or antigen binding fragment thereof described herein and a T-cell redirecting therapy (e.g., a T-cell engaging molecule, CAR-expressing T-cell) described herein, wherein the anti-IL-2Rβγ heavy chain-only antibody enhances an anti-cancer effect associated with administration of the T cell redirecting therapy.

Also within the scope of the disclosure are kits comprising the therapeutic agents used in the methods of the present disclosure and instructions for use. Kits typically include a label indicating the intended use of the contents of the kit. The term “label” as used herein includes any writing, or recorded material supplied on or with a kit, or which otherwise accompanies a kit.

Illustratively, in some embodiments, the present disclosure provides kits comprising a pharmaceutical composition disclosed herein and instructions for using the pharmaceutical composition to prepare and deliver the T-cell redirecting therapy (e.g., the bispecific T-cell engaging molecule, CAR-expressing T-cell) and/or the anti-IL-2Rβγ heavy chain-only antibody or antigen-binding fragment thereof for treating cancer in a subject in need thereof. In embodiments in which the pharmaceutical composition is provided in a lyophilized or dry powder form, the kit may comprise a diluent and instructions for reconstituting the pharmaceutical composition prior to administration. In certain embodiments, the kits may further comprise one or more vials of intravenous solution stabilizer (IVSS) and instructions for using the IVSS for pre-treatment of IV bags prior to dilution of the pharmaceutical composition for delivery to the patient.

Additionally, some embodiments of the present disclosure provide a kit comprising a combination product as described herein, wherein the combination product is present in a single container, or the components of the combination product are present individually, the kit optionally further comprising at least one of a group comprising instructions for use, a device for administration of the combination product or a component thereof, at least one separately packed medium for reconstitution, and a pharmaceutical carrier suitable for use with at least one of the combination partners.

Example embodiments now being fully described, it will be apparent to one of ordinary skill in the art that various changes and modifications can be made without departing from the spirit or scope of the disclosure.

### EXAMPLES

### Materials and Methods:

### Construct Names

The following table (Table 11) provides shorthand nomenclature for six bispecific heavy chain-only antibody constructs evaluated herein:

TABLE 11
Shorthand Nomenclature

| Shorthand Name | Sequence Name |
|---|---|
| BsAb-1 | IL2RB_F09C**IL2RG_F16A |
| BsAb-2 | IL2RB_F09G**IL2RG_F16B |
| BsAb-3 | IL2RB_F09G**IL2RG_F16C |
| BsAb-4 | IL2RB_F09G**IL2RG_F18A |
| BsAb-5 | IL2RB_F09K**IL2RG_F16B |
| BsAb-6 | IL2RB_F18E**IL2RG_F16A |

### Immunizations, Next-Generation Sequencing Clonotype Analysis, and Cloning

Methods essentially as described in Harris et al. Front Immunol. 2018 Apr. 24; 9:889(60). In brief, UniRat animals were immunized using standard adjuvants (Complete Freunds or Titermax/Ribi) along with recombinant protein antigens in a 48-day protocol or DNA immunizations. For protein immunizations, boosts consisted of 10 μg of recombinant protein injected into each leg of each animal with the appropriate adjuvant. In the case of DNA immunizations, gold particles were coated with vectors containing cDNA of the target antigen, which were subsequently administered subcutaneously every 7 days, using a gene gun. Plasma samples were collected post-immunization to assess serum titers against the antigen by ELISA.

After approximately 7 weeks (protein antigen) or 10 weeks (DNA antigen) of immunization, draining lymph nodes were harvested and total RNA was isolated. Ig heavy chain sequences were amplified using first strand cDNA synthesis and 5′ RACE by PCR, following methods similar to those previously described in Harris et al. Front Immunol. 2018 Apr. 24; 9:889 and then purified by gel extraction.

Next-generation sequencing was completed using the MiSeq platform (Illumina) with 2×300 paired-end reads. To enable multiplexing of samples, indexing labels were added by primer extension. Approximately 100,000 paired reads covered each sample, and those that showed alignment of less than 20 nucleotides to a human Ig locus were discarded. Merged forward and reverse reads of VH regions were translated into open reading frames and framework and CDR regions identified by IGBLAST (https://www.ncbi.nlm.nih.gov/igblast/). Clonotypes (defined by CDR3 protein sequences with at least 80% sequence similarity) were determined for samples using agglomerative clustering. CDR3 clonotypes were ranked by the percent of total reads in a sample defined by that clonotype. Those with the greatest abundance were prioritized for high-throughput cloning into an expression vector containing a CH1-deleted human IgG1 Fc region and validated by Sanger sequencing. Plasmids were transformed into E. coli grown in LB culture media and then purified to enable transient transfection of HEK 293 cells in 96-well format. Following several days of expression, supernatants were harvested and clarified by centrifugation.

### High-Throughput ELISA

Methods are essentially as described in Harris et al. Front Immunol. 2018 Apr. 24; 9:889. Briefly, recombinant proteins were coated overnight at 4° C. in 96-well plates using BupH Carbonate-Bicarbonate buffer (human IL-2Rβ, Acrobiosystems; cynomolgus IL-2Rβ, Sino Biological). Plates were then washed with TBST (20 mM Tris, 150 mM NaCl, 0.05% Tween-20, pH 7.6) and blocked with blocking buffer (TBST with 1% dry milk powder). HEK 293 supernatants were diluted 1:100 in blocking buffer and added to antigen-coated plates. Detection of bound molecules was accomplished using an HRP-labeled anti-human Ig secondary antibody together with chemiluminescent substrate.

Luminescence was quantified (SpectraMax i3X, Molecular devices) and the signal for each well was normalized by dividing by the average background luminescence of antigen-coated wells that had been incubated with supernatant from untransfected HEK 293 cells.

### Cell Lines and PBMCs

MO7e cells were obtained from DSMZ and were grown in RPMI medium containing 10% Fetal Bovine Serum (FBS), 1% Penicillin/Streptomycin, and 10 ng/mL rhGM-CSF. HSC-F cells were obtained from The Nonhuman Primate Reagent Resource and cultured in RPMI medium supplemented with 20% FBS, 1% Penicillin/Streptomycin and 55 μM β-Mercaptoethanol. 293-F were obtained from Gibco and grown according to their recommendations.

For creating stable cell lines expressing human IL-2Rβ or cynomolgus IL-2Rβ, expression constructs carried the full-length cDNA for the antigen and a NeoR selection cassette. Each expression construct was then linearized and used to electroporate CHO cells. Three days after transfection, cells were put under selection for 3-6 weeks using Geneticin treatments. At the end of the selection period, all untransfected and negative control cell lines were killed, while all transfected pools showed regrowth as expected for successfully transfected pools. Four pools of each target were then assayed by flow cytometry for binding to a positive control antibody. The culture media for the CHO cells was EX-CELL® 325 PF CHO media containing 8 mM L-glutamine, 0.1 μg/L IGF-1, 5% dialyzed FBS, 0.45 mg/mL geneticin, and 0.45 mg/mL hygromycin. The cells were grown in suspension and maintained at a concentration between 0.5×10⁶/mL to 2×10⁶/mL.

Human PBMCs were isolated in-house from fresh leukapheresis packs (StemCell) by Ficoll® Paque Premium (GE Healthcare Life Sciences) density gradient centrifugation.

### Cell Binding by Flow Cytometry

All washes and dilutions of cells, antibodies, and reagents were performed using flow buffer (1X PBS, 1% BSA, 0.1% NaN3, pH 7.4). Staining was performed in a round-bottom 96-well plate (Corning) seeded at 100,000 cells/well, and all incubations were performed at 4° C. or on ice. For primary and secondary screens, the cells were incubated for 30 minutes with pre-diluted test antibodies/antibody fragments (secondary screen and dose-curves) or 1:5 diluted HEK 293 supernatants containing antibodies/antibody fragments (for primary screens and diversity screens) in a total volume of 50 μL. The cells were washed twice with 200 μL flow buffer. The cells were then incubated for 30 minutes with detection antibody (Goat F(ab′)2 Anti-Human IgG-PE, Southern Biotech) at 0.625 μg/mL in flow buffer. Following 2 more washes, the cells were resuspended in a final volume of 150 μL of flow buffer. The cells were analyzed on a BD FACSCelesta or a Guava easyCyte 8-HT flow cytometer. At least 3000 events were collected, and PE geometric mean fluorescence intensity was plotted as a fold over background (cells incubated with secondary detection antibody only). In some secondary screens involving human or cynomolgus PBMCs, an additional CD4 antibody (BioLegend) and/or CD8 antibody (BioLegend) was included to further characterize cell binding.

pSTAT5 Detection by Flow Cytometry

For detection of pSTAT5 by flow cytometry, PBMCs were prepared from either frozen whole blood (cynomolgus) or frozen LeukoPak (human). Cells were thawed, washed twice with complete RPMI medium, and resuspended at 5×10⁶ cells/mL. 100 μL/well of these cells was then transferred to a sterile, round-bottom, 96-well plate (Corning) and sealed with an AeraSeal™ (Excel Scientific). The plate was then incubated at 37° C. and 5% CO₂ for 1 hour. After the incubation, 100 μL of pre-diluted antibodies (or IL-2/IL-2 variant) was added to the appropriate wells. A final concentration of 10 nM IL-2 (R&D Systems) was used in control wells to ensure detectable pSTAT5. The plate was then resealed and returned to the incubator for an additional 1 hour. After the incubation, the cells were centrifuged and washed twice with PBS pre-chilled to 4° C. The cells were then blocked with Human TruStain FcX (BioLegend) and then subsequently stained for 30 minutes with Fixable Viability Dye (Invitrogen) and antibodies against CD3, CD4, CD8, CD25, and/or CD56. After staining, the cells were again centrifuged and washed twice with pre-chilled PBS. The cells were then fixed with the addition of 200 μL/well Fixation Buffer (BioLegend) and incubated at room temp for 30 minutes. After fixation, the cells were centrifuged and washed twice with Flow Buffer (1X PBS, 1% BSA, 0.1% NaN3, pH 7.4). Next, the cells were permeabilized by resuspending in 200 μL/well True-Phos buffer (BioLegend) pre-chilled to −20° C. and transferred to a −20° C. freezer overnight. The following morning, the cells were centrifuged, washed twice with flow buffer, and subsequently stained for 30 minutes with anti-pSTAT5 (BD Biosciences). After two additional washes, the cells were resuspended in 125 μL/well flow buffer and acquired on a BD FACSCelesta.

### Ki67 Detection by Flow Cytometry

For detection of Ki67 by flow cytometry, frozen human PBMCs (previously isolated in-house from a LeukoPak) were thawed and rested overnight in complete RPMI medium at 1×10⁶ cells/mL. The morning of the assay, the PBMCs were washed with complete RPMI and resuspended at le6 cells/mL. Then, to each well of a sterile 96-well plate, 100 μL of PBMCs, 50 μL of 0.16X ImmunoCult (StemCellTech), and 50 μL of diluted antibody or rhIL-2 (R&D Systems) was added. 0.5X ImmunoCult was used for staining controls to ensure detectable Ki67 and CD25 signal for compensation. The plate was then covered and incubated at 37° C. and 5% C02. After 3 days, the media was refreshed with 100 μL/well of the corresponding concentration antibody and ImmunoCult and then returned to the incubator. After 3 more days (6 days total), the cells were centrifuged and washed twice with PBS pre-chilled to 4° C. The cells were then blocked with Human TruStain FcX (BioLegend) and then subsequently stained for 30 minutes with Fixable Viability Dye (Invitrogen) and antibodies against CD3, CD4, CD8, CD25, and/or CD56. After staining, the cells were again centrifuged and washed twice with pre-chilled PBS. The cells were then fixed and permeabilized for 1 hour with 200 μL/well FoxP3/Transcription Factor Staining Buffer working solution (Invitrogen). After permeabilization, the cells were centrifuged, washed twice with permeabilization buffer, and subsequently stained for 30 minutes with anti-FoxP3 (BioLegend) and anti-Ki67 (BioLegend). After two additional washes, the cells were resuspended in 125 μL/well flow buffer and acquired on a BD FACSCelesta.

### Whole Blood Cytokine Release Assay

Cytokine secretion was detected using fresh human whole blood (heparinized) obtained from AllCells. The following method was adapted from B. Wolf et al. Cytokine 60 (2012) 828-837(61). 12.5 μL of 20× concentrated (diluted in 1×PBS) test article was added to each well of a sterile 96-well round bottom plate. To this, 237.5 μL of fresh, human whole blood was added to each well with minimal pipetting to reduce non-specific activation. The plate was the covered and incubated at 37° C. and 5% C02 overnight. The following morning, the plate was centrifugated at 1800×g for 10 minutes and then 50 μL of serum was transferred to a 96-well microplate. The serum was then immediately tested by MSD (#K15010K-1 or a custom U-Plex plate) or frozen at −80° C. for later testing.

### Mouse Pharmacokinetic (PK) Evaluation

The PK of BsAb-1 and BsAb-2 were each evaluated in 6 male BALB/c mice following a single tail vein injection of 1 mg/kg (n=3*6 groups, Aragen Biosciences, Morgan Hill, CA). Serum samples were collected at selected time points over the course of 14 days post-dose.

The PK of BsAb-5 was evaluated in two groups of nine female BALB/c mice following a single tail vein injection of 1 mg/kg or 10 mg/kg (9 mice per dosing group, CrownBio, San Diego, CA). Serum samples were collected at selected time points over the course of 14 days post-dose.

### Mouse Accelerated GVHD Study

Each immune-compromised NSG mouse (8-9 weeks old from Charles River, France) was irradiated with 1.5 Gy on study day −1. Mice were divided into 4 groups (n=5) and 2 independent experiments were conducted using 2 different PBMC donors. On study day 0, each mouse was adoptively transferred IV with 20 million human PBMCs from one of the 2 donors and each mouse was treated with either vehicle control (100 μL), 22 μg rhTL-2 (350,000 UI/mice, Proleukin, Novartis) daily, 1 mg/kg BsAb-1 twice a week or 1 mg/kg BsAb-2 twice a week. GVHD was assessed by measuring weight loss over time in all animals. Animals were euthanized when body weight loss of 20% was observed.

In the second experiment, NSG mice (8-9 weeks old from Charles River, France) were irradiated with 1.5 Gy on study day −1. Mice were divided into 4 groups and 2 independent experiments were conducted using 2 different PBMC donors. On study day 0, each mouse was adoptively transferred IV with 20 million CSFE-labelled human PBMCs from one of the 2 donors and each mouse was treated with either vehicle control (100 μL) (n=7), 22 μg rhIL-2 (350,000 UI/mice) daily (n=6), 1 mg/kg BsAb-1 twice a week (n=6) or 1 mg/kg BsAb-2 twice a week (n=6). All animals were sacrificed on study day 5.

Immunophenotyping of the engrafted PBMCs by flow cytometry was performed on single-cell suspensions prepared from the mouse spleen on the day of sacrifice. The method of detection was largely the same as the above method for Ki67 detection by flow cytometry, but with different panels of antibodies to better distinguish the human PBMCs from the host cells. Cells were surface stained with anti-human CD45, CD3, CD4, CD8, CD25, CD16, CD19, and/or CD69. Following fixation and permeabilization, some of the cells were stained with anti-human FoxP3. The samples were then collected on a flow cytometer and analyzed using FlowJo analysis software.

For BsAb-5, 10 million PBMCs were transferred instead of 20 million, and mice were divided into 3 groups.

### Cynomolgus Pharmacodynamic (PD) Study

The PD profiles of BsAb-1 and BsAb-2 were evaluated in twelve 2-4 years old naive cynomolgus monkeys following a single IV (slow bolus) dose of 0.03, 0.1 or 0.3 mg/kg. Each treatment group contained 1 male and 1 female cynomolgus monkey (Charles River Lab, USA, Reno, NV). Blood samples were collected at selected time points for 21 days after dosing for analyses of hematology, serum chemistry, cytokines, and PD endpoints. After study termination, animals from the study were returned to the general colony. All procedures were approved by CRL IACUC and were performed in compliance with the Animal Welfare Act, the Guide for Care and Use of Laboratory Animals and the Office of Laboratory Animal Welfare.

### Cynomolgus Blood Immunophenotyping

A portion of the blood from each collected time point was used for immunophenotyping and quantification by flow cytometry. The method for Ki67 detection by flow cytometry was largely the same as described above, but with a different panel of cyno-reactive antibodies. Cells were surface stained with antibodies against CD3, CD4, CD8, CD20, CD25, and CD159a. After fixation and permeabilization, the cells were stained with antibodies against FoxP3 and Ki67. The samples were then collected on a flow cytometer and analyzed using FlowJo analysis software.

Simultaneously, a portion of each blood sample was transferred to BD TruCount tubes and stained with CD45 for real time quantification of peripheral blood cell absolute counts. The cell subset percentages from the above blood analysis were applied to the total cell numbers from the corresponding TruCount tube.

aIgG4 ELISA

Serum concentrations of BsAb-1 and BsAb-2 in mouse serum were determined using an antigen capture ELISA. All washes and dilutions were performed with freshly made TBS-T (Accuris). All volumes should be assumed to be 100 μL/well except for coating, blocking and washing, which are at 200 μL/well. The night before the assay, Nunc MaxiSorp™ flat-bottom plates (Invitrogen) were coated with recombinant human IL2Rγ protein diluted to 1 μg/mL in carbonate-bicarbonate buffer (Thermo Scientific) and left at 4° C. The next day, the plates were washed 5 times and then blocked with 1% BSA for 30 minutes. The plates were washed once and then multiple dilutions of the serum samples were added, along with a reference standard. Stocks of known concentration for BsAb-1 and BsAb-2 were used to make the standard curve. After 1 hour at room temp, the plate was washed 8 times and then biotinylated anti-human IgG4-Fc (MABTECH) diluted to 3 μg/mL was added. The plates were incubated at room temperature for another 30 minutes and then washed again 8 times. Next, the plates were incubated for 30 minutes with HRP-Streptavidin (Thermo Scientific) diluted 1:4000. Following an additional 8 washes, the plates were incubated in the dark for 6 minutes with room-temperature 1-Step Ultra TMB (Thermo Scientific). The reaction was stopped with 100 μL/well 2 N sulfuric acid. Absorbance was assessed at 450 nm and 570 nm.

### Protein Expression and Purification

Monospecific antibody fragments were expressed in ExpiCHO cells following the manufacturer's instructions (ThermoFisher A29133, Standard Protocol). Clarified supernatants were harvested on day 7 and purified using Protein A magnetic beads, using the KingFisher Flex Platform (ThermoFisher). Antibodies were eluted in 0.1 M citrate, 0.1 M NaCl, 10% glycerol, 10% sucrose, pH 3.5.

To express bispecific antibody fragments, ExpiCHO cells were transfected with two expression vectors (knob and hole vectors, knob vectors contain C-terminal His-tag) and were expressed in the ExpiCHO cells according to manufacturer's instructions using the high titer protocol. Clarified supernatants were harvested and the antibodies were purified by IMAC (Ni Sepharose® Excel, Cytive Life Sciences), using an imidazole gradient for elution. The IL-2Rβγ bispecific heavy chain-only antibody containing fractions were pooled, concentrated, and further purified on cation exchange to remove any product-related impurities (Mono S® 10/100 GL column (Cytiva Life Sciences)). All antibody fragments were analyzed by SEC-UPLC and SDS-PAGE to confirm their size and purity.

The cynomolgus IL-2Rγ sequence was obtained from Uniprot.org (UniProt Accession ID: G7Q2Z6), and the extracellular domain (aa Metl-Asn254) was cloned into a proprietary vector containing the endogenous leader sequence and a C-terminal His-tag. The IL-2Rγ reagent was expressed in ExpiCHO cells, according to the vendors instructions (high titer protocol, ThermoFisher). Cells were harvested on day 8 and supernatant was run on SDS-PAGE (NuPAGE 4-12% Bis Tris Gel) to verify target protein expression. Clarified harvest was purified by IMAC using Ni-Sepharose Excel resin (Cytiva Life Sciences), using an imidazole gradient for elution. The peaks were pooled and quantified using QiaXpert (Qiagen).

The cloning, expression, and purification of mutant IL-2 protein (T3A, F42A, Y45A, L72G, C125A) was completed at Lake Pharma. A C-terminal His-tag was added to enable purification by IMAC using standard procedures and elution with an imidazole gradient.

### Octet-Based Off-Rate Measurements

All off-rate measurements were performed on an Octet Qk384 instrument (ForteBio), in 96-well microplates at 25° C. using anti-human IgG Fc capture (AHC, 18-5005) sensors with a shake speed of 1000 rpm. For off-rate determination, the antibodies/antibody fragments were loaded on the AHC sensors at 5 μg/mL. Following a short baseline in kinetics buffer (0.02% Tween20, 0.1% BSA, 0.05% sodium azide, 1X PBS). Offrate measurements were done for the following: human IL-2Rβ (AcroBiosystems), human IL-2Ry (Sino Biological), cynomolgus IL-2RP (Sino Biological), cynomolgus IL-2Rγ (expressed and purified in house using ExpiCHO expression system followed by Ni-NTA His-tag purification), mouse IL-2Rγ (Sino Biological), mouse IL-2Rβ (Sino Biological), human IL-2Rα (Sino Biological), IL-4R (Sino Biological), IL-7R (Sino Biological), IL-9R (R&D Systems) and IL-21R (Sino Biological). The following antibodies were used as positive controls to verify target binding and reagent quality: anti-human IL-9R (R&D Systems), anti-human IL-21R (R&D Systems), anti-human IL-7R (R&D Systems) and anti-human IL-4Ra (R&D Systems). The loaded sensors were then submerged in wells containing antigen at 100 nM concentration for association step. Dissociation was monitored in kinetics buffer. The capture surfaces were regenerated for 60 s. ForteBio data analysis software was used to fit the data to a 1:1 binding model to extract an association rate and dissociation rate.

### Octet-Based Kinetics Measurements

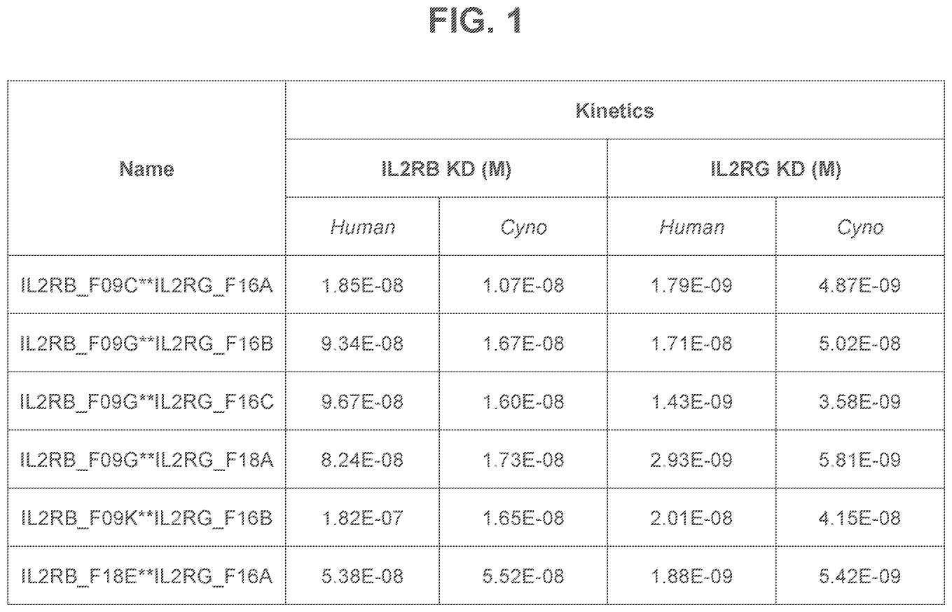

All kinetics measurement experiments were performed on a ForteBio Octet Qk384 instrument using anti-human Fc capture (AHC, 18-5005) sensors. The bispecific heavy chain-only antibodies and antigens were diluted to final concentrations in Kinetics buffer (0.02% Tween20, 0.1% BSA, 0.05% sodium azide, 1X PBS). Kinetics measurements were against the following antigens: human IL-2RB (AcroBiosystems), human TL-2RG (AcroBiosystems), cynomolgus IL-2RB (Sino Biological), cynomolgus IL-2RG (expressed and purified in house using ExpiCHO expression system followed by Ni-NTA his-tag purification). The antibodies were loaded on the AHC sensors at 5 μg/mL for maximum loading. Following a short baseline in Kinetics buffer, the sensors were exposed to a series of analyte concentrations (7.8 nM to 500 nM) for association step and background subtraction was used to correct for sensor drifting. Dissociation was monitored in Kinetics buffer. The capture surfaces were regenerated for 60s. All experiments were performed with shaking at 1000 rpm. ForteBio's data analysis software was used to fit the data to a 1:1 binding model to extract an association rate and dissociation rate. The KD was calculated using the ratio kd/ka. Kinetics data for six bispecific heavy chain-only antibody constructs is provided in FIG. 1.

### Biophysical Characterization Assay (Tm, Tagg)

Tm and Tagg were measured on the UNcle platform. Briefly, 9 μL of each sample was loaded in duplicate in a Uni (UNcle cassette) and run with a thermal ramp from 20° C. to 70° C. at a constant rate of 1° C./min. UNcle Analysis 3.1 software, was used to calculate the Tm of each sample using the first derivative of the barycentric mean (BCM) of the fluorescence intensity. The Tagg for each sample was calculated using the intensity of scattered light at 266 nm.

### Thermal Stress and Stability Characterization

Bispecific heavy-chain only antibodies were concentrated to 10 mg/mL in 20 mM citrate and 0.1 M NaCl pH 6.2. Presence of high and low molecular weight species (% HMW and % LMW) was determined before and after temperature stress for 1 month at 2-8° C. and 37° C. by SEC on an analytical ThermoFisher UltiMate™ 3000 UPLC.

### Example 1: Identification of IL-2Rβγ Bispecific Heavy Chain-Only Antibody Combinations with Agonist Activity

The activation of the IL-2 receptor complex triggers a signaling cascade that results in the phosphorylation of STAT5 (pSTAT5), translocation of pSTAT5 dimers to the nucleus, and transcription of STAT5-regulated genes (M. Rickert, et al., Science 308, 1477-1480 (2005); G. C. Sim, et al., Cytokine Growth F R 25, 377-390 (2014)). As a primary assay to determine if bispecific heavy chain-only antibodies targeting the beta and gamma subunits of IL-2R could induce activation of IL-2R signaling, 5 anti-IL-2Rβ binding arms from unique CDR3 families and 5 anti-IL-2Rγ binding arms from unique CDR3 families were combined to make 25 bispecific heavy chain-only antibodies for conducting an all-by-all screen of agonist activity. The bispecific heavy chain-only antibodies were expressed on a silenced and stabilized human IgG4 Fc (CH1 domain deleted) using knobs-into-holes technology to facilitate heavy-chain heterodimer formation, with a single anti-IL-2Rγ VH on the knob arm and a single anti-IL-2Rβ VH on the hole arm (J. B. B. Ridgway et al., Protein Eng Des Sel 9, 617-621 (1996); S. M. Canfield, et al., J Exp Medicine 173, 1483-1491 (1991); D. Xu et al., Cell Immunol 200, 16-26 (2000); J. W. Bloom et al., Protein Sci 6, 407-415 (1997); M. P. Reddy et al., J Immunol 164, 1925-1933 (2000); A. M. Merchant et al., Nat Biotechnol 16, 677-681 (1998)).

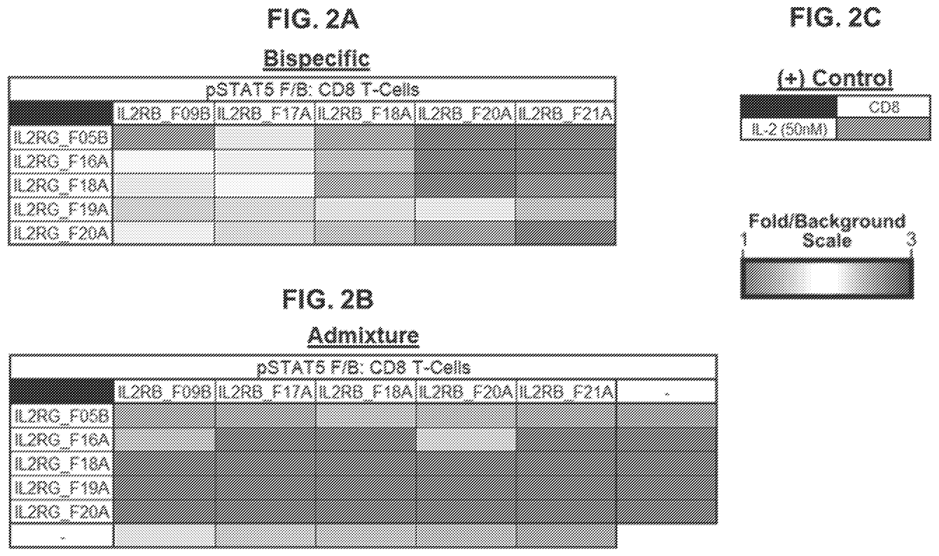

A phospho-flow cytometry assay was used to measure and compare the phosphorylation of STAT5 by the 25 IL-2Rβγ combinations compared to rhIL-2 on human CD8+ T-cells. STAT5 phosphorylation was not observed with any of the anti-IL-2Rβ or anti-IL-2Rγ monospecific heavy chain-only antibodies. Similarly, STAT5 phosphorylation was also not observed when anti-IL-2Rβ and anti-IL-2Rγ monospecific heavy chain-only antibodies were tested as a mixture in the pSTAT5 assay (FIG. 2, Panel B). In contrast, the bispecific heavy chain-only antibodies with one anti-IL-2Rβ arm and one anti-IL-2Rγ arm exhibited varying levels of agonist activity, summarized in FIG. 2, Panel A. Interestingly, the ability to induce phosphorylation of STAT5 agonist activity seemed highly dependent on the anti-IL-2Rβ arm present in the bispecific combination, while the degree of agonism appeared to be dependent on the anti-IL-2Rγ arm. Control data is shown in FIG. 2, panel C.

To identify heavy chain-only antibodies with a greater range of agonist activity, a secondary diversity screen was initiated to survey other unique VH sequences in 3 of the 4 lead CDR3 clonotype families identified in the bispecific screen for STAT5 activity. These additional VH sequences were selected from the lead CDR3 clonotype families and contain sequence variation in CDR1, CDR2, and framework regions. In total, an additional 157 unique family members underwent a second round of high-throughput gene assembly and expression and were assessed for binding to IL-2R expressing cells. For IL-2Rβ, an additional 33 IL-2Rβ family F09 members and an additional 22 IL-2Rβ family F18 members that bound to human and cynomolgus IL-2Rβ cells were identified in the diversity screen. A further 29 IL-2RT family F16 members were identified that bound to human and cynomolgus IL-2Rγ recombinant protein and on cells in the diversity screen. This large and diverse set of novel IL-2R binding heavy chain-only antibodies enabled subsequent efforts to identify a set of lead IL-2Rβγ bispecific combinations with a range of functional activity.

### Example 2: In Vitro Characterization of IL-2Rβγ Bispecific Heavy Chain-Only Antibodies

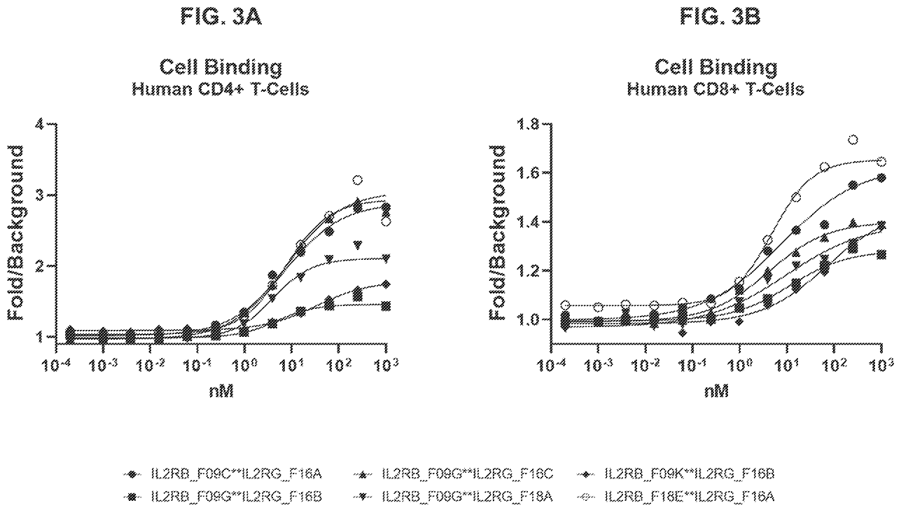

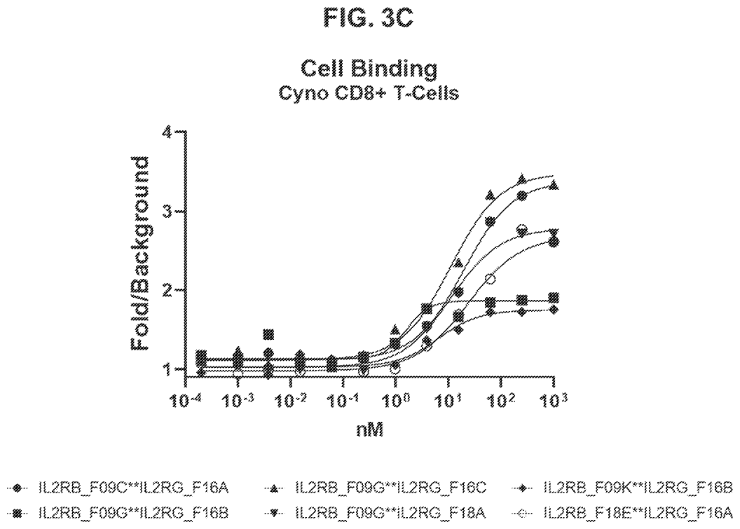

Based on the primary and secondary binding screening results as well as the STAT5 phosphorylation seen in the all-by-all bispecific heavy chain-only antibody screen, 6 IL-2Rβγ bispecific heavy chain-only antibodies were selected for additional in vitro characterization. The 6 IL-2Rβγ bispecific heavy chain-only antibodies bound efficiently to both human and cynomolgus T-cells with a range of EC50 values (FIG. 3, panels A-C). None of the 6 bispecific heavy chain-only antibodies bound to other common gamma chain partners (IL-4R, IL-7R, IL-9R or IL-21R) or IL-2Rα by Octet off-rate analysis.

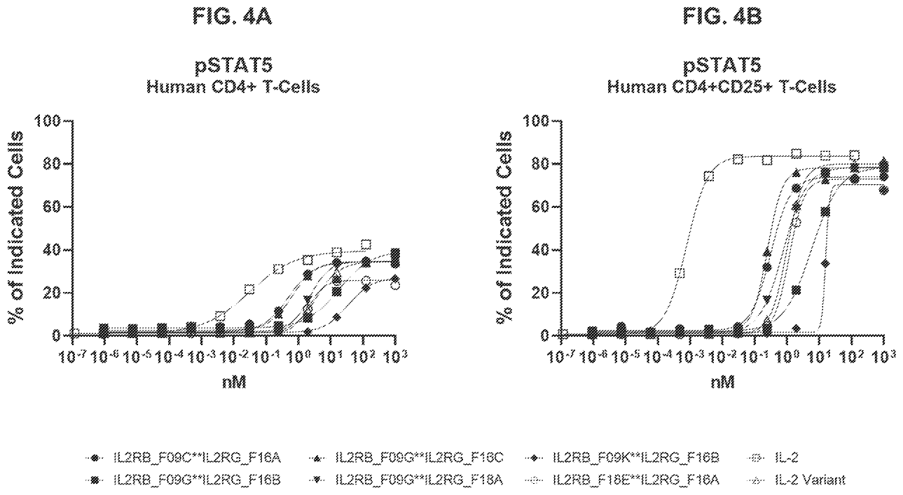

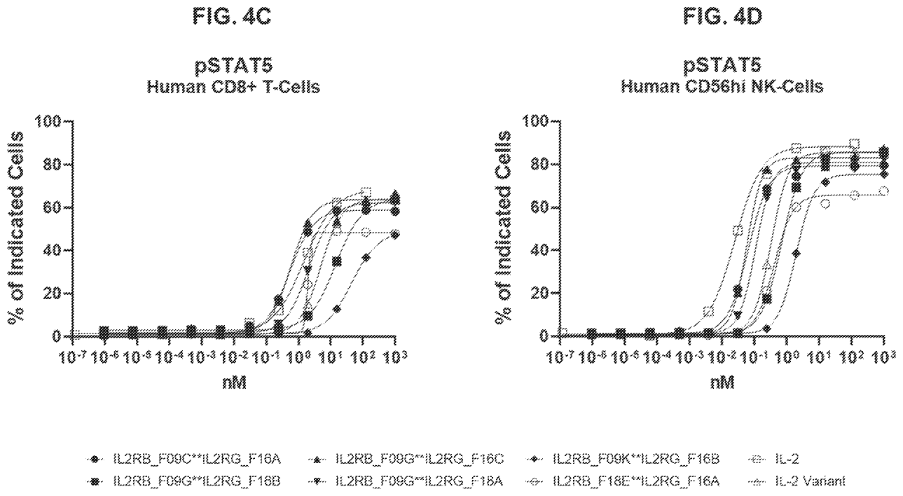

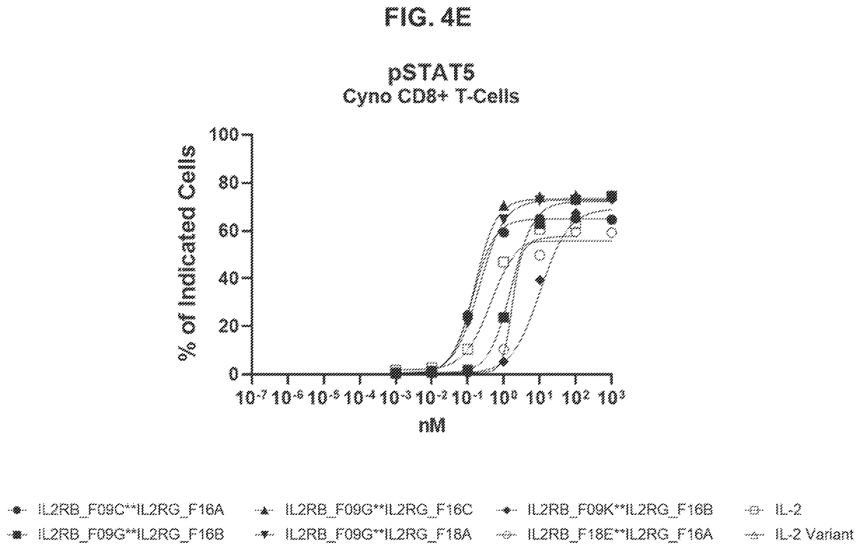

The ability of the IL-2Rβγ bispecific heavy chain-only antibodies to stimulate IL-2R signaling in human CD4+ T, CD8+ T, and NK-cells was confirmed by a dose-dependent increase of STAT5 phosphorylation compared to rhIL-2 and an rhIL-2 variant which contains mutations (F42A, Y45A, L72G) that have been shown to disrupt binding to IL-2Rα while retaining the ability to bind and activate the intermediate affinity IL-2Rβγ receptor (FIG. 4, Panels A-D) (C. Klein et al., Oncoimmunology 6:3 e1277306 (2017)). On CD8+ T-cells, the bispecific heavy chain-only antibodies exhibit a range of EC50 values in the pSTAT5 assay, with multiple constructs (BsAb-1, BsAb-3, BsAb-4) showing near equivalent activity with rhIL-2 and the rhIL-2 variant (FIG. 4, Panel C). However, this is in stark contrast to the level of pSTAT5 in CD4+CD25+FoxP3+T-regulatory cells, where the bispecific heavy chain-only antibodies show significantly lower potency compared to rhIL-2 on cells that express high levels of IL-2Rα (FIG. 4, Panel B). Thus, IL-2Rβγ bispecific heavy chain-only antibodies avoid the preferential activation of T-regs, which is a key functional criterion for these molecules. All 6 bispecific heavy chain-only antibodies were also confirmed to activate IL-2R signaling on cynomolgus T-cells, establishing cynomolgus monkeys as a suitable non-human primate model in subsequent studies (FIG. 4, Panel E).

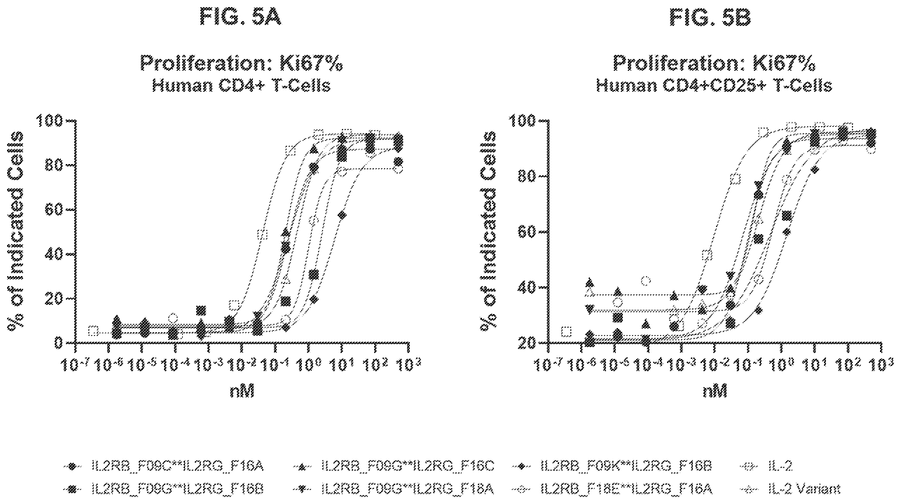

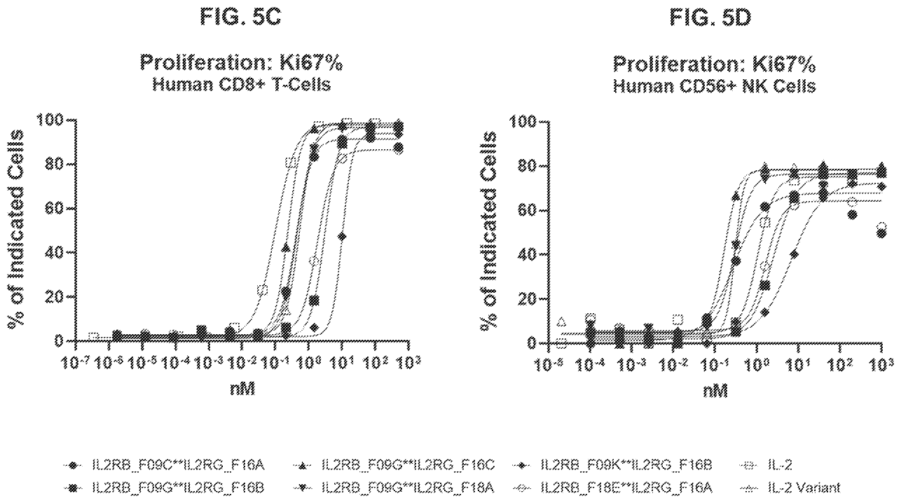

To further compare the functional activity among the 6 bispecific heavy chain-only antibodies and rhIL-2, a cell proliferation assay was performed. In response to treatment with the bispecific IL-2R agonist heavy chain-only antibodies, or the IL-2 cytokine controls, immune effector cells (T and NK-cells derived from healthy donor PBMCs) demonstrated dose dependent proliferation (FIG. 5, Panels A-D). While a range of potencies was observed in the proliferation of CD8⁺ T-cells and NK-cells in PBMCs treated with the IL-2Rβγ bispecific heavy chain-only antibodies, several (BsAb-1, BsAb-3, BsAb-4) showed induction of proliferation at levels similar to rhIL-2 and the rhIL-2 variant control, while all molecules achieved similar levels of maximum proliferation (FIG. 5, Panels C-D). In contrast, rhIL-2 was more active than the bispecific agonist heavy chain-only antibodies and the rhIL-2 variant control on CD4+cells (including T-regs) (FIG. 5, Panels A-B).

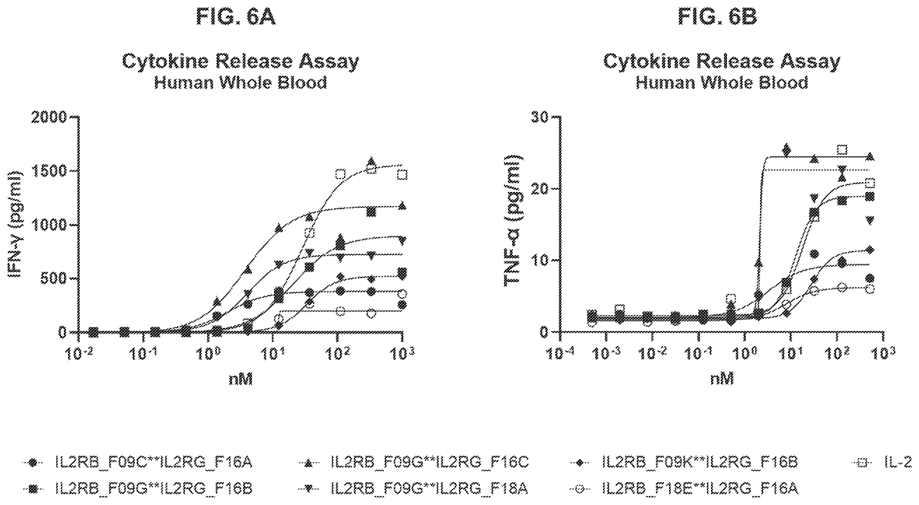

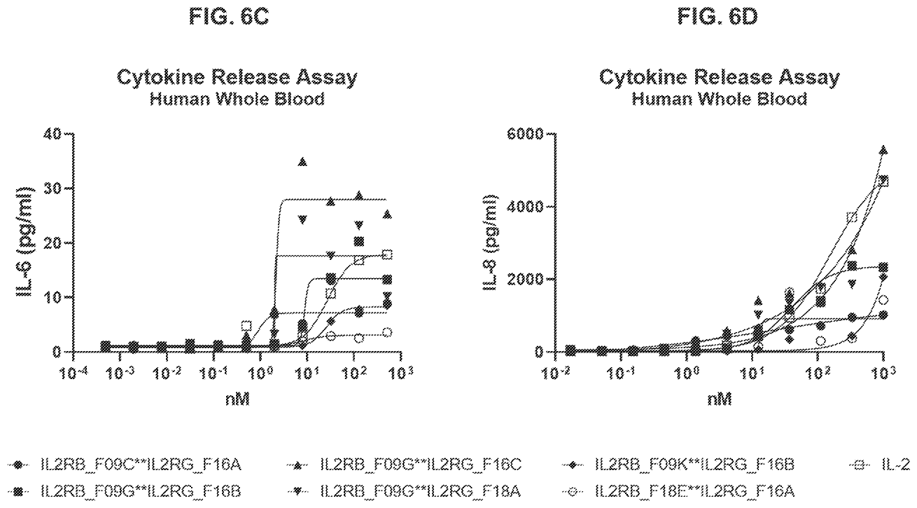

Cytokine release profiles of the bispecific IL-2R agonist heavy chain-only antibodies compared to rhIL-2 were assessed in an ex vivo human whole blood assay. After a 24-hour incubation in the presence of the IL-2Rβγ bispecific heavy chain-only antibodies or rhIL-2, a dose-dependent increase in IFN-γ, TNF-α, IL-6, and IL-8 was observed for all test articles (FIG. 6, Panels A-D). Two of the bispecific heavy chain-only antibodies (BsAb-3 and BsAb-4) induced cytokine levels (max concentration or EC50) at or above that of rhIL-2 in all tested cytokines, but the remaining four induced levels lower than the cytokine control.

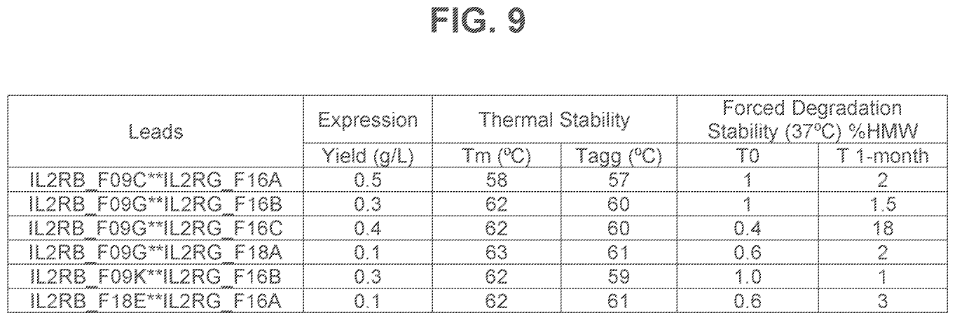

In summary, six bispecific IL-2Rβγ heavy chain-only antibodies were identified with a range of agonist activity. BsAb-1 demonstrates agonist activity at a similar level to that seen with rhIL-2 in immune effector cells measured by phosphorylation of STAT5 and in the proliferation assay. In contrast, in the same in vitro assays, BsAb-2 shows reduced potency compared to rhIL-2 and BsAb-1. Both antibodies showed low aggregation measured by SEC, had favorable melting temperatures, and were stable at 37° C. for one month (FIG. 9). These results combined with the favorable cytokine release profiles of BsAb-1 and BsAb-2 led to the selection of these two bispecific heavy chain-only antibodies for further in vivo characterization.

### Example 3: In Vivo Characterization of IL-2Rβγ Bispecific Heavy Chain-Only Antibodies BsAb-1 and BsAb-2

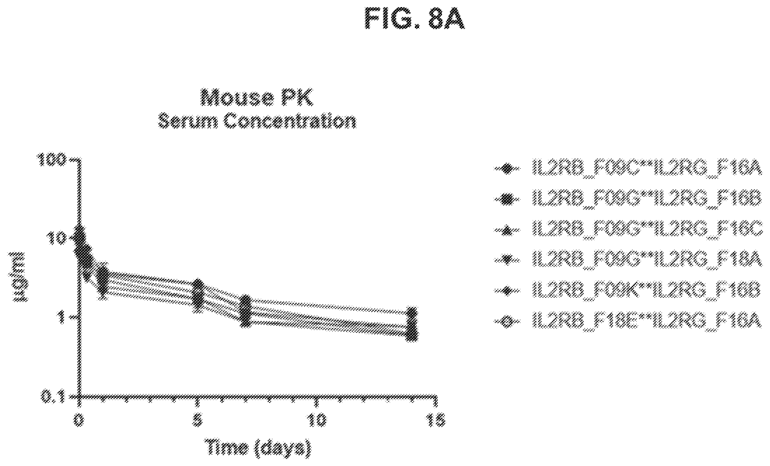

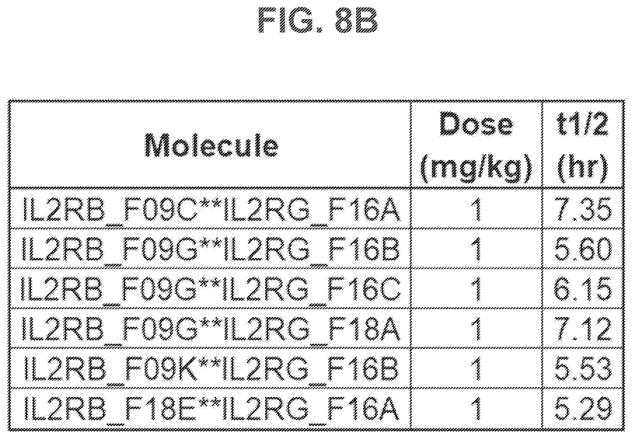

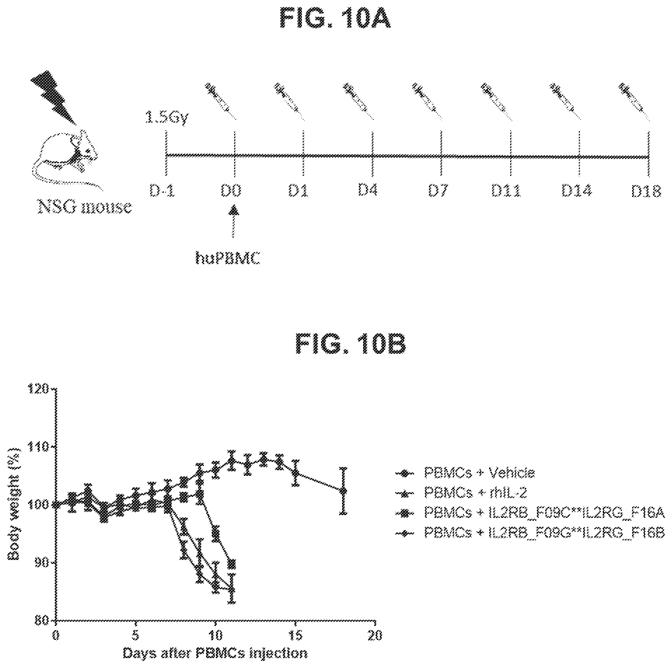

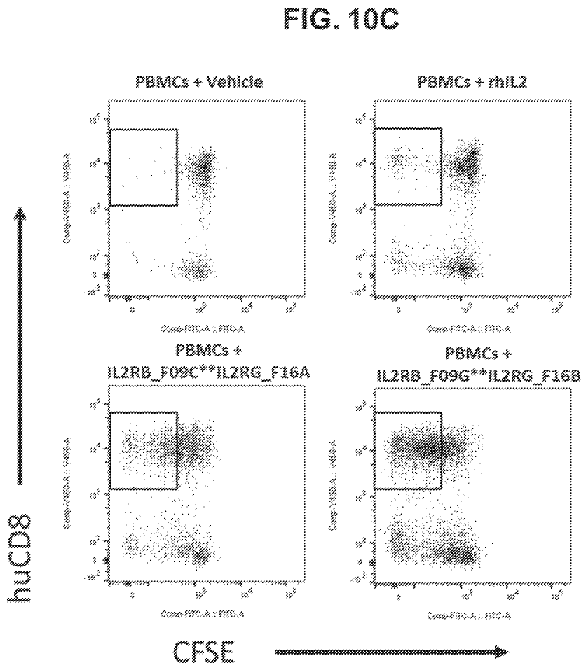

Prior to conducting in vivo functional studies, the in vivo stability and pharmacokinetics of the bispecific antibodies were measured in mice. The observed 5⁻⁷ day half-life of each bispecific antibody is consistent with the half-life of a human IgG4 antibody in mice (FIG. 8, Panels A-B) (R. Deng et al., Mabs 3, 61-66 (2011)). To assess the in vivo functional activity of the bispecific molecules, an accelerated graph versus host disease (GVHD) model was used to compare the functional activity of BsAb-1, BsAb-2, and rhIL-2 (FIG. 10, Panels A-C). In the first experiment, irradiated NSG mice were engrafted with human PBMCs, and the mice were subsequently treated with either vehicle, rhIL-2 daily, or one of the two bispecific agonist antibodies twice a week until sacrifice. As expected, animals treated with the vehicle control showed onset of GVHD, measured by body weight loss, around day 20 and were sacrificed with 20% body weight loss at approximately day 35. In contrast, the bispecific IL-2Rβγ agonist antibodies (BsAb-1, BsAb-2) as well as rhIL-2-treated animals exhibited onset of GVHD at approximately day 8 and were sacrificed with 20% body weight loss between days 9 and 13, indicating an acceleration of GVHD compared to the vehicle control, consistent with the enhanced activation of immune effector cells in treated mice (FIG. 10, Panel B).

A second study was conducted to directly measure the ability of BsAb-1 and BsAb-2 to stimulate the proliferation of immune effector cells in vivo. Similar to the first experiment, irradiated NSG mice were engrafted with human PBMCs that were labeled with CSFE and treated with vehicle, rhIL-2, BsAb-1, or BsAb-2. After day 5 of treatment, spleens were harvested and the proliferation of CD8+ T and CD4+ T-cells was compared between the 4 treatment groups by measuring CSFE staining in the different lymphocyte populations. BsAb-1 and BsAb-2 both showed significantly more proliferating CD8⁺ T-cells compared to rhIL-2 and the vehicle control (FIG. 10, Panel C). CD4+ T-cells were expanded to a lesser extent; however, a significant increase in proliferating CD4+ T-cells was seen in BsAb-2 treated mice compared to the vehicle control.

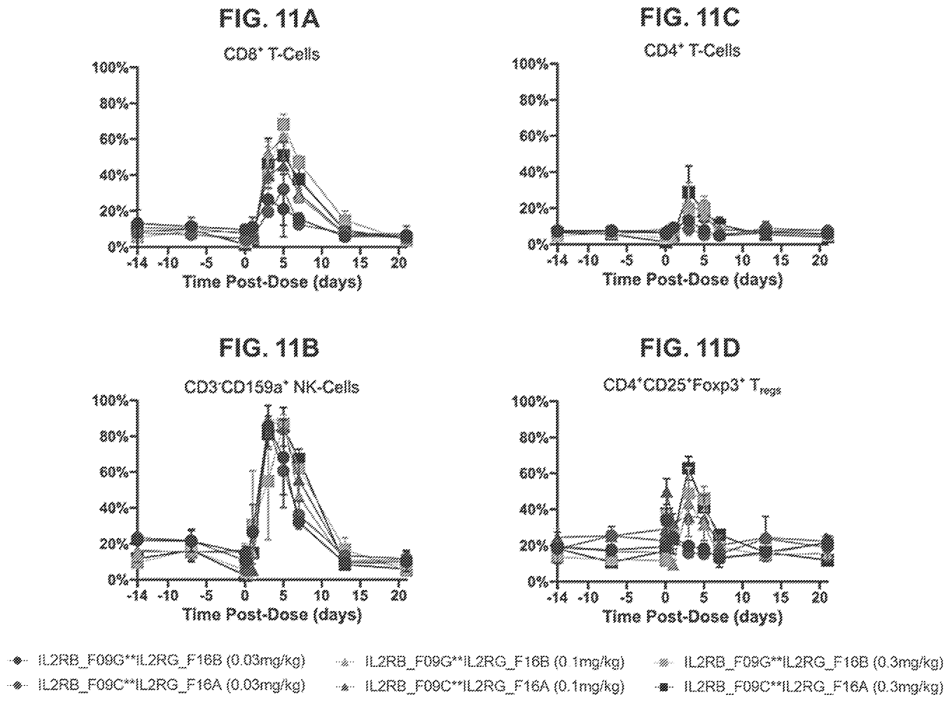

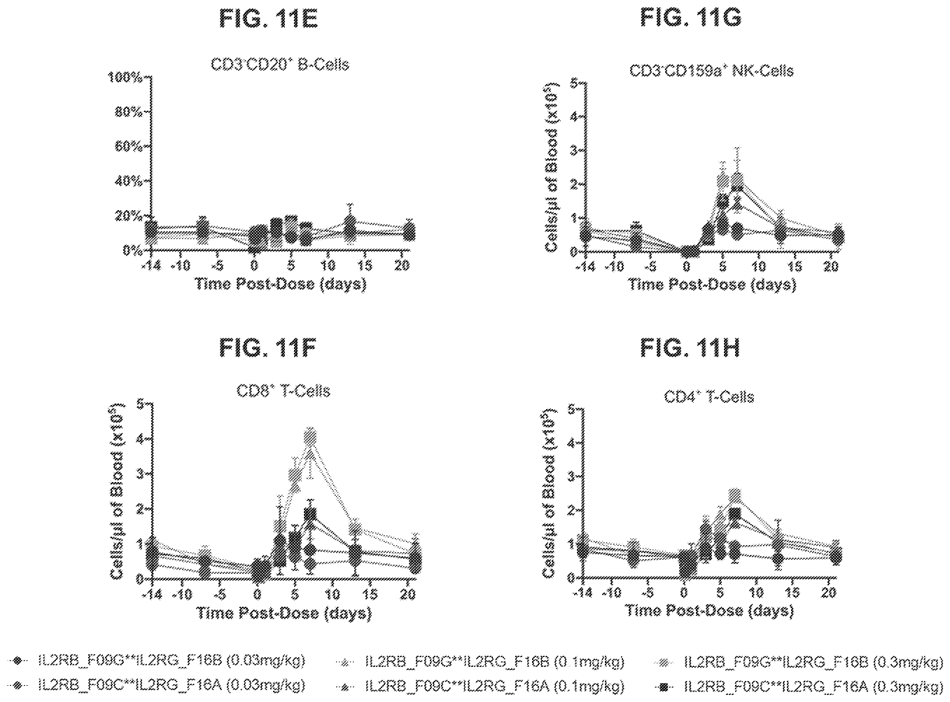

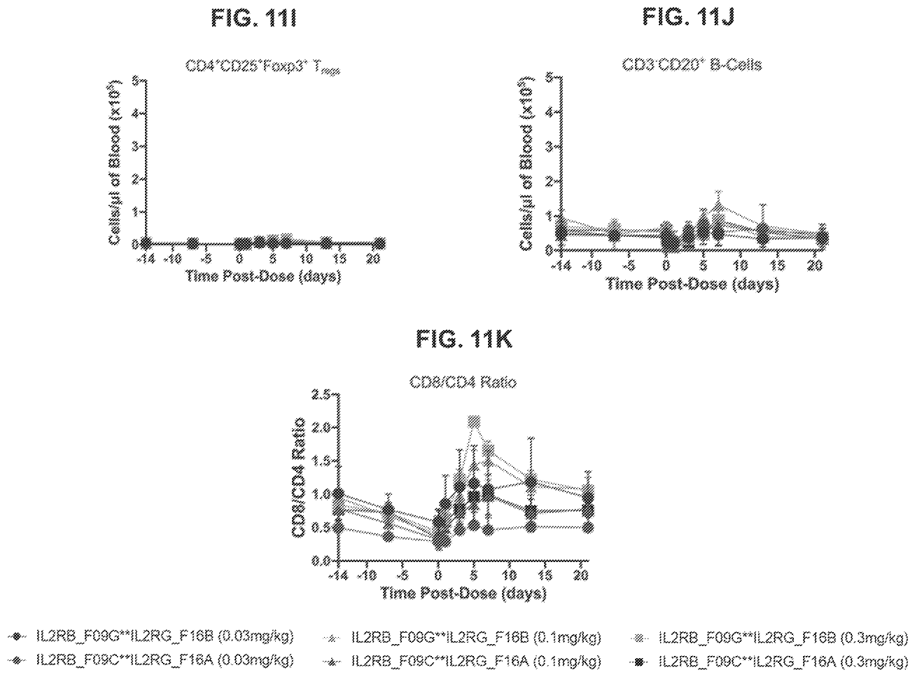

An important aspect of the preclinical evaluation of the bispecific heavy chain-only antibody agonists was establishing cynomolgus monkeys as an appropriate in vivo model for measuring the pharmacodynamics of the molecules. To determine human and cynomolgus functional equivalency, the bispecific heavy chain-only antibodies were confirmed to activate pSTAT5 signaling ex vivo in cynomolgus primary T-cells at a similar level as seen in primary human T-cells (FIG. 4, Panels C and E). After establishing functional equivalency between human and cynomolgus, a non-GLP cynomolgus study was conducted to further investigate the activity of BsAb-1 and BsAb-2 in vivo in a non-human primate model. The two bispecific agonist heavy chain-only antibodies were administered to cynomolgus monkeys in groups of 2 that received a single intravenous (slow bolus) dose of 0.03, 0.1 or 0.3 mg/kg of either BsAb-1 or BsAb-2. At all doses with both molecules, a marked expansion of peripheral CD8+T and NK-cells was observed (FIG. 11). After an initial transient drop in lymphocyte numbers, CD8+T, NK-cells, and to a lesser degree, CD4+ T-cells, showed dose dependent proliferation and expansion in the blood, peaking around day 4⁻⁷ before returning to baseline levels around day 14 (FIG. 11, Panels A-C, F-H). Importantly, no pronounced expansion of CD4+CD25+FoxP3+T-regulatory cells was seen, consistent with the bispecific agonist heavy chain-only antibodies avoiding preferential activation of the trimeric IL-2 receptor (FIG. 11, Panels D, I). This effect was further confirmed by the ratio of CD8+:CD4+ T-cells which was skewed in favor of the CD8+ T-cell subset (FIG. 11, Panel K). Moreover, the IL-2Rβγ agonist heavy chain-only antibodies were well tolerated in the monkeys at all dose levels tested, with no indication of vascular leak syndrome or other overt toxicities.

### Example 4: In Vivo Characterization of IL-2Rβγ Bispecific Heavy Chain-Only Antibody BsAb-5

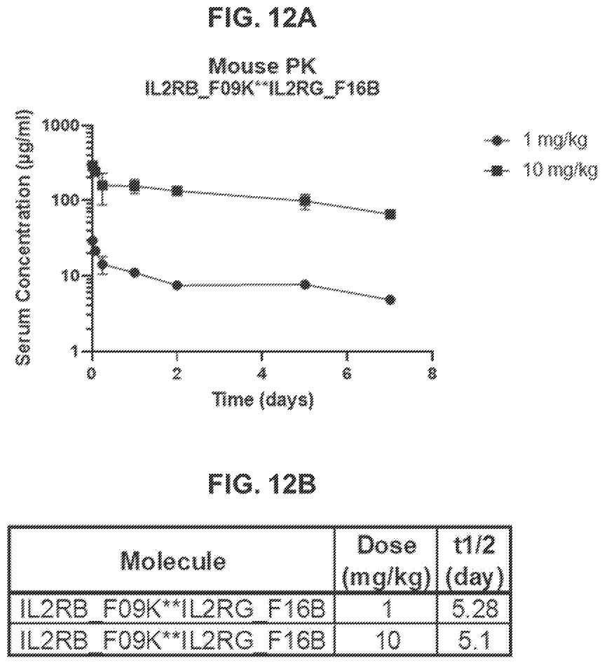

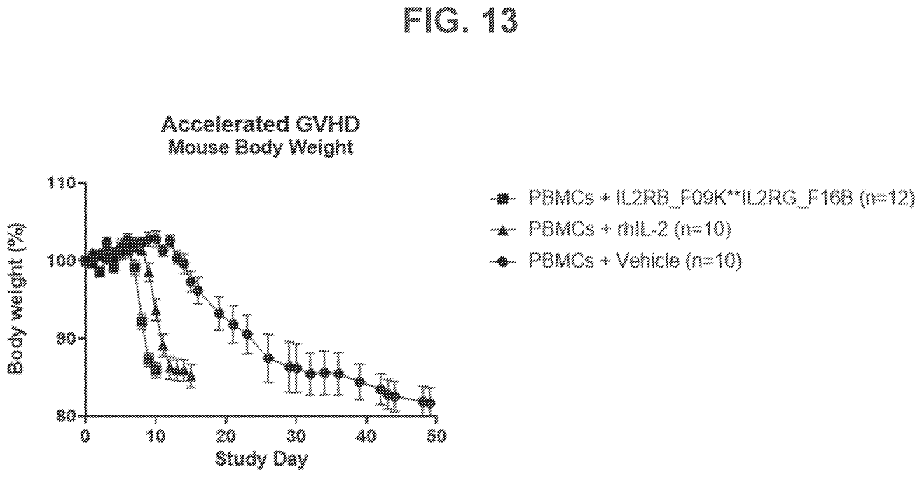

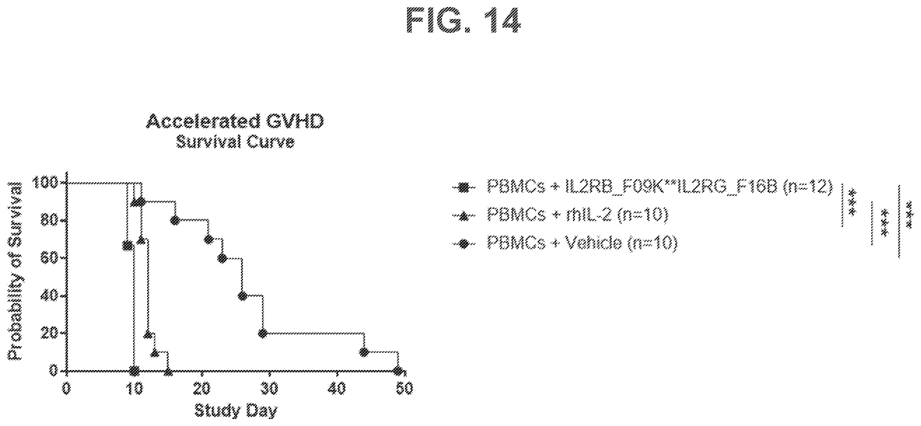

Prior to conducting in vivo functional studies, the in vivo stability and pharmacokinetics of the bispecific antibodies were measured in mice at two dose-levels: 1 mg/kg and 10 mg/kg. The observed 5-day half-life of BsAb-5 (IL2RB_F09K**IL2RG_F16B) is consistent with the half-life of a human IgG4 antibody in mice (FIG. 12, Panels A and B) (R. Deng et al., Mabs 3, 61-66 (2011)). To assess the in vivo functional activity of the bispecific antibody, an accelerated graph versus host disease (GVHD) model was used to compare the functional activity of BsAb-5 and rhIL-2 (FIGS. 13-14). In the first experiment, irradiated NSG mice were engrafted with human PBMCs, and the mice were subsequently treated with either vehicle, rhIL-2 daily, or BsAb-5 twice a week until sacrifice. As expected, animals treated with the vehicle control showed onset of GVHD, measured by body weight loss, around day 20 and were sacrificed with 20% body weight loss at approximately day 35. In contrast, BsAb-5-treated animals, as well as rhIL-2-treated animals, exhibited onset of GVHD at approximately day 8 and were sacrificed with 20% body weight loss between days 9 and 13, indicating an acceleration of GVHD compared to the vehicle control, consistent with the enhanced activation of immune effector cells in treated mice (FIG. 14).

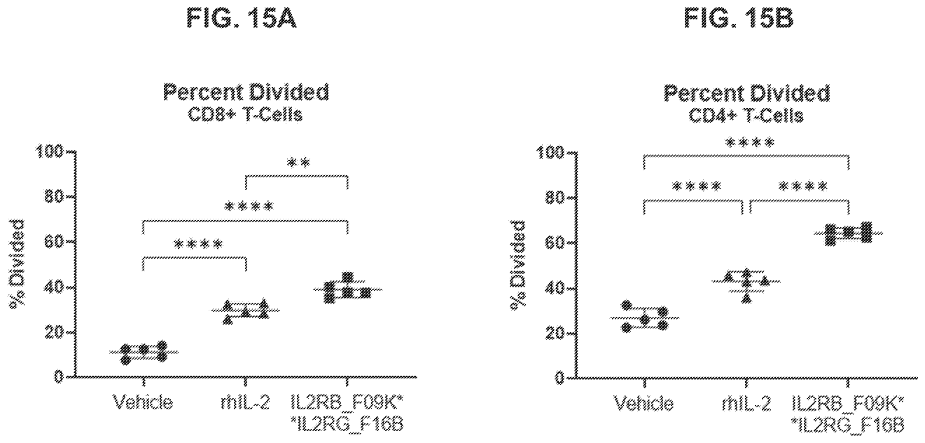

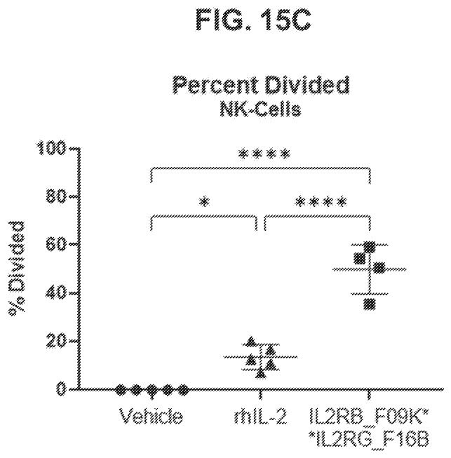

A second study was conducted to directly measure the ability of BsAb-5 to stimulate the proliferation of immune effector cells in vivo. Similar to the first experiment, irradiated NSG mice were engrafted with human PBMCs that were labeled with CPD450 and treated with vehicle, rhIL-2, or BsAb-5. After day 5 of treatment, spleens were harvested and the proliferation of CD8+ T-cells, CD4+ T-cells, and NK-cells were compared between the 3 treatment groups by measuring CPD450 dilutions in the different populations. In all three measured cell types, BsAb-5 induced significantly more proliferation than rhIL-2 or the vehicle control (FIG. 15, Panels A-C).

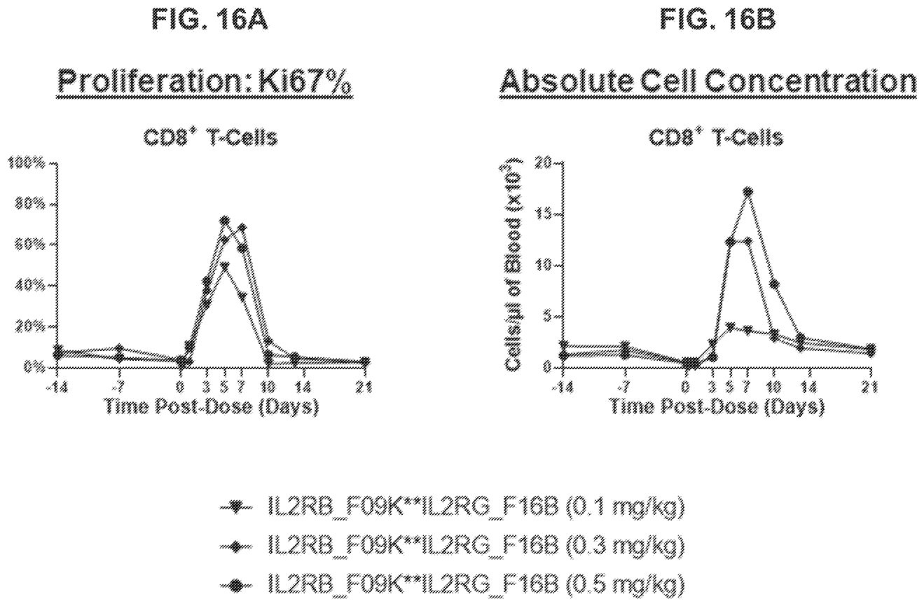

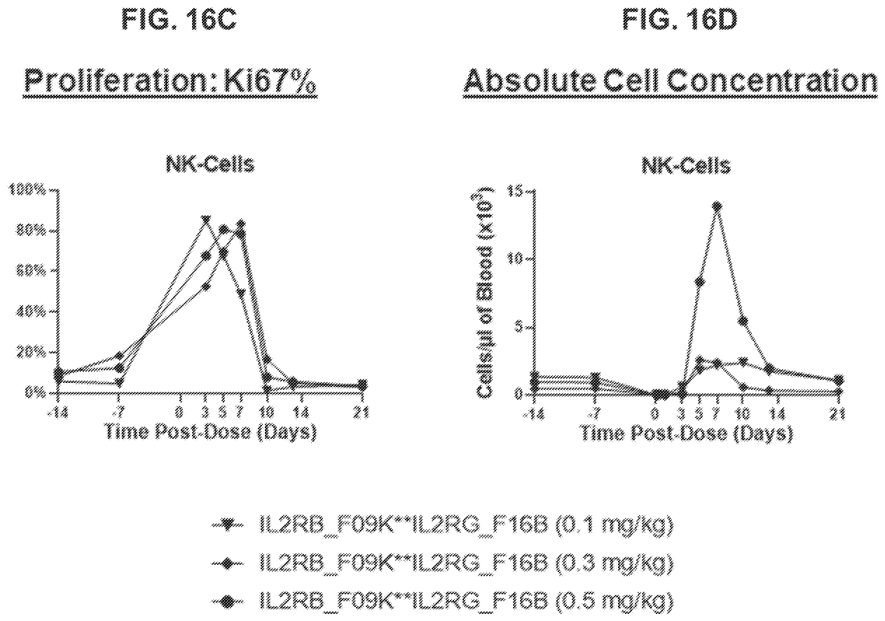

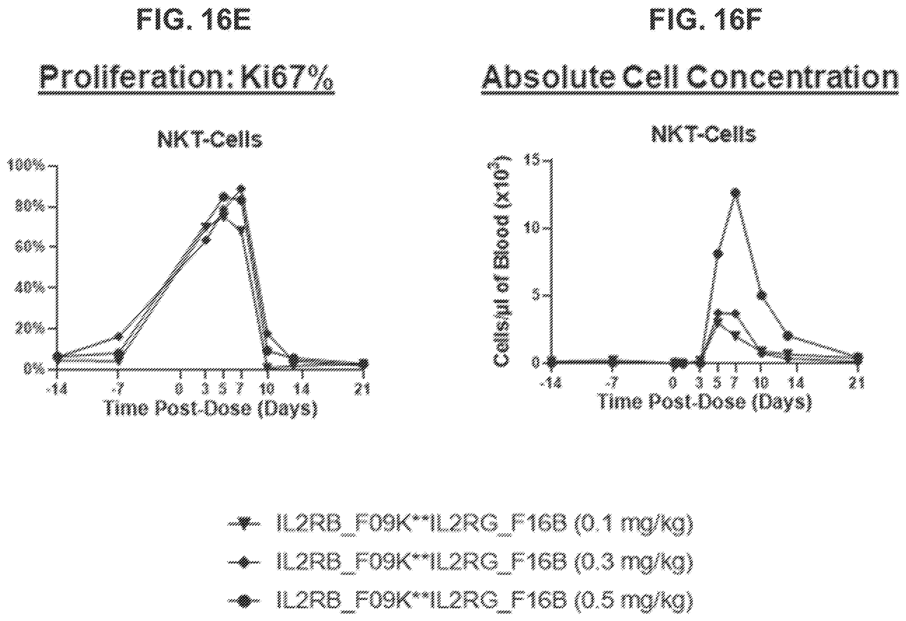

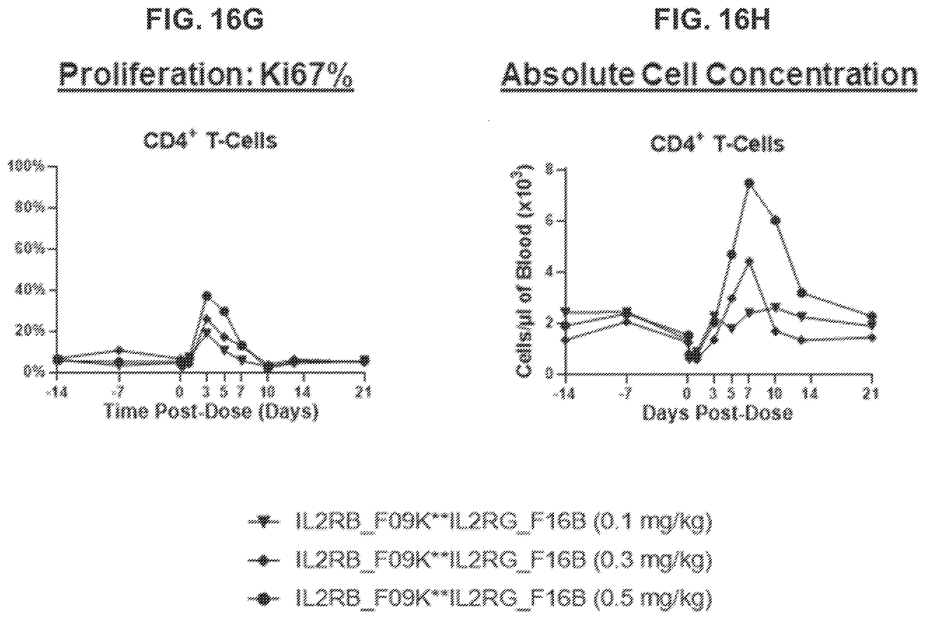

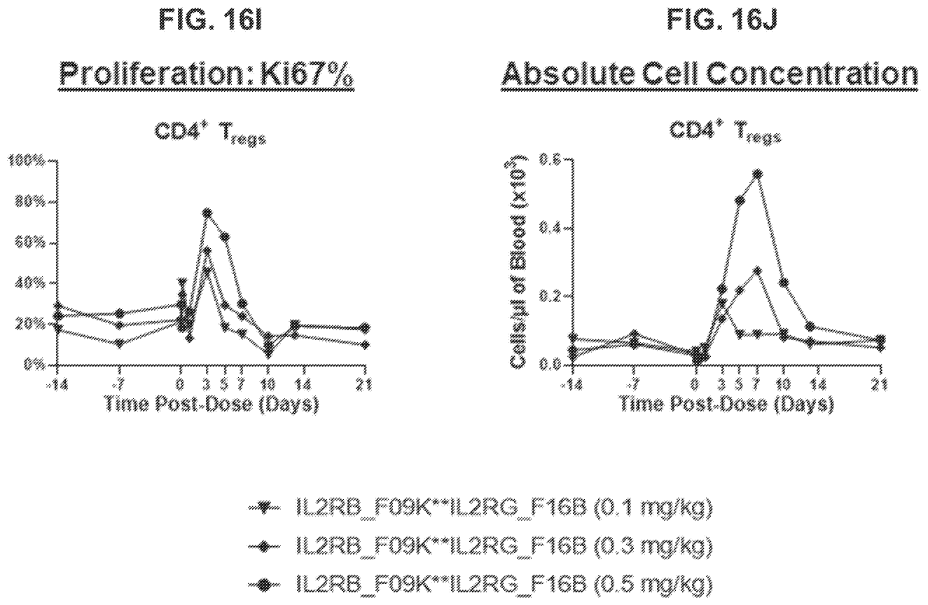

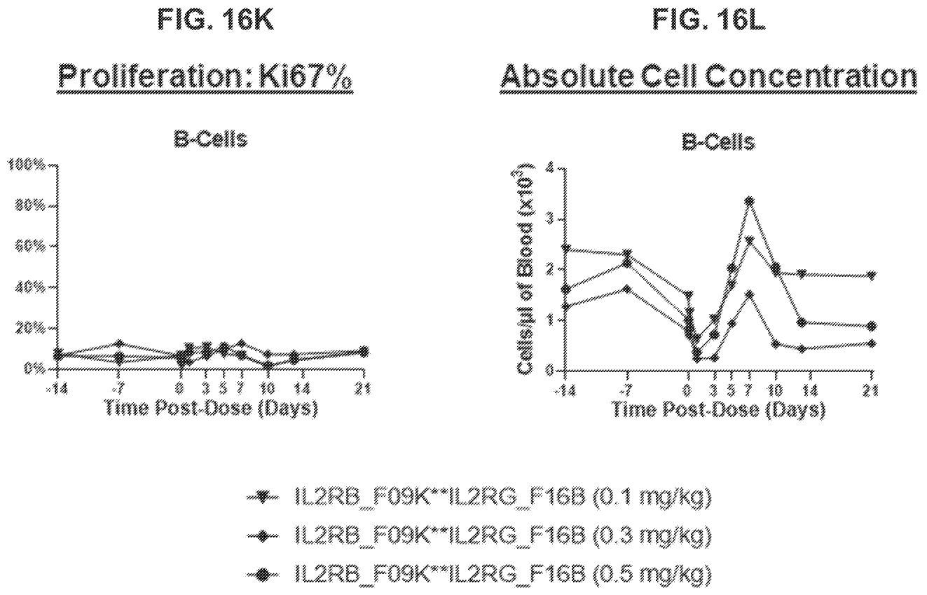

An important aspect of the preclinical evaluation of the bispecific heavy chain-only antibody agonists was establishing cynomolgus monkeys as an appropriate in vivo model for measuring the pharmacodynamics of the molecules. To determine human and cynomolgus functional equivalency, the bispecific heavy chain-only antibodies were confirmed to activate pSTAT5 signaling ex vivo in cynomolgus primary T-cells at a similar level as seen in primary human T-cells (FIG. 4, Panels C and E). After establishing functional equivalency between human and cynomolgus, a non-GLP cynomolgus study was conducted to further investigate the activity of BsAb-5 in vivo in a non-human primate model. BsAb-5 was administered to cynomolgus monkeys in a single intravenous (slow bolus) dose of 0.1, 0.3, or 0.5 mg/kg. The monkeys were dosed in groups of 2 for the first two dose levels, and in a group of 4 for the 0.5 mg/kg dose level. At all dose levels, a marked expansion of peripheral CD8+T-, NK-, and NKT-cells was observed (FIG. 16, Panels A-F). After an initial transient drop in lymphocyte numbers, CD8+T-, NK-, NKT-cells, and to a lesser degree, CD4+T-cells, showed dose dependent proliferation and expansion in the blood, peaking around day 4⁻⁷ before returning to baseline levels around day 14 (FIG. 16, Panels A-H). Importantly, no preferential expansion of CD4+CD25+Foxp3+T-regulatory cells was observed, consistent with the bispecific agonist antibodies avoiding preferential activation of the trimeric IL-2 receptor (FIG. 16, Panels I-J). Additionally, B-cells, which serve as a useful negative control due to their lack of IL-2 receptor expression, did not proliferate in response to BsAb-5 (FIG. 16, Panels K-L). Moreover, the IL-2Rβγ agonist heavy-chain only antibodies were well tolerated in the monkeys up to 0.5 mg/kg, with no indication of vascular leak syndrome or other overt toxicities.

### Example 5: In Vitro Combination Studies Using BsAb-5

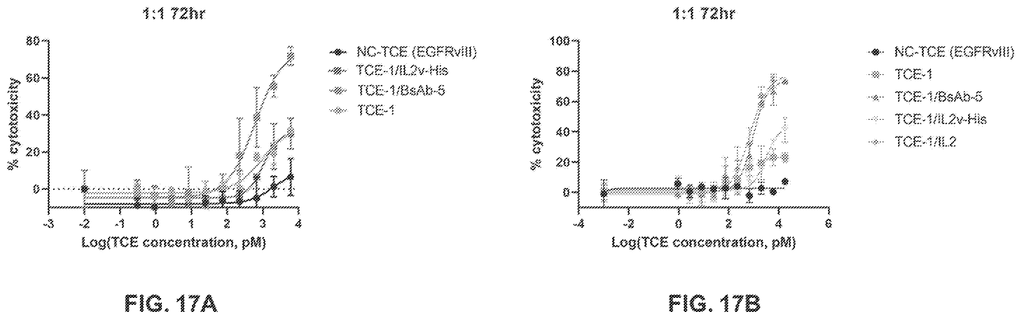

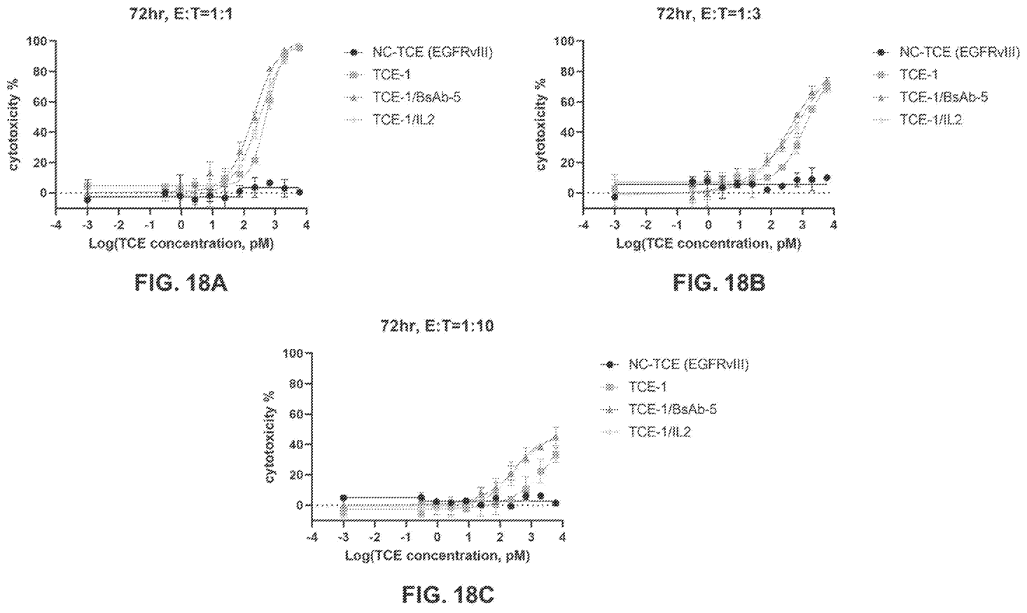

The ability of anti-IL-2Rβγ heavy chain-only antibodies to increase maximum target cell killing associated with a bispecific T-cell engaging molecule that specifically binds human CD3 and human EpCAM (TCE-1) was investigated using an in vitro T-cell dependent cellular cytotoxicity (TDCC) assay (FIG. 17, panels A and B; FIG. 18, panels A-C). In the TDCC assay, target cells (H82_Luc, SHP77 Luc) were co-cultured at 37° C. with pre-activated human T-cells for 72 hrs at specific effector:target (E:T) ratios (1:1, 1:3, 1:5, 1:10) and increasing TCE-1 concentrations, with and without addition of BsAb-5 (30 nM) or recombinant IL-2 (0.3 nM). A negative control molecule (NC-TCE) was also used in some TDCC assays. A total of 10,000 or 12,000 target cells per well were used in a 96-well plate format. Target cell viability was determined in the Steady-Glo® Luciferase Assay System by measuring Relative Luminescence units (RLU) using the BioTek Neo2 Microplate reader. Cytotoxicity was calculated using the formula:

[1−(RLU(T CELLS+TARGET CELLS+TCE−1))/(RLU(T CELLS+TARGET CELLS))]×100

BsAb-5 increased killing of both H82_Luc and SHTP77 Luc cells relative to TCE-1 only at low E:T ratio, similar to IL-2 control molecules (IL2, IL2v-His).

### Example 6: In Vivo Combination Study of TCE-1 and BsAb-5 in SHTP77 Luc Model in Female NSG Mice

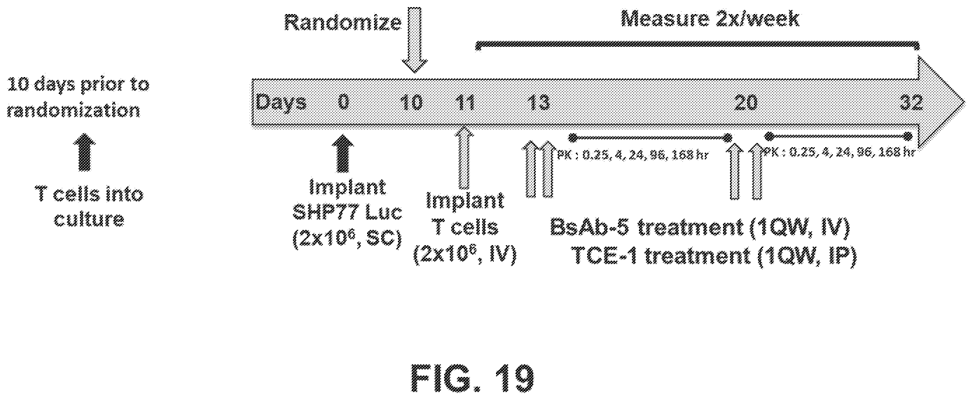

An in vivo combination study of TCE-1 and BsAb-5 was performed in a SHP77 Luc77 model in female NSG mice. A schematic depicting the timing of various events in the study is shown in FIG. 19.

For efficacy studies, female NOD-scid IL2Rgnull (NSG) mice, age 6⁻⁸ weeks (Jackson Labs), were subcutaneously engrafted with SIP-77 human tumor cells (2×10⁶ cells per mouse in 1:1 RPMI media and Matrigel, Corning) in the right flank on study day 0. On study day 11, mice were randomized into treatment groups of n=10 based on tumor volume as measured by digital calipers (tumor volume=L ×W ×H, expressed in mm³). On study day 12, mice were engrafted with 20 million pan CD3+human T cells, which were expanded in vitro for 7-10 days in the presence of rhIL-2 (1Ong/mL, Stem Cell Technologies) and T cell activator beads (CD3/CD28/CD2, Stem Cell Technologies) in Immunocult-XF media (Stem Cell Technologies). On study day 13, mice were treated with BsAb-5 (100 μg/kg, given intravenously in 20 mM citrate 100 mM NaCl pH 6.2 vehicle), TCE-1 (10 μg/kg, given intraperitoneally in DPBS), or isotype control antibodies, as indicated in the treatment groups. Mice were dosed once weekly for a total of 2 doses. Serum samples were collected via retro-orbital bleed at 0.25, 4, 24, 96 and 168 hours post-first and second-dose and analyzed for PK (BsAb-5) by ELISA and serum cytokine levels (Mesoscale Discovery, MSD). Tumor volume and body weight were measured twice weekly, and mice were removed from study once tumor volume exceeded 10% of the mouse body weight (-2000 mm³). Tumor growth inhibition was assessed once the first mouse was removed from the study. Statistical analysis was performed using IVEA mixed linear effects with Tukey's all groups comparison. P values of <0.05 were considered statistically significant.

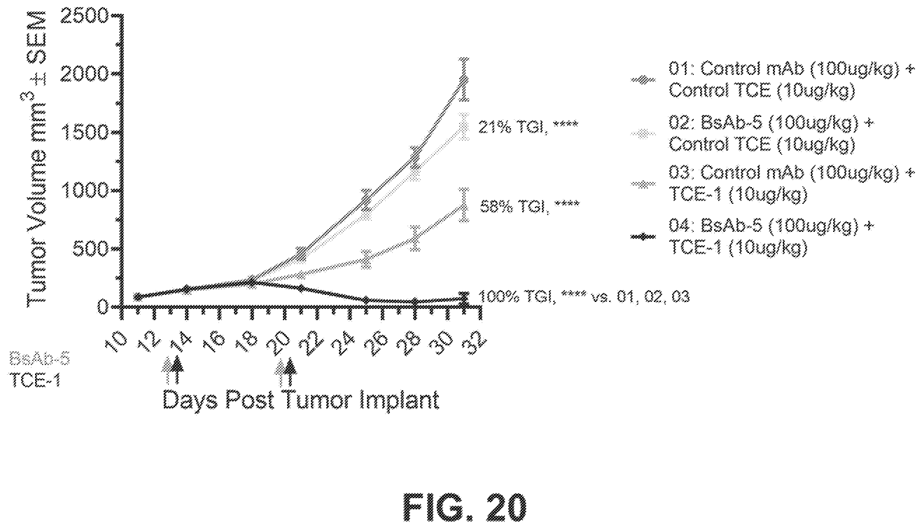

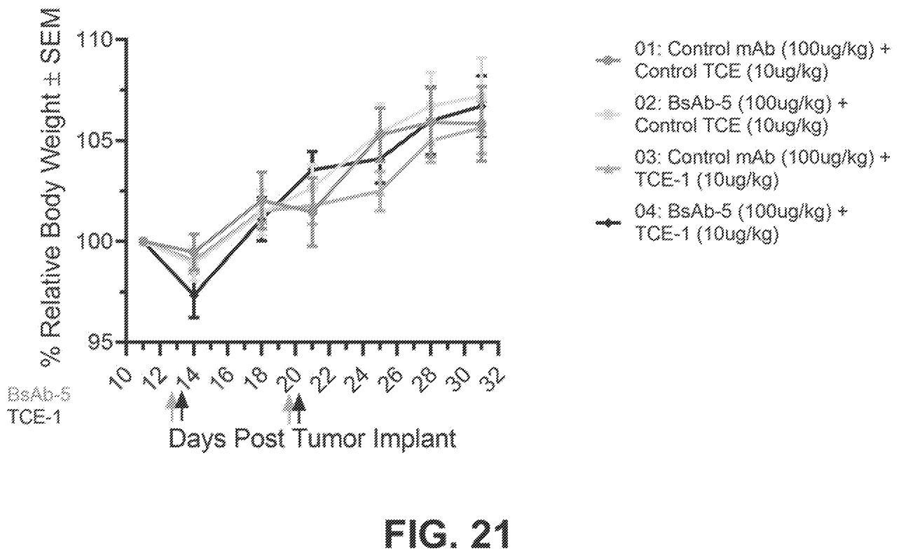

FIG. 20 depicts tumor volume between 11 and 31 days post tumor implantation. The lowest tumor volumes were seen in mice treated with a combination of BsAb-5 and TCE-1. Treatment with the combination therapy was well-tolerated, with mice showing similar changes in body weight across all treatment groups (FIG. 21).

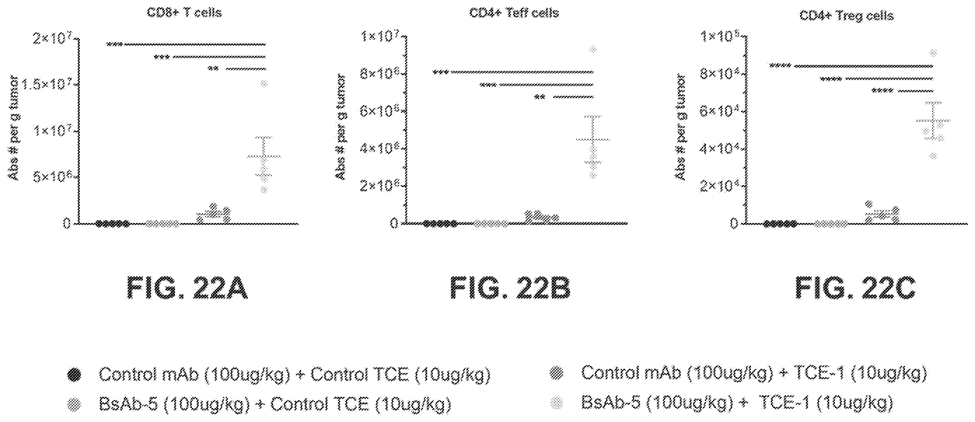

For pharmacodynamic studies, female NOD-scid IL2Rgnull (NSG) mice were subcutaneously engrafted with SIP-77 human tumor cells (2×10⁶ cells per mouse in 1:1 RPMI media and Matrigel) in the right flank on study day 0. On study day 14, mice were randomized into treatment groups of n=5 based on tumor volume and engrafted with 20 million pan CD3+human T cells. On study day 17, mice were treated with BsAb-5 (100 μg/kg, given intravenously in 20 mM citrate 100 mM NaCl pH 6.2 vehicle), TCE-1 (10 μg/kg, given intraperitoneally in DPBS), or isotype control antibodies, as indicated in the treatment groups. Mice were dosed once weekly for a total of 2 doses. Serum samples were collected via cardiac puncture at 1 hour post-second dose and analyzed for PK (BsAb-5) by ELISA and serum cytokine levels by MSD assay. Tumor and spleen tissue was harvested 1 hour post-second dose and human T cells were quantified from single cell suspensions and characterized by flow cytometry. Cells were surface stained with anti-human CD45, CD3, CD4, CD8, CD25, PD-1, Tim3, and/or TIGIT. Following fixation and permeabilization, cells were then stained with anti-human FoxP3, Ki-67, and Granzyme B. The samples were then collected on a flow cytometer and analyzed using FlowJo analysis software. Data are expressed as absolute number of cells per gram of tumor tissue, or per spleen (FIG. 22, panels A-C). Statistical analysis was performed using GraphPad Prism (one-way ANOVA) with Tukey's all groups comparison. P values of <0.05 were considered statistically significant.

While non-limiting example embodiments of the present disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
a VH complementarity determining region one (CDR1) sequence having at most two amino acid modifications relative to any one of SEQ ID NOs: 1-3; and
a VH CDR2 sequence having at most two amino acid modifications relative to any one of SEQ ID NOs: 4-6; and
a VH CDR3 sequence having at most two amino acid modifications relative to any one of SEQ ID NOs: 7-10; and
a second VH region that specifically binds to IL-2Rγ, comprising:
a VH CDR1 sequence having at most two amino acid modifications relative to any one of SEQ ID NOs: 15-16; and
a VH CDR2 sequence having at most two amino acid modifications relative to any one of SEQ ID NOs: 17-19; and
a VH CDR3 sequence having at most two amino acid modifications relative to any one of SEQ ID NOs: 20-21.

2. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
a VH complementarity determining region one (CDR1) sequence having at most two amino acid modifications relative to any one of SEQ ID NOs: 1-3; and
a VH CDR2 sequence having at most two amino acid modifications relative to any one of SEQ ID NOs: 4-6; and
a VH CDR3 sequence having at most two amino acid modifications relative to any one of SEQ ID NOs: 7-10; and
a second VH region that specifically binds to IL-2Rγ, comprising:
a VH CDR1 sequence having at most two amino acid modifications relative to any one of SEQ ID NOs: 15-16; and/or
a VH CDR2 sequence having at most two amino acid modifications relative to any one of SEQ ID NOs: 17-19; and/or
a VH CDR3 sequence having at most two amino acid modifications relative to any one of SEQ ID NOs: 20-21.

3. The method according to claim 1 or claim 2, wherein each amino acid modification, if any, is a conservative amino acid substitution.

4. The method according to any one of claims 1 to 3, wherein the first VH region comprises:
a VH CDR1 comprising a sequence chosen from SEQ ID NOs: 1-3; and
a VH CDR2 comprising a sequence chosen from SEQ ID NOs: 4-6; and
a VH CDR3 comprising a sequence chosen from SEQ ID NOs: 7-10.

5. The method according to any one of claims 1 to 4, wherein the first VH region comprises:
(a) a VH CDR1, a VH CDR2, and a VH CDR3 comprising the sequences of SEQ ID NOs: 1, 4, and 7, respectively; or
(b) a VH CDR1, a VH CDR2, and a VH CDR3 comprising the sequences of SEQ ID NOs: 1, 4, and 8, respectively; or
(c) a VH CDR1, a VH CDR2, and a VH CDR3 comprising the sequences of SEQ ID NOs: 2, 5, and 9, respectively; or
(d) a VH CDR1, a VH CDR2, and a VH CDR3 comprising the sequences of SEQ ID NOs: 3, 6, and 10, respectively.

6. The method according to any one of claims 1 to 5, wherein the second VH region comprises:
a VH CDR1 comprising a sequence chosen from SEQ ID NOs: 15-16; and
a VH CDR2 comprising a sequence chosen from SEQ ID NOs: 17-19; and
a VH CDR3 comprising a sequence chosen from SEQ ID NOs: 20-21.

7. The method according to any one of claims 1 to 6, wherein the second VH region comprises:
(a) a VH CDR1, a VH CDR2, and a VH CDR3 comprising the sequences of SEQ ID NOs: 15, 17, and 20, respectively; or
(b) a VH CDR1, a VH CDR2, and a VH CDR3 comprising the sequences of SEQ ID NOs: 15, 18, and 20, respectively; or
(c) a VH CDR1, a VH CDR2, and a VH CDR3 comprising the sequences of SEQ ID NOs: 16, 18, and 20, respectively; or
(d) a VH CDR1, a VH CDR2, and a VH CDR3 comprising the sequences of SEQ ID NOs: 15, 19, and 21, respectively.

8. The method according to any one of claims 1 to 7, wherein:
the first VH region comprises a VH CDR1, a VH CDR2, and a VH CDR3 comprising the sequences of SEQ ID NOs: 1, 4, and 7, respectively; and
the second VH region comprises a VH CDR1, a VH CDR2, and a VH CDR3 comprising the sequences of SEQ ID NOs: 15, 17, and 20, respectively.

9. The method according to any one of claims 1 to 7, wherein:
the first VH region comprises a VH CDR1, a VH CDR2, and a VH CDR3 comprising the sequences of SEQ ID NOs: 1, 4, and 8, respectively; and
the second VH region comprises a VH CDR1, a VH CDR2, and a VH CDR3 comprising the sequences of SEQ ID NOs: 15, 18, and 20, respectively.

10. The method according to any one of claims 1 to 7, wherein:
the first VH region comprises a VH CDR1, a VH CDR2, and a VH CDR3 comprising the sequences of SEQ ID NOs: 1, 4, and 8, respectively; and
the second VH region comprises a VH CDR1, a VH CDR2, and a VH CDR3 comprising the sequences of SEQ ID NOs: 16, 18, and 20, respectively.

11. The method according to any one of claims 1 to 7, wherein:
the first VH region comprises a VH CDR1, a VH CDR2, and a VH CDR3 comprising the sequences of SEQ ID NOs: 1, 4, and 8, respectively; and
the second VH region comprises a VH CDR1, a VH CDR2, and a VH CDR3 comprising the sequences of SEQ ID NOs: 15, 19, and 21, respectively.

12. The method according to any one of claims 1 to 7, wherein:
the first VH region comprises a VH CDR1, a VH CDR2, and a VH CDR3 comprising the sequences of SEQ ID NOs: 2, 5, and 9, respectively; and
the second VH region comprises a VH CDR1, a VH CDR2, and a VH CDR3 comprising the sequences of SEQ ID NOs: 15, 18, and 20, respectively.

13. The method according to any one of claims 1 to 7, wherein:
the first VH region comprises a VH CDR1, a VH CDR2, and a VH CDR3 comprising the sequences of SEQ ID NOs: 3, 6, and 10, respectively; and
the second VH region comprises a VH CDR1, a VH CDR2, and a VH CDR3 comprising the sequences of SEQ ID NOs: 15, 17, and 20, respectively.

14. A method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
(1) (a) a VH complementarity determining region one (CDR1) comprising the sequence:
(SEQ ID NO: 29)
GFTFSX₁YG
wherein X₁ is S or T; and
(b) a VH CDR2 comprising the sequence:
(SEQ ID NO: 30)
ISYDGSNX₂
wherein X₂ is K or R; and
(c) a VH CDR3 comprising the sequence:
(SEQ ID NO: 31)
ARDLDYDX₃LTGDPVGGFDI
wherein X₃ is V or I; or
(2) (a) a VH CDR1 comprising the sequence:
(SEQ ID NO: 26)
GGSISSSX₁W
wherein X₁ is D or N;
(b) a VH CDR2 comprising the sequence:
(SEQ ID NO: 27)
IX₂HSGST
wherein X₂ is D or S; and
(c) a V_{H} CDR3 comprising the sequence:
(SEQ ID NO: 28)
X₃RGX₄WELX₅DAFDI
wherein X₃ is G or A; X₄ is S or Q; and X₅ is S or T; and
a second VH region that specifically binds to IL-2Rγ, comprising:
(1) (a) a VH CDR1 comprising the sequence:
(SEQ ID NO: 32)
GFX₁X₂X₃X₄YY
wherein X₁ is T or I; X₂ is F or V; X₃ is S, N, or G; and X₄ is D or N; and
(b) a VH CDR2 comprising the sequence:
(SEQ ID NO: 33)
ISX₅SGX₆X₇I
wherein X₅ is S or N; X₆ is D, S, G, or N; and X₇ is T or I; and
(c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).

15. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, comprising:
(1) (a) a VH complementarity determining region one (CDR1) comprising the sequence:
(SEQ ID NO: 29)
GFTFSX₁YG
wherein X₁ is S or T; and
(b) a VH CDR2 comprising the sequence:
(SEQ ID NO: 30)
ISYDGSNX₂
wherein X₂ is K or R; and
(c) a VH CDR3 comprising the sequence:
(SEQ ID NO: 31)
ARDLDYDX₃LTGDPVGGFDI
wherein X₃ is V or I; or
(2) (a) a VH CDR1 comprising the sequence:
(SEQ ID NO: 26)
GGSISSSX₁W
wherein X₁ is D or N;
(b) a VH CDR2 comprising the sequence:
(SEQ ID NO: 27)
IX₂HSGST
wherein X₂ is D or S; and
(c) a VH CDR3 comprising the sequence:
(SEQ ID NO: 28)
X₃RGX₄WELX₅DAFDI
wherein X₃ is G or A; X₄ is S or Q; and X₅ is S or T; and
a second VH region that specifically binds to IL-2Rγ, comprising:
(1) (a) a VH CDR1 comprising the sequence:
(SEQ ID NO: 32)
GFX₁X₂X₃X4YY
wherein X₁ is T or I; X₂ is F or V; X₃ is S, N, or G; and X₄ is D or N; and
(b) a VH CDR2 comprising the sequence:
(SEQ ID NO: 33)
ISX₅SGX₆X₇I
wherein X₅ is S or N; X₆ is D, S, G, or N; and X₇ is T or I; and
(c) a VH CDR3 comprising the sequence ARGDAVSITGDY (SEQ ID NO: 20); or
(2) a VH CDR1 comprising the sequence GFTFSDYY (SEQ ID NO: 15); a VH CDR2 comprising the sequence ISSSGTTT (SEQ ID NO: 19), and a VH CDR3 comprising the sequence ARGAAVAPGFDS (SEQ ID NO: 21).

16. A method of treating cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 95% sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 95% sequence identity to the VH CDRs 1, 2, and 3 of any one of SEQ ID NOs: 22-25.

17. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, in which the full set of VH complementarity determining regions (CDRs) 1, 2, and 3 (combined) has at least 95% sequence identity to the VH CDRs 1, 2, and 3 of any one of SEQ ID NOs: 11-14; and
a second VH region that specifically binds to IL-2Rγ, in which the full set of VH complementarity determining regions (CDRs) 1, 2, and 3 (combined) has at least 95% sequence identity to the VH CDRs 1, 2, and 3 of any one of SEQ ID NOs: 22-25.

18. The method according to claim 16 or claim 17, wherein:
the first VH region comprises the VH CDR1, VH CDR2, and VH CDR3 of any one of SEQ ID NOs: 11-14; and/or
the second VH region comprises the VH CDR1, VH CDR2, and VH CDR3 of any one of SEQ ID NOs: 22-25.

19. The method according to any one of claims 1 to 18, wherein:
the VH CDR1, VH CDR2, and VH CDR3 sequences of the first VH region are present in a human VH framework; and/or
the VH CDR1, VH CDR2, and VH CDR3 sequences of the second VH region are present in a human VH framework.

20. A method of treating a cancer in a subject in need thereof, comprising administering to the subject an anti-IL-2Rβγ heavy chain-only antibody or antigen-binding fragment thereof in combination with a T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 95% sequence identity to the sequence of any one of SEQ ID NOs: 11-14; and
a second VH region that specifically binds to IL-2Rγ, having at least 95% sequence identity to the sequence of any one of SEQ ID NOs: 22-25.

21. A method of enhancing an anti-cancer effect associated with administration of a T-cell redirecting therapy in a subject diagnosed with cancer, comprising administering to the subject an anti-IL-2Rβγ heavy chain-only antibody or antigen-binding fragment thereof in combination with the T-cell redirecting therapy, wherein the anti-IL-2Rβγ heavy chain-only antibody or antigen-binding fragment thereof comprises:
a first heavy chain variable (VH) region that specifically binds to IL-2Rβ, having at least 95% sequence identity to the sequence of any one of SEQ ID NOs: 11-14; and
a second VH region that specifically binds to IL-2Rγ, having at least 95% sequence identity to the sequence of any one of SEQ ID NOs: 22-25.

22. The method according to claim 20 or claim 21, wherein:
the first VH region comprises a sequence chosen from any one of SEQ ID NOs: 11-14; and/or
the second VH region comprises a sequence chosen from any one of SEQ ID NOs: 22-25.

23. The method according to any one of claims 20 to 22, wherein:
the first VH region comprises the sequence of SEQ ID NO: 11; and
the second VH region comprises the sequence of SEQ ID NO: 22.

24. The method according to any one of claims 20 to 22, wherein:
the first VH region comprises the sequence of SEQ ID NO: 12; and
the second VH region comprises the sequence of SEQ ID NO: 23.

25. The method according to any one of claims 20 to 22, wherein:
the first VH region comprises the sequence of SEQ ID NO: 12; and
the second VH region comprises the sequence of SEQ ID NO: 24.

26. The method according to any one of claims 20 to 22, wherein:
the first VH region comprises the sequence of SEQ ID NO: 12; and
the second VH region comprises the sequence of SEQ ID NO: 25.

27. The method according to any one of claims 20 to 22, wherein:
the first VH region comprises the sequence of SEQ ID NO: 13; and
the second VH region comprises the sequence of SEQ ID NO: 23.

28. The method according to any one of claims 20 to 22, wherein:
the first VH region comprises the sequence of SEQ ID NO: 14; and
the second VH region comprises the sequence of SEQ ID NO: 22.

29. The method according to any one of claims 1 to 28, wherein the anti-IL-2Rβγ heavy chain-only antibody or antigen-binding fragment thereof further comprises a Fc region.

30. The method according to any one of claims 1 to 29, wherein the anti-IL-2Rβγ heavy chain-only antibody or antigen-binding fragment thereof further comprises a variant Fc region.

31. The method according to claim 30, wherein the variant Fc region comprises heterodimerizing alterations.

32. The method according to any one of claims 29 to 31, wherein the Fc region is a silenced Fc region.

33. The method according to any one of claims 1 to 32, wherein the T-cell redirecting therapy is a bispecific T-cell engaging molecule.

34. The method according to claim 33, wherein the bispecific T-cell engaging molecule comprises a first domain that specifically binds to a target cancer cell antigen and a second domain that specifically binds to human CD3.

35. The method according to claim 34, wherein the target cancer cell antigen is chosen from EpCAM, CEA, CD19, CD33, CD70, EGFRvIII, FLT3, GPRC5D, DLL3, BCMA, PSMA, STEAP1, STEAP2, MUC16, MUC17, and CLDN18.2.

36. The method according to any one of claims 33 to 35, wherein the bispecific T-cell engaging molecule further comprises a half-life extension domain.

37. The method according to any one of claims 33 to 36, wherein the bispecific T-cell engaging molecule is a three-chain antibody like molecule.

38. The method according to any one of claims 1 to 32, wherein the T-cell redirecting therapy is a chimeric antigen receptor (CAR)-expressing T-cell.

39. The method according to claim 38, wherein the CAR-expressing T-cell comprises a first domain that specifically binds to a target cancer cell antigen, a transmembrane domain, and an intracellular signalling domain.

40. The method according to claim 39, wherein the target cancer cell antigen is chosen from EpCAM, CEA, CD19, CD33, CD70, EGFRvIII, FLT3, GPRC5D, DLL3, BCMA, PSMA, STEAP1, STEAP2, MUC16, MUC17, and CLDN18.2.

41. The method according to any one of claims 1 to 40, wherein:
at least one dose of the anti-IL-2Rβγ heavy chain-only antibody or antigen-binding fragment thereof is administered to the subject prior to a first dose of the T-cell redirecting therapy; or
at least one dose of the T-cell redirecting therapy is administered to the subject prior to a first dose of the anti-IL-2Rβγ heavy chain-only antibody or antigen-binding fragment thereof.

42. The method according to any one of claims 1 to 41, wherein the method comprises administering the anti-IL-2Rβγ heavy chain-only antibody or antigen-binding fragment thereof in combination with the T-cell redirecting therapy in one or more treatment cycles.

43. The method according to claim 42, wherein each of the one or more treatment cycles comprises a single dose of the anti-IL-2Rβγ heavy chain-only antibody or antigen-binding fragment thereof and a single dose of the T-cell redirecting therapy.

44. The method according to claim 42, wherein each of the one or more treatment cycles comprises multiple doses of the anti-IL-2Rβγ heavy chain-only antibody or antigen-binding fragment thereof and a single dose of the T-cell redirecting therapy.

45. The method according to claim 42, wherein each of the one or more treatment cycles comprises a single dose of the anti-IL-2Rβγ heavy chain-only antibody or antigen-binding fragment thereof and multiple doses of the T-cell redirecting therapy.

46. The method according to any one of claims 1 to 45, wherein the cancer is a hematologic cancer.

47. The method according to any one of claims 1 to 46, wherein the cancer is chosen from acute myeloid leukemia, acute lymphocytic leukemia, chronic lymphocytic leukemia, chronic myeloid leukemia, multiple myeloma, diffuse large B-cell lymphoma, Burkitt lymphoma, and non-Hodgkin lymphoma.

48. The method according to any one of claims 1 to 45, wherein the cancer is chosen from prostate cancer, non-small cell lung cancer, small-cell lung cancer, renal cell carcinoma, hepatocellular carcinoma, bladder cancer, testicular cancer, colorectal cancer, esophageal cancer, glioblastoma, head and neck cancer, pancreatic cancer, breast cancer, gastric cancer, gastroesophageal junction cancer, bone cancer, ovarian cancer, endometrial cancer, and melanoma.


SEQUENCE LISTING (68 sequences)

SEQ ID NO: 1 - protein, 9 aa, synthetic construct
  REGION 1..9: note = source = /note="Description of Artificial Sequence: Synthetic peptide"
GGSISSSDW

SEQ ID NO: 2 - protein, 9 aa, synthetic construct
  REGION 1..9: note = source = /note="Description of Artificial Sequence: Synthetic peptide"
GGSISSSNW

SEQ ID NO: 3 - protein, 8 aa, synthetic construct
  REGION 1..8: note = source = /note="Description of Artificial Sequence: Synthetic peptide"
GFTFSSYG

SEQ ID NO: 4 - protein, 7 aa, synthetic construct
  REGION 1..7: note = source = /note="Description of Artificial Sequence: Synthetic peptide"
IDHSGST

SEQ ID NO: 5 - protein, 7 aa, synthetic construct
  REGION 1..7: note = source = /note="Description of Artificial Sequence: Synthetic peptide"
ISHSGST

SEQ ID NO: 6 - protein, 8 aa, synthetic construct
  REGION 1..8: note = source = /note="Description of Artificial Sequence: Synthetic peptide"
ISYDGSNK

SEQ ID NO: 7 - protein, 13 aa, synthetic construct
  REGION 1..13: note = source = /note="Description of Artificial Sequence: Synthetic peptide"
GRGSWELSDAFDI

SEQ ID NO: 8 - protein, 13 aa, synthetic construct
  REGION 1..13: note = source = /note="Description of Artificial Sequence: Synthetic peptide"
ARGSWELTDAFDI

SEQ ID NO: 9 - protein, 13 aa, synthetic construct
  REGION 1..13: note = source = /note="Description of Artificial Sequence: Synthetic peptide"
GRGSWELTDAFDI

SEQ ID NO: 10 - protein, 19 aa, synthetic construct
  REGION 1..19: note = source = /note="Description of Artificial Sequence: Synthetic peptide"
ARDLDYDVLTGDPVGGFDI

SEQ ID NO: 11 - protein, 120 aa, synthetic construct
  REGION 1..120: note = source = /note="Description of Artificial Sequence: Synthetic polypeptide"
QVQLQESGPGLVKPSGTLSLTCAVSGGSISSSDWWSWVRQPPGKGLEWIGEIDHSGSTNYNPSLMSRVTISVDKSKNQFSLKLSSVTAADTAVYFCGRGSWELSDAFDIRGQGTLVTVSS

SEQ ID NO: 12 - protein, 120 aa, synthetic construct
  REGION 1..120: note = source = /note="Description of Artificial Sequence: Synthetic polypeptide"
QVQLQESGPGLVKSSETLSLTCTVSGGSISSSDWWSWVRQPPGKGLEWIGEIDHSGSTNYNPSLMSRVTISVDKSKNQFSLKLSSVTAADTAVYFCARGSWELTDAFDIRGQGTLVTVSS

SEQ ID NO: 13 - protein, 120 aa, synthetic construct
  REGION 1..120: note = source = /note="Description of Artificial Sequence: Synthetic polypeptide"
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSS

SEQ ID NO: 14 - protein, 126 aa, synthetic construct
  REGION 1..126: note = source = /note="Description of Artificial Sequence: Synthetic polypeptide"
QVQLVESGGGVVQPGRSLRLSCAASGFTFSSYGMHWVRQAPGKEREWVAVISYDGSNKYYTDSVKGRFTISRDNSKNTLYLEMNSLRAEDTAVYYCARDLDYDVLTGDPVGGFDIWGQGTLVTVSS

SEQ ID NO: 15 - protein, 8 aa, synthetic construct
  REGION 1..8: note = source = /note="Description of Artificial Sequence: Synthetic peptide"
GFTFSDYY

SEQ ID NO: 16 - protein, 8 aa, synthetic construct
  REGION 1..8: note = source = /note="Description of Artificial Sequence: Synthetic peptide"
GFTFNDYY

SEQ ID NO: 17 - protein, 8 aa, synthetic construct
  REGION 1..8: note = source = /note="Description of Artificial Sequence: Synthetic peptide"
ISSSGDTI

SEQ ID NO: 18 - protein, 8 aa, synthetic construct
  REGION 1..8: note = source = /note="Description of Artificial Sequence: Synthetic peptide"
ISSSGSTI

SEQ ID NO: 19 - protein, 8 aa, synthetic construct
  REGION 1..8: note = source = /note="Description of Artificial Sequence: Synthetic peptide"
ISSSGTTT

SEQ ID NO: 20 - protein, 12 aa, synthetic construct
  REGION 1..12: note = source = /note="Description of Artificial Sequence: Synthetic peptide"
ARGDAVSITGDY

SEQ ID NO: 21 - protein, 12 aa, synthetic construct
  REGION 1..12: note = source = /note="Description of Artificial Sequence: Synthetic peptide"
ARGAAVAPGFDS

SEQ ID NO: 22 - protein, 119 aa, synthetic construct
  REGION 1..119: note = source = /note="Description of Artificial Sequence: Synthetic polypeptide"
QVQLVESGGGLVKPGGSLRLSCAASGFTFSDYYMSWIRQAPGKGLEWVSSISSSGDTIYYADSVQGRFTLSRDNAENSLFLQMNSLRAEDTAVYYCARGDAVSITGDYRGQGTLVTVSS

SEQ ID NO: 23 - protein, 119 aa, synthetic construct
  REGION 1..119: note = source = /note="Description of Artificial Sequence: Synthetic polypeptide"
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSS

SEQ ID NO: 24 - protein, 119 aa, synthetic construct
  REGION 1..119: note = source = /note="Description of Artificial Sequence: Synthetic polypeptide"
QVQLVESGGGLVKPGGSLRLSCAASGFTFNDYYMSWIRQAPGKGLEWVSHISSSGSTIYYADSVKGRFTVSRDNANNSLYLQMHSLRAEDTAVYYCARGDAVSITGDYRGQGTLVTVSS

SEQ ID NO: 25 - protein, 119 aa, synthetic construct
  REGION 1..119: note = source = /note="Description of Artificial Sequence: Synthetic polypeptide"
QVQLVESGGDLVKPGGSLRLSCAASGFTFSDYYMSWLRQAPGKELEWVSHISSSGTTTYYADSVEGRFTITRDNAKNSLYLQMNSLRAEDTAVYYCARGAAVAPGFDSRGQGTLVTVSS

SEQ ID NO: 26 - protein, 9 aa, synthetic construct
  VARIANT 8: note = Xaa is D or N
  SITE 1..9: note = /note="Variant residues given in the sequence have no preference with respect to those in the annotations for variant positions"
  REGION 1..9: note = source = /note="Description of Artificial Sequence: Synthetic peptide"
GGSISSSXW

SEQ ID NO: 27 - protein, 7 aa, synthetic construct
  VARIANT 2: note = Xaa is D or S
  SITE 1..7: note = /note="Variant residues given in the sequence have no preference with respect to those in the annotations for variant positions"
  REGION 1..7: note = source = /note="Description of Artificial Sequence: Synthetic peptide"
IXHSGST

SEQ ID NO: 28 - protein, 13 aa, synthetic construct
  VARIANT 1: note = Xaa is G or A
  VARIANT 4: note = Xaa is S or Q
  VARIANT 8: note = Xaa is S or T
  SITE 1..13: note = /note="Variant residues given in the sequence have no preference with respect to those in the annotations for variant positions"
  REGION 1..13: note = source = /note="Description of Artificial Sequence: Synthetic peptide"
XRGXWELXDAFDI

SEQ ID NO: 29 - protein, 8 aa, synthetic construct
  VARIANT 6: note = Xaa is S or T
  SITE 1..8: note = /note="Variant residues given in the sequence have no preference with respect to those in the annotations for variant positions"
  REGION 1..8: note = source = /note="Description of Artificial Sequence: Synthetic peptide"
GFTFSXYG

SEQ ID NO: 30 - protein, 8 aa, synthetic construct
  VARIANT 8: note = Xaa is K or R
  SITE 1..8: note = /note="Variant residues given in the sequence have no preference with respect to those in the annotations for variant positions"
  REGION 1..8: note = source = /note="Description of Artificial Sequence: Synthetic peptide"
ISYDGSNX

SEQ ID NO: 31 - protein, 19 aa, synthetic construct
  VARIANT 8: note = Xaa is V or I
  SITE 1..19: note = /note="Variant residues given in the sequence have no preference with respect to those in the annotations for variant positions"
  REGION 1..19: note = source = /note="Description of Artificial Sequence: Synthetic peptide"
ARDLDYDXLTGDPVGGFDI

SEQ ID NO: 32 - protein, 8 aa, synthetic construct
  VARIANT 3: note = Xaa is T or I
  VARIANT 4: note = Xaa is F or V
  VARIANT 5: note = Xaa is S, N, or G
  VARIANT 6: note = Xaa is D or N
  SITE 1..8: note = /note="Variant residues given in the sequence have no preference with respect to those in the annotations for variant positions"
  REGION 1..8: note = source = /note="Description of Artificial Sequence: Synthetic peptide"
GFXXXXYY

SEQ ID NO: 33 - protein, 8 aa, synthetic construct
  VARIANT 3: note = Xaa is S or N
  VARIANT 6: note = Xaa is D, S, G, or N
  VARIANT 7: note = Xaa is T or I
  SITE 1..8: note = /note="Variant residues given in the sequence have no preference with respect to those in the annotations for variant positions"
  REGION 1..8: note = source = /note="Description of Artificial Sequence: Synthetic peptide"
ISXSGXXI

SEQ ID NO: 34 - protein, 349 aa, synthetic construct
  REGION 1..349: note = source = /note="Description of Artificial Sequence: Synthetic polypeptide"
QVQLQESGPGLVKSSETLSLTCTVSGGSISSSDWWSWVRQPPGKGLEWIGEIDHSGSTNYNPSLMSRVTISVDKSKNQFSLKLSSVTAADTAVYFCARGSWELTDAFDIRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 35 - protein, 348 aa, synthetic construct
  REGION 1..348: note = source = /note="Description of Artificial Sequence: Synthetic polypeptide"
QVQLVESGGGLVKPGGSLRLSCAASGFTFNDYYMSWIRQAPGKGLEWVSHISSSGSTIYYADSVKGRFTVSRDNANNSLYLQMHSLRAEDTAVYYCARGDAVSITGDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 349 aa, synthetic construct; identical to SEQ ID NO: 34
  REGION 1..349: note = source = /note="Description of Artificial Sequence: Synthetic polypeptide"

SEQ ID NO: 37 - protein, 348 aa, synthetic construct
  REGION 1..348: note = source = /note="Description of Artificial Sequence: Synthetic polypeptide"
QVQLVESGGDLVKPGGSLRLSCAASGFTFSDYYMSWLRQAPGKELEWVSHISSSGTTTYYADSVEGRFTITRDNAKNSLYLQMNSLRAEDTAVYYCARGAAVAPGFDSRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 39 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 40 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 41 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 42 - protein, 330 aa, Homo sapiens [sequence omitted]

SEQ ID NO: 43 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 6 aa, synthetic construct
  REGION 1..6: note = source = /note="Description of Artificial Sequence: Synthetic peptide"
QSVSSN

SEQ ID NO: 45 - skipped

SEQ ID NO: 46 - protein, 9 aa, synthetic construct
  REGION 1..9: note = source = /note="Description of Artificial Sequence: Synthetic peptide"
QQYNNWPWT

SEQ ID NO: 47 - protein, 107 aa, synthetic construct
  REGION 1..107: note = source = /note="Description of Artificial Sequence: Synthetic polypeptide"
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 48 - protein, 214 aa, synthetic construct
  REGION 1..214: note = source = /note="Description of Artificial Sequence: Synthetic polypeptide"
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 5 aa, synthetic construct
  REGION 1..5: note = source = /note="Description of Artificial Sequence: Synthetic peptide"
GGGGS

SEQ ID NO: 50 - protein, 10 aa, synthetic construct
  REGION 1..10: note = source = /note="Description of Artificial Sequence: Synthetic peptide"
GGGGSGGGGS

SEQ ID NO: 51 - protein, 330 aa, synthetic construct
  REGION 1..330: note = source = /note="Description of Artificial Sequence: Synthetic polypeptide"
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 327 aa, synthetic construct
  REGION 1..327: note = source = /note="Description of Artificial Sequence: Synthetic polypeptide"
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 349 aa, synthetic construct
  REGION 1..349: note = source = /note="Description of Artificial Sequence: Synthetic polypeptide"
QVQLQESGPGLVKPSGTLSLTCAVSGGSISSSDWWSWVRQPPGKGLEWIGEIDHSGSTNYNPSLMSRVTISVDKSKNQFSLKLSSVTAADTAVYFCGRGSWELSDAFDIRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 54 - protein, 13 aa, Homo sapiens
ESKYGPPCPSCPA

SEQ ID NO: 55 - protein, 13 aa, synthetic construct
  REGION 1..13: note = source = /note="Description of Artificial Sequence: Synthetic peptide"
ESKYGPPCPPCPA

SEQ ID NO: 56 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 57 - protein, 110 aa, synthetic construct
  REGION 1..110: note = source = /note="Description of Artificial Sequence: Synthetic polypeptide"
APEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 58 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 107 aa, synthetic construct
  REGION 1..107: note = source = /note="Description of Artificial Sequence: Synthetic polypeptide"
GQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 107 aa, synthetic construct
  REGION 1..107: note = source = /note="Description of Artificial Sequence: Synthetic polypeptide"
GQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 61 - protein, 348 aa, synthetic construct
  REGION 1..348: note = source = /note="Description of Artificial Sequence: Synthetic polypeptide"
QVQLVESGGGLVKPGGSLRLSCAASGFTFSDYYMSWIRQAPGKGLEWVSSISSSGDTIYYADSVQGRFTLSRDNAENSLFLQMNSLRAEDTAVYYCARGDAVSITGDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 62 - protein, 349 aa, synthetic construct; identical to SEQ ID NO: 34
  REGION 1..349: note = source = /note="Description of Artificial Sequence: Synthetic polypeptide"

SEQ ID NO: 63 - protein, 348 aa, synthetic construct
  REGION 1..348: note = source = /note="Description of Artificial Sequence: Synthetic polypeptide"
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARGDAVSITGDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 349 aa, synthetic construct
  REGION 1..349: note = source = /note="Description of Artificial Sequence: Synthetic polypeptide"
QVQLQESSPGLVKPSETLSLTCTVSGGSISSSNWWSWVRQPPGKGLEWIGEISHSGSTNYNPSLKSRVTISVDKSKNQFSLRLSSVTAADTAVYFCGRGSWELTDAFDIRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 65 - protein, 348 aa, synthetic construct; identical to SEQ ID NO: 63
  REGION 1..348: note = source = /note="Description of Artificial Sequence: Synthetic polypeptide"

SEQ ID NO: 66 - protein, 355 aa, synthetic construct
  REGION 1..355: note = source = /note="Description of Artificial Sequence: Synthetic polypeptide"
QVQLVESGGGVVQPGRSLRLSCAASGFTFSSYGMHWVRQAPGKEREWVAVISYDGSNKYYTDSVKGRFTISRDNSKNTLYLEMNSLRAEDTAVYYCARDLDYDVLTGDPVGGFDIWGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 67 - protein, 348 aa, synthetic construct; identical to SEQ ID NO: 61
  REGION 1..348: note = source = /note="Description of Artificial Sequence: Synthetic polypeptide"

SEQ ID NO: 68 - protein, 50 aa, synthetic construct
  SITE 1..50: note = /note="This sequence may encompass 1-10 'Gly Gly Gly Gly Ser' repeating units"
  REGION 1..50: note = source = /note="Description of Artificial Sequence: Synthetic polypeptide"
GGGGSGGGGSGGGGSGGGGSGGGGSGGGGSGGGGSGGGGSGGGGSGGGGS